(19) United States
(12) Patent Application Publication
(10) Pub. No.: US 2021/0087552 A1
(43) Pub. Date: Mar. 25, 2021

(54) Methods, Compositions, and Systems for Classification of Genetic Variants of Unknown Significance

(71) Applicant: Laboratory Corporation of America Holdings, Burlington, NC (US)

(72) Inventors: Stanley Letovsky, Milton, MA (US); Anjen Chenn, Burlington, NC (US)

(21) Appl. No.: 17/025,011

(22) Filed: Sep. 18, 2020

Related U.S. Application Data
(60) Provisional application No. 62/902,704, filed on Sep. 19, 2019.

(51) Int. Cl. C12N 15/10; G16B 20/20; G16B 50/00

(52) U.S. Cl. CPC C12N 15/1082; G16B 50/00; G16B 20/20; C12N 15/1072

(57) ABSTRACT

Disclosed are methods, compositions, and systems for classification of genetic variants of unknown significance (VUS). For example, disclosed is an in vitro method for assessing the functional effect of a somatic variation in a target sequence comprising obtaining a biological sample from a subject, performing a genotyping assay on the biological sample to identify a variant of unknown significance at a target sequence, generating a population of cells containing the nucleotide modification at the target sequence, and determining if the population of cells containing the nucleotide modification exhibit at least one different functional characteristic as compared to a population of cells not containing the nucleotide modification. The method may also include generating a database of the plurality of variants of unknown significance and comparing patient samples to the database to make diagnostic determinations and treatment decisions.

### RELATED APPLICATIONS

This application claims priority to U.S. Provisional Application No. 62/902,704, filed on Sep. 19, 2019. The entire content of said provisional application is herein incorporated by reference for all purposes.

### FIELD OF INVENTION

This application is directed to methods, compositions, and systems for assessing and classifying genetic variants of unknown significance.

### BACKGROUND

Cancer cells accumulate genetic variations not present in a patient's healthy cells, and these variations may influence a patient's response to anticancer drugs. Many genetic variants that dispose a patient to sensitivity or resistance to particular drugs have been identified, and DNA sequencing is now commonly applied to biological samples from cancer patients to identify the presence of these variants. Despite progress made in identifying which variants may influence drug response, samples often exhibit “variants of unknown significance” (VUSs). The significance of VUSs with respect to drug response cannot be predicted. A functional assay to assess the impact of a VUS on drug response could provide more actionable information in such cases. Such functional assays can be performed as needed, when variants are encountered in patient samples, or proactively, using high throughput mutagenesis methods to compile a database of characterized variants.

### SUMMARY

The inventions disclosed herein relate to methods, compositions, and systems for assessing and classifying genetic variants of unknown significance (VUSs). The methods, compositions, and systems may be embodied in a variety of ways.

In some embodiments, disclosed is an in vitro method for assessing the functional effect of a somatic mutation in a target sequence comprising obtaining a biological sample from a subject. The method may comprise performing a genotyping assay on the biological sample to identify a variant of unknown significance at a target sequence. The method may further comprise generating a population of cells containing the nucleotide modification at the target sequence. The method may further comprise determining if the population of cells containing the nucleotide modification exhibits at least one different functional characteristic as compared to a population of cells not containing the nucleotide modification. In some embodiments, the target sequence is within a gene associated with chemosensitivity. In some embodiments, the functional characteristic is chemosensitivity.

In some embodiments, the method of generating a population of cells containing the nucleotide modification at the target sequence comprises expanding a cell line derived from the biological sample taken from the subject. In some embodiments, the method further comprises treating the subject based on the at least one different functional characteristic exhibited by the population of cells containing the nucleotide modification.

Also disclosed is a method for treating a subject, comprising: obtaining a biological sample from the subject; performing a genotyping assay on the biological sample to identify a variant in a target sequence; providing a database of variants of unknown significance correlating variants in the target sequence with potential chemosensitivity; and determining, based on the variant detected, and the correlation with the database whether the treatment option should be performed.

Also disclosed is an in vitro method for assessing the functional effect of a genetic variant in a target sequence comprising introducing a plurality of nucleotide modifications, each comprising an individual variant of unknown significance, at a plurality of sites in a target sequence. The method may further comprise determining for each of the plurality of variants of unknown significance, whether the nucleotide change is associated with a change in a functional characteristic for the target sequence. In alternate embodiments, the variants are assessed individually at each target.

Also disclosed is an in vitro method for assessing the impact of a variant of unknown significance in a target sequence on chemosensitivity, the method comprising providing a plurality of a repair oligonucleotides, each comprising a portion of the target sequence and each individually containing a nucleotide modification of a variant of unknown significance at a different position of the target sequence; providing a library of Cas9 guide RNAs (gRNAs) that individually recognize a portion of the target sequence recognized by a defined group of the repair oligonucleotides; co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence; confirming the presence of cells containing at least one of the nucleotide modifications from the plurality of repair oligonucleotides in the population of cells; and determining if the cells containing at least one of the nucleotide modifications exhibit different chemosensitivity than cells not containing the nucleotide modification.

Also disclosed is a method of determining a treatment option for a subject comprising obtaining a biological sample from the subject; performing a genotyping assay on the biological sample to identify a variant in a target sequence; providing a database correlating variants of unknown significance in the target sequence with a diagnosis; and determining a treatment option for the subject based on the variant detected and the correlation with the database.

Also disclosed is a composition comprising a library of cells made by the methods disclosed herein and comprising a plurality of nucleotide modifications corresponding to VUSs at known positions in a target sequence. In some embodiments, at least some of the plurality of nucleotide modifications have been assessed and correlated with an effect on a function of the target sequence.

Also disclosed are systems made by the methods disclosed herein comprising a plurality of nucleotide variants of unknown significance at known positions in the target sequence. In some embodiments, at least some of the plurality of nucleotide variants have been assessed and correlated with an effect on a function of the target sequence.

In various embodiments of the methods, compositions and systems of the invention, and as discussed further herein, the biological sample is cell-free nucleic acid, a liquid biopsy, blood, bone marrow, urine, lymph, another bodily fluid, or a tissue sample. In an embodiment, the biological sample includes genetic material from a cancerous cell. In some embodiments, generating a population of cells containing the nucleotide modification at the target sequence comprises: providing a repair oligonucleotide, wherein the repair oligonucleotide comprises the sequence of the variant of unknown significance; providing a Cas9 guide RNA (gRNA) that individually recognize a portion of the gene recognized by the repair oligonucleotide; co-transfecting a population of cells with (i) an expression system capable of expressing Cas9 and the guide RNA, and (ii) the repair oligonucleotide and guide RNA, wherein the expression system is capable of introducing the oligonucleotide having the nucleotide modification into the target sequence in the population of cells; and confirming the presence of cells containing the nucleotide modification at the target sequence. Additionally and/or alternatively, in some embodiments, the method of generating a population of cells containing the nucleotide modification at the target sequence comprises expanding a cell line derived from the biological sample taken from the subject. In some embodiments, the method further comprises treating the subject based on the at least one different functional characteristic exhibited by the population of cells containing the nucleotide modification.

### BRIEF DESCRIPTION OF THE DRAWINGS

FIG. 1 shows an illustrative example of an in vitro method for assessing the functional effect of a somatic variation in a target sequence.

FIG. 2 shows an illustrative embodiment of a system in which certain embodiments of the technology may be implemented.

### DETAILED DESCRIPTION

The following description recites various aspects and embodiments of the present compositions and methods. No particular embodiment is intended to define the scope of the compositions and methods. Rather, the embodiments merely provide non-limiting examples of various methods and systems that are at least included within the scope of the compositions and methods. The description is to be read from the perspective of one of ordinary skill in the art; therefore, information well known to the skilled artisan is not necessarily included.

### Definitions

The present invention now will be described more fully hereinafter. The invention may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the invention described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

A “modified nucleotide” or “edited nucleotide” refers to a nucleotide sequence of interest that comprises at least one alteration when compared to its non-modified nucleotide sequence. Such “alterations” include, for example: substitution of at least one nucleotide, a deletion of at least one nucleotide, an insertion of at least one nucleotide, or any combination thereof.

As used herein, a “variant of unknown significance” is variation in a genetic sequence for which the associated phenotype is unknown. This phenotype can be related to various aspects of clinical significance including, but not limited to, disease risk and/or likely susceptibility to or resistance to treatments.

As used herein, “chemosensitivity” refers to susceptibility to treatment with chemical and/or therapeutic agents.

Assessing Functional Effects of VUSs

The present invention relates to methods, compositions, and systems for assessing and classifying genetic variants of unknown significance (VUSs). The methods, compositions, and systems may be embodied in a variety of ways.

In certain embodiments, disclosed is an in vitro method for assessing the functional effect of a somatic variation in a target sequence comprising: (a) obtaining a biological sample from a subject; (b) performing a genotyping assay on the biological sample to identify a variant of unknown significance (VUS) at a target sequence; (c) generating a population of cells containing the nucleotide modification corresponding to at least one VUS at the target sequence; and (d) determining if the population of cells containing the nucleotide modification exhibit at least one different functional characteristic as compared to a population of cells not containing the nucleotide modification.

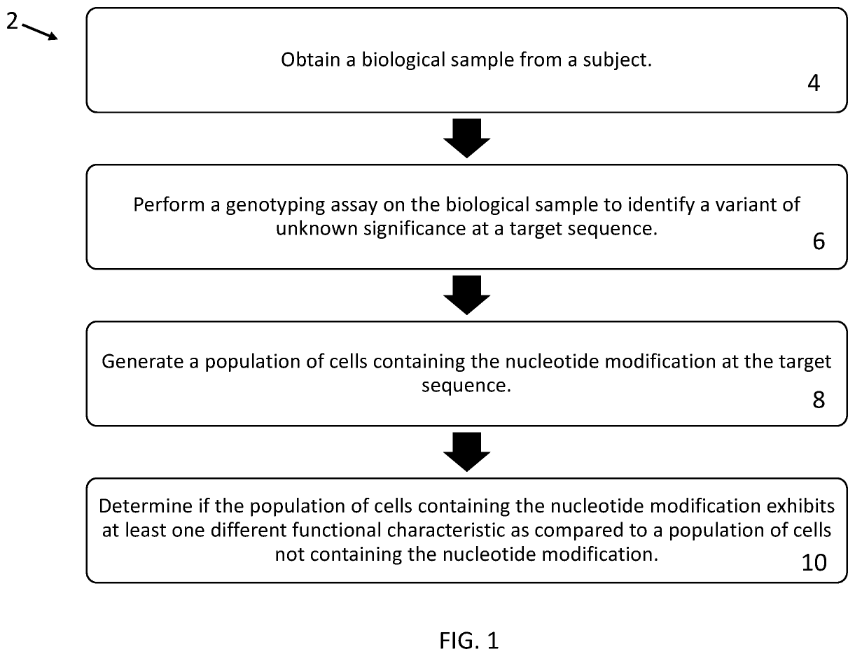

Thus, as schematically depicted in FIG. 1, in certain embodiments disclosed is an in vitro method (2) for assessing the functional effect of a somatic variation in a target sequence comprising: (a) obtaining a biological sample from a subject (4); (b) performing a genotyping assay on the biological sample to identify a variant of unknown significance (VUS) at a target sequence (6); (c) generating a population of cells containing the nucleotide modification corresponding to at least one VUS at the target sequence (8); and (d) determining if the population of cells containing the nucleotide modification exhibit at least one different functional characteristic as compared to a population of cells not containing the nucleotide modification (10).

The functional characteristic may be any functional characteristic. In some embodiments, the functional characteristic is one of clinical significance. In some embodiments, the target sequence is within a gene associated with chemosensitivity. In some embodiments, the functional characteristic is chemosensitivity. For example, in one embodiment, the functional characteristic includes chemosensitivity to anticancer agents, including but not limited to chemotherapies and targeted therapies such as gefitinib or erlotinib. In some embodiments, other functional characteristics, such as resistance to an antibiotic, cell viability, the propensity for metastasis of cancer cells, and the like, may be evaluated.

The biological sample may be from a subject who is asymptomatic, or may be from a subject who is exhibiting symptoms of a disease. Any type of biological sample may be used. For example, in various embodiments, the biological sample is cell-free nucleic acid, a biopsy including a liquid biopsy, blood, bone marrow, urine, lymph, another bodily fluid, or a tissue sample. In some embodiments, the biological sample includes genetic material from a cancerous cell.

In some embodiments, the step of generating a population of cells containing the nucleotide modification at the target sequence comprises: (a) providing a repair oligonucleotide, wherein the repair oligonucleotide comprises the sequence of the variant of unknown significance; (b) providing a Cas9 guide RNA (gRNA) that individually recognize a portion of the gene recognized by the repair oligonucleotide; (c) co-transfecting a population of cells with (i) an expression system capable of expressing Cas9 and the guide RNA, and (ii) the repair oligonucleotide and guide RNA, wherein the expression system is capable of introducing the oligonucleotide having the nucleotide modification corresponding to at least one VUS into the target sequence in the population of cells; and (d) confirming the presence of cells containing the nucleotide modification corresponding to at least one VUS at the target sequence.

In some embodiments, the method also comprises generating a population of cells containing the nucleotide modification corresponding to at least one VUS at the target sequence. In some embodiments, the method also comprises expanding a cell line derived from the biological sample taken from the subject.

As used herein, a “target sequence” is the sequence that is being analyzed to determine how certain VUSs correlate with phenotype. Target nucleic acid sequences include any nucleic acid sequence in genomic DNA. As used herein, the target sequence may be part or all of a “gene of interest,” or may encompass other nucleic acid sequences such as introns, regulatory regions, promoters and the like. In certain embodiments, the target nucleic acid sequence is mammalian genomic DNA. These include, but are not limited to, unknown sequences, entire genes or portions thereof, introns, exons, a polymorphic sequence, a sequence containing a sequence rearrangement, an insertion in the genomic sequence, a deletion in the genomic sequence, one or more highly repetitive sequences, one or more regulatory regions of a gene, etc.

DNA including the target sequence or gene of interest can be DNA directly isolated from a subject and/or may comprise a cell line. As used throughout, the term “subject” refers to an individual. Preferably, the subject is a mammal such as a primate, and, more preferably, a human of any age, including a newborn or a child. Optionally, the genomic DNA is from a human subject. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). In some cases, the subject is a “patient.” As used herein, a patient is someone under medical care.

DNA can also be isolated from the tissue and/or cells of a subject, including tissue and/or cells from a cadaver. Therefore, forensic applications of the methods and compositions provided herein are also provided. Genomic DNA can also be isolated from eukaryotic cells, prokaryotic cells, animal cells, plant cells, fungal cells and the like. As used herein, an “isolated nucleic acid” refers to a nucleic acid that is substantially free from the materials with which the nucleic acid is normally associated in nature or in culture.

The methods provided herein are not limited to genomic DNA as the methods can also be used to fragment and analyze any isolated double-stranded DNA, including but not limited to synthetic DNA, cell-free DNA, complementary DNA (cDNA), plasmid DNA, viral DNA, YAC clones, BAC clones, mitochondrial DNA, and the like.

CRISPR/Cas9

As used herein, a “gRNA” or “guide RNA” is a single RNA sequence that interacts with Cas9 and specifically binds, or hybridizes to, a nucleic acid sequence in the target DNA, such that the gRNA and the Cas9 co-localize to the nucleic acid sequence in the target DNA. Each gRNA includes a first nucleotide sequence that hybridizes to a nucleic acid sequence in the DNA (e.g., genomic DNA containing a target sequence of interest). The first nucleotide sequence includes a crRNA sequence that hybridizes to the target nucleic acid and provides sequence specificity, and a tracrRNA sequence that hybridizes to the crRNA. Each gRNA also includes a second nucleotide sequence that interacts with or binds to Cas9.

In certain embodiments, each gRNA is complementary to a unique pre-defined nucleic acid sequence (i.e., a “target sequence that contains a VUS” or a portion thereof). In some embodiments, the length of the gRNA is between about 10 to about 200 nucleotides. Therefore, the length of the gRNA can be about 10, 20, 30, 40, 50, 60, 70, 80, 90, 100, 110, 120, 130, 140, 150, 160, 170, 180, 190, 200, or any length in between these lengths. It is understood that the gRNA does not have to be complementary to the entire nucleic acid sequence as long as the gRNA can hybridize to the nucleic acid and Cas9 can bind to the nucleic acid sequence in a site-specific manner. One of skill in the art would know how to vary the length of complementarity in order to increase binding specificity and/or decrease offsite binding of the gRNA and/or Cas9.

As used herein, the term “complementary” or “complementarity” refers to base pairing between nucleotides or nucleic acids. Complementary nucleotides are determined by the base present in the DNA (or RNA), generally, adenine (A) pairs with thymine (T) (or uracil in RNA), and guanine (G) and pairs with cytosine (C).

In some embodiments, the genomic DNA is contacted with a plurality of gRNA pairs to generate multiple DNA mutations. Each gRNA may hybridize to different nucleic acid sequences in the genomic DNA. As used herein, “multiple” or “plurality” means two or more. Each gRNA in the plurality of gRNAs binds to a unique site in the genomic DNA. Thus, no two RNAs in the plurality of gRNAs hybridize to the same nucleic acid sequence in the genomic DNA.

As used herein, the term “Cas9” means a Cas9 protein or a fragment thereof present in any bacterial species that encodes a Type II CRISPR/Cas9 system. See e.g., Makarova et al. Nature Reviews, Microbiology, 9: 467-477 (2011), including supplemental information, hereby incorporated by reference in its entirety. For example, the Cas9 protein or a fragment thereof can be from Streptococcus pyogenes. Full-length Cas9 is an endonuclease that contains a recognition domain and two nuclease domains (HNH and RuvC, respectively). In the amino acid sequence, HNH is linearly continuous, whereas RuvC is separated into three regions, one left (downstream) of the recognition domain, and the other two right (upstream) of the recognition domain flanking the HNH domain. Cas9 from Streptococcus pyogenes is targeted to a genomic site in a cell by interacting with a guide RNA that hybridizes to a 20-nucleotide DNA sequence that immediately precedes an NGG motif recognized by Cas9. This results in cleavage of the genomic DNA. As used throughout, the term “cleavage” refers to a reaction that breaks the phosphodiester bonds between two adjacent nucleotides in both strands of a double-stranded DNA molecule such that a double-stranded break occurs in the DNA molecule. The terms “Cas9 cleavage,” “CRISPR cleavage,” and “CRISPR/Cas9 cleavage” are used interchangeably throughout.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence, or any combination thereof.

Cas9 cleavage is asymmetric leaving a blunt end 3′ to the sgRNA and a recessed sticky end 5′ of the sgRNA. Since Cas9 interacts with gRNAs on opposite strands of the genomic DNA, cleavage results in a genomic fragment with two sticky ends that can be modified for further purification and analysis.

In particular embodiments, the guide RNA and Cas endonuclease are capable of forming a complex that enables the Cas endonuclease to introduce a double strand break at a DNA target site. In some embodiments of the invention the variable target domain is 12, 13, 14, 15, 16, 17, 18, 19, 20, 21, 22, 23, 24, 25, 26, 27, 28, 29 or 30 nucleotides in length. In one embodiment, the guide RNA comprises a crRNA (or crRNA fragment) and a tracrRNA (or tracrRNA fragment) of the type II CRISPR/Cas system that can form a complex with a type II Cas endonuclease, wherein the guide RNA/Cas endonuclease complex can direct the Cas endonuclease to a genomic target site, enabling the Cas endonuclease to introduce a double strand break into the genomic target site.

Nucleotide sequence modification of the guide polynucleotide, VT domain, and/or CER domain can be selected from, but is not limited to, the group consisting of a 5′ cap, a 3′ polyadenylated tail, a riboswitch sequence, a stability control sequence, a sequence that forms a dsRNA duplex, a modification or sequence that targets the guide polynucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature may be at least one of a modified or regulated stability, a subcellular targeting, tracking, a fluorescent label, a binding site for a protein or protein complex, modified binding affinity to complementary target sequence, modified resistance to cellular degradation, and increased cellular permeability.

Optionally, any of the methods provided herein can further include the step of amplifying or generating more copies of the target nucleic acid, using a portion of a genomic fragment including the target nucleic acid as a template. Optionally, any of the methods described herein can further include the step of cloning the target sequence into a vector. For example, and not to be limiting, the target sequence can be cloned into a plasmid, a fosmid, a cosmid, a bacteriophage vector, a BAC vector or a YAC vector. Optionally, a nucleic acid molecule containing the target sequence can be modified to include additional sequences, for example adapters, that facilitate cloning into a vector.

Once a double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements are possible and common (Siebert and Puchta, (2002) Plant Cell 14:1121-31; Pacher et al., (2007) Genetics 175:21-9). A double-strand break can also be repaired by homologous recombination (HR) between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which requires extended sequence homology between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932.

Optionally, the modified target sequences (e.g., modified using Cas9 or other methods) are extracted and isolated from a sample, prior to analysis. Methods for analyzing nucleic acids are known in the art. These include, but are not limited to, DNA sequencing, hybridization assays using probes complementary to specific sites in the genomic fragment (for example, a probe complementary to a mutation in the genomic fragment), microarray assays, primer extension assays, polymerase chain reaction (PCR) assays, ligase chain reaction assays, mismatch cleavage assays, branched DNA assays, amplification-refractory mutation system (ARMS) assays, and invasive cleavage assays for identification of SNPs. In some embodiments, DNA sequencing is used. In some embodiments, the Cas9-modified target sequence may be compared to a reference sequence. In other embodiments, differences relative to a reference sequence may be identified in the Cas9-generated DNA fragment(s).

Sequencing methods include, but are not limited to, Sanger sequencing, pyrosequencing, massively parallel signature sequencing, nanopore DNA sequencing, single molecule real-time sequencing (SMRT) (Pacific Biosciences, Menlo Park, Calif.), ion semiconductor sequencing, ligation sequencing, sequencing by synthesis (Illumina, San Diego, Ca), polony sequencing, solid phase sequencing, DNA nanoball sequencing, heliscope single molecule sequencing, mass spectroscopy sequencing, DNA microarray sequencing and any other DNA sequencing method identified in the future.

In some embodiments, the methods provided herein further include determining and modifying the DNA methylation status at one or more sites in the genomic fragment. See, for example, Flusberg et al. “Direct detection of DNA methylation during single-molecule, real-time sequencing,” Nat. Methods 7(6): 461-465 (2010); and Rhoads and Au, “PacBio sequencing and Its Applications,” Genomics, Proteomics &Bioinformatics 13(5): 278-289 (2015), both incorporated herein in their entireties by this reference.

In some embodiments, the methods provided herein further include modifying the haplotype of the target nucleic acid. Methods for determining haplotypes are known in the art. A haplotype may be used by clinicians, researchers and others to correlate haplotype sequences to disease states, for example, cancer, neurological disorders, autoimmune disorders, degenerative disorders, etc. A haplotype sequence may be used to diagnose a disease and/or a stage of a disease or disorder. A haplotype sequence may also be used to assess whether a subject is or is not at risk for development of a disease or disorder. Further, certain haplotype sequences may be correlated to treatment regimens for a particular disease or disorder. In certain embodiments, the haplotype of a human leukocyte antigen (HLA) gene sequence is modified. HLA typing is important for tissue and cell transplantation, autoimmune disease association studies, and drug hypersensitivity research, to name a few. See, for example, Hosomichi et al. “The impact of next-generation sequencing technologies on HLA research,” Journal of Human Genetics 60: 665-673 (2015); and Nelson et al. “An integrated genotyping approach for HLA and other complex genetic systems,” Human Immunology 12: 928-938 (2015), both incorporated herein by this reference.

In some embodiments, the method further comprises treating the subject based on the at least one different functional characteristic exhibited by the population of cells containing the nucleotide modification.

Modification of Target Sequences

In other embodiments, disclosed are in vitro methods for assessing the functional effect of a genetic variant in a target sequences comprising introducing a plurality of nucleotide modifications, each comprising an individual variant of unknown significance, at a plurality of sites in a target sequence; and determining for each of the plurality of variants of unknown significance, whether the nucleotide change is associated with a change in a functional characteristic for the target sequence. The methods may further comprise generating a database of the plurality of variants of unknown significance.

In an embodiment, the plurality of variants of unknown significance are generated using saturation genome editing. In an embodiment, this plurality of variants comprises a library of cells for assessing the functional effect of a somatic variation in a target sequence. The library comprises one or more populations of cells each containing a nucleotide modification at a target sequence, wherein the nucleotide modification exhibits at least one different functional characteristic as compared to a population of cells not containing the nucleotide modification. The library of genome edits may be made by a variety of methods known in the art. In certain embodiments, a CRISPR/Cas 9 system as disclosed herein is used. Or, other systems may be used. For example, in certain embodiments, transfection with an overexpression plasmid containing the variant gene may be assessed for gain of function (e.g., resistance) testing.

For example, in one embodiment the variants of unknown significance may be made by: providing a plurality of a repair oligonucleotides, each comprising a portion of the target sequence and each individually containing a nucleotide modification at a different position of the target sequence; providing a library of Cas9 guide RNAs (gRNAs) that individually recognize a portion of the target sequence recognized by a defined group of the repair oligonucleotides; co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into target sequence; confirming the presence of cells containing at least one of the nucleotide modifications from the plurality of repair oligonucleotides in the population of cells; and determining if the cells containing at least one of the nucleotide modifications exhibit at least one different functional characteristic as compared to cells not containing the nucleotide modification.

The VUS may be a novel (previously undescribed) variant, or may be a variant of unknown significance that is a previously unidentified, or a mutation that was previously identified.

In certain embodiments, the variant of unknown significance is determined from a biological sample from a subject. The biological sample may be from a subject who is asymptomatic or may be from a subject who is exhibiting symptoms of a disease. In some embodiments, the biological sample is cell-free nucleic acid, a solid tissue biopsy, a liquid biopsy, blood, urine, lymph, another bodily fluid, or a tissue sample. In some embodiments, the biological sample includes genetic material from a cancerous cell. Or, the biological sample may comprise a virus (e.g., HIV, HCV, and the like). For example, the method may comprise obtaining a biological sample from a subject; and predicting the effect of the variant of unknown significance in the subject.

In certain embodiments, the method may further comprise the steps of: generating a database of nucleotide modifications with putative different functional characteristics; and using the database to predict a patient's prognosis wherein the patient has a genetic variant in the target sequence that is the same as a nucleotide modification in the database.

The functional characteristic may be any functional characteristic of clinical significance. For example, in one embodiment, the functional characteristic includes chemosensitivity to anticancer agents, including chemotherapies and targeted therapies such as gefitinib or erlotinib. Or other functional characteristics, such as resistance to an antibiotic, cell viability, the propensity for metastasis of cancer cells, and the like, may be evaluated.

A variety of methods may be used to generate a library of repair oligonucleotides. In one embodiment, saturation mutagenesis is used.

In certain embodiments, disclosed is an in vitro method for assessing the impact of a variant of unknown significance in a target sequence on chemosensitivity. For example, in certain embodiments the method may comprise the steps of: (a) providing a plurality of repair oligonucleotides, each comprising a portion of the target sequence and each individually containing a nucleotide modification at a different position of the target sequence; (b) providing a library of Cas9 guide RNAs (gRNAs) that individually recognize a portion of the target sequence recognized by a defined group of the repair oligonucleotides; (c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence; (d) confirming the presence of cells containing at least one of the nucleotide modifications from the plurality of repair oligonucleotides in the population of cells; and (e) determining if the cells containing at the nucleotide modifications exhibit different chemosensitivity than cells not containing the nucleotide modification.

As used herein, a “target sequence” is the sequence that is being analyzed to determine how certain VUS correlate with phenotype. Target nucleic acid sequences include any nucleic acid sequence in genomic DNA. As used herein, the target sequence may be part or all of a “gene of interest,” or may encompass other nucleic acid sequences such as introns, regulatory regions, promoters and the like. The target sequence may be part of a genomic DNA. In certain embodiments, the target nucleic acid sequence is mammalian genomic DNA. These include, but are not limited to, unknown sequences, entire genes or portions thereof, introns, exons, a polymorphic sequence, a sequence containing a sequence rearrangement, an insertion in the genomic sequence, a deletion in the genomic sequence, one or more highly repetitive sequences, one or more regulatory regions of a gene, etc.

DNA including the target sequence or gene of interest can be DNA directly isolated from a subject and/or may comprise a cell line. As used throughout, the term “subject” refers to an individual. Preferably, the subject is a mammal such as a primate, and, more preferably, a human of any age, including a newborn or a child. Optionally, the genomic DNA is from a human subject. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). In some cases, the subject is a “patient.” As used herein, a patient is someone under medical care.

DNA can also be isolated from the tissue and/or cells of subject, including tissue and/or cells from a cadaver. Therefore, forensic applications of the methods and compositions provided herein are also provided. Genomic DNA can also be isolated from eukaryotic cells, prokaryotic cells, animal cells, plant cells, fungal cells and the like. As used herein, an “isolated nucleic acid” refers to a nucleic acid that is substantially free from the materials with which the nucleic acid is normally associated in nature or in culture.

The methods provided herein are not limited to genomic DNA as the methods can also be used to fragment and analyze any isolated double-stranded DNA, including but not limited to synthetic DNA, complementary DNA (cDNA), plasmid DNA, viral DNA, YAC clones, BAC clones, mitochondrial DNA, and the like.

CRISPR/Cas9

In certain embodiments, each gRNA is complementary to a unique pre-defined nucleic acid sequence (i.e., a “target sequence” or a portion thereof). In some embodiments, the length of the gRNA is between about 10 to about 200 nucleotides. Therefore, the length of the gRNA can be about 10, 20, 30, 40, 50, 60, 70, 80, 90, 100, 110, 120, 130, 140, 150, 160, 170, 180, 190, 200, or any length in between these lengths. It is understood that the gRNA does not have to be complementary to the entire nucleic acid sequence as long as the gRNA can hybridize to the nucleic acid and Cas9 can bind to the nucleic acid sequence in a site-specific manner. One of skill in the art would know how to vary the length of complementarity in order to increase binding specificity and/or decrease offsite binding of the gRNA and/or Cas9.

In some embodiments, the genomic DNA is contacted with multiple gRNA pairs to generate multiple DNA mutations. Each gRNA may hybridize to different nucleic acid sequences in the genomic DNA. As used herein, “multiple” means two or more. Each gRNA in the multiple gRNAs binds to a unique site in the genomic DNA. Thus, no two RNAs in the multiple gRNAs hybridize to the same nucleic acid sequence in the genomic DNA.

In particular embodiments, the guide RNA and Cas endonuclease are capable of forming a complex that enables the Cas endonuclease to introduce a double strand break at a DNA target site. In some embodiments of the invention the variable target domain is 12, 13, 14, 15, 16, 17, 18, 19, 20, 21, 22, 23, 24, 25, 26, 27, 28, 29 or 30 nucleotides in length. In one embodiment, the guide RNA comprises a crRNA (or crRNA fragment) and a tracrRNA (or tracrRNA fragment) of the type II CRISPR/Cas system that can form a complex with a type II Cas endonuclease, wherein the guide RNA/Cas endonuclease complex can direct the Cas endonuclease to a genomic target site, enabling the Cas endonuclease to introduce a double strand break into the genomic target site.

Optionally, any of the methods provided herein can further include the step of amplifying or generating more copies of the target nucleic acid, using a portion of a genomic fragment including the target nucleic acid as a template. Optionally, any of the methods described herein can further include the step of cloning the target sequence into a vector. For example, and not to be limiting, the target sequence can be cloned into a plasmid, a fosmid, a cosmid, a bacteriophage vector, a BAC vector or a YAC vector. Optionally, a nucleic acid molecule containing the target sequence can be modified to include additional sequences, for example adapters, that facilitate cloning into a vector.

Optionally, the modified target sequences (e.g., modified by Cas9 or other methods) are extracted and isolated from a sample, prior to analysis. Methods for analyzing nucleic acids are known in the art. These include, but are not limited to, DNA sequencing, hybridization assays using probes complementary to specific sites in the genomic fragment (for example, a probe complementary to a mutation in the genomic fragment), microarray assays, primer extension assays, polymerase chain reaction (PCR) assays, ligase chain reaction assays, mismatch cleavage assays, branched DNA assays, amplification-refractory mutation system (ARMS) assays, and invasive cleavage assays for identification of SNPs. In some embodiments, the Cas9-modified target sequence may be compared to a reference sequence. In other embodiments, differences relative to a reference sequence may be identified in the Cas9-generated DNA fragment(s).

Sequencing methods include, but are not limited to, Sanger sequencing, pyrosequencing, massively parallel signature sequencing, nanopore DNA sequencing, single molecule real-time sequencing (SMRT) (Pacific Biosciences, Menlo Park, Calif.), ion semiconductor sequencing, ligation sequencing, sequencing by synthesis (Illumina, San Diego, Ca), polony sequencing, solid phase sequencing, DNA nanoball sequencing, heliscope single molecule sequencing, mass spectroscopy sequencing, DNA microarray sequencing and any other DNA sequencing method identified in the future.

In some embodiments, the methods provided herein further include determining and modifying the DNA methylation status at one or more sites in the genomic fragment. See, for example, Flusberg et al. “Direct detection of DNA methylation during single-molecule, real-time sequencing,” Nat. Methods 7(6): 461-465 (2010); and Rhoads and Au, “PacBio sequencing and Its Applications,” Genomics, Proteomics &Bioinformatics 13(5): 278-289 (2015), both incorporated herein in their entireties by this reference.

In some embodiments, the methods provided herein further include modifying the haplotype of the target nucleic acid. Methods for determining haplotypes are known in the art. A haplotype may be used by clinicians, researchers and others to correlate haplotype sequences to disease states, for example, cancer, neurological disorders, autoimmune disorders, degenerative disorders, etc. A haplotype sequence may be used to diagnose a disease and/or a stage of a disease or disorder. A haplotype sequence may also be used to assess whether a subject is or is not at risk for development of a disease or disorder. Further, certain haplotype sequences may be correlated to treatment regimens for a particular disease or disorder. In certain embodiments, the haplotype of a human leukocyte antigen (HLA) gene sequence is modified. HLA typing is important for tissue and cell transplantation, autoimmune disease association studies, and drug hypersensitivity research, to name a few. See, for example, Hosomichi et al. “The impact of next-generation sequencing technologies on HLA research,” Journal of Human Genetics 60: 665-673 (2015); and Nelson et al. “An integrated genotyping approach for HLA and other complex genetic systems,” Human Immunology 12: 928-938 (2015), both incorporated herein by this reference.

Methods of Diagnosis and Treating

Also disclosed are methods of diagnosing and/or treating subjects (e.g. patients who may be diagnosed with a disease). The disease may, in certain embodiments, be related to mutations in a target sequence.

For example, in certain embodiments the method may include the step of obtaining a biological sample from a subject; performing a genotyping assay on the biological sample to identify a variant in a target sequence; providing a database comprising a plurality of VUSs, correlating VUSs in the target sequence with a diagnosis; determining that the sample includes one of the database VUSs; and determining a diagnosis based on the variant detected, and the correlation with the database.

Alternatively and/or additionally, the method may comprise: obtaining a biological sample from the subject; performing a genotyping assay on the biological sample to identify a variant in a target sequence; providing a database comprising a plurality of VUSs; determining that the sample includes one of the database VUSs; correlating VUSs in the target sequence with the relative success of a treatment option; and determining, based on the variant detected, and the correlation with the database, whether the treatment option should be performed.

The disease may be any disease suspected to be related to the target sequence. In some embodiments, the disease may be cancer. For example, in an embodiment, disclosed is a method for treating a patient, wherein the patient is suffering from cancer, the method comprising the steps of: determining whether the patient is chemosensitive to a therapeutic by: (a) obtaining a biological sample from a patient; (b) performing a genotyping assay on the biological sample to identify a variant of unknown significance at a target sequence; (c) providing a repair oligonucleotide, wherein the repair oligonucleotide comprises the sequence of the variant of unknown significance; (d) providing a system to introduce the VUS into a population of cells; a (f) confirming the presence of cells containing the nucleotide modification comprising at least one VUS at the target sequence; and (e) determining if the cells containing the nucleotide modification comprising at least one VUS exhibit different chemosensitivity than cells not containing the nucleotide modification comprising at least one VUS; and (f) if the cells containing the nucleotide modification comprising at least one VUS are chemosensitive to a therapeutic, then administering said therapeutic to the patient. In certain embodiments, step (d) may comprise providing a Cas9 guide RNA (gRNA) that individually recognize a portion of the gene recognized by the repair oligonucleotide and co-transfecting a population of cells with (i) an expression system capable of expressing Cas9 and the guide RNA, and (ii) the repair oligonucleotide and guide RNA, wherein the expression system is capable of introducing the oligonucleotide having the nucleotide modification comprising at least one VUS into the target sequence in the population of cells;

A variety of sample types may be used. In certain embodiments the biological sample is cell-free nucleic acid, a solid tissue biopsy, a liquid biopsy, blood, urine, lymph, another bodily fluid, or a tissue sample. In an embodiment, the biological sample includes genetic material from a cancerous cell.

Compositions

Also disclosed herein are compositions. In certain embodiments, the compositions may be used for determining the function of a VUS and/or or for developing therapeutic protocols. For example, in certain embodiments, disclosed is a composition comprising a library of cells comprising a plurality of nucleotide variants of unknown significance (VUS) at known positions in the target sequence. This library of cells may be used for assessing the functional effect of a somatic variation in a target sequence. In certain embodiments, the library of cells may also comprise one or more populations of cells each containing a nucleotide modification at a target sequence, wherein the nucleotide modification exhibits at least one different functional characteristic as compared to a population of cells not containing the nucleotide modification. In certain embodiments, at least some of the VUSs may be previously uncharacterized. In certain embodiments, at least some of the plurality of nucleotide variants have been assessed for an effect on a function of the target sequence as disclosed herein.

The library may be generated by introducing various VUSs at a specific target or a plurality of targets in the genome. In certain embodiments a Cas9 system may be used. In other embodiments, other targeting methods (e.g., saturation mutagenesis) may be used. For example, in some embodiments, the library may be generated by (a) providing a plurality of a repair oligonucleotides, each comprising a portion of the target sequence and each individually containing a nucleotide modification at a different position of the target sequence; (b) providing a library of Cas9 guide RNAs (gRNA) that individually recognize a portion of the target sequence recognized by a defined group of the repair oligonucleotides; c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence; (d) confirming the presence of cells containing at least one of the nucleotide modifications from the plurality of repair oligonucleotides in the population of cells; and (e) determining if the cells containing at least one of the nucleotide modifications exhibit different a different functional characteristic than cells not containing the nucleotide modification.

Systems

Also disclosed are systems for performing any of the steps of the methods disclosed herein. The system may for example comprise: (a) a station for obtaining a biological sample from a subject; (b) a station for performing a genotyping assay on the biological sample to identify a variant of unknown significance (VUS) at a target sequence; (c) a station for generating a population of cells containing the nucleotide modification corresponding to at least one VUS at the target sequence; and (d) a station for determining if the population of cells containing the nucleotide modification exhibit at least one different functional characteristic as compared to a population of cells not containing the nucleotide modification. Each of the stations may be a single station or a collection of stations.

Also disclosed are systems comprising the components of the methods disclosed herein. Thus, disclosed is a system comprising a plurality of nucleotide variants at known positions in a target sequence. In an embodiment, at least some of the plurality of nucleotide variants have been assessed for an effect on a function of the target sequence. For example, the system may comprise a database of variants and/or a composition comprising library of cells comprising such VUSs. At least some of the stations and/or components of the system may be implemented at least in part using a computer and/or computer-implemented instructions (e.g., software) as described in more detail herein.

Computer Systems and Computer Program Products

Certain processes and methods described herein often cannot be performed without a computer, microprocessor, software, module or other machine. At least certain steps of methods described herein, or systems described herein, may be computer-implemented, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein is a computer system configured to perform the any of the embodiments of the methods, or particular steps of any of the methods for assessing the functional effect of a genetic variant in a target sequence, and/or developing methods of treatment and/or developing compositions comprising a VUS library of cells or database disclosed herein. In some embodiments, this invention provides a system for assessing the functional effect of a genetic variant in a target sequence comprising one or more processors and non-transitory machine readable storage medium and/or memory coupled to one or more processors, and the memory or the non-transitory machine readable storage medium encoded with a set of instructions configured to perform a process.

Also provided herein are computer readable storage media with an executable program stored thereon, where the program instructs a microprocessor to perform any of the methods or method steps, and/or developing methods of treatment and/or developing compositions comprising a VUS library of cells or database described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

In some embodiments, the invention provides a non-transitory machine readable storage medium comprising program instructions that when executed by one or more processors cause the one or more processors to perform any of the methods disclosed herein.

Thus, also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Sequence reads, counts, levels and/or measurements sometimes are referred to as “data” or “data sets.” In some embodiments, data or data sets can be characterized by one or more features or variables (e.g., sequence based (e.g., GC content, specific nucleotide sequence, the like), function specific (e.g., expressed genes, cancer genes, the like), location based (genome specific, chromosome specific, portion or portion-specific), the like and combinations thereof). In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct methods described herein. Using machines, software and interfaces, a user may enter, request, query or determine options for using particular information, programs or processes, which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, simulated data is generated by an in silico process and the simulated data serves as data that can be input via an input device. The term “in silico” refers to research and experiments performed using a computer.

A system may include software useful for performing a process or part of a process described herein, and software can include one or more modules for performing such processes (e.g., sequencing module, logic processing module, and data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein.

A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A module can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, and/or padding), providing identification (e.g., identifying a genetic variation/genetic alteration), categorizing, plotting, and/or determining an outcome, for example. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product may be embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, FORTRAN, and the like. In some embodiments, an algorithm can be configured or modified to include margin of errors, statistical analysis, statistical significance, and/or comparison to other information or data sets (e.g., applicable when using, for example, algorithms to determine correlation of a VUS to a therapeutic index or profile such as a fixed cutoff algorithm, a dynamic clustering algorithm, or an individual polymorphic nucleic acid target threshold algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

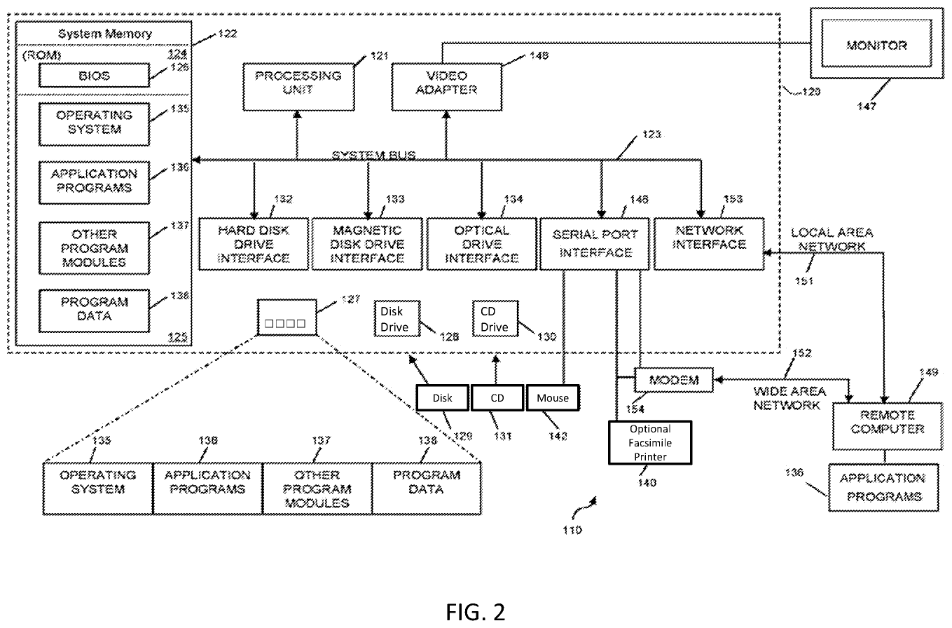

FIG. 2 illustrates a non-limiting example of a computing environment 110 in which various systems, methods, algorithms, and data structures described herein may be implemented. The computing environment 110 is only one example of a suitable computing environment and is not intended to suggest any limitation as to the scope of use or functionality of the systems, methods, and data structures described herein. Neither should computing environment 110 be interpreted as having any dependency or requirement relating to any one or combination of components illustrated in computing environment 110. A subset of systems, methods, and data structures shown in FIG. 2 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The operating environment 110 of FIG. 2 includes a general purpose computing device in the form of a computer 120, including a processing unit 121, a system memory 122, and a system bus 123 that operatively couples various system components including the system memory 122 to the processing unit 121. There may be only one or there may be more than one processing unit 121, such that the processor of computer 120 includes a single central-processing unit (CPU), or a plurality of processing units, commonly referred to as a parallel processing environment. The computer 120 may be a conventional computer, a distributed computer, or any other type of computer.

The system bus 123 may be any of several types of bus structures including a memory bus or memory controller, a peripheral bus, and a local bus using any of a variety of bus architectures. The system memory may also be referred to as simply the memory, and includes read only memory (ROM) 124 and random access memory (RAM). A basic input/output system (BIOS) 126, containing the basic routines that help to transfer information between elements within the computer 120, such as during start-up, is stored in ROM 124. The computer 120 may further include a hard disk drive interface 127 for reading from and writing to a hard disk, not shown, a magnetic disk drive 128 for reading from or writing to a removable magnetic disk 129, and an optical disk drive 130 for reading from or writing to a removable optical disk 131 such as a CD ROM or other optical media.

The hard disk drive 127, magnetic disk drive 128, and optical disk drive 130 may be connected to the system bus 123 by a hard disk drive interface 132, a magnetic disk drive interface 133, and an optical disk drive interface 134, respectively. The drives and their associated computer-readable media provide nonvolatile storage of computer-readable instructions, data structures, program modules and other data for the computer 120. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 129, optical disk 131, ROM 124, or RAM, including an operating system 135, one or more application programs 136, other program modules 137, and program data 138. A user may enter commands and information into the personal computer 120 through input devices such as a keyboard 140 and pointing device 142. Other input devices (not shown) may include a microphone, joystick, game pad, satellite dish, scanner, or the like. These and other input devices are often connected to the processing unit 121 through a serial port interface 146 that is coupled to the system bus, but may be connected by other interfaces, such as a parallel port, game port, or a universal serial bus (USB). A monitor 147 or other type of display device may be connected to the system bus 123 via an interface, such as a video adapter 148. In addition to the monitor, computers typically include other peripheral output devices (not shown), such as speakers and printers.

The computer 120 may operate in a networked environment using logical connections to one or more remote computers, such as remote computer 149. These logical connections may be achieved by a communication device coupled to or a part of the computer 120, or in other manners. The remote computer 149 may be another computer, a server, a router, a network PC, a client, a peer device or other common network node, and typically includes many or all of the elements described above relative to the computer 120, although only a memory storage device 150 has been illustrated in FIG. 2. The logical connections depicted in FIG. 2 include a local-area network (LAN) 151 and a wide-area network (WAN) 152. Such networking environments are commonplace in office networks, enterprise-wide computer networks, intranets and the Internet, which all are types of networks.

When used in a LAN-networking environment, the computer 120 is connected to the local network 151 through a network interface or adapter 153, which is one type of communications device. When used in a WAN-networking environment, the computer 120 often includes a modem 154, a type of communications device, or any other type of communications device for establishing communications over the wide area network 152. The modem 154, which may be internal or external, is connected to the system bus 123 via the serial port interface 146. In a networked environment, program modules depicted relative to the personal computer 120, or portions thereof, may be stored in the remote memory storage device. It is appreciated that the network connections shown are non-limiting examples and other communications devices for establishing a communications link between computers may be used.

The following examples of specific embodiments of the invention are offered for illustrative purposes only, and are not intended to limit the scope of the invention in any way.

### EXAMPLES

### Example 1—Saturation Genome Editing

In certain embodiments, saturation genome editing of a target of interest utilizes cell lines that express a gene comprising the target of interest. For example, in one example the CIVIL K562 cell line is used to assess the effect on Gleevec sensitivity of VUS mutations in the kinase domain of the chimeric BCR/Abl (Philadelphia chromosome) gene. The P315I variant is known to confer resistance and can serve as a positive control.

To generate systematic modification of a target of interest, a library of repair oligonucleotides is generated. In an embodiment, oligo pools for single exons may be generated and PCR amplified and cloned (e.g., into plasmids) with homology arms to mediate genomic integration. Such libraries are essentially SNV libraries. In some cases, the library molecule may also include a fixed substitution at the target site to reduce re-cutting by Cas9 after successful HDR (Findlay et al., bioRxiv, Apr. 5, 20182018).

Cells may be transfected with the SNV library and Cas9/gRNA plasmid to generate a VUS library of individual cells that each comprise a single VUS. Cells may then be selected by antibiotic resistance or other plasmid-based selection methods. Variant frequencies are then quantified by targeted amplification and deep sequencing of the edited exon from VUS library genomic DNA. Upon identification of SUVs, cells may then be analyzed to determine how the mutation affects the cell's phenotype. For example, cells may be treated (e.g., with a therapeutic agent) to see how certain mutations are correlated to resistance or sensitivity to the therapeutic agent. The data may then be compiled and used to determine mutations that are clinically significant from those mutations that do not have an adverse effect.

### Example 2—Database Identification of Potential Mutations

An analysis of genes and variants known to impact resistance or sensitivity in myeloid disorders was performed. Table 1 and Table 2 show drug-gene interactions. Every nonzero cell in this table is a potential candidate for the disclosed method if a VUS is found in that gene and the drug is under consideration for that patient. Thus, Table 1 shows the correlation of individual mutations (n=349) in ABL1, ASXL1, BRAF, CDKN2A, CEBPA, CSF3R, DNMT3A, EZH2, FLT3, IDH1, IDH2, JAK2, KIT, KMT2A, KRAS, NF1, NPM1, NRAS, PDGFRA, PML PTPN11, TET2, and TP53 with resistance or susceptibility to any one or more of the chemotherapeutic agents listed. The chemotherapeutic agents and non-limiting combinations of chemotherapeutic agents include: 5-azacytidine, 5-azacytidine/sorafenib, 5-fluorouracil/irinotecan/leucovorin/oxaliplatin, 5-fluorouracil/leucovorin/oxaliplatin, abiraterone, afatinib, aflibercept, alectinib, alemtuzumab, alemtuzumab/rituximab, AMG 337, anti-CD20 antibody/idelalisib, anti-EGFR antibody, arsenic trioxide, atezolizumab, axitinib, bevacizumab, bevacizumab/cetuximab, bevacizumab/erlotinib, bosutinib, BRAF inhibitor, BRAF inhibitor/MEK inhibitor, brigatinib, cabazitaxel, cabozantinib, carboplatin/docetaxel, carboplatin/etoposide, carboplatin/gemcitabine, carboplatin/paclitaxel, carboplatin/pemetrexed, cetuximab, cisplatin, cisplatin/docetaxel, cisplatin/etoposide, cisplatin/gemcitabine, cisplatin/paclitaxel, cisplatin/pemetrexed, clofarabine, cobimetinib, cobimetinib/vemurafenib, copanlisib, crizotinib, cyclophosphamide, cyclophosphamide/doxorubicin/prednisone/vincristine, cyclophosphamide/fludarabine, cytarabine, cytarabine/daunorubicin, dabrafenib, darafenib/trametinib, darafenib/trametinib/vemurafenib, dasatinib, daunorubicin, decitabine, decitabine/sorafenib, dexamethasone, docetaxel, docetaxel/gemcitabine, doxorubicin, EGFR tyrosine kinase inhibitor, enasidenib, enzalutamide, erlotinib, etoposide, filgrastim, fludarabine phosphate, fluoropyrimidine, fluoropyrimidine/oxaliplatin, gefitnib, gemcitabine/vinorelbine, ibrutinib, idelalisib, idelalisib/ofatumumab, idelalisib/rituximab, imatinib, ipilimumab/nivolaumab, lenvatinib, lorlatinib, MAP kinase inhibitor, methotrexate, methylprednisolone, midostaurin, mitoxantrone, nilotinib, nintedanib, nivolumab, obinutuzumab, ofatumumab, olaratumab, omacetaxine mepesuccinate, osimertinib, pantitumumab, pazopanib, PEG-interferon alfa-2a, pembrolizumab, pexidartinib, PI 3-kinase inhibitor, platinum chemotherapy regimen/vinorelbine, ponatinib, prednisolone, prednisone, quizartinib, ramucirumab, regorafenib, rituximab, rituximab/venetoclax, rociletinib, ruxolitinib, selumetinib, sorafenib, sunitinib, temozolomide, trametinib, tretinoin, trifluridine, vemurafenib, venetoclax, vincristine, and/or vinorelbine. It can be seen that in some cases, a mutation (e.g., ABL1 c.944C>T) was associated with resistance to more than one chemotherapeutic agent (e.g., cytarabine, dabrafenib, daunorubicin, and dexamethasone, among others). Additionally and/or alternatively, resistance or sensitivity to a single chemotherapeutic agent (e.g., bosutinib) may be associated with multiple mutations in a gene (e.g., ABL1). During the analysis, additional information relating to the nature of the mutations, the type of cancer, the origin of the cancer (germline or somatic), whether a mutation is associated with loss of function (LoF) or gain of function (GoF), and the type of treatment was collected. Specificity levels of the treatments, i.e. whether they were exact matches to a specific variant, a specific position, or a gene, were recorded. However, the specificity classifications could be over-inclusive. For example, if the treatment in question acts on a certain tyrosine kinase, but the variant codes for another tyrosine kinase, the treatment that is gene specific may still be listed as a candidate.

Table 2 provides an analysis of individual mutations (n=666) in ABL1, ASXL1, BRAF, CSF3R, DNMT3A, EZH2, FLT3, IDH2, JAK2, KIT, KRAS, NF1, NRAS, PDGFRA, PML PTPN11, and TET2 with resistance or susceptibility to any one or more of the chemotherapeutic agents listed. These chemotherapeutic agents and non-limiting combinations of chemotherapeutic agents include: 5-azacytidine, afatinib, arsenic trioxide, axitinib, bosutinib, brigatinib, cabozantinib, carboplatin/paclitaxel, cetuximab, cobimetinib, cobimetinib/vemurafenib, copanlisib, dabrafenib, dabrafenib/trametinib, dasatinib, decitabine, EGFR tyrosine kinase inhibitor, enasidenib, erlotinib, filgrastim, gefitnib, imatinib, lenvatinib, midostaurin, nilotinib, nintedanib, nivolumab, olaratumab, panitumumab, pazopanib, PEG-interferon alfa-2a, platinum chemotherapy regimen/vinorelbine, ponatinib, regorafenib, ruxolitinib, sorafenib, sunitinib, temozolomide, trametinib, and/or vemurafenib. It can be seen that in some cases, a mutation (e.g., ABL1 c.944C>T) is associated with resistance to one or more agents (e.g., bosutinib, dasatinib, imatinib, and nilotinib, among others. During the analysis, additional information relating to the nature of the mutations, the type of cancer or type of disease, the origin of the cancer (germline or somatic), whether the mutation is associated with loss of function (LoF) or gain of function (GoF), and the type of treatment was collected Specificity levels of the treatments, i.e. whether they were exact matches to a specific variant, a specific position, or a gene, were recorded. As with the data collected in Table 1, the resistance or susceptibility determination of a gene-specific agent may be over-inclusive. Additionally, data was collected regarding whether the agency was FDA, EMA, NCCN, or AMP.

TABLE 1

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| ABL1 | c.1328T > C | | | | | | | | | | | | | | | |
| ABL1 | c.763G > A | | | | | | | | | | | | | | | |
| ABL1 | c.764A > T | | | | | | | | | | | | | | | |
| ABL1 | c.944C > T | | | | | | | | | | | | | | | |
| ABL1 | c.991A > G | | | | | | | | | | | | | | | |
| ASXL1 | c.1153G > A | | | | | | | | | | | | | | | |
| ASXL1 | c.1162G > A | | | | | | | | | | | | | | | |
| ASXL1 | c.1210C > T | | | | | | | | | | | | | | | |
| ASXL1 | c.1276A > T | | | | | | | | | | | | | | | |
| ASXL1 | c.1633C > T | | | | | | | | | | | | | | | |
| ASXL1 | c.1749G > A | | | | | | | | | | | | | | | |
| ASXL1 | c.1772dupA | | | | | | | | | | | | | | | |
| ASXL1 | c.1773C > G | | | | | | | | | | | | | | | |
| ASXL1 | c.1842_1847delinsACAGG | | | | | | | | | | | | | | | |
| ASXL1 | c.1895_1896delinsAA | | | | | | | | | | | | | | | |
| ASXL1 | c.1925G > C | | | | | | | | | | | | | | | |
| ASXL1 | c.1926_1927delAG | | | | | | | | | | | | | | | |
| ASXL1 | c.1926delA | | | | | | | | | | | | | | | |
| ASXL1 | c.1935dupT | | | | | | | | | | | | | | | |
| ASXL1 | c.1947dupA | | | | | | | | | | | | | | | |
| ASXL1 | c.2122C > T | | | | | | | | | | | | | | | |
| ASXL1 | c.2128G > T | | | | | | | | | | | | | | | |
| ASXL1 | c.2129delG | | | | | | | | | | | | | | | |
| ASXL1 | c.2238delC | | | | | | | | | | | | | | | |
| ASXL1 | c.2269C > T | | | | | | | | | | | | | | | |
| ASXL1 | c.2364_2374del11 | | | | | | | | | | | | | | | |
| ASXL1 | c.2404dupG | | | | | | | | | | | | | | | |
| ASXL1 | c.2416_2417dupAC | | | | | | | | | | | | | | | |
| ASXL1 | c.2439delC | | | | | | | | | | | | | | | |
| ASXL1 | c.2461delG | | | | | | | | | | | | | | | |
| ASXL1 | c.2468delT | | | | | | | | | | | | | | | |
| ASXL1 | c.2473A > T | | | | | | | | | | | | | | | |
| ASXL1 | c.2929C > T | | | | | | | | | | | | | | | |
| ASXL1 | c.2974_2975delCT | | | | | | | | | | | | | | | |
| ASXL1 | c.3072_3076dupAAAGG | | | | | | | | | | | | | | | |
| ASXL1 | c.3083C > A | | | | | | | | | | | | | | | |
| ASXL1 | c.3306G > T | | | | | | | | | | | | | | | |
| ASXL1 | c.3501delC | | | | | | | | | | | | | | | |
| ASXL1 | c.4189G > A | | | | | | | | | | | | | | | |
| ASXL1 | c.4343A > G | | | | | | | | | | | | | | | |

| | | | | | | | | | | | | | BRAF | |
|---|---|---|---|---|---|---|---|---|---|---|---|---|---|---|
| | | anti- | | | | | | | | | | | inhibitor/ | |
| | | EGFR | arsenic | | | | | bevacizumab/ | bevacizumab/ | | | BRAF | MEK | |
| | | antibody | trioxide | atezolizumab | axitinib | bevacizumab | | cetuximab | erlotinib | bosutinib | | inhibitor | inhibitor | brigatinib |
| Gene | Variant | X | ✓ | ✓ | ✓ | ✓ | X | X | X | ✓ | X | ✓ | ✓ | ✓ |
| ABL1 | c.1328T > C | | | | | | | | | 2 | | | | |
| ABL1 | c.763G > A | | | | | | | | | 2 | | | | |
| ABL1 | c.764A > T | | | | | | | | | 2 | | | | |
| ABL1 | c.944C > T | | | | | | | | | 3 | 1 | | | |
| ABL1 | c.991A > G | | | | | | | | | 2 | | | | |
| ASXL1 | c.1153G > A | | | | | | | | | | | | | |
| ASXL1 | c.1162G > A | | | | | | | | | | | | | |
| ASXL1 | c.1210C > T | | | | | | | | | | | | | |
| ASXL1 | c.1276A > T | | | | | | | | | | | | | |
| ASXL1 | c.1633C > T | | | | | | | | | | | | | |
| ASXL1 | c.1749G > A | | | | | | | | | | | | | |
| ASXL1 | c.1772dupA | | | | | | | | | | | | | |
| ASXL1 | c.1773C > G | | | | | | | | | | | | | |
| ASXL1 | c.1842_1847delinsACAGG | | | | | | | | | | | | | |
| ASXL1 | c.1895_1896delinsAA | | | | | | | | | | | | | |
| ASXL1 | c.1925G > C | | | | | | | | | | | | | |
| ASXL1 | c.1926_1927delAG | | | | | | | | | | | | | |
| ASXL1 | c.1926delA | | | | | | | | | | | | | |
| ASXL1 | c.1935dupT | | | | | | | | | | | | | |
| ASXL1 | c.1947dupA | | | | | | | | | | | | | |
| ASXL1 | c.2122C > T | | | | | | | | | | | | | |
| ASXL1 | c.2128G > T | | | | | | | | | | | | | |
| ASXL1 | c.2129delG | | | | | | | | | | | | | |
| ASXL1 | c.2238delC | | | | | | | | | | | | | |
| ASXL1 | c.2269C > T | | | | | | | | | | | | | |
| ASXL1 | c.2364_2374del11 | | | | | | | | | | | | | |
| ASXL1 | c.2404dupG | | | | | | | | | | | | | |
| ASXL1 | c.2416_2417dupAC | | | | | | | | | | | | | |
| ASXL1 | c.2439delC | | | | | | | | | | | | | |
| ASXL1 | c.2461delG | | | | | | | | | | | | | |
| ASXL1 | c.2468delT | | | | | | | | | | | | | |
| ASXL1 | c.2473A > T | | | | | | | | | | | | | |
| ASXL1 | c.2929C > T | | | | | | | | | | | | | |
| ASXL1 | c.2974_2975delCT | | | | | | | | | | | | | |
| ASXL1 | c.3072_3076dupAAAGG | | | | | | | | | | | | | |
| ASXL1 | c.3083C > A | | | | | | | | | | | | | |
| ASXL1 | c.3306G > T | | | | | | | | | | | | | |
| ASXL1 | c.3501delC | | | | | | | | | | | | | |
| ASXL1 | c.4189G > A | | | | | | | | | | | | | |
| ASXL1 | c.4343A > G | | | | | | | | | | | | | |

| | | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|---|
| | | cabazitaxel | cabozantinib | docetaxel | etoposide | gemcitabine | paclitaxel | | pemetrexed | cetuximab | | cisplatin | docetaxel | etoposide | gemcitabine |
| Gene | Variant | X | ✓ | ✓ | ✓ | ✓ | ✓ | X | ✓ | ✓ | X | X | ✓ | ✓ | ✓ |
| ABL1 | c.1328T > C | | | | | | | | | | | | | | |
| ABL1 | c.763G > A | | | | | | | | | | | | | | |
| ABL1 | c.764A > T | | | | | | | | | | | | | | |
| ABL1 | c.944C > T | | | | | | | | | | | | | | |
| ABL1 | c.991A > G | | | | | | | | | | | | | | |
| ASXL1 | c.1153G > A | | | | | | | | | | | | | | |
| ASXL1 | c.1162G > A | | | | | | | | | | | | | | |
| ASXL1 | c.1210C > T | | | | | | | | | | | | | | |
| ASXL1 | c.1276A > T | | | | | | | | | | | | | | |
| ASXL1 | c.1633C > T | | | | | | | | | | | | | | |
| ASXL1 | c.1749G > A | | | | | | | | | | | | | | |
| ASXL1 | c.1772dupA | | | | | | | | | | | | | | |
| ASXL1 | c.1773C > G | | | | | | | | | | | | | | |
| ASXL1 | c.1842_1847delinsACAGG | | | | | | | | | | | | | | |
| ASXL1 | c.1895_1896delinsAA | | | | | | | | | | | | | | |
| ASXL1 | c.1925G > C | | | | | | | | | | | | | | |
| ASXL1 | c.1926_1927delAG | | | | | | | | | | | | | | |
| ASXL1 | c.1926delA | | | | | | | | | | | | | | |
| ASXL1 | c.1935dupT | | | | | | | | | | | | | | |
| ASXL1 | c.1947dupA | | | | | | | | | | | | | | |
| ASXL1 | c.2122C > T | | | | | | | | | | | | | | |
| ASXL1 | c.2128G > T | | | | | | | | | | | | | | |
| ASXL1 | c.2129delG | | | | | | | | | | | | | | |
| ASXL1 | c.2238delC | | | | | | | | | | | | | | |
| ASXL1 | c.2269C > T | | | | | | | | | | | | | | |
| ASXL1 | c.2364_2374del11 | | | | | | | | | | | | | | |
| ASXL1 | c.2404dupG | | | | | | | | | | | | | | |
| ASXL1 | c.2416_2417dupAC | | | | | | | | | | | | | | |
| ASXL1 | c.2439delC | | | | | | | | | | | | | | |
| ASXL1 | c.2461delG | | | | | | | | | | | | | | |
| ASXL1 | c.2468delT | | | | | | | | | | | | | | |
| ASXL1 | c.2473A > T | | | | | | | | | | | | | | |
| ASXL1 | c.2929C > T | | | | | | | | | | | | | | |
| ASXL1 | c.2974_2975delCT | | | | | | | | | | | | | | |
| ASXL1 | c.3072_3076dupAAAGG | | | | | | | | | | | | | | |
| ASXL1 | c.3083C > A | | | | | | | | | | | | | | |
| ASXL1 | c.3306G > T | | | | | | | | | | | | | | |
| ASXL1 | c.3501delC | | | | | | | | | | | | | | |
| ASXL1 | c.4189G > A | | | | | | | | | | | | | | |
| ASXL1 | c.4343A > G | | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| ABL1 | c.1328T > C | | | | | | | | | | | |
| ABL1 | c.763G > A | | | | | | | | 1 | | | 1 |
| ABL1 | c.764A > T | | | | | | | | 1 | | | 1 |
| ABL1 | c.944C > T | | | | | | | | 1 | | | 1 |
| ABL1 | c.991A > G | | | | | | | | | | | |
| ASXL1 | c.1153G > A | | | | | | | | | | | |
| ASXL1 | c.1162G > A | | | | | | | | | | | |
| ASXL1 | c.1210C > T | | | | | | | | | | | |
| ASXL1 | c.1276A > T | | | | | | | | | | | |
| ASXL1 | c.1633C > T | | | | | | | | | | | |
| ASXL1 | c.1749G > A | | | | | | | | | | | |
| ASXL1 | c.1772dupA | | | | | | | | | | | |
| ASXL1 | c.1773C > G | | | | | | | | | | | |
| ASXL1 | c.1842_1847delinsACAGG | | | | | | | | | | | |
| ASXL1 | c.1895_1896delinsAA | | | | | | | | | | | |
| ASXL1 | c.1925G > C | | | | | | | | | | | |
| ASXL1 | c.1926_1927delAG | | | | | | | | | | | |
| ASXL1 | c.1926delA | | | | | | | | | | | |
| ASXL1 | c.1935dupT | | | | | | | | | | | |
| ASXL1 | c.1947dupA | | | | | | | | | | | |
| ASXL1 | c.2122C > T | | | | | | | | | | | |
| ASXL1 | c.2128G > T | | | | | | | | | | | |
| ASXL1 | c.2129delG | | | | | | | | | | | |
| ASXL1 | c.2238delC | | | | | | | | | | | |
| ASXL1 | c.2269C > T | | | | | | | | | | | |
| ASXL1 | c.2364_2374del11 | | | | | | | | | | | |
| ASXL1 | c.2404dupG | | | | | | | | | | | |
| ASXL1 | c.2416_2417dupAC | | | | | | | | | | | |
| ASXL1 | c.2439delC | | | | | | | | | | | |
| ASXL1 | c.2461delG | | | | | | | | | | | |
| ASXL1 | c.2468delT | | | | | | | | | | | |
| ASXL1 | c.2473A > T | | | | | | | | | | | |
| ASXL1 | c.2929C > T | | | | | | | | | | | |
| ASXL1 | c.2974_2975delCT | | | | | | | | | | | |
| ASXL1 | c.3072_3076dupAAAGG | | | | | | | | | | | |
| ASXL1 | c.3083C > A | | | | | | | | | | | |
| ASXL1 | c.3306G > T | | | | | | | | | | | |
| ASXL1 | c.3501delC | | | | | | | | | | | |
| ASXL1 | c.4189G > A | | | | | | | | | | | |
| ASXL1 | c.4343A > G | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| ABL1 | c.1328T > C | | | | | | | 2 | | | | | | |
| ABL1 | c.763G > A | | | | | | | 2 | 1 | | 1 | | | 1 |
| ABL1 | c.764A > T | | | | | | | 2 | 1 | | 1 | | | 1 |
| ABL1 | c.944C > T | | | 1 | | | | 2 | 3 | | 1 | | | 1 |
| ABL1 | c.991A > G | | | | | | | 2 | | | | | | |
| ASXL1 | c.1153G > A | | | | | | | | | | | | | |
| ASXL1 | c.1162G > A | | | | | | | | | | | | | |
| ASXL1 | c.1210C > T | | | | | | | | | | | | | |
| ASXL1 | c.1276A > T | | | | | | | | | | | | | |
| ASXL1 | c.1633C > T | | | | | | | | | | | | | |
| ASXL1 | c.1749G > A | | | | | | | | | | | | | |
| ASXL1 | c.1772dupA | | | | | | | | | | | | | |
| ASXL1 | c.1773C > G | | | | | | | | | | | | | |
| ASXL1 | c.1842_1847delinsACAGG | | | | | | | | | | | | | |
| ASXL1 | c.1895_1896delinsAA | | | | | | | | | | | | | |
| ASXL1 | c.1925G > C | | | | | | | | | | | | | |
| ASXL1 | c.1926_1927delAG | | | | | | | | | | | | | |
| ASXL1 | c.1926delA | | | | | | | | | | | | | |
| ASXL1 | c.1935dupT | | | | | | | | | | | | | |
| ASXL1 | c.1947dupA | | | | | | | | | | | | | |
| ASXL1 | c.2122C > T | | | | | | | | | | | | | |
| ASXL1 | c.2128G > T | | | | | | | | | | | | | |
| ASXL1 | c.2129delG | | | | | | | | | | | | | |
| ASXL1 | c.2238delC | | | | | | | | | | | | | |
| ASXL1 | c.2269C > T | | | | | | | | | | | | | |
| ASXL1 | c.2364_2374del11 | | | | | | | | | | | | | |
| ASXL1 | c.2404dupG | | | | | | | | | | | | | |
| ASXL1 | c.2416_2417dupAC | | | | | | | | | | | | | |
| ASXL1 | c.2439delC | | | | | | | | | | | | | |
| ASXL1 | c.2461delG | | | | | | | | | | | | | |
| ASXL1 | c.2468delT | | | | | | | | | | | | | |
| ASXL1 | c.2473A > T | | | | | | | | | | | | | |
| ASXL1 | c.2929C > T | | | | | | | | | | | | | |
| ASXL1 | c.2974_2975delCT | | | | | | | | | | | | | |
| ASXL1 | c.3072_3076dupAAAGG | | | | | | | | | | | | | |
| ASXL1 | c.3083C > A | | | | | | | | | | | | | |
| ASXL1 | c.3306G > T | | | | | | | | | | | | | |
| ASXL1 | c.3501delC | | | | | | | | | | | | | |
| ASXL1 | c.4189G > A | | | | | | | | | | | | | |
| ASXL1 | c.4343A > G | | | | | | | | | | | | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| ABL1 | c.1328T > C | | | | | | | | | | | |
| ABL1 | c.763G > A | | | | | | | | | | | |
| ABL1 | c.764A > T | | | | | | | | | | | |
| ABL1 | c.944C > T | | | | | | | | | | | |
| ABL1 | c.991A > G | | | | | | | | | | | |
| ASXL1 | c.1153G > A | | | | | | | | | | | |
| ASXL1 | c.1162G > A | | | | | | | | | | | |
| ASXL1 | c.1210C > T | | | | | | | | | | | |
| ASXL1 | c.1276A > T | | | | | | | | | | | |
| ASXL1 | c.1633C > T | | | | | | | | | | | |
| ASXL1 | c.1749G > A | | | | | | | | | | | |
| ASXL1 | c.1772dupA | | | | | | | | | | | |
| ASXL1 | c.1773C > G | | | | | | | | | | | |
| ASXL1 | c.1842_1847delinsACAGG | | | | | | | | | | | |
| ASXL1 | c.1895_1896delinsAA | | | | | | | | | | | |
| ASXL1 | c.1925G > C | | | | | | | | | | | |
| ASXL1 | c.1926_1927delAG | | | | | | | | | | | |
| ASXL1 | c.1926delA | | | | | | | | | | | |
| ASXL1 | c.1935dupT | | | | | | | | | | | |
| ASXL1 | c.1947dupA | | | | | | | | | | | |
| ASXL1 | c.2122C > T | | | | | | | | | | | |
| ASXL1 | c.2128G > T | | | | | | | | | | | |
| ASXL1 | c.2129delG | | | | | | | | | | | |
| ASXL1 | c.2238delC | | | | | | | | | | | |
| ASXL1 | c.2269C > T | | | | | | | | | | | |
| ASXL1 | c.2364_2374del11 | | | | | | | | | | | |
| ASXL1 | c.2404dupG | | | | | | | | | | | |
| ASXL1 | c.2416_2417dupAC | | | | | | | | | | | |
| ASXL1 | c.2439delC | | | | | | | | | | | |
| ASXL1 | c.2461delG | | | | | | | | | | | |
| ASXL1 | c.2468delT | | | | | | | | | | | |
| ASXL1 | c.2473A > T | | | | | | | | | | | |
| ASXL1 | c.2929C > T | | | | | | | | | | | |
| ASXL1 | c.2974_2975delCT | | | | | | | | | | | |
| ASXL1 | c.3072_3076dupAAAGG | | | | | | | | | | | |
| ASXL1 | c.3083C > A | | | | | | | | | | | |
| ASXL1 | c.3306G > T | | | | | | | | | | | |
| ASXL1 | c.3501delC | | | | | | | | | | | |
| ASXL1 | c.4189G > A | | | | | | | | | | | |
| ASXL1 | c.4343A > G | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| ABL1 | c.1328T > C | | | | | | | | | | 2 | 1 | |
| ABL1 | c.763G > A | | | | | | | | | | 2 | 2 | |
| ABL1 | c.764A > T | | | | | | | | | | 2 | 2 | |
| ABL1 | c.944C > T | | | | | | | | | | 2 | 3 | |
| ABL1 | c.991A > G | | | | | | | | | | 2 | 1 | |
| ASXL1 | c.1153G > A | | | | | | | | | | | | |
| ASXL1 | c.1162G > A | | | | | | | | | | | | |
| ASXL1 | c.1210C > T | | | | | | | | | | | | |
| ASXL1 | c.1276A > T | | | | | | | | | | | | |
| ASXL1 | c.1633C > T | | | | | | | | | | | | |
| ASXL1 | c.1749G > A | | | | | | | | | | | | |
| ASXL1 | c.1772dupA | | | | | | | | | | | | |
| ASXL1 | c.1773C > G | | | | | | | | | | | | |
| ASXL1 | c.1842_1847delinsACAGG | | | | | | | | | | | | |
| ASXL1 | c.1895_1896delinsAA | | | | | | | | | | | | |
| ASXL1 | c.1925G > C | | | | | | | | | | | | |
| ASXL1 | c.1926_1927delAG | | | | | | | | | | | | |
| ASXL1 | c.1926delA | | | | | | | | | | | | |
| ASXL1 | c.1935dupT | | | | | | | | | | | | |
| ASXL1 | c.1947dupA | | | | | | | | | | | | |
| ASXL1 | c.2122C > T | | | | | | | | | | | | |
| ASXL1 | c.2128G > T | | | | | | | | | | | | |
| ASXL1 | c.2129delG | | | | | | | | | | | | |
| ASXL1 | c.2238delC | | | | | | | | | | | | |
| ASXL1 | c.2269C > T | | | | | | | | | | | | |
| ASXL1 | c.2364_2374del11 | | | | | | | | | | | | |
| ASXL1 | c.2404dupG | | | | | | | | | | | | |
| ASXL1 | c.2416_2417dupAC | | | | | | | | | | | | |
| ASXL1 | c.2439delC | | | | | | | | | | | | |
| ASXL1 | c.2461delG | | | | | | | | | | | | |
| ASXL1 | c.2468delT | | | | | | | | | | | | |
| ASXL1 | c.2473A > T | | | | | | | | | | | | |
| ASXL1 | c.2929C > T | | | | | | | | | | | | |
| ASXL1 | c.2974_2975delCT | | | | | | | | | | | | |
| ASXL1 | c.3072_3076dupAAAGG | | | | | | | | | | | | |
| ASXL1 | c.3083C > A | | | | | | | | | | | | |
| ASXL1 | c.3306G > T | | | | | | | | | | | | |
| ASXL1 | c.3501delC | | | | | | | | | | | | |
| ASXL1 | c.4189G > A | | | | | | | | | | | | |
| ASXL1 | c.4343A > G | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | | | |
| | | lenalidomide | | rituximab | lenvatinib | lorlatinib | inhibitor | methotrexate | methylprednisolone | midostaurin | mitoxantrone |
| Gene | Variant | ✓ | X | ✓ | ✓ | X | ✓ | X | X | ✓ | X |
| ABL1 | c.1328T > C | | | | | | | | | | |
| ABL1 | c.763G > A | | | | | | | 1 | 1 | | |
| ABL1 | c.764A > T | | | | | | | 1 | 1 | | |
| ABL1 | c.944C > T | | | | | | | 1 | 1 | | |
| ABL1 | c.991A > G | | | | | | | | | | |
| ASXL1 | c.1153G > A | | | | | | | | | | |
| ASXL1 | c.1162G > A | | | | | | | | | | |
| ASXL1 | c.1210C > T | | | | | | | | | | |
| ASXL1 | c.1276A > T | | | | | | | | | | |
| ASXL1 | c.1633C > T | | | | | | | | | | |
| ASXL1 | c.1749G > A | | | | | | | | | | |
| ASXL1 | c.1772dupA | | | | | | | | | | |
| ASXL1 | c.1773C > G | | | | | | | | | | |
| ASXL1 | c.1842_1847delinsACAGG | | | | | | | | | | |
| ASXL1 | c.1895_1896delinsAA | | | | | | | | | | |
| ASXL1 | c.1925G > C | | | | | | | | | | |
| ASXL1 | c.1926_1927delAG | | | | | | | | | | |
| ASXL1 | c.1926delA | | | | | | | | | | |
| ASXL1 | c.1935dupT | | | | | | | | | | |
| ASXL1 | c.1947dupA | | | | | | | | | | |
| ASXL1 | c.2122C > T | | | | | | | | | | |
| ASXL1 | c.2128G > T | | | | | | | | | | |
| ASXL1 | c.2129delG | | | | | | | | | | |
| ASXL1 | c.2238delC | | | | | | | | | | |
| ASXL1 | c.2269C > T | | | | | | | | | | |
| ASXL1 | c.2364_2374del11 | | | | | | | | | | |
| ASXL1 | c.2404dupG | | | | | | | | | | |
| ASXL1 | c.2416_2417dupAC | | | | | | | | | | |
| ASXL1 | c.2439delC | | | | | | | | | | |
| ASXL1 | c.2461delG | | | | | | | | | | |
| ASXL1 | c.2468delT | | | | | | | | | | |
| ASXL1 | c.2473A > T | | | | | | | | | | |
| ASXL1 | c.2929C > T | | | | | | | | | | |
| ASXL1 | c.2974_2975delCT | | | | | | | | | | |
| ASXL1 | c.3072_3076dupAAAGG | | | | | | | | | | |
| ASXL1 | c.3083C > A | | | | | | | | | | |
| ASXL1 | c.3306G > T | | | | | | | | | | |
| ASXL1 | c.3501delC | | | | | | | | | | |
| ASXL1 | c.4189G > A | | | | | | | | | | |
| ASXL1 | c.4343A > G | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| ABL1 | c.1328T > C | 2 | | | | | | | | |
| ABL1 | c.763G > A | 2 | 2 | | | | | | | |
| ABL1 | c.764A > T | 2 | 2 | | | | | | | |
| ABL1 | c.944C > T | 3 | 3 | | | | | | 1 | |
| ABL1 | c.991A > G | 2 | | | | | | | | |
| ASXL1 | c.1153G > A | | | | | | | | | |
| ASXL1 | c.1162G > A | | | | | | | | | |
| ASXL1 | c.1210C > T | | | | | | | | | |
| ASXL1 | c.1276A > T | | | | | | | | | |
| ASXL1 | c.1633C > T | | | | | | | | | |
| ASXL1 | c.1749G > A | | | | | | | | | |
| ASXL1 | c.1772dupA | | | | | | | | | |
| ASXL1 | c.1773C > G | | | | | | | | | |
| ASXL1 | c.1842_1847delinsACAGG | | | | | | | | | |
| ASXL1 | c.1895_1896delinsAA | | | | | | | | | |
| ASXL1 | c.1925G > C | | | | | | | | | |
| ASXL1 | c.1926_1927delAG | | | | | | | | | |
| ASXL1 | c.1926delA | | | | | | | | | |
| ASXL1 | c.1935dupT | | | | | | | | | |
| ASXL1 | c.1947dupA | | | | | | | | | |
| ASXL1 | c.2122C > T | | | | | | | | | |
| ASXL1 | c.2128G > T | | | | | | | | | |
| ASXL1 | c.2129delG | | | | | | | | | |
| ASXL1 | c.2238delC | | | | | | | | | |
| ASXL1 | c.2269C > T | | | | | | | | | |
| ASXL1 | c.2364_2374del11 | | | | | | | | | |
| ASXL1 | c.2404dupG | | | | | | | | | |
| ASXL1 | c.2416_2417dupAC | | | | | | | | | |
| ASXL1 | c.2439delC | | | | | | | | | |
| ASXL1 | c.2461delG | | | | | | | | | |
| ASXL1 | c.2468delT | | | | | | | | | |
| ASXL1 | c.2473A > T | | | | | | | | | |
| ASXL1 | c.2929C > T | | | | | | | | | |
| ASXL1 | c.2974_2975delCT | | | | | | | | | |
| ASXL1 | c.3072_3076dupAAAGG | | | | | | | | | |
| ASXL1 | c.3083C > A | | | | | | | | | |
| ASXL1 | c.3306G > T | | | | | | | | | |
| ASXL1 | c.3501delC | | | | | | | | | |
| ASXL1 | c.4189G > A | | | | | | | | | |
| ASXL1 | c.4343A > G | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alfa-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| ABL1 | c.1328T > C | | | | | | | | | 2 | | | |
| ABL1 | c.763G > A | | | | | | | | | 2 | 1 | 1 | |
| ABL1 | c.764A > T | | | | | | | | | 2 | 1 | 1 | |
| ABL1 | c.944C > T | | | | | | | | | 3 | 1 | 1 | |
| ABL1 | c.991A > G | | | | | | | | | 2 | | | |
| ASXL1 | c.1153G > A | | | | 1 | | | | | | | | |
| ASXL1 | c.1162G > A | | | | 1 | | | | | | | | |
| ASXL1 | c.1210C > T | | | | 1 | | | | | | | | |
| ASXL1 | c.1276A > T | | | | 1 | | | | | | | | |
| ASXL1 | c.1633C > T | | | | 1 | | | | | | | | |
| ASXL1 | c.1749G > A | | | | 1 | | | | | | | | |
| ASXL1 | c.1772dupA | | | | 1 | | | | | | | | |
| ASXL1 | c.1773C > G | | | | 1 | | | | | | | | |
| ASXL1 | c.1842_1847delinsACAGG | | | | 1 | | | | | | | | |
| ASXL1 | c.1895_1896delinsAA | | | | 1 | | | | | | | | |
| ASXL1 | c.1925G > C | | | | 1 | | | | | | | | |
| ASXL1 | c.1926_1927delAG | | | | 1 | | | | | | | | |
| ASXL1 | c.1926delA | | | | 1 | | | | | | | | |
| ASXL1 | c.1935dupT | | | | 1 | | | | | | | | |
| ASXL1 | c.1947dupA | | | | 1 | | | | | | | | |
| ASXL1 | c.2122C > T | | | | 1 | | | | | | | | |
| ASXL1 | c.2128G > T | | | | 1 | | | | | | | | |
| ASXL1 | c.2129delG | | | | 1 | | | | | | | | |
| ASXL1 | c.2238delC | | | | 1 | | | | | | | | |
| ASXL1 | c.2269C > T | | | | 1 | | | | | | | | |
| ASXL1 | c.2364_2374del11 | | | | 1 | | | | | | | | |
| ASXL1 | c.2404dupG | | | | 1 | | | | | | | | |
| ASXL1 | c.2416_2417dupAC | | | | 1 | | | | | | | | |
| ASXL1 | c.2439delC | | | | 1 | | | | | | | | |
| ASXL1 | c.2461delG | | | | 1 | | | | | | | | |
| ASXL1 | c.2468delT | | | | 1 | | | | | | | | |
| ASXL1 | c.2473A > T | | | | 1 | | | | | | | | |
| ASXL1 | c.2929C > T | | | | 1 | | | | | | | | |
| ASXL1 | c.2974_2975delCT | | | | 1 | | | | | | | | |
| ASXL1 | c.3072_3076dupAAAGG | | | | 1 | | | | | | | | |
| ASXL1 | c.3083C > A | | | | 1 | | | | | | | | |
| ASXL1 | c.3306G > T | | | | 1 | | | | | | | | |
| ASXL1 | c.3501delC | | | | 1 | | | | | | | | |
| ASXL1 | c.4189G > A | | | | 1 | | | | | | | | |
| ASXL1 | c.4343A > G | | | | 1 | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| ABL1 | c.1328T > C | | | 2 | | | | | | | | |
| ABL1 | c.763G > A | | | 2 | | | | | | | | |
| ABL1 | c.764A > T | | | 2 | | | | | | | | |
| ABL1 | c.944C > T | | | 2 | | | | | | | | |
| ABL1 | c.991A > G | | | 2 | | | | | | | | |
| ASXL1 | c.1153G > A | | | | | | | | | | | |
| ASXL1 | c.1162G > A | | | | | | | | | | | |
| ASXL1 | c.1210C > T | | | | | | | | | | | |
| ASXL1 | c.1276A > T | | | | | | | | | | | |
| ASXL1 | c.1633C > T | | | | | | | | | | | |
| ASXL1 | c.1749G > A | | | | | | | | | | | |
| ASXL1 | c.1772dupA | | | | | | | | | | | |
| ASXL1 | c.1773C > G | | | | | | | | | | | |
| ASXL1 | c.1842_1847delinsACAGG | | | | | | | | | | | |
| ASXL1 | c.1895_1896delinsAA | | | | | | | | | | | |
| ASXL1 | c.1925G > C | | | | | | | | | | | |
| ASXL1 | c.1926_1927delAG | | | | | | | | | | | |
| ASXL1 | c.1926delA | | | | | | | | | | | |
| ASXL1 | c.1935dupT | | | | | | | | | | | |
| ASXL1 | c.1947dupA | | | | | | | | | | | |
| ASXL1 | c.2122C > T | | | | | | | | | | | |
| ASXL1 | c.2128G > T | | | | | | | | | | | |
| ASXL1 | c.2129delG | | | | | | | | | | | |
| ASXL1 | c.2238delC | | | | | | | | | | | |
| ASXL1 | c.2269C > T | | | | | | | | | | | |
| ASXL1 | c.2364_2374del11 | | | | | | | | | | | |
| ASXL1 | c.2404dupG | | | | | | | | | | | |
| ASXL1 | c.2416_2417dupAC | | | | | | | | | | | |
| ASXL1 | c.2439delC | | | | | | | | | | | |
| ASXL1 | c.2461delG | | | | | | | | | | | |
| ASXL1 | c.2468delT | | | | | | | | | | | |
| ASXL1 | c.2473A > T | | | | | | | | | | | |
| ASXL1 | c.2929C > T | | | | | | | | | | | |
| ASXL1 | c.2974_2975delCT | | | | | | | | | | | |
| ASXL1 | c.3072_3076dupAAAGG | | | | | | | | | | | |
| ASXL1 | c.3083C > A | | | | | | | | | | | |
| ASXL1 | c.3306G > T | | | | | | | | | | | |
| ASXL1 | c.3501delC | | | | | | | | | | | |
| ASXL1 | c.4189G > A | | | | | | | | | | | |
| ASXL1 | c.4343A > G | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| ABL1 | c.1328T > C | | | 2 | | | | | | | | |
| ABL1 | c.763G > A | | | 2 | | | | | | | 1 | |
| ABL1 | c.764A > T | | | 2 | | | | | | | 1 | |
| ABL1 | c.944C > T | | | 2 | | | | | | | 1 | |
| ABL1 | c.991A > G | | | 2 | | | | | | | | |
| ASXL1 | c.1153G > A | | | | | | | | | | | |
| ASXL1 | c.1162G > A | | | | | | | | | | | |
| ASXL1 | c.1210C > T | | | | | | | | | | | |
| ASXL1 | c.1276A > T | | | | | | | | | | | |
| ASXL1 | c.1633C > T | | | | | | | | | | | |
| ASXL1 | c.1749G > A | | | | | | | | | | | |
| ASXL1 | c.1772dupA | | | | | | | | | | | |
| ASXL1 | c.1773C > G | | | | | | | | | | | |
| ASXL1 | c.1842_1847delinsACAGG | | | | | | | | | | | |
| ASXL1 | c.1895_1896delinsAA | | | | | | | | | | | |
| ASXL1 | c.1925G > C | | | | | | | | | | | |
| ASXL1 | c.1926_1927delAG | | | | | | | | | | | |
| ASXL1 | c.1926delA | | | | | | | | | | | |
| ASXL1 | c.1935dupT | | | | | | | | | | | |
| ASXL1 | c.1947dupA | | | | | | | | | | | |
| ASXL1 | c.2122C > T | | | | | | | | | | | |
| ASXL1 | c.2128G > T | | | | | | | | | | | |
| ASXL1 | c.2129delG | | | | | | | | | | | |
| ASXL1 | c.2238delC | | | | | | | | | | | |
| ASXL1 | c.2269C > T | | | | | | | | | | | |
| ASXL1 | c.2364_2374del11 | | | | | | | | | | | |
| ASXL1 | c.2404dupG | | | | | | | | | | | |
| ASXL1 | c.2416_2417dupAC | | | | | | | | | | | |
| ASXL1 | c.2439delC | | | | | | | | | | | |
| ASXL1 | c.2461delG | | | | | | | | | | | |
| ASXL1 | c.2468delT | | | | | | | | | | | |
| ASXL1 | c.2473A > T | | | | | | | | | | | |
| ASXL1 | c.2929C > T | | | | | | | | | | | |
| ASXL1 | c.2974_2975delCT | | | | | | | | | | | |
| ASXL1 | c.3072_3076dupAAAGG | | | | | | | | | | | |
| ASXL1 | c.3083C > A | | | | | | | | | | | |
| ASXL1 | c.3306G > T | | | | | | | | | | | |
| ASXL1 | c.3501delC | | | | | | | | | | | |
| ASXL1 | c.4189G > A | | | | | | | | | | | |
| ASXL1 | c.4343A > G | | | | | | | | | | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| BRAF | c.1332G > A | | | 1 | | | | | | 1 | | | | | | |
| BRAF | c.1405G > C | | | 1 | | | | | | 1 | | | | | | |
| BRAF | c.1454T > G | | | 1 | | | | | | 1 | | | | | | |
| BRAF | c.1742A > G | | | 1 | | | | | | 1 | | | | | | |
| BRAF | c.1781A > G | | | 1 | | | | | | 1 | | | | | | |
| BRAF | c.1782T > A | | | 1 | | | | | | 1 | | | | | | |
| BRAF | c.1799T > A | | | 1 | | 1 | | | | 1 | | | | | | |
| CDKN2A | c.371G > C | | | | | | | | | | 1 | | | | | |
| CEBPA | c.129delC | | | | | | | | | | | | | | | |
| CEBPA | c.413A > T | | | | | | | | | | | | | | | |
| CEBPA | c.566C > A | | | | | | | | | | | | | | | |
| CEBPA | c.568T > C | | | | | | | | | | | | | | | |
| CEBPA | c.68dupC | | | | | | | | | | | | | | | |
| CEBPA | c.690G > T | | | | | | | | | | | | | | | |
| CEBPA | c.724G > A | | | | | | | | | | | | | | | |
| CEBPA | c.952_955delACCA | | | | | | | | | | | | | | | |
| CEBPA | c.952A > T | | | | | | | | | | | | | | | |
| CEBPA | c.953_956delCCAG | | | | | | | | | | | | | | | |
| CEBPA | c.971T > C | | | | | | | | | | | | | | | |
| CSF3R | c.1853C > T | | | | | | | | | | | | | | | |
| CSF3R | c.2041 − | | | | | | | | | | | | | | | |
| | 8C > T | | | | | | | | | | | | | | | |
| CSF3R | c.2087T > C | | | | | | | | | | | | | | | |
| CSF3R | c.2326C > T | | | | | | | | | | | | | | | |
| CSF3R | c.2503G > A | | | | | | | | | | | | | | | |
| DNMT3A | c.1155G > A | | | | | | | | | | | | | | | |
| DNMT3A | c.1204C > T | | | | | | | | | | | | | | | |
| DNMT3A | c.1502A > G | | | | | | | | | | | | | | | |
| DNMT3A | c.1540delT | | | | | | | | | | | | | | | |
| DNMT3A | c.1668G > T | | | | | | | | | | | | | | | |
| DNMT3A | c.1904G > A | | | | | | | | | | | | | | | |
| DNMT3A | c.1907T > A | | | | | | | | | | | | | | | |
| DNMT3A | c.1924G > A | | | | | | | | | | | | | | | |
| DNMT3A | c.1937 − | | | | | | | | | | | | | | | |
| | 10G > T | | | | | | | | | | | | | | | |
| DNMT3A | c.2007dupC | | | | | | | | | | | | | | | |
| DNMT3A | c.2084T > G | | | | | | | | | | | | | | | |
| DNMT3A | c.2141C > T | | | | | | | | | | | | | | | |
| DNMT3A | c.2172dupC | | | | | | | | | | | | | | | |
| DNMT3A | c.2193_2195delCTT | | | | | | | | | | | | | | | |
| DNMT3A | c.2206C > T | | | | | | | | | | | | | | | |
| DNMT3A | c.2210T > G | | | | | | | | | | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| BRAF | c.1332G > A | 1 | | 1 | | 1 | | | | | | 1 | 1 | | |
| BRAF | c.1405G > C | 1 | | 1 | | 1 | | | | | | 1 | 1 | | |
| BRAF | c.1454T > G | 1 | | 1 | | 1 | | | | | | 1 | 1 | | |
| BRAF | c.1742A > G | 1 | | 1 | | 1 | | | | | | 1 | 1 | | |
| BRAF | c.1781A > G | 1 | | 1 | | 1 | | | | | | 1 | 1 | | |
| BRAF | c.1782T > A | 1 | | | | 1 | | | | | | 1 | 1 | | |
| BRAF | c.1799T > A | 1 | | 1 | | 2 | | | | | | 1 | 1 | | |
| CDKN2A | c.371G > C | | | | | | | | | | | | | | |
| CEBPA | c.129delC | | | | | | | | | | | | | | |
| CEBPA | c.413A > T | | | | | | | | | | | | | | |
| CEBPA | c.566C > A | | | | | | | | | | | | | | |
| CEBPA | c.568T > C | | | | | | | | | | | | | | |
| CEBPA | c.68dupC | | | | | | | | | | | | | | |
| CEBPA | c.690G > T | | | | | | | | | | | | | | |
| CEBPA | c.724G > A | | | | | | | | | | | | | | |
| CEBPA | c.952_955delACCA | | | | | | | | | | | | | | |
| CEBPA | c.952A > T | | | | | | | | | | | | | | |
| CEBPA | c.953_956delCCAG | | | | | | | | | | | | | | |
| CEBPA | c.971T > C | | | | | | | | | | | | | | |
| CSF3R | c.1853C > T | | | | | | | | | | | | | | |
| CSF3R | c.2041 − | | | | | | | | | | | | | | |
| | 8C > T | | | | | | | | | | | | | | |
| CSF3R | c.2087T > C | | | | | | | | | | | | | | |
| CSF3R | c.2326C > T | | | | | | | | | | | | | | |
| CSF3R | c.2503G > A | | | | | | | | | | | | | | |
| DNMT3A | c.1155G > A | | | | | | | | | | | | | | |
| DNMT3A | c.1204C > T | | | | | | | | | | | | | | |
| DNMT3A | c.1502A > G | | | | | | | | | | | | | | |
| DNMT3A | c.1540delT | | | | | | | | | | | | | | |
| DNMT3A | c.1668G > T | | | | | | | | | | | | | | |
| DNMT3A | c.1904G > A | | | | | | | | | | | | | | |
| DNMT3A | c.1907T > A | | | | | | | | | | | | | | |
| DNMT3A | c.1924G > A | | | | | | | | | | | | | | |
| DNMT3A | c.1937 − | | | | | | | | | | | | | | |
| | 10G > T | | | | | | | | | | | | | | |
| DNMT3A | c.2007dupC | | | | | | | | | | | | | | |
| DNMT3A | c.2084T > G | | | | | | | | | | | | | | |
| DNMT3A | c.2141C > T | | | | | | | | | | | | | | |
| DNMT3A | c.2172dupC | | | | | | | | | | | | | | |
| DNMT3A | c.2193_2195delCTT | | | | | | | | | | | | | | |
| DNMT3A | c.2206C > T | | | | | | | | | | | | | | |
| DNMT3A | c.2210T > G | | | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| BRAF | c.1332G > A | | | | | | | | | 1 | | | | |
| BRAF | c.1405G > C | | | | | | | | | 1 | | | | |
| BRAF | c.1454T > G | | | | | | | | | 1 | | | | |
| BRAF | c.1742A > G | | | | | | | | | 2 | | | | |
| BRAF | c.1781A > G | | | | | | | | | 2 | | | | |
| BRAF | c.1782T > A | | | | | | | | | 2 | | | | |
| BRAF | c.1799T > A | | 1 | 1 | 1 | 1 | | 1 | 1 | 2 | | 1 | 1 | 1 |
| CDKN2A | c.371G > C | | | | | | | | | | | | | |
| CEBPA | c.129delC | | | | | | | | | | | | | |
| CEBPA | c.413A > T | | | | | | | | | | | | | |
| CEBPA | c.566C > A | | | | | | | | | | | | | |
| CEBPA | c.568T > C | | | | | | | | | | | | | |
| CEBPA | c.68dupC | | | | | | | | | | | | | |
| CEBPA | c.690G > T | | | | | | | | | | | | | |
| CEBPA | c.724G > A | | | | | | | | | | | | | |
| CEBPA | c.952_955delACCA | | | | | | | | | | | | | |
| CEBPA | c.952A > T | | | | | | | | | | | | | |
| CEBPA | c.953_956delCCAG | | | | | | | | | | | | | |
| CEBPA | c.971T > C | | | | | | | | | | | | | |
| CSF3R | c.1853C > T | | | | | | | | | | | | | |
| CSF3R | c.2041 − | | | | | | | | | | | | | |
| | 8C > T | | | | | | | | | | | | | |
| CSF3R | c.2087T > C | | | | | | | | | | | | | |
| CSF3R | c.2326C > T | | | | | | | | | | | | | |
| CSF3R | c.2503G > A | | | | | | | | | | | | | |
| DNMT3A | c.1155G > A | | | | | | | | | | | | | |
| DNMT3A | c.1204C > T | | | | | | | | | | | | | |
| DNMT3A | c.1502A > G | | | | | | | | | | | | | |
| DNMT3A | c.1540delT | | | | | | | | | | | | | |
| DNMT3A | c.1668G > T | | | | | | | | | | | | | |
| DNMT3A | c.1904G > A | | | | | | | | | | | | | |
| DNMT3A | c.1907T > A | | | | | | | | | | | | | |
| DNMT3A | c.1924G > A | | | | | | | | | | | | | |
| DNMT3A | c.1937 − | | | | | | | | | | | | | |
| | 10G > T | | | | | | | | | | | | | |
| DNMT3A | c.2007dupC | | | | | | | | | | | | | |
| DNMT3A | c.2084T > G | | | | | | | | | | | | | |
| DNMT3A | c.2141C > T | | | | | | | | | | | | | |
| DNMT3A | c.2172dupC | | | | | | | | | | | | | |
| DNMT3A | c.2193_2195delCTT | | | | | | | | | | | | | |
| DNMT3A | c.2206C > T | | | | | | | | | | | | | |
| DNMT3A | c.2210T > G | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| BRAF | c.1332G > A | | | | | | | | | | | |
| BRAF | c.1405G > C | | | | | | | | | | | |
| BRAF | c.1454T > G | | | | | | | | | | | |
| BRAF | c.1742A > G | | | | | | | | | | | |
| BRAF | c.1781A > G | | | | | | | | | | | |
| BRAF | c.1782T > A | | | | | | | | | | | |
| BRAF | c.1799T > A | 1 | 1 | | 1 | 4 | | | | | | |
| CDKN2A | c.371G > C | | | | | | | | | | | |
| CEBPA | c.129delC | | | | | | | | | | | |
| CEBPA | c.413A > T | | | | | | | | | | | |
| CEBPA | c.566C > A | | | | | | | | | | | |
| CEBPA | c.568T > C | | | | | | | | | | | |
| CEBPA | c.68dupC | | | | | | | | | | | |
| CEBPA | c.690G > T | | | | | | | | | | | |
| CEBPA | c.724G > A | | | | | | | | | | | |
| CEBPA | c.952_955delACCA | | | | | | | | | | | |
| CEBPA | c.952A > T | | | | | | | | | | | |
| CEBPA | c.953_956delCCAG | | | | | | | | | | | |
| CEBPA | c.971T > C | | | | | | | | | | | |
| CSF3R | c.1853C > T | | | | | | | | | | | |
| CSF3R | c.2041 − | | | | | | | | | | | |
| | 8C > T | | | | | | | | | | | |
| CSF3R | c.2087T > C | | | | | | | | | | | |
| CSF3R | c.2326C > T | | | | | | | | | | | |
| CSF3R | c.2503G > A | | | | | | | | | | | |
| DNMT3A | c.1155G > A | | | | | | | | | | | |
| DNMT3A | c.1204C > T | | | | | | | | | | | |
| DNMT3A | c.1502A > G | | | | | | | | | | | |
| DNMT3A | c.1540delT | | | | | | | | | | | |
| DNMT3A | c.1668G > T | | | | | | | | | | | |
| DNMT3A | c.1904G > A | | | | | | | | | | | |
| DNMT3A | c.1907T > A | | | | | | | | | | | |
| DNMT3A | c.1924G > A | | | | | | | | | | | |
| DNMT3A | c.1937 − | | | | | | | | | | | |
| | 10G > T | | | | | | | | | | | |
| DNMT3A | c.2007dupC | | | | | | | | | | | |
| DNMT3A | c.2084T > G | | | | | | | | | | | |
| DNMT3A | c.2141C > T | | | | | | | | | | | |
| DNMT3A | c.2172dupC | | | | | | | | | | | |
| DNMT3A | c.2193_2195delCTT | | | | | | | | | | | |
| DNMT3A | c.2206C > T | | | | | | | | | | | |
| DNMT3A | c.2210T > G | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| BRAF | c.1332G > A | | 3 | 1 | 1 | 1 | | | | | | | | |
| BRAF | c.1405G > C | | 3 | 1 | 1 | 1 | | | | | | | | |
| BRAF | c.1454T > G | | 3 | 1 | 1 | 1 | | | | | | | | |
| BRAF | c.1742A > G | | 3 | 1 | 1 | 1 | | | | | | | | |
| BRAF | c.1781A > G | | 3 | 1 | 1 | 1 | | | | | | | | |
| BRAF | c.1782T > A | | 2 | 1 | | 1 | | | | | | | | |
| BRAF | c.1799T > A | | 4 | 1 | 5 | 1 | | | | | | | | |
| CDKN2A | c.371G > C | | | | | | | | | | | | | |
| CEBPA | c.129delC | | | | | | | | | | | | | |
| CEBPA | c.413A > T | | | | | | | | | | | | | |
| CEBPA | c.566C > A | | | | | | | | | | | | | |
| CEBPA | c.568T > C | | | | | | | | | | | | | |
| CEBPA | c.68dupC | | | | | | | | | | | | | |
| CEBPA | c.690G > T | | | | | | | | | | | | | |
| CEBPA | c.724G > A | | | | | | | | | | | | | |
| CEBPA | c.952_955delACCA | | | | | | | | | | | | | |
| CEBPA | c.952A > T | | | | | | | | | | | | | |
| CEBPA | c.953_956delCCAG | | | | | | | | | | | | | |
| CEBPA | c.971T > C | | | | | | | | | | | | | |
| CSF3R | c.1853C > T | | | | | | | | | | | | | |
| CSF3R | c.2041 − | | | | | | | | | | | | | |
| | 8C > T | | | | | | | | | | | | | |
| CSF3R | c.2087T > C | | | | | | | | | | | | | |
| CSF3R | c.2326C > T | | | | | | | | | | | | | |
| CSF3R | c.2503G > A | | | | | | | | | | | | | |
| DNMT3A | c.1155G > A | 1 | | | | | | | | 1 | | 2 | | |
| DNMT3A | c.1204C > T | 1 | | | | | | | | 1 | | 2 | | |
| DNMT3A | c.1502A > G | 1 | | | | | | | | 1 | | 2 | | |
| DNMT3A | c.1540delT | 1 | | | | | | | | 1 | | 2 | | |
| DNMT3A | c.1668G > T | | | | | | | | | 1 | | 2 | | |
| DNMT3A | c.1904G > A | 1 | | | | | | | | 1 | | 2 | | |
| DNMT3A | c.1907T > A | | | | | | | | | 1 | | 2 | | |
| DNMT3A | c.1924G > A | | | | | | | | | 1 | | 2 | | |
| DNMT3A | c.1937 − | 1 | | | | | | | | 1 | | 2 | | |
| | 10G > T | | | | | | | | | | | | | |
| DNMT3A | c.2007dupC | 1 | | | | | | | | 1 | | 2 | | |
| DNMT3A | c.2084T > G | 1 | | | | | | | | 1 | | 2 | | |
| DNMT3A | c.2141C > T | | | | | | | | | 1 | | 2 | | |
| DNMT3A | c.2172dupC | | | | | | | | | 1 | | 2 | | |
| DNMT3A | c.2193_2195delCTT | 1 | | | | | | | | 1 | | 2 | | |
| DNMT3A | c.2206C > T | 1 | | | | | | | | 1 | | 2 | | |
| DNMT3A | c.2210T > G | | | | | | | | | 1 | | 2 | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| BRAF | c.1332G > A | | | | | | | | | | | |
| BRAF | c.1405G > C | | | | 1 | | | | | | | |
| BRAF | c.1454T > G | | | | | | | | | | | |
| BRAF | c.1742A > G | | | | | | | | | | | |
| BRAF | c.1781A > G | | | | | | | | | | | |
| BRAF | c.1782T > A | | | | | | | | | | | |
| BRAF | c.1799T > A | | 1 | | | | | | | | | |
| CDKN2A | c.371G > C | | | | | | | | | | | |
| CEBPA | c.129delC | | | | | | | | | | | |
| CEBPA | c.413A > T | | | | | | | | | | | |
| CEBPA | c.566C > A | | | | | | | | | | | |
| CEBPA | c.568T > C | | | | | | | | | | | |
| CEBPA | c.68dupC | | | | | | | | | | | |
| CEBPA | c.690G > T | | | | | | | | | | | |
| CEBPA | c.724G > A | | | | | | | | | | | |
| CEBPA | c.952_955delACCA | | | | | | | | | | | |
| CEBPA | c.952A > T | | | | | | | | | | | |
| CEBPA | c.953_956delCCAG | | | | | | | | | | | |
| CEBPA | c.971T > C | | | | | | | | | | | |
| CSF3R | c.1853C > T | | | | | | | | | | 1 | |
| CSF3R | c.2041 − | | | | | | | | | | 1 | |
| | 8C > T | | | | | | | | | | | |
| CSF3R | c.2087T > C | | | | | | | | | | 1 | |
| CSF3R | c.2326C > T | | | | | | | | | | 1 | |
| CSF3R | c.2503G > A | | | | | | | | | | 1 | |
| DNMT3A | c.1155G > A | | | | | | | | | | | |
| DNMT3A | c.1204C > T | | | | | | | | | | | |
| DNMT3A | c.1502A > G | | | | | | | | | | | |
| DNMT3A | c.1540delT | | | | | | | | | | | |
| DNMT3A | c.1668G > T | | | | | | | | | | | |
| DNMT3A | c.1904G > A | | | | | | | | | | | |
| DNMT3A | c.1907T > A | | | | | | | | | | | |
| DNMT3A | c.1924G > A | | | | | | | | | | | |
| DNMT3A | c.1937 − | | | | | | | | | | | |
| | 10G > T | | | | | | | | | | | |
| DNMT3A | c.2007dupC | | | | | | | | | | | |
| DNMT3A | c.2084T > G | | | | | | | | | | | |
| DNMT3A | c.2141C > T | | | | | | | | | | | |
| DNMT3A | c.2172dupC | | | | | | | | | | | |
| DNMT3A | c.2193_2195delCTT | | | | | | | | | | | |
| DNMT3A | c.2206C > T | | | | | | | | | | | |
| DNMT3A | c.2210T > G | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| BRAF | c.1332G > A | 1 | | | | | | | | | | 1 | 1 |
| BRAF | c.1405G > C | 1 | 1 | | | | | | | | | 1 | 1 |
| BRAF | c.1454T > G | 1 | 1 | | | | | | | | | 1 | 1 |
| BRAF | c.1742A > G | 1 | 1 | | | | | | | | | 1 | 1 |
| BRAF | c.1781A > G | 1 | 1 | | | | | | | | | 1 | 1 |
| BRAF | c.1782T > A | 1 | 1 | 1 | | | | | | | | | 1 |
| BRAF | c.1799T > A | 1 | 1 | | | 1 | | | | | | 2 | 2 |
| CDKN2A | c.371G > C | | | | | | | | | | | | |
| CEBPA | c.129delC | | | | | | | | | | | | |
| CEBPA | c.413A > T | | | | | | | | | | | | |
| CEBPA | c.566C > A | | | | | | | | | | | | |
| CEBPA | c.568T > C | | | | | | | | | | | | |
| CEBPA | c.68dupC | | | | | | | | | | | | |
| CEBPA | c.690G > T | | | | | | | | | | | | |
| CEBPA | c.724G > A | | | | | | | | | | | | |
| CEBPA | c.952_955delACCA | | | | | | | | | | | | |
| CEBPA | c.952A > T | | | | | | | | | | | | |
| CEBPA | c.953_956delCCAG | | | | | | | | | | | | |
| CEBPA | c.971T > C | | | | | | | | | | | | |
| CSF3R | c.1853C > T | | | | | | | | | | | | |
| CSF3R | c.2041 − | | | | | | | | | | | | |
| | 8C > T | | | | | | | | | | | | |
| CSF3R | c.2087T > C | | | | | | | | | | | | |
| CSF3R | c.2326C > T | | | | | | | | | | | | |
| CSF3R | c.2503G > A | | | | | | | | | | | | |
| DNMT3A | c.1155G > A | | | | | | | | | | | | |
| DNMT3A | c.1204C > T | | | | | | | | | | | | |
| DNMT3A | c.1502A > G | | | | | | | | | | | | |
| DNMT3A | c.1540delT | | | | | | | | | | | | |
| DNMT3A | c.1668G > T | | | | | | | | | | | | |
| DNMT3A | c.1904G > A | | | | | | | | | | | | |
| DNMT3A | c.1907T > A | | | | | | | | | | | | |
| DNMT3A | c.1924G > A | | | | | | | | | | | | |
| DNMT3A | c.1937 − | | | | | | | | | | | | |
| | 10G > T | | | | | | | | | | | | |
| DNMT3A | c.2007dupC | | | | | | | | | | | | |
| DNMT3A | c.2084T > G | | | | | | | | | | | | |
| DNMT3A | c.2141C > T | | | | | | | | | | | | |
| DNMT3A | c.2172dupC | | | | | | | | | | | | |
| DNMT3A | c.2193_2195delCTT | | | | | | | | | | | | |
| DNMT3A | c.2206C > T | | | | | | | | | | | | |
| DNMT3A | c.2210T > G | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | | | |
| | | lenalidomide | | rituximab | lenvatinib | lorlatinib | inhibitor | methotrexate | methylprednisolone | midostaurin | mitoxantrone |
| Gene | Variant | ✓ | X | ✓ | ✓ | X | ✓ | X | X | ✓ | X |
| BRAF | c.1332G > A | | | | | | | | | | |
| BRAF | c.1405G > C | | | | | | | | | | |
| BRAF | c.1454T > G | | | | | | | | | | |
| BRAF | c.1742A > G | | | | | | | | | | |
| BRAF | c.1781A > G | | | | | | | | | | |
| BRAF | c.1782T > A | | | | | | | | | | |
| BRAF | c.1799T > A | | | | | | 1 | | | | |
| CDKN2A | c.371G > C | | | | | | | | | | |
| CEBPA | c.129delC | | | | | | | | | | |
| CEBPA | c.413A > T | | | | | | | | | | |
| CEBPA | c.566C > A | | | | | | | | | | |
| CEBPA | c.568T > C | | | | | | | | | | |
| CEBPA | c.68dupC | | | | | | | | | | |
| CEBPA | c.690G > T | | | | | | | | | | |
| CEBPA | c.724G > A | | | | | | | | | | |
| CEBPA | c.952_955delACCA | | | | | | | | | | |
| CEBPA | c.952A > T | | | | | | | | | | |
| CEBPA | c.953_956delCCAG | | | | | | | | | | |
| CEBPA | c.971T > C | | | | | | | | | | |
| CSF3R | c.1853C > T | | | | | | | | | | |
| CSF3R | c.2041 − | | | | | | | | | | |
| | 8C > T | | | | | | | | | | |
| CSF3R | c.2087T > C | | | | | | | | | | |
| CSF3R | c.2326C > T | | | | | | | | | | |
| CSF3R | c.2503G > A | | | | | | | | | | |
| DNMT3A | c.1155G > A | | | | | | | | | | |
| DNMT3A | c.1204C > T | | | | | | | | | | |
| DNMT3A | c.1502A > G | | | | | | | | | | |
| DNMT3A | c.1540delT | | | | | | | | | | |
| DNMT3A | c.1668G > T | | | | | | | | | | |
| DNMT3A | c.1904G > A | | | | | | | | | | |
| DNMT3A | c.1907T > A | | | | | | | | | | |
| DNMT3A | c.1924G > A | | | | | | | | | | |
| DNMT3A | c.1937 − | | | | | | | | | | |
| | 10G > T | | | | | | | | | | |
| DNMT3A | c.2007dupC | | | | | | | | | | |
| DNMT3A | c.2084T > G | | | | | | | | | | |
| DNMT3A | c.2141C > T | | | | | | | | | | |
| DNMT3A | c.2172dupC | | | | | | | | | | |
| DNMT3A | c.2193_2195delCTT | | | | | | | | | | |
| DNMT3A | c.2206C > T | | | | | | | | | | |
| DNMT3A | c.2210T > G | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| BRAF | c.1332G > A | | | | 2 | | | | | |
| BRAF | c.1405G > C | | | | 2 | | | | | |
| BRAF | c.1454T > G | | | | 2 | | | | | |
| BRAF | c.1742A > G | | | | 2 | | | | | |
| BRAF | c.1781A > G | | | | 2 | | | | | |
| BRAF | c.1782T > A | | | | 1 | | | | | |
| BRAF | c.1799T > A | | | | 4 | | | | | 1 |
| CDKN2A | c.371G > C | | | | | | | | | |
| CEBPA | c.129delC | | | | | | | | | |
| CEBPA | c.413A > T | | | | | | | | | |
| CEBPA | c.566C > A | | | | | | | | | |
| CEBPA | c.568T > C | | | | | | | | | |
| CEBPA | c.68dupC | | | | | | | | | |
| CEBPA | c.690G > T | | | | | | | | | |
| CEBPA | c.724G > A | | | | | | | | | |
| CEBPA | c.952_955delACCA | | | | | | | | | |
| CEBPA | c.952A > T | | | | | | | | | |
| CEBPA | c.953_956delCCAG | | | | | | | | | |
| CEBPA | c.971T > C | | | | | | | | | |
| CSF3R | c.1853C > T | | | | | | | | | |
| CSF3R | c.2041 − | | | | | | | | | |
| | 8C > T | | | | | | | | | |
| CSF3R | c.2087T > C | | | | | | | | | |
| CSF3R | c.2326C > T | | | | | | | | | |
| CSF3R | c.2503G > A | | | | | | | | | |
| DNMT3A | c.1155G > A | | | | | | | | | |
| DNMT3A | c.1204C > T | | | | | | | | | |
| DNMT3A | c.1502A > G | | | | | | | | | |
| DNMT3A | c.1540delT | | | | | | | | | |
| DNMT3A | c.1668G > T | | | | | | | | | |
| DNMT3A | c.1904G > A | | | | | | | | | |
| DNMT3A | c.1907T > A | | | | | | | | | |
| DNMT3A | c.1924G > A | | | | | | | | | |
| DNMT3A | c.1937 − | | | | | | | | | |
| | 10G > T | | | | | | | | | |
| DNMT3A | c.2007dupC | | | | | | | | | |
| DNMT3A | c.2084T > G | | | | | | | | | |
| DNMT3A | c.2141C > T | | | | | | | | | |
| DNMT3A | c.2172dupC | | | | | | | | | |
| DNMT3A | c.2193_2195delCTT | | | | | | | | | |
| DNMT3A | c.2206C > T | | | | | | | | | |
| DNMT3A | c.2210T > G | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alpha-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| BRAF | c.1332G > A | | | | | 1 | | | | | | | |
| BRAF | c.1405G > C | | | | | 1 | | | | | | | |
| BRAF | c.1454T > G | | | | | 1 | | | | | | | |
| BRAF | c.1742A > G | | | | | 1 | | | | | | | |
| BRAF | c.1781A > G | | | | | 1 | | | | | | | |
| BRAF | c.1782T > A | | | | | | | | | | | | |
| BRAF | c.1799T > A | 1 | 2 | | | 1 | | | | | | | |
| CDKN2A | c.371G > C | | | | | | | | | | | | |
| CEBPA | c.129delC | | | | | | | | | | | | |
| CEBPA | c.413A > T | | | | | | | | | | | | |
| CEBPA | c.566C > A | | | | | | | | | | | | |
| CEBPA | c.568T > C | | | | | | | | | | | | |
| CEBPA | c.68dupC | | | | | | | | | | | | |
| CEBPA | c.690G > T | | | | | | | | | | | | |
| CEBPA | c.724G > A | | | | | | | | | | | | |
| CEBPA | c.952_955delACCA | | | | | | | | | | | | |
| CEBPA | c.952A > T | | | | | | | | | | | | |
| CEBPA | c.953_956delCCAG | | | | | | | | | | | | |
| CEBPA | c.971T > C | | | | | | | | | | | | |
| CSF3R | c.1853C > T | | | | | | | | | | | | |
| CSF3R | c.2041 − | | | | | | | | | | | | |
| | 8C > T | | | | | | | | | | | | |
| CSF3R | c.2087T > C | | | | | | | | | | | | |
| CSF3R | c.2326C > T | | | | | | | | | | | | |
| CSF3R | c.2503G > A | | | | | | | | | | | | |
| DNMT3A | c.1155G > A | | | | 1 | | | | | | | | |
| DNMT3A | c.1204C > T | | | | 1 | | | | | | | | |
| DNMT3A | c.1502A > G | | | | 1 | | | | | | | | |
| DNMT3A | c.1540delT | | | | 1 | | | | | | | | |
| DNMT3A | c.1668G > T | | | | | | | | | | | | |
| DNMT3A | c.1904G > A | | | | 1 | | | | | | | | |
| DNMT3A | c.1907T > A | | | | | | | | | | | | |
| DNMT3A | c.1924G > A | | | | | | | | | | | | |
| DNMT3A | c.1937 − | | | | 1 | | | | | | | | |
| | 10G > T | | | | | | | | | | | | |
| DNMT3A | c.2007dupC | | | | 1 | | | | | | | | |
| DNMT3A | c.2084T > G | | | | 1 | | | | | | | | |
| DNMT3A | c.2141C > T | | | | | | | | | | | | |
| DNMT3A | c.2172dupC | | | | | | | | | | | | |
| DNMT3A | c.2193_2195delCTT | | | | 1 | | | | | | | | |
| DNMT3A | c.2206C > T | | | | 1 | | | | | | | | |
| DNMT3A | c.2210T > G | | | | | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| BRAF | c.1332G > A | | 1 | 3 | | | | | | | 3 | |
| BRAF | c.1405G > C | | 1 | 3 | | | | | | | 3 | |
| BRAF | c.1454T > G | | 1 | 3 | | | | | | | 3 | |
| BRAF | c.1742A > G | | 1 | 3 | | | | | | | 3 | |
| BRAF | c.1781A > G | | 1 | 3 | | | | | | | 3 | |
| BRAF | c.1782T > A | | 1 | 3 | | | | | | | 3 | |
| BRAF | c.1799T > A | | 1 | 3 | | | | | | | 3 | |
| CDKN2A | c.371G > C | | | | | | | | | | | |
| CEBPA | c.129delC | 1 | | | | | | | | | | |
| CEBPA | c.413A > T | 1 | | | | | | | | | | |
| CEBPA | c.566C > A | 1 | | | | | | | | | | |
| CEBPA | c.568T > C | 1 | | | | | | | | | | |
| CEBPA | c.68dupC | 1 | | | | | | | | | | |
| CEBPA | c.690G > T | 1 | | | | | | | | | | |
| CEBPA | c.724G > A | 1 | | | | | | | | | | |
| CEBPA | c.952_955delACCA | 1 | | | | | | | | | | |
| CEBPA | c.952A > T | 1 | | | | | | | | | | |
| CEBPA | c.953_956delCCAG | 1 | | | | | | | | | | |
| CEBPA | c.971T > C | 1 | | | | | | | | | | |
| CSF3R | c.1853C > T | | | | | | | | | | | |
| CSF3R | c.2041 − | | | | | | | | | | | |
| | 8C > T | | | | | | | | | | | |
| CSF3R | c.2087T > C | | | | | | | | | | | |
| CSF3R | c.2326C > T | | | | | | | | | | | |
| CSF3R | c.2503G > A | | | | | | | | | | | |
| DNMT3A | c.1155G > A | | | | | | | | | | | |
| DNMT3A | c.1204C > T | | | | | | | | | | | |
| DNMT3A | c.1502A > G | | | | | | | | | | | |
| DNMT3A | c.1540delT | | | | | | | | | | | |
| DNMT3A | c.1668G > T | | | | | | | | | | | |
| DNMT3A | c.1904G > A | | | | | | | | | | | |
| DNMT3A | c.1907T > A | | | | | | | | | | | |
| DNMT3A | c.1924G > A | | | | | | | | | | | |
| DNMT3A | c.1937 − | | | | | | | | | | | |
| | 10G > T | | | | | | | | | | | |
| DNMT3A | c.2007dupC | | | | | | | | | | | |
| DNMT3A | c.2084T > G | | | | | | | | | | | |
| DNMT3A | c.2141C > T | | | | | | | | | | | |
| DNMT3A | c.2172dupC | | | | | | | | | | | |
| DNMT3A | c.2193_2195delCTT | | | | | | | | | | | |
| DNMT3A | c.2206C > T | | | | | | | | | | | |
| DNMT3A | c.2210T > G | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| BRAF | c.1332G > A | | | | | | 1 | 2 | 1 | | | |
| BRAF | c.1405G > C | | | | | | 1 | 2 | 1 | | | |
| BRAF | c.1454T > G | | | | | | 1 | 2 | 1 | | | |
| BRAF | c.1742A > G | | | | | | 1 | 2 | 1 | | | |
| BRAF | c.1781A > G | | | | | | 1 | 2 | 1 | | | |
| BRAF | c.1782T > A | | | | | | 1 | 2 | 1 | | | |
| BRAF | c.1799T > A | | | | 4 | | 1 | 5 | 2 | | | |
| CDKN2A | c.371G > C | | | | | | | | | | | |
| CEBPA | c.129delC | | | | | | | | | | | |
| CEBPA | c.413A > T | | | | | | | | | | | |
| CEBPA | c.566C > A | | | | | | | | | | | |
| CEBPA | c.568T > C | | | | | | | | | | | |
| CEBPA | c.68dupC | | | | | | | | | | | |
| CEBPA | c.690G > T | | | | | | | | | | | |
| CEBPA | c.724G > A | | | | | | | | | | | |
| CEBPA | c.952_955delACCA | | | | | | | | | | | |
| CEBPA | c.952A > T | | | | | | | | | | | |
| CEBPA | c.953_956delCCAG | | | | | | | | | | | |
| CEBPA | c.971T > C | | | | | | | | | | | |
| CSF3R | c.1853C > T | | | | | | | | | | | |
| CSF3R | c.2041 − | | | | | | | | | | | |
| | 8C > T | | | | | | | | | | | |
| CSF3R | c.2087T > C | | | | | | | | | | | |
| CSF3R | c.2326C > T | | | | | | | | | | | |
| CSF3R | c.2503G > A | | | | | | | | | | | |
| DNMT3A | c.1155G > A | | | | | | | | | | | |
| DNMT3A | c.1204C > T | | | | | | | | | | | |
| DNMT3A | c.1502A > G | | | | | | | | | | | |
| DNMT3A | c.1540delT | | | | | | | | | | | |
| DNMT3A | c.1668G > T | | | | | | | | | | | |
| DNMT3A | c.1904G > A | | | | | | | | | | | |
| DNMT3A | c.1907T > A | | | | | | | | | | | |
| DNMT3A | c.1924G > A | | | | | | | | | | | |
| DNMT3A | c.1937 − | | | | | | | | | | | |
| | 10G > T | | | | | | | | | | | |
| DNMT3A | c.2007dupC | | | | | | | | | | | |
| DNMT3A | c.2084T > G | | | | | | | | | | | |
| DNMT3A | c.2141C > T | | | | | | | | | | | |
| DNMT3A | c.2172dupC | | | | | | | | | | | |
| DNMT3A | c.2193_2195delCTT | | | | | | | | | | | |
| DNMT3A | c.2206C > T | | | | | | | | | | | |
| DNMT3A | c.2210T > G | | | | | | | | | | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| DNMT3A | c.2261T > C | | | | | | | | | | | | | | | |
| DNMT3A | c.2341G > T | | | | | | | | | | | | | | | |
| DNMT3A | c.2446C > T | | | | | | | | | | | | | | | |
| DNMT3A | c.2478G > C | | | | | | | | | | | | | | | |
| DNMT3A | c.2507G > C | | | | | | | | | | | | | | | |
| DNMT3A | c.2598 − | | | | | | | | | | | | | | | |
| | 7C > T | | | | | | | | | | | | | | | |
| DNMT3A | c.2644C > T | | | | | | | | | | | | | | | |
| DNMT3A | c.2645G > A | | | | | | | | | | | | | | | |
| DNMT3A | c.2662C > G | | | | | | | | | | | | | | | |
| DNMT3A | c.2677T > C | | | | | | | | | | | | | | | |
| DNMT3A | c.2696G > A | | | | | | | | | | | | | | | |
| DNMT3A | c.2702T > G | | | | | | | | | | | | | | | |
| DNMT3A | c.2732G > A | | | | | | | | | | | | | | | |
| DNMT3A | c.559A > G | | | | | | | | | | | | | | | |
| DNMT3A | c.639 + | | | | | | | | | | | | | | | |
| | 6G > C | | | | | | | | | | | | | | | |
| DNMT3A | c.745C > T | | | | | | | | | | | | | | | |
| DNMT3A | c.918G > T | | | | | | | | | | | | | | | |
| DNMT3A | c.939G > A | | | | | | | | | | | | | | | |
| DNMT3A | c.990G > A | | | | | | | | | | | | | | | |
| DNMT3A | c.998delA | | | | | | | | | | | | | | | |
| EZH2 | c.118 − | | | | | | | | | | | | | | | |
| | 3A > T | | | | | | | | | | | | | | | |
| EZH2 | c.1322A > G | | | | | | | | | | | | | | | |
| EZH2 | c.1574A > G | | | | | | | | | | | | | | | |
| EZH2 | c.1673 − | | | | | | | | | | | | | | | |
| | 5T > A | | | | | | | | | | | | | | | |
| EZH2 | c.1721A > T | | | | | | | | | | | | | | | |
| EZH2 | c.1865C > T | | | | | | | | | | | | | | | |
| EZH2 | c.1967C > T | | | | | | | | | | | | | | | |
| EZH2 | c.1A > T | | | | | | | | | | | | | | | |
| EZH2 | c.2035G > A | | | | | | | | | | | | | | | |
| EZH2 | c.2050C > G | | | | | | | | | | | | | | | |
| EZH2 | c.2068C > T | | | | | | | | | | | | | | | |
| EZH2 | c.2084C > T | | | | | | | | | | | | | | | |
| EZH2 | c.2110 + | | | | | | | | | | | | | | | |
| | 1G > A | | | | | | | | | | | | | | | |
| EZH2 | c.2187dupT | | | | | | | | | | | | | | | |
| EZH2 | c.2196 − | | | | | | | | | | | | | | | |
| | 1G > C | | | | | | | | | | | | | | | |
| EZH2 | c.434T > C | | | | | | | | | | | | | | | |
| EZH2 | c.449T > C | | | | | | | | | | | | | | | |
| EZH2 | c.455delA | | | | | | | | | | | | | | | |
| EZH2 | c.458A > G | | | | | | | | | | | | | | | |
| EZH2 | c.538C > T | | | | | | | | | | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| DNMT3A | c.2261T > C | | | | | | | | | | | | | | |
| DNMT3A | c.2341G > T | | | | | | | | | | | | | | |
| DNMT3A | c.2446C > T | | | | | | | | | | | | | | |
| DNMT3A | c.2478G > C | | | | | | | | | | | | | | |
| DNMT3A | c.2507G > C | | | | | | | | | | | | | | |
| DNMT3A | c.2598 − | | | | | | | | | | | | | | |
| | 7C > T | | | | | | | | | | | | | | |
| DNMT3A | c.2644C > T | | | | | | | | | | | | | | |
| DNMT3A | c.2645G > A | | | | | | | | | | | | | | |
| DNMT3A | c.2662C > G | | | | | | | | | | | | | | |
| DNMT3A | c.2677T > C | | | | | | | | | | | | | | |
| DNMT3A | c.2696G > A | | | | | | | | | | | | | | |
| DNMT3A | c.2702T > G | | | | | | | | | | | | | | |
| DNMT3A | c.2732G > A | | | | | | | | | | | | | | |
| DNMT3A | c.559A > G | | | | | | | | | | | | | | |
| DNMT3A | c.639 + | | | | | | | | | | | | | | |
| | 6G > C | | | | | | | | | | | | | | |
| DNMT3A | c.745C > T | | | | | | | | | | | | | | |
| DNMT3A | c.918G > T | | | | | | | | | | | | | | |
| DNMT3A | c.939G > A | | | | | | | | | | | | | | |
| DNMT3A | c.990G > A | | | | | | | | | | | | | | |
| DNMT3A | c.998delA | | | | | | | | | | | | | | |
| EZH2 | c.118 − | | | | | | | | | | | | | | |
| | 3A > T | | | | | | | | | | | | | | |
| EZH2 | c.1322A > G | | | | | | | | | | | | | | |
| EZH2 | c.1574A > G | | | | | | | | | | | | | | |
| EZH2 | c.1673 − | | | | | | | | | | | | | | |
| | 5T > A | | | | | | | | | | | | | | |
| EZH2 | c.1721A > T | | | | | | | | | | | | | | |
| EZH2 | c.1865C > T | | | | | | | | | | | | | | |
| EZH2 | c.1967C > T | | | | | | | | | | | | | | |
| EZH2 | c.1A > T | | | | | | | | | | | | | | |
| EZH2 | c.2035G > A | | | | | | | | | | | | | | |
| EZH2 | c.2050C > G | | | | | | | | | | | | | | |
| EZH2 | c.2068C > T | | | | | | | | | | | | | | |
| EZH2 | c.2084C > T | | | | | | | | | | | | | | |
| EZH2 | c.2110 + | | | | | | | | | | | | | | |
| | 1G > A | | | | | | | | | | | | | | |
| EZH2 | c.2187dupT | | | | | | | | | | | | | | |
| EZH2 | c.2196 − | | | | | | | | | | | | | | |
| | 1G > C | | | | | | | | | | | | | | |
| EZH2 | c.434T > C | | | | | | | | | | | | | | |
| EZH2 | c.449T > C | | | | | | | | | | | | | | |
| EZH2 | c.455delA | | | | | | | | | | | | | | |
| EZH2 | c.458A > G | | | | | | | | | | | | | | |
| EZH2 | c.538C > T | | | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| DNMT3A | c.2261T > C | | | | | | | | | | | | | |
| DNMT3A | c.2341G > T | | | | | | | | | | | | | |
| DNMT3A | c.2446C > T | | | | | | | | | | | | | |
| DNMT3A | c.2478G > C | | | | | | | | | | | | | |
| DNMT3A | c.2507G > C | | | | | | | | | | | | | |
| DNMT3A | c.2598 − | | | | | | | | | | | | | |
| | 7C > T | | | | | | | | | | | | | |
| DNMT3A | c.2644C > T | | | | | | | | | | | | | |
| DNMT3A | c.2645G > A | | | | | | | | | | | | | |
| DNMT3A | c.2662C > G | | | | | | | | | | | | | |
| DNMT3A | c.2677T > C | | | | | | | | | | | | | |
| DNMT3A | c.2696G > A | | | | | | | | | | | | | |
| DNMT3A | c.2702T > G | | | | | | | | | | | | | |
| DNMT3A | c.2732G > A | | | | | | | | | | | | | |
| DNMT3A | c.559A > G | | | | | | | | | | | | | |
| DNMT3A | c.639 + | | | | | | | | | | | | | |
| | 6G > C | | | | | | | | | | | | | |
| DNMT3A | c.745C > T | | | | | | | | | | | | | |
| DNMT3A | c.918G > T | | | | | | | | | | | | | |
| DNMT3A | c.939G > A | | | | | | | | | | | | | |
| DNMT3A | c.990G > A | | | | | | | | | | | | | |
| DNMT3A | c.998delA | | | | | | | | | | | | | |
| EZH2 | c.118 − | | | | | | | | | | | | | |
| | 3A > T | | | | | | | | | | | | | |
| EZH2 | c.1322A > G | | | | | | | | | | | | | |
| EZH2 | c.1574A > G | | | | | | | | | | | | | |
| EZH2 | c.1673 − | | | | | | | | | | | | | |
| | 5T > A | | | | | | | | | | | | | |
| EZH2 | c.1721A > T | | | | | | | | | | | | | |
| EZH2 | c.1865C > T | | | | | | | | | | | | | |
| EZH2 | c.1967C > T | | | | | | | | | | | | | |
| EZH2 | c.1A > T | | | | | | | | | | | | | |
| EZH2 | c.2035G > A | | | | | | | | | | | | | |
| EZH2 | c.2050C > G | | | | | | | | | | | | | |
| EZH2 | c.2068C > T | | | | | | | | | | | | | |
| EZH2 | c.2084C > T | | | | | | | | | | | | | |
| EZH2 | c.2110 + | | | | | | | | | | | | | |
| | 1G > A | | | | | | | | | | | | | |
| EZH2 | c.2187dupT | | | | | | | | | | | | | |
| EZH2 | c.2196 − | | | | | | | | | | | | | |
| | 1G > C | | | | | | | | | | | | | |
| EZH2 | c.434T > C | | | | | | | | | | | | | |
| EZH2 | c.449T > C | | | | | | | | | | | | | |
| EZH2 | c.455delA | | | | | | | | | | | | | |
| EZH2 | c.458A > G | | | | | | | | | | | | | |
| EZH2 | c.538C > T | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| DNMT3A | c.2261T > C | | | | | | | | | | | |
| DNMT3A | c.2341G > T | | | | | | | | | | | |
| DNMT3A | c.2446C > T | | | | | | | | | | | |
| DNMT3A | c.2478G > C | | | | | | | | | | | |
| DNMT3A | c.2507G > C | | | | | | | | | | | |
| DNMT3A | c.2598 − | | | | | | | | | | | |
| | 7C > T | | | | | | | | | | | |
| DNMT3A | c.2644C > T | | | | | | | | | | | |
| DNMT3A | c.2645G > A | | | | | | | | | | | |
| DNMT3A | c.2662C > G | | | | | | | | | | | |
| DNMT3A | c.2677T > C | | | | | | | | | | | |
| DNMT3A | c.2696G > A | | | | | | | | | | | |
| DNMT3A | c.2702T > G | | | | | | | | | | | |
| DNMT3A | c.2732G > A | | | | | | | | | | | |
| DNMT3A | c.559A > G | | | | | | | | | | | |
| DNMT3A | c.639 + | | | | | | | | | | | |
| | 6G > C | | | | | | | | | | | |
| DNMT3A | c.745C > T | | | | | | | | | | | |
| DNMT3A | c.918G > T | | | | | | | | | | | |
| DNMT3A | c.939G > A | | | | | | | | | | | |
| DNMT3A | c.990G > A | | | | | | | | | | | |
| DNMT3A | c.998delA | | | | | | | | | | | |
| EZH2 | c.118 − | | | | | | | | | | | |
| | 3A > T | | | | | | | | | | | |
| EZH2 | c.1322A > G | | | | | | | | | | | |
| EZH2 | c.1574A > G | | | | | | | | | | | |
| EZH2 | c.1673 − | | | | | | | | | | | |
| | 5T > A | | | | | | | | | | | |
| EZH2 | c.1721A > T | | | | | | | | | | | |
| EZH2 | c.1865C > T | | | | | | | | | | | |
| EZH2 | c.1967C > T | | | | | | | | | | | |
| EZH2 | c.1A > T | | | | | | | | | | | |
| EZH2 | c.2035G > A | | | | | | | | | | | |
| EZH2 | c.2050C > G | | | | | | | | | | | |
| EZH2 | c.2068C > T | | | | | | | | | | | |
| EZH2 | c.2084C > T | | | | | | | | | | | |
| EZH2 | c.2110 + | | | | | | | | | | | |
| | 1G > A | | | | | | | | | | | |
| EZH2 | c.2187dupT | | | | | | | | | | | |
| EZH2 | c.2196 − | | | | | | | | | | | |
| | 1G > C | | | | | | | | | | | |
| EZH2 | c.434T > C | | | | | | | | | | | |
| EZH2 | c.449T > C | | | | | | | | | | | |
| EZH2 | c.455delA | | | | | | | | | | | |
| EZH2 | c.458A > G | | | | | | | | | | | |
| EZH2 | c.538C > T | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| DNMT3A | c.2261T > C | 1 | | | | | | | | 1 | | 2 | | |
| DNMT3A | c.2341G > T | 1 | | | | | | | | 1 | | 2 | | |
| DNMT3A | c.2446C > T | 1 | | | | | | | | 1 | | 2 | | |
| DNMT3A | c.2478G > C | 1 | | | | | | | | 1 | | 2 | | |
| DNMT3A | c.2507G > C | 1 | | | | | | | | 1 | | 2 | | |
| DNMT3A | c.2598 − | 1 | | | | | | | | 1 | | 2 | | |
| | 7C > T | | | | | | | | | | | | | |
| DNMT3A | c.2644C > T | 1 | | | | | | | | 1 | | 2 | | |
| DNMT3A | c.2645G > A | | | | | | | | | 1 | | 2 | | |
| DNMT3A | c.2662C > G | 1 | | | | | | | | 1 | | 2 | | |
| DNMT3A | c.2677T > C | | | | | | | | | 1 | | 2 | | |
| DNMT3A | c.2696G > A | 1 | | | | | | | | 1 | | 2 | | |
| DNMT3A | c.2702T > G | | | | | | | | | 1 | | 2 | | |
| DNMT3A | c.2732G > A | 1 | | | | | | | | 1 | | 2 | | |
| DNMT3A | c.559A > G | 1 | | | | | | | | 1 | | 2 | | |
| DNMT3A | c.639 + | 1 | | | | | | | | 1 | | 2 | | |
| | 6G > C | | | | | | | | | | | | | |
| DNMT3A | c.745C > T | 1 | | | | | | | | 1 | | 2 | | |
| DNMT3A | c.918G > T | | | | | | | | | 1 | | 2 | | |
| DNMT3A | c.939G > A | | | | | | | | | 1 | | 2 | | |
| DNMT3A | c.990G > A | | | | | | | | | 1 | | 2 | | |
| DNMT3A | c.998delA | 1 | | | | | | | | 1 | | 2 | | |
| EZH2 | c.118 − | | | | | | | | | | | | | |
| | 3A > T | | | | | | | | | | | | | |
| EZH2 | c.1322A > G | | | | | | | | | | | | | |
| EZH2 | c.1574A > G | | | | | | | | | | | | | |
| EZH2 | c.1673 − | | | | | | | | | | | | | |
| | 5T > A | | | | | | | | | | | | | |
| EZH2 | c.1721A > T | | | | | | | | | | | | | |
| EZH2 | c.1865C > T | | | | | | | | | | | | | |
| EZH2 | c.1967C > T | | | | | | | | | | | | | |
| EZH2 | c.1A > T | | | | | | | | | | | | | |
| EZH2 | c.2035G > A | | | | | | | | | | | | | |
| EZH2 | c.2050C > G | | | | | | | | | | | | | |
| EZH2 | c.2068C > T | | | | | | | | | | | | | |
| EZH2 | c.2084C > T | | | | | | | | | | | | | |
| EZH2 | c.2110 + | | | | | | | | | | | | | |
| | 1G > A | | | | | | | | | | | | | |
| EZH2 | c.2187dupT | | | | | | | | | | | | | |
| EZH2 | c.2196 − | | | | | | | | | | | | | |
| | 1G > C | | | | | | | | | | | | | |
| EZH2 | c.434T > C | | | | | | | | | | | | | |
| EZH2 | c.449T > C | | | | | | | | | | | | | |
| EZH2 | c.455delA | | | | | | | | | | | | | |
| EZH2 | c.458A > G | | | | | | | | | | | | | |
| EZH2 | c.538C > T | | | | | | | | | | | | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| DNMT3A | c.2261T > C | | | | | | | | | | | |
| DNMT3A | c.2341G > T | | | | | | | | | | | |
| DNMT3A | c.2446C > T | | | | | | | | | | | |
| DNMT3A | c.2478G > C | | | | | | | | | | | |
| DNMT3A | c.2507G > C | | | | | | | | | | | |
| DNMT3A | c.2598 − | | | | | | | | | | | |
| | 7C > T | | | | | | | | | | | |
| DNMT3A | c.2644C > T | | | | | | | | | | | |
| DNMT3A | c.2645G > A | | | | | | | | | | | |
| DNMT3A | c.2662C > G | | | | | | | | | | | |
| DNMT3A | c.2677T > C | | | | | | | | | | | |
| DNMT3A | c.2696G > A | | | | | | | | | | | |
| DNMT3A | c.2702T > G | | | | | | | | | | | |
| DNMT3A | c.2732G > A | | | | | | | | | | | |
| DNMT3A | c.559A > G | | | | | | | | | | | |
| DNMT3A | c.639 + | | | | | | | | | | | |
| | 6G > C | | | | | | | | | | | |
| DNMT3A | c.745C > T | | | | | | | | | | | |
| DNMT3A | c.918G > T | | | | | | | | | | | |
| DNMT3A | c.939G > A | | | | | | | | | | | |
| DNMT3A | c.990G > A | | | | | | | | | | | |
| DNMT3A | c.998delA | | | | | | | | | | | |
| EZH2 | c.118 − | | | | | | | | | | | |
| | 3A > T | | | | | | | | | | | |
| EZH2 | c.1322A > G | | | | | | | | | | | |
| EZH2 | c.1574A > G | | | | | | | | | | | |
| EZH2 | c.1673 − | | | | | | | | | | | |
| | 5T > A | | | | | | | | | | | |
| EZH2 | c.1721A > T | | | | | | | | | | | |
| EZH2 | c.1865C > T | | | | | | | | | | | |
| EZH2 | c.1967C > T | | | | | | | | | | | |
| EZH2 | c.1A > T | | | | | | | | | | | |
| EZH2 | c.2035G > A | | | | | | | | | | | |
| EZH2 | c.2050C > G | | | | | | | | | | | |
| EZH2 | c.2068C > T | | | | | | | | | | | |
| EZH2 | c.2084C > T | | | | | | | | | | | |
| EZH2 | c.2110 + | | | | | | | | | | | |
| | 1G > A | | | | | | | | | | | |
| EZH2 | c.2187dupT | | | | | | | | | | | |
| EZH2 | c.2196 − | | | | | | | | | | | |
| | 1G > C | | | | | | | | | | | |
| EZH2 | c.434T > C | | | | | | | | | | | |
| EZH2 | c.449T > C | | | | | | | | | | | |
| EZH2 | c.455delA | | | | | | | | | | | |
| EZH2 | c.458A > G | | | | | | | | | | | |
| EZH2 | c.538C > T | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| DNMT3A | c.2261T > C | | | | | | | | | | | | |
| DNMT3A | c.2341G > T | | | | | | | | | | | | |
| DNMT3A | c.2446C > T | | | | | | | | | | | | |
| DNMT3A | c.2478G > C | | | | | | | | | | | | |
| DNMT3A | c.2507G > C | | | | | | | | | | | | |
| DNMT3A | c.2598 − | | | | | | | | | | | | |
| | 7C > T | | | | | | | | | | | | |
| DNMT3A | c.2644C > T | | | | | | | | | | | | |
| DNMT3A | c.2645G > A | | | | | | | | | | | | |
| DNMT3A | c.2662C > G | | | | | | | | | | | | |
| DNMT3A | c.2677T > C | | | | | | | | | | | | |
| DNMT3A | c.2696G > A | | | | | | | | | | | | |
| DNMT3A | c.2702T > G | | | | | | | | | | | | |
| DNMT3A | c.2732G > A | | | | | | | | | | | | |
| DNMT3A | c.559A > G | | | | | | | | | | | | |
| DNMT3A | c.639 + | | | | | | | | | | | | |
| | 6G > C | | | | | | | | | | | | |
| DNMT3A | c.745C > T | | | | | | | | | | | | |
| DNMT3A | c.918G > T | | | | | | | | | | | | |
| DNMT3A | c.939G > A | | | | | | | | | | | | |
| DNMT3A | c.990G > A | | | | | | | | | | | | |
| DNMT3A | c.998delA | | | | | | | | | | | | |
| EZH2 | c.118 − | | | | | | | | | | | | |
| | 3A > T | | | | | | | | | | | | |
| EZH2 | c.1322A > G | | | | | | | | | | | | |
| EZH2 | c.1574A > G | | | | | | | | | | | | |
| EZH2 | c.1673 − | | | | | | | | | | | | |
| | 5T > A | | | | | | | | | | | | |
| EZH2 | c.1721A > T | | | | | | | | | | | | |
| EZH2 | c.1865C > T | | | | | | | | | | | | |
| EZH2 | c.1967C > T | | | | | | | | | | | | |
| EZH2 | c.1A > T | | | | | | | | | | | | |
| EZH2 | c.2035G > A | | | | | | | | | | | | |
| EZH2 | c.2050C > G | | | | | | | | | | | | |
| EZH2 | c.2068C > T | | | | | | | | | | | | |
| EZH2 | c.2084C > T | | | | | | | | | | | | |
| EZH2 | c.2110 + | | | | | | | | | | | | |
| | 1G > A | | | | | | | | | | | | |
| EZH2 | c.2187dupT | | | | | | | | | | | | |
| EZH2 | c.2196 − | | | | | | | | | | | | |
| | 1G > C | | | | | | | | | | | | |
| EZH2 | c.434T > C | | | | | | | | | | | | |
| EZH2 | c.449T > C | | | | | | | | | | | | |
| EZH2 | c.455delA | | | | | | | | | | | | |
| EZH2 | c.458A > G | | | | | | | | | | | | |
| EZH2 | c.538C > T | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | methylpred- | | |
| | | lenalidomide | | rituximab | lenvatinib | lorlatinib | inhibitor | methotrexate | nisolone | midostaurin | mitoxantrone |
| Gene | Variant | ✓ | X | ✓ | ✓ | X | ✓ | X | X | ✓ | X |
| DNMT3A | c.2261T > C | | | | | | | | | | |
| DNMT3A | c.2341G > T | | | | | | | | | | |
| DNMT3A | c.2446C > T | | | | | | | | | | |
| DNMT3A | c.2478G > C | | | | | | | | | | |
| DNMT3A | c.2507G > C | | | | | | | | | | |
| DNMT3A | c.2598 − | | | | | | | | | | |
| | 7C > T | | | | | | | | | | |
| DNMT3A | c.2644C > T | | | | | | | | | | |
| DNMT3A | c.2645G > A | | | | | | | | | | |
| DNMT3A | c.2662C > G | | | | | | | | | | |
| DNMT3A | c.2677T > C | | | | | | | | | | |
| DNMT3A | c.2696G > A | | | | | | | | | | |
| DNMT3A | c.2702T > G | | | | | | | | | | |
| DNMT3A | c.2732G > A | | | | | | | | | | |
| DNMT3A | c.559A > G | | | | | | | | | | |
| DNMT3A | c.639 + | | | | | | | | | | |
| | 6G > C | | | | | | | | | | |
| DNMT3A | c.745C > T | | | | | | | | | | |
| DNMT3A | c.918G > T | | | | | | | | | | |
| DNMT3A | c.939G > A | | | | | | | | | | |
| DNMT3A | c.990G > A | | | | | | | | | | |
| DNMT3A | c.998delA | | | | | | | | | | |
| EZH2 | c.118 − | | | | | | | | | | |
| | 3A > T | | | | | | | | | | |
| EZH2 | c.1322A > G | | | | | | | | | | |
| EZH2 | c.1574A > G | | | | | | | | | | |
| EZH2 | c.1673 − | | | | | | | | | | |
| | 5T > A | | | | | | | | | | |
| EZH2 | c.1721A > T | | | | | | | | | | |
| EZH2 | c.1865C > T | | | | | | | | | | |
| EZH2 | c.1967C > T | | | | | | | | | | |
| EZH2 | c.1A > T | | | | | | | | | | |
| EZH2 | c.2035G > A | | | | | | | | | | |
| EZH2 | c.2050C > G | | | | | | | | | | |
| EZH2 | c.2068C > T | | | | | | | | | | |
| EZH2 | c.2084C > T | | | | | | | | | | |
| EZH2 | c.2110 + | | | | | | | | | | |
| | 1G > A | | | | | | | | | | |
| EZH2 | c.2187dupT | | | | | | | | | | |
| EZH2 | c.2196 − | | | | | | | | | | |
| | 1G > C | | | | | | | | | | |
| EZH2 | c.434T > C | | | | | | | | | | |
| EZH2 | c.449T > C | | | | | | | | | | |
| EZH2 | c.455delA | | | | | | | | | | |
| EZH2 | c.458A > G | | | | | | | | | | |
| EZH2 | c.538C > T | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| DNMT3A | c.2261T > C | | | | | | | | | |
| DNMT3A | c.2341G > T | | | | | | | | | |
| DNMT3A | c.2446C > T | | | | | | | | | |
| DNMT3A | c.2478G > C | | | | | | | | | |
| DNMT3A | c.2507G > C | | | | | | | | | |
| DNMT3A | c.2598 − | | | | | | | | | |
| | 7C > T | | | | | | | | | |
| DNMT3A | c.2644C > T | | | | | | | | | |
| DNMT3A | c.2645G > A | | | | | | | | | |
| DNMT3A | c.2662C > G | | | | | | | | | |
| DNMT3A | c.2677T > C | | | | | | | | | |
| DNMT3A | c.2696G > A | | | | | | | | | |
| DNMT3A | c.2702T > G | | | | | | | | | |
| DNMT3A | c.2732G > A | | | | | | | | | |
| DNMT3A | c.559A > G | | | | | | | | | |
| DNMT3A | c.639 + | | | | | | | | | |
| | 6G > C | | | | | | | | | |
| DNMT3A | c.745C > T | | | | | | | | | |
| DNMT3A | c.918G > T | | | | | | | | | |
| DNMT3A | c.939G > A | | | | | | | | | |
| DNMT3A | c.990G > A | | | | | | | | | |
| DNMT3A | c.998delA | | | | | | | | | |
| EZH2 | c.118 − | | | | | | | | | |
| | 3A > T | | | | | | | | | |
| EZH2 | c.1322A > G | | | | | | | | | |
| EZH2 | c.1574A > G | | | | | | | | | |
| EZH2 | c.1673 − | | | | | | | | | |
| | 5T > A | | | | | | | | | |
| EZH2 | c.1721A > T | | | | | | | | | |
| EZH2 | c.1865C > T | | | | | | | | | |
| EZH2 | c.1967C > T | | | | | | | | | |
| EZH2 | c.1A > T | | | | | | | | | |
| EZH2 | c.2035G > A | | | | | | | | | |
| EZH2 | c.2050C > G | | | | | | | | | |
| EZH2 | c.2068C > T | | | | | | | | | |
| EZH2 | c.2084C > T | | | | | | | | | |
| EZH2 | c.2110 + | | | | | | | | | |
| | 1G > A | | | | | | | | | |
| EZH2 | c.2187dupT | | | | | | | | | |
| EZH2 | c.2196 − | | | | | | | | | |
| | 1G > C | | | | | | | | | |
| EZH2 | c.434T > C | | | | | | | | | |
| EZH2 | c.449T > C | | | | | | | | | |
| EZH2 | c.455delA | | | | | | | | | |
| EZH2 | c.458A > G | | | | | | | | | |
| EZH2 | c.538C > T | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alpha-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| DNMT3A | c.2261T > C | | | | 1 | | | | | | | | |
| DNMT3A | c.2341G > T | | | | 1 | | | | | | | | |
| DNMT3A | c.2446C > T | | | | 1 | | | | | | | | |
| DNMT3A | c.2478G > C | | | | 1 | | | | | | | | |
| DNMT3A | c.2507G > C | | | | 1 | | | | | | | | |
| DNMT3A | c.2598 − | | | | 1 | | | | | | | | |
| | 7C > T | | | | | | | | | | | | |
| DNMT3A | c.2644C > T | | | | 1 | | | | | | | | |
| DNMT3A | c.2645G > A | | | | | | | | | | | | |
| DNMT3A | c.2662C > G | | | | 1 | | | | | | | | |
| DNMT3A | c.2677T > C | | | | | | | | | | | | |
| DNMT3A | c.2696G > A | | | | 1 | | | | | | | | |
| DNMT3A | c.2702T > G | | | | | | | | | | | | |
| DNMT3A | c.2732G > A | | | | 1 | | | | | | | | |
| DNMT3A | c.559A > G | | | | 1 | | | | | | | | |
| DNMT3A | c.639 + | | | | 1 | | | | | | | | |
| | 6G > C | | | | | | | | | | | | |
| DNMT3A | c.745C > T | | | | 1 | | | | | | | | |
| DNMT3A | c.918G > T | | | | | | | | | | | | |
| DNMT3A | c.939G > A | | | | | | | | | | | | |
| DNMT3A | c.990G > A | | | | | | | | | | | | |
| DNMT3A | c.998delA | | | | 1 | | | | | | | | |
| EZH2 | c.118 − | | | | 1 | | | | | | | | |
| | 3A > T | | | | | | | | | | | | |
| EZH2 | c.1322A > G | | | | 1 | | | | | | | | |
| EZH2 | c.1574A > G | | | | 1 | | | | | | | | |
| EZH2 | c.1673 − | | | | 1 | | | | | | | | |
| | 5T > A | | | | | | | | | | | | |
| EZH2 | c.1721A > T | | | | 1 | | | | | | | | |
| EZH2 | c.1865C > T | | | | 1 | | | | | | | | |
| EZH2 | c.1967C > T | | | | 1 | | | | | | | | |
| EZH2 | c.1A > T | | | | 1 | | | | | | | | |
| EZH2 | c.2035G > A | | | | 1 | | | | | | | | |
| EZH2 | c.2050C > G | | | | 1 | | | | | | | | |
| EZH2 | c.2068C > T | | | | 1 | | | | | | | | |
| EZH2 | c.2084C > T | | | | 1 | | | | | | | | |
| EZH2 | c.2110 + | | | | 1 | | | | | | | | |
| | 1G > A | | | | | | | | | | | | |
| EZH2 | c.2187dupT | | | | 1 | | | | | | | | |
| EZH2 | c.2196 − | | | | 1 | | | | | | | | |
| | 1G > C | | | | | | | | | | | | |
| EZH2 | c.434T > C | | | | 1 | | | | | | | | |
| EZH2 | c.449T > C | | | | 1 | | | | | | | | |
| EZH2 | c.455delA | | | | 1 | | | | | | | | |
| EZH2 | c.458A > G | | | | 1 | | | | | | | | |
| EZH2 | c.538C > T | | | | 1 | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| DNMT3A | c.2261T > C | | | | | | | | | | | |
| DNMT3A | c.2341G > T | | | | | | | | | | | |
| DNMT3A | c.2446C > T | | | | | | | | | | | |
| DNMT3A | c.2478G > C | | | | | | | | | | | |
| DNMT3A | c.2507G > C | | | | | | | | | | | |
| DNMT3A | c.2598 − | | | | | | | | | | | |
| | 7C > T | | | | | | | | | | | |
| DNMT3A | c.2644C > T | | | | | | | | | | | |
| DNMT3A | c.2645G > A | | | | | | | | | | | |
| DNMT3A | c.2662C > G | | | | | | | | | | | |
| DNMT3A | c.2677T > C | | | | | | | | | | | |
| DNMT3A | c.2696G > A | | | | | | | | | | | |
| DNMT3A | c.2702T > G | | | | | | | | | | | |
| DNMT3A | c.2732G > A | | | | | | | | | | | |
| DNMT3A | c.559A > G | | | | | | | | | | | |
| DNMT3A | c.639 + | | | | | | | | | | | |
| | 6G > C | | | | | | | | | | | |
| DNMT3A | c.745C > T | | | | | | | | | | | |
| DNMT3A | c.918G > T | | | | | | | | | | | |
| DNMT3A | c.939G > A | | | | | | | | | | | |
| DNMT3A | c.990G > A | | | | | | | | | | | |
| DNMT3A | c.998delA | | | | | | | | | | | |
| EZH2 | c.118 − | | | | | | | | | | | |
| | 3A > T | | | | | | | | | | | |
| EZH2 | c.1322A > G | | | | | | | | | | | |
| EZH2 | c.1574A > G | | | | | | | | | | | |
| EZH2 | c.1673 − | | | | | | | | | | | |
| | 5T > A | | | | | | | | | | | |
| EZH2 | c.1721A > T | | | | | | | | | | | |
| EZH2 | c.1865C > T | | | | | | | | | | | |
| EZH2 | c.1967C > T | | | | | | | | | | | |
| EZH2 | c.1A > T | | | | | | | | | | | |
| EZH2 | c.2035G > A | | | | | | | | | | | |
| EZH2 | c.2050C > G | | | | | | | | | | | |
| EZH2 | c.2068C > T | | | | | | | | | | | |
| EZH2 | c.2084C > T | | | | | | | | | | | |
| EZH2 | c.2110 + | | | | | | | | | | | |
| | 1G > A | | | | | | | | | | | |
| EZH2 | c.2187dupT | | | | | | | | | | | |
| EZH2 | c.2196 − | | | | | | | | | | | |
| | 1G > C | | | | | | | | | | | |
| EZH2 | c.434T > C | | | | | | | | | | | |
| EZH2 | c.449T > C | | | | | | | | | | | |
| EZH2 | c.455delA | | | | | | | | | | | |
| EZH2 | c.458A > G | | | | | | | | | | | |
| EZH2 | c.538C > T | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| DNMT3A | c.2261T > C | | | | | | | | | | | |
| DNMT3A | c.2341G > T | | | | | | | | | | | |
| DNMT3A | c.2446C > T | | | | | | | | | | | |
| DNMT3A | c.2478G > C | | | | | | | | | | | |
| DNMT3A | c.2507G > C | | | | | | | | | | | |
| DNMT3A | c.2598 − | | | | | | | | | | | |
| | 7C > T | | | | | | | | | | | |
| DNMT3A | c.2644C > T | | | | | | | | | | | |
| DNMT3A | c.2645G > A | | | | | | | | | | | |
| DNMT3A | c.2662C > G | | | | | | | | | | | |
| DNMT3A | c.2677T > C | | | | | | | | | | | |
| DNMT3A | c.2696G > A | | | | | | | | | | | |
| DNMT3A | c.2702T > G | | | | | | | | | | | |
| DNMT3A | c.2732G > A | | | | | | | | | | | |
| DNMT3A | c.559A > G | | | | | | | | | | | |
| DNMT3A | c.639 + | | | | | | | | | | | |
| | 6G > C | | | | | | | | | | | |
| DNMT3A | c.745C > T | | | | | | | | | | | |
| DNMT3A | c.918G > T | | | | | | | | | | | |
| DNMT3A | c.939G > A | | | | | | | | | | | |
| DNMT3A | c.990G > A | | | | | | | | | | | |
| DNMT3A | c.998delA | | | | | | | | | | | |
| EZH2 | c.118 − | | | | | | | | | | | |
| | 3A > T | | | | | | | | | | | |
| EZH2 | c.1322A > G | | | | | | | | | | | |
| EZH2 | c.1574A > G | | | | | | | | | | | |
| EZH2 | c.1673 − | | | | | | | | | | | |
| | 5T > A | | | | | | | | | | | |
| EZH2 | c.1721A > T | | | | | | | | | | | |
| EZH2 | c.1865C > T | | | | | | | | | | | |
| EZH2 | c.1967C > T | | | | | | | | | | | |
| EZH2 | c.1A > T | | | | | | | | | | | |
| EZH2 | c.2035G > A | | | | | | | | | | | |
| EZH2 | c.2050C > G | | | | | | | | | | | |
| EZH2 | c.2068C > T | | | | | | | | | | | |
| EZH2 | c.2084C > T | | | | | | | | | | | |
| EZH2 | c.2110 + | | | | | | | | | | | |
| | 1G > A | | | | | | | | | | | |
| EZH2 | c.2187dupT | | | | | | | | | | | |
| EZH2 | c.2196 − | | | | | | | | | | | |
| | 1G > C | | | | | | | | | | | |
| EZH2 | c.434T > C | | | | | | | | | | | |
| EZH2 | c.449T > C | | | | | | | | | | | |
| EZH2 | c.455delA | | | | | | | | | | | |
| EZH2 | c.458A > G | | | | | | | | | | | |
| EZH2 | c.538C > T | | | | | | | | | | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| EZH2 | c.72dupG | | | | | | | | | | | | | | | |
| EZH2 | c.862C > T | | | | | | | | | | | | | | | |
| EZH2 | c.894dupT | | | | | | | | | | | | | | | |
| EZH2 | c.965A > G | | | | | | | | | | | | | | | |
| FLT3 | c.1359C > A | | 1 | | | | | | | | | | | | | |
| FLT3 | c.1715A > G | | 1 | | | | | | | | | | | | | |
| FLT3 | c.1765T > A | | 1 | | | | | | | | | | | | | |
| FLT3 | c.1769T > C | | 1 | | | | | | | | | | | | | |
| FLT3 | c.1836T > C | | 1 | | | | | | | | | | | | | |
| FLT3 | c.2503G > C | | 1 | | | | | | | | | | | | | |
| FLT3 | c.2503G > T | | 1 | | | | | | | | | | | | | |
| FLT3 | c.2508_2510delCAT | | 1 | | | | | | | | | | | | | |
| FLT3 | c.2510T > A | | 1 | | | | | | | | | | | | | |
| FLT3 | c.2522A > T | | 1 | | | | | | | | | | | | | |
| FLT3 | c.2524T > G | | 1 | | | | | | | | | | | | | |
| FLT3 | c.2525A > G | | 1 | | | | | | | | | | | | | |
| IDH1 | c.394C > T | | | | | | | | | | | | | | | |
| IDH1 | c.395G > A | | | | | | | | | | | | | | | |
| IDH2 | c.419G > A | | | | | | | | | | | | | | | |
| IDH2 | c.515G > A | | | | | | | | | | | | | | | |
| JAK2 | c.1541C > T | | | | | | | | | | | | | | | |
| JAK2 | c.1624_1629delAATGAA | | | | | | | | | | | | | | | |
| JAK2 | c.1710C > T | | | | | | | | | | | | | | | |
| JAK2 | c.1849G > T | | | | | | | | | | | | | | | |
| KIT | c.106C > T | | | | | | | | | | | | | | | |
| KIT | c.1647 + | | | | | | | | | | | | | | | |
| | 7A > C | | | | | | | | | | | | | | | |
| KIT | c.2394C > T | | | | | | | | | | | | | | | |
| KIT | c.2447A > T | | | | | | | | | | | | | | | |
| KIT | c.287C > T | | | | | | | | | | | | | | | |
| KMT2A | c.6698_6699delinsTT | | | | | | | | | | | | | | | |
| KRAS | c.173C > T | | | 1 | | 1 | | | 1 | 1 | | | | | | |
| KRAS | c.179G > A | | | 1 | | 1 | | | 1 | 1 | | | | | | |
| KRAS | c.182A > C | | | 1 | 1 | 1 | | | 1 | 1 | | | | | | |
| KRAS | c.182A > G | | | 1 | 1 | 1 | | | 1 | 1 | | | | | | |
| KRAS | c.183A > T | | | 1 | 1 | 1 | | | 1 | 1 | | | | | | |
| KRAS | c.350A > G | | | 1 | | 1 | | | 1 | 1 | | | | | | |
| KRAS | c.35G > A | | | 1 | | 1 | 1 | | | 1 | | | | | 1 | |
| KRAS | c.35G > C | | | 1 | | 1 | | | 1 | 1 | | | | | 1 | |
| KRAS | c.35G > T | | | 1 | | 1 | 1 | | | 1 | | | | | 1 | |
| KRAS | c.38G > A | | | 1 | | 1 | 1 | | | 1 | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| EZH2 | c.72dupG | | | | | | | | | | | | | | |
| EZH2 | c.862C > T | | | | | | | | | | | | | | |
| EZH2 | c.894dupT | | | | | | | | | | | | | | |
| EZH2 | c.965A > G | | | | | | | | | | | | | | |
| FLT3 | c.1359C > A | | | | | | | | | | | | | 1 | |
| FLT3 | c.1715A > G | | | | | | | | | | | | | 1 | |
| FLT3 | c.1765T > A | | | | | | | | | | | | | 1 | |
| FLT3 | c.1769T > C | | | | | | | | | | | | | 1 | |
| FLT3 | c.1836T > C | | | | | | | | | | | | | 1 | |
| FLT3 | c.2503G > C | | | | | | | | | | | | | 1 | |
| FLT3 | c.2503G > T | | | | | | | | | | | | | 1 | |
| FLT3 | c.2508_2510delCAT | | | | | | | | | | | | | 1 | |
| FLT3 | c.2510T > A | | | | | | | | | | | | | 1 | |
| FLT3 | c.2522A > T | | | | | | | | | | | | | 1 | |
| FLT3 | c.2524T > G | | | | | | | | | | | | | 1 | |
| FLT3 | c.2525A > G | | | | | | | | | | | | | 1 | |
| IDH1 | c.394C > T | | | | | | | | | | | | | | |
| IDH1 | c.395G > A | | | | | | | | | | | | | | |
| IDH2 | c.419G > A | | | | | | | | | | | | | | |
| IDH2 | c.515G > A | | | | | | | | | | | | | | |
| JAK2 | c.1541C > T | | | | | | | | | | | | | | |
| JAK2 | c.1624_1629delAATGAA | | | | | | | | | | | | | | |
| JAK2 | c.1710C > T | | | | | | | | | | | | | | |
| JAK2 | c.1849G > T | | | | | | | | | | | | | | |
| KIT | c.106C > T | | | | 2 | | | | | | | | | | |
| KIT | c.1647 + | | | | 2 | | | | | | | | | | |
| | 7A > C | | | | | | | | | | | | | | |
| KIT | c.2394C > T | | | | 2 | | | | | | | | | | |
| KIT | c.2447A > T | | | | 2 | | | | | | | | | | |
| KIT | c.287C > T | | | | 2 | | | | | | | | | | |
| KMT2A | c.6698_6699delinsTT | | | | | | | | | | | | | | |
| KRAS | c.173C > T | 1 | | 1 | | 2 | | | | | | | | | |
| KRAS | c.179G > A | 1 | | 1 | | 2 | | | | | | | | | |
| KRAS | c.182A > C | 1 | | 1 | | 2 | | | | | | | | | |
| KRAS | c.182A > G | 1 | | 1 | | 2 | | | | | | | | | |
| KRAS | c.183A > T | 1 | | 1 | | 2 | | | | | | | | | |
| KRAS | c.350A > G | 1 | | 1 | | 2 | | | | | | | | | |
| KRAS | c.35G > A | | | | | 2 | 1 | 1 | 1 | | | | | | |
| KRAS | c.35G > C | | | 1 | | 2 | | | | | | | | | |
| KRAS | c.35G > T | | | | | 2 | 1 | 1 | 1 | | | | | | |
| KRAS | c.38G > A | | | | | 2 | 1 | 1 | 1 | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| EZH2 | c.72dupG | | | | | | | | | | | | | |
| EZH2 | c.862C > T | | | | | | | | | | | | | |
| EZH2 | c.894dupT | | | | | | | | | | | | | |
| EZH2 | c.965A > G | | | | | | | | | | | | | |
| FLT3 | c.1359C > A | 2 | | | | | | | | | | | | |
| FLT3 | c.1715A > G | 2 | | | | | | | | | | | | |
| FLT3 | c.1765T > A | 2 | | | | | | | | | | | | |
| FLT3 | c.1769T > C | 2 | | | | | | | | | | | | |
| FLT3 | c.1836T > C | 2 | | | | | | | | | | | | |
| FLT3 | c.2503G > C | 2 | | | | | | | | | | | | |
| FLT3 | c.2503G > T | 2 | | | | | | | | | | | | |
| FLT3 | c.2508_2510delCAT | 2 | | | | | | | | | | | | |
| FLT3 | c.2510T > A | 2 | | | | | | | | | | | | |
| FLT3 | c.2522A > T | 2 | | | | | | | | | | | | |
| FLT3 | c.2524T > G | 2 | | | | | | | | | | | | |
| FLT3 | c.2525A > G | 2 | | | | | | | | | | | | |
| IDH1 | c.394C > T | | | | | | | | | | | | | |
| IDH1 | c.395G > A | | | | | | | | | | | | | |
| IDH2 | c.419G > A | | | | | | | | | | | | | |
| IDH2 | c.515G > A | | | | | | | | | | | | | |
| JAK2 | c.1541C > T | | | | | | | | | | | | | |
| JAK2 | c.1624_1629delAATGAA | | | | | | | | | | | | | |
| JAK2 | c.1710C > T | | | | | | | | | | | | | |
| JAK2 | c.1849G > T | | | | | | | | | | | | | |
| KIT | c.106C > T | 2 | | | | | | | | | | | | |
| KIT | c.1647 + | 2 | | | | | | | | | | | | |
| | 7A > C | | | | | | | | | | | | | |
| KIT | c.2394C > T | 2 | | | | | | | | | | | | |
| KIT | c.2447A > T | 2 | | | | | | | | | | | | |
| KIT | c.287C > T | 2 | | | | | | | | | | | | |
| KMT2A | c.6698_6699delinsTT | | | | | | | | | | | | | |
| KRAS | c.173C > T | | | | | | | | 1 | 3 | 1 | | | |
| KRAS | c.179G > A | | | | | | | | 1 | 3 | 1 | | | |
| KRAS | c.182A > C | | | | | | | | 1 | 4 | 1 | | | |
| KRAS | c.182A > G | | | | | | | | 1 | 4 | 1 | | | |
| KRAS | c.183A > T | | | | | | | | 1 | 4 | 1 | | | |
| KRAS | c.350A > G | | | | | | | | 1 | 4 | 1 | | | |
| KRAS | c.35G > A | | | | | 1 | 1 | | 1 | 5 | 1 | | | |
| KRAS | c.35G > C | | | | | | 1 | | 1 | 5 | 1 | | | |
| KRAS | c.35G > T | | | | | 1 | 1 | | 1 | 5 | 1 | | | |
| KRAS | c.38G > A | | | | | 1 | 1 | | 1 | 5 | 1 | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| EZH2 | c.72dupG | | | | | | | | | | | |
| EZH2 | c.862C > T | | | | | | | | | | | |
| EZH2 | c.894dupT | | | | | | | | | | | |
| EZH2 | c.965A > G | | | | | | | | | | | |
| FLT3 | c.1359C > A | | | | | | | | | | | |
| FLT3 | c.1715A > G | | | | | | | | | | | |
| FLT3 | c.1765T > A | | | | | | | | | | | |
| FLT3 | c.1769T > C | | | | | | | | | | | |
| FLT3 | c.1836T > C | | | | | | | | | | | |
| FLT3 | c.2503G > C | | | | | | | | | | | |
| FLT3 | c.2503G > T | | | | | | | | | | | |
| FLT3 | c.2508_2510delCAT | | | | | | | | | | | |
| FLT3 | c.2510T > A | | | | | | | | | | | |
| FLT3 | c.2522A > T | | | | | | | | | | | |
| FLT3 | c.2524T > G | | | | | | | | | | | |
| FLT3 | c.2525A > G | | | | | | | | | | | |
| IDH1 | c.394C > T | | | | | | | | | | | |
| IDH1 | c.395G > A | | | | | | | | | | | |
| IDH2 | c.419G > A | | | | | | | | | | | |
| IDH2 | c.515G > A | | | | | | | | | | | |
| JAK2 | c.1541C > T | | | | | | | | | | | |
| JAK2 | c.1624_1629delAATGAA | | | | | | | | | | | |
| JAK2 | c.1710C > T | | | | | | | | | | | |
| JAK2 | c.1849G > T | | | | | | | | | | | |
| KIT | c.106C > T | | | | | | | | | | | 1 |
| KIT | c.1647 + | | | | | | | | | | | 1 |
| | 7A > C | | | | | | | | | | | |
| KIT | c.2394C > T | | | | | | | | | | | 1 |
| KIT | c.2447A > T | | | | | | | 1 | | | | 1 |
| KIT | c.287C > T | | | | | | | | | | | 1 |
| KMT2A | c.6698_6699delinsTT | | | | | | | | | | | |
| KRAS | c.173C > T | | | | | | | | | | | |
| KRAS | c.179G > A | | | | | | | | | | | |
| KRAS | c.182A > C | | | | | | | | | | | |
| KRAS | c.182A > G | | | | | | | | | | | |
| KRAS | c.183A > T | | | | | | | | | | | |
| KRAS | c.350A > G | | | | | | | | | | | |
| KRAS | c.35G > A | | | | | | | 1 | | | | |
| KRAS | c.35G > C | | | | | | | 1 | | | | |
| KRAS | c.35G > T | | | | | | | 1 | | | | |
| KRAS | c.38G > A | | | | | | | 1 | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| EZH2 | c.72dupG | | | | | | | | | | | | | |
| EZH2 | c.862C > T | | | | | | | | | | | | | |
| EZH2 | c.894dupT | | | | | | | | | | | | | |
| EZH2 | c.965A > G | | | | | | | | | | | | | |
| FLT3 | c.1359C > A | 1 | | | | | | | | 1 | | | 1 | |
| FLT3 | c.1715A > G | 1 | | | | | | | | 1 | | | 1 | |
| FLT3 | c.1765T > A | | | | | | | | | 1 | | | 1 | |
| FLT3 | c.1769T > C | | | | | | | | | 1 | | | 1 | |
| FLT3 | c.1836T > C | | | | | | | | | 1 | | | 1 | |
| FLT3 | c.2503G > C | 1 | | | | | | | | 1 | | | 1 | |
| FLT3 | c.2503G > T | | | | | | | | | 1 | | | 1 | |
| FLT3 | c.2508_2510delCAT | 1 | | | | | | | | 1 | | | 1 | |
| FLT3 | c.2510T > A | 1 | | | | | | | | 1 | | | 1 | |
| FLT3 | c.2522A > T | | | | | | | | | 1 | | | 1 | |
| FLT3 | c.2524T > G | | | | | | | | | 1 | | | 1 | |
| FLT3 | c.2525A > G | 1 | | | | | | | | 1 | | | 1 | |
| IDH1 | c.394C > T | | | | | | | | | | | | | |
| IDH1 | c.395G > A | | | | | | | | | | | | | |
| IDH2 | c.419G > A | | | | | | | | | | | | | |
| IDH2 | c.515G > A | | | | | | | | | | | | | |
| JAK2 | c.1541C > T | | | | | | | | | | | | | |
| JAK2 | c.1624_1629delAATGAA | | | | | | | | | | | | | |
| JAK2 | c.1710C > T | | | | | | | | | | | | | |
| JAK2 | c.1849G > T | | | | | | | | | | | | | |
| KIT | c.106C > T | | | | | | | 2 | | | | | | |
| KIT | c.1647 + | | | | | | | 2 | | | | | | |
| | 7A > C | | | | | | | | | | | | | |
| KIT | c.2394C > T | | | | | | | 2 | | | | | | |
| KIT | c.2447A > T | | | | | | | 2 | | | | | | |
| KIT | c.287C > T | | | | | | | 2 | | | | | | |
| KMT2A | c.6698_6699delinsTT | 1 | | | | | | | | | | | | |
| KRAS | c.173C > T | | | | | | | | | | | | | |
| KRAS | c.179G > A | | | | | | | | | | | | | |
| KRAS | c.182A > C | | | 1 | | | | | | | | | | |
| KRAS | c.182A > G | | | 1 | | | | | | | | | | |
| KRAS | c.183A > T | | | 1 | | | | | | | | | | |
| KRAS | c.350A > G | | | | | | | | | | | | | |
| KRAS | c.35G > A | | | 1 | | | | | | | | | | |
| KRAS | c.35G > C | | | 1 | | | | | | | | | | |
| KRAS | c.35G > T | | | 1 | | | | | | | | | | |
| KRAS | c.38G > A | | | | | | | | | | | | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| EZH2 | c.72dupG | | | | | | | | | | | |
| EZH2 | c.862C > T | | | | | | | | | | | |
| EZH2 | c.894dupT | | | | | | | | | | | |
| EZH2 | c.965A > G | | | | | | | | | | | |
| FLT3 | c.1359C > A | | | | | | | | | | | |
| FLT3 | c.1715A > G | | | | | | | | | | | |
| FLT3 | c.1765T > A | | | | | | | | | | | |
| FLT3 | c.1769T > C | | | | | | | | | | | |
| FLT3 | c.1836T > C | | | | | | | | | | | |
| FLT3 | c.2503G > C | | | | | | | | | | | |
| FLT3 | c.2503G > T | | | | | | | | | | | |
| FLT3 | c.2508_2510delCAT | | | | | | | | | | | |
| FLT3 | c.2510T > A | | | | | | | | | | | |
| FLT3 | c.2522A > T | | | | | | | | | | | |
| FLT3 | c.2524T > G | | | | | | | | | | | |
| FLT3 | c.2525A > G | | | | | | | | | | | |
| IDH1 | c.394C > T | | | | | | | | | | | |
| IDH1 | c.395G > A | | | | | | | | | | | |
| IDH2 | c.419G > A | | | | | 1 | | | | | | |
| IDH2 | c.515G > A | | | | | 1 | | | | | | |
| JAK2 | c.1541C > T | | | | | | | | | | | |
| JAK2 | c.1624_1629delAATGAA | | | | | | | | | | | |
| JAK2 | c.1710C > T | | | | | | | | | | | |
| JAK2 | c.1849G > T | | | | | | | | | | | |
| KIT | c.106C > T | | | | | | | | | | | |
| KIT | c.1647 + | | | | | | | | | | | |
| | 7A > C | | | | | | | | | | | |
| KIT | c.2394C > T | | | | | | | | | | | |
| KIT | c.2447A > T | | | | | | | | | | | |
| KIT | c.287C > T | | | | | | | | | | | |
| KMT2A | c.6698_6699delinsTT | | | | | | | | | | | |
| KRAS | c.173C > T | | | | 2 | | | | 2 | | | |
| KRAS | c.179G > A | | | | 2 | | | | 2 | | | |
| KRAS | c.182A > C | | | | 2 | | | | 2 | | | |
| KRAS | c.182A > G | | | | 2 | | | | 2 | | | |
| KRAS | c.183A > T | | | | 2 | | | | 2 | | | |
| KRAS | c.350A > G | | | | 2 | | | | 2 | | | |
| KRAS | c.35G > A | | | | 3 | | | 1 | 1 | | | |
| KRAS | c.35G > C | | | | 3 | | | | 2 | | | |
| KRAS | c.35G > T | | | | 3 | | | 1 | 1 | | | |
| KRAS | c.38G > A | | | | 2 | | | 1 | 1 | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| EZH2 | c.72dupG | | | | | | | | | | | | |
| EZH2 | c.862C > T | | | | | | | | | | | | |
| EZH2 | c.894dupT | | | | | | | | | | | | |
| EZH2 | c.965A > G | | | | | | | | | | | | |
| FLT3 | c.1359C > A | | | | | | | | | | | | |
| FLT3 | c.1715A > G | | | | | | | | | | | | |
| FLT3 | c.1765T > A | | | | | | | | | | | | |
| FLT3 | c.1769T > C | | | | | | | | | | | | |
| FLT3 | c.1836T > C | | | | | | | | | | | | |
| FLT3 | c.2503G > C | | | | | | | | | | | | |
| FLT3 | c.2503G > T | | | | | | | | | | | | |
| FLT3 | c.2508_2510delCAT | | | | | | | | | | | | |
| FLT3 | c.2510T > A | | | | | | | | | | | | |
| FLT3 | c.2522A > T | | | | | | | | | | | | |
| FLT3 | c.2524T > G | | | | | | | | | | | | |
| FLT3 | c.2525A > G | | | | | | | | | | | | |
| IDH1 | c.394C > T | | | | | | | | | | | | |
| IDH1 | c.395G > A | | | | | | | | | | | | |
| IDH2 | c.419G > A | | | | | | | | | | | | |
| IDH2 | c.515G > A | | | | | | | | | | | | |
| JAK2 | c.1541C > T | | | | | | | | | | | | |
| JAK2 | c.1624_1629delAATGAA | | | | | | | | | | | | |
| JAK2 | c.1710C > T | | | | | | | | | | | | |
| JAK2 | c.1849G > T | | | | | | | | | | | | |
| KIT | c.106C > T | | | | | | | | | | 3 | | |
| KIT | c.1647 + | | | | | | | | | | 3 | | |
| | 7A > C | | | | | | | | | | | | |
| KIT | c.2394C > T | | | | | | | | | | 3 | | |
| KIT | c.2447A > T | | | | | | | | | | 3 | 2 | |
| KIT | c.287C > T | | | | | | | | | | 3 | | |
| KMT2A | c.6698_6699delinsTT | | | | | | | | | | | | |
| KRAS | c.173C > T | 1 | 1 | | 1 | | | | | | | | |
| KRAS | c.179G > A | 1 | 1 | | 1 | | | | | | | | |
| KRAS | c.182A > C | 1 | 1 | | 1 | | | | | | | | |
| KRAS | c.182A > G | 1 | 1 | | 1 | | | | | | | | |
| KRAS | c.183A > T | 1 | 1 | | 1 | | | | | | | | |
| KRAS | c.350A > G | 1 | 1 | | 1 | | | | | | | | |
| KRAS | c.35G > A | 1 | 1 | 1 | 1 | | | | | | | | |
| KRAS | c.35G > C | 1 | 1 | | 2 | | | | | | | | |
| KRAS | c.35G > T | 1 | 1 | 1 | 1 | | | | | | | | |
| KRAS | c.38G > A | 1 | 1 | 1 | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | | | |
| | | lenalidomide | | rituximab | lenvatinib | lorlatinib | inhibitor | methotrexate | methylprednisolone | midostaurin | mitoxantrone |
| Gene | Variant | ✓ | X | ✓ | ✓ | X | ✓ | X | X | ✓ | X |
| EZH2 | c.72dupG | | | | | | | | | | |
| EZH2 | c.862C > T | | | | | | | | | | |
| EZH2 | c.894dupT | | | | | | | | | | |
| EZH2 | c.965A > G | | | | | | | | | | |
| FLT3 | c.1359C > A | | | | | | | | | 3 | |
| FLT3 | c.1715A > G | | | | | | | | | 3 | |
| FLT3 | c.1765T > A | | | | | | | | | 2 | |
| FLT3 | c.1769T > C | | | | | | | | | 2 | |
| FLT3 | c.1836T > C | | | | | | | | | 2 | |
| FLT3 | c.2503G > C | | | | | | | | | 3 | |
| FLT3 | c.2503G > T | | | | | | | | | 2 | |
| FLT3 | c.2508_2510delCAT | | | | | | | | | 3 | |
| FLT3 | c.2510T > A | | | | | | | | | 3 | |
| FLT3 | c.2522A > T | | | | | | | | | 2 | |
| FLT3 | c.2524T > G | | | | | | | | | 2 | |
| FLT3 | c.2525A > G | | | | | | | | | 3 | |
| IDH1 | c.394C > T | | | | | | | | | | |
| IDH1 | c.395G > A | | | | | | | | | | |
| IDH2 | c.419G > A | | | | | | | | | | |
| IDH2 | c.515G > A | | | | | | | | | | |
| JAK2 | c.1541C > T | | | | | | | | | | |
| JAK2 | c.1624_1629delAATGAA | | | | | | | | | | |
| JAK2 | c.1710C > T | | | | | | | | | | |
| JAK2 | c.1849G > T | | | | | | | | | | |
| KIT | c.106C > T | | | | 2 | | | | | 1 | |
| KIT | c.1647 + | | | | 2 | | | | | 1 | |
| | 7A > C | | | | | | | | | | |
| KIT | c.2394C > T | | | | 2 | | | | | 1 | |
| KIT | c.2447A > T | | | | 2 | | | | | 1 | |
| KIT | c.287C > T | | | | 2 | | | | | 1 | |
| KMT2A | c.6698_6699delinsTT | | | | | | | | | | |
| KRAS | c.173C > T | | | | | | | | | | |
| KRAS | c.179G > A | | | | | | | | | | |
| KRAS | c.182A > C | | | | | | | | | | |
| KRAS | c.182A > G | | | | | | | | | | |
| KRAS | c.183A > T | | | | | | | | | | |
| KRAS | c.350A > G | | | | | | | | | | |
| KRAS | c.35G > A | | | | | | | | | | |
| KRAS | c.35G > C | | | | | | | | | | |
| KRAS | c.35G > T | | | | | | | | | | |
| KRAS | c.38G > A | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| EZH2 | c.72dupG | | | | | | | | | |
| EZH2 | c.862C > T | | | | | | | | | |
| EZH2 | c.894dupT | | | | | | | | | |
| EZH2 | c.965A > G | | | | | | | | | |
| FLT3 | c.1359C > A | | | 2 | | | | | | |
| FLT3 | c.1715A > G | | | 2 | | | | | | |
| FLT3 | c.1765T > A | | | 2 | | | | | | |
| FLT3 | c.1769T > C | | | 2 | | | | | | |
| FLT3 | c.1836T > C | | | 2 | | | | | | |
| FLT3 | c.2503G > C | | | 2 | | | | | | |
| FLT3 | c.2503G > T | | | 2 | | | | | | |
| FLT3 | c.2508_2510delCAT | | | 2 | | | | | | |
| FLT3 | c.2510T > A | | | 2 | | | | | | |
| FLT3 | c.2522A > T | | | 2 | | | | | | |
| FLT3 | c.2524T > G | | | 2 | | | | | | |
| FLT3 | c.2525A > G | | | 2 | | | | | | |
| IDH1 | c.394C > T | | | | | | | | | |
| IDH1 | c.395G > A | | | | | | | | | |
| IDH2 | c.419G > A | | | | | | | | | |
| IDH2 | c.515G > A | | | | | | | | | |
| JAK2 | c.1541C > T | | | | | | | | | |
| JAK2 | c.1624_1629delAATGAA | | | | | | | | | |
| JAK2 | c.1710C > T | | | | | | | | | |
| JAK2 | c.1849G > T | | | | | | | | | |
| KIT | c.106C > T | 2 | | | | | | | | |
| KIT | c.1647 + | 2 | | | | | | | | |
| | 7A > C | | | | | | | | | |
| KIT | c.2394C > T | 2 | | | | | | | | |
| KIT | c.2447A > T | 2 | | | | | | | | |
| KIT | c.287C > T | 2 | | | | | | | | |
| KMT2A | c.6698_6699delinsTT | | | | | | | | | |
| KRAS | c.173C > T | | | | | | | | | |
| KRAS | c.179G > A | | | | | | | | | |
| KRAS | c.182A > C | | | | | | | | | |
| KRAS | c.182A > G | | | | | | | | | |
| KRAS | c.183A > T | | | | | | | | | |
| KRAS | c.350A > G | | | | | | | | | |
| KRAS | c.35G > A | | | | | | | | | 1 |
| KRAS | c.35G > C | | | | | | | | | 1 |
| KRAS | c.35G > T | | | | | | | | | 1 |
| KRAS | c.38G > A | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alpha-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| EZH2 | c.72dupG | | | | 1 | | | | | | | | |
| EZH2 | c.862C > T | | | | 1 | | | | | | | | |
| EZH2 | c.894dupT | | | | 1 | | | | | | | | |
| EZH2 | c.965A > G | | | | 1 | | | | | | | | |
| FLT3 | c.1359C > A | | | | | | | | | 2 | | | |
| FLT3 | c.1715A > G | | | | | | | | | 2 | | | |
| FLT3 | c.1765T > A | | | | | | | | | 2 | | | |
| FLT3 | c.1769T > C | | | | | | | | | 2 | | | |
| FLT3 | c.1836T > C | | | | | | | | | 2 | | | |
| FLT3 | c.2503G > C | | | | | | 1 | | | 2 | | | |
| FLT3 | c.2503G > T | | | | | | 1 | | | 2 | | | |
| FLT3 | c.2508_2510delCAT | | | | | | | | | 2 | | | |
| FLT3 | c.2510T > A | | | | | | | | | 2 | | | |
| FLT3 | c.2522A > T | | | | | | | | | 2 | | | |
| FLT3 | c.2524T > G | | | | | | | | | 2 | | | |
| FLT3 | c.2525A > G | | | | | | | | | 2 | | | |
| IDH1 | c.394C > T | | | | 1 | | | | | | | | |
| IDH1 | c.395G > A | | | | | | | | | | | | |
| IDH2 | c.419G > A | | | | | | | | | | | | |
| IDH2 | c.515G > A | | | | | | | | | | | | |
| JAK2 | c.1541C > T | | | | | | | | | | | | |
| JAK2 | c.1624_1629delAATGAA | | | | | | | | | | | | |
| JAK2 | c.1710C > T | | | | | | | | | | | | |
| JAK2 | c.1849G > T | | | | | | | | | | | | |
| KIT | c.106C > T | | | 2 | | | | | | 2 | | | |
| KIT | c.1647 + | | | 2 | | | | | | 2 | | | |
| | 7A > C | | | | | | | | | | | | |
| KIT | c.2394C > T | | | 2 | | | | | | 2 | | | |
| KIT | c.2447A > T | | | 2 | | | | | | 2 | | | |
| KIT | c.287C > T | | | 2 | | | | | | 2 | | | |
| KMT2A | c.6698_6699delinsTT | | | | | | | | | | | | |
| KRAS | c.173C > T | 1 | 2 | | | | | | 1 | | | | |
| KRAS | c.179G > A | 1 | 2 | | | | | | 1 | | | | |
| KRAS | c.182A > C | 1 | 4 | | | | | | 1 | | | | 1 |
| KRAS | c.182A > G | 1 | 4 | | | | | | 1 | | | | 1 |
| KRAS | c.183A > T | 1 | 4 | | | | | | 1 | | | | 1 |
| KRAS | c.350A > G | 1 | 4 | | | | | | 1 | | | | |
| KRAS | c.35G > A | 1 | 5 | | | | | 1 | 1 | | | | |
| KRAS | c.35G > C | 1 | 5 | | | | | 1 | 1 | | | | |
| KRAS | c.35G > T | 1 | 5 | | | | | 1 | 1 | | | | |
| KRAS | c.38G > A | 1 | 5 | | | | | | 1 | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| EZH2 | c.72dupG | | | | | | | | | | | |
| EZH2 | c.862C > T | | | | | | | | | | | |
| EZH2 | c.894dupT | | | | | | | | | | | |
| EZH2 | c.965A > G | | | | | | | | | | | |
| FLT3 | c.1359C > A | | | | | | | | | | 2 | |
| FLT3 | c.1715A > G | | | | | | | | | | 2 | |
| FLT3 | c.1765T > A | | | | | | | | | | 2 | |
| FLT3 | c.1769T > C | | | | | | | | | | 2 | |
| FLT3 | c.1836T > C | | | | | | | | | | 2 | |
| FLT3 | c.2503G > C | 1 | | | | | | | | | 2 | 1 |
| FLT3 | c.2503G > T | 1 | | | | | | | | | 2 | 1 |
| FLT3 | c.2508_2510delCAT | | | | | | | | | | 2 | |
| FLT3 | c.2510T > A | | | | | | | | | | 2 | |
| FLT3 | c.2522A > T | | | | | | | | | | 2 | 1 |
| FLT3 | c.2524T > G | | | | | | | | | | 2 | |
| FLT3 | c.2525A > G | | | | | | | | | | 2 | |
| IDH1 | c.394C > T | | | | | | | | | | | |
| IDH1 | c.395G > A | | | | | | | | | | | |
| IDH2 | c.419G > A | | | | | | | | | | | |
| IDH2 | c.515G > A | | | | | | | | | | | |
| JAK2 | c.1541C > T | | | | | | | | 1 | | | |
| JAK2 | c.1624_1629delAATGAA | | | | | | | | 1 | | | |
| JAK2 | c.1710C > T | | | | | | | | 1 | | | |
| JAK2 | c.1849G > T | | | | | | | | 1 | | | |
| KIT | c.106C > T | | | 2 | | | | | | | 2 | |
| KIT | c.1647 + | | | 2 | | | | | | | 2 | |
| | 7A > C | | | | | | | | | | | |
| KIT | c.2394C > T | | | 2 | | | | | | | 2 | |
| KIT | c.2447A > T | | | 2 | | | | | | | 2 | |
| KIT | c.287C > T | | | 2 | | | | | | | 2 | |
| KMT2A | c.6698_6699delinsTT | | | | | | | | | | | |
| KRAS | c.173C > T | | 1 | 1 | | | | | | 1 | | |
| KRAS | c.179G > A | | 1 | 1 | | | | | | 1 | | |
| KRAS | c.182A > C | | 1 | 1 | | | | 1 | | 1 | | |
| KRAS | c.182A > G | | 1 | 1 | | | | 1 | | 1 | | |
| KRAS | c.183A > T | | 1 | 1 | | | | 1 | | 1 | | |
| KRAS | c.350A > G | | 1 | 1 | | | | | | 1 | | |
| KRAS | c.35G > A | | 1 | 1 | | | | | | 1 | | |
| KRAS | c.35G > C | | 1 | 1 | | | | 1 | | 1 | | |
| KRAS | c.35G > T | | 1 | 1 | | | | | | 1 | | |
| KRAS | c.38G > A | | 1 | 1 | | | | | | 1 | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| EZH2 | c.72dupG | | | | | | | | | | | |
| EZH2 | c.862C > T | | | | | | | | | | | |
| EZH2 | c.894dupT | | | | | | | | | | | |
| EZH2 | c.965A > G | | | | | | | | | | | |
| FLT3 | c.1359C > A | 2 | | | | | | | | | | |
| FLT3 | c.1715A > G | 2 | | | | | | | | | | |
| FLT3 | c.1765T > A | 2 | | | | | | | | | | |
| FLT3 | c.1769T > C | 2 | | | | | | | | | | |
| FLT3 | c.1836T > C | 2 | | | | | | | | | | |
| FLT3 | c.2503G > C | 2 | | | | 1 | | | | | | |
| FLT3 | c.2503G > T | 2 | | | | | | | | | | |
| FLT3 | c.2508_2510delCAT | 2 | | | | | | | | | | |
| FLT3 | c.2510T > A | 2 | | | | | | | | | | |
| FLT3 | c.2522A > T | 2 | | | | | | | | | | |
| FLT3 | c.2524T > G | 2 | | | | | | | | | | |
| FLT3 | c.2525A > G | 2 | | | | | | | | | | |
| IDH1 | c.394C > T | | | 1 | | | | | | | | |
| IDH1 | c.395G > A | | | 1 | | | | | | | | |
| IDH2 | c.419G > A | | | | | | | | | | | |
| IDH2 | c.515G > A | | | | | | | | | | | |
| JAK2 | c.1541C > T | | | | | | | | | | | |
| JAK2 | c.1624_1629delAATGAA | | | | | | | | | | | |
| JAK2 | c.1710C > T | | | | | | | | | | | |
| JAK2 | c.1849G > T | | | | | | | | | | | |
| KIT | c.106C > T | 2 | | | | | | | | | | |
| KIT | c.1647 + | 2 | | | | | | | | | | |
| | 7A > C | | | | | | | | | | | |
| KIT | c.2394C > T | 2 | | | | | | | | | | |
| KIT | c.2447A > T | 2 | 1 | | | | | | | | | |
| KIT | c.287C > T | 2 | | | | | | | | | | |
| KMT2A | c.6698_6699delinsTT | | | | | | | | | | | |
| KRAS | c.173C > T | | | | | | 1 | | | | | 1 |
| KRAS | c.179G > A | | | | | | 1 | | | | | 1 |
| KRAS | c.182A > C | | | | | | 1 | | | | | 1 |
| KRAS | c.182A > G | | | | | | 1 | | | | | 1 |
| KRAS | c.183A > T | | | | | | 1 | | | | | 1 |
| KRAS | c.350A > G | | | | | | 1 | | | | | 1 |
| KRAS | c.35G > A | | | | | | 2 | | | | | 1 |
| KRAS | c.35G > C | | | | | | 1 | | | | | 1 |
| KRAS | c.35G > T | | | | | | 2 | | | | | 1 |
| KRAS | c.38G > A | | | | | | 2 | | | | | 1 |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| NF1 | c.1308G > A | | | | | | | | | | | | | | | |
| NF1 | c.1750A > T | | | | | | | | | | | | | | | |
| NF1 | c.1870T > C | | | | | | | | | | | | | | | |
| NF1 | c.2033dupC | | | | | | | | | | | | | | | |
| NF1 | c.2193C > T | | | | | | | | | | | | | | | |
| NF1 | c.2618G > A | | | | | | | | | | | | | | | |
| NF1 | c.3198 − | | | | | | | | | | | | | | | |
| | 2A > T | | | | | | | | | | | | | | | |
| NF1 | c.3200A > T | | | | | | | | | | | | | | | |
| NF1 | c.3250C > A | | | | | | | | | | | | | | | |
| NF1 | c.3651T > G | | | | | | | | | | | | | | | |
| NF1 | c.3686A > G | | | | | | | | | | | | | | | |
| NF1 | c.3827G > A | | | | | | | | | | | | | | | |
| NF1 | c.4269 + | | | | | | | | | | | | | | | |
| | 22_4269 + | | | | | | | | | | | | | | | |
| | 25delGCTA | | | | | | | | | | | | | | | |
| NF1 | c.4553T > A | | | | | | | | | | | | | | | |
| NF1 | c.480 − | | | | | | | | | | | | | | | |
| | 7T > A | | | | | | | | | | | | | | | |
| NF1 | c.499_502delTGTT | | | | | | | | | | | | | | | |
| NF1 | c.5242C > T | | | | | | | | | | | | | | | |
| NF1 | c.5843A > T | | | | | | | | | | | | | | | |
| NF1 | c.6109A > G | | | | | | | | | | | | | | | |
| NF1 | c.7806 + | | | | | | | | | | | | | | | |
| | 3A > C | | | | | | | | | | | | | | | |
| NF1 | c.7908 − | | | | | | | | | | | | | | | |
| | 8delT | | | | | | | | | | | | | | | |
| NPM1 | c.860_863dupTCTG | | | | | | | | | | | | | | | |
| NPM1 | c.863_864insCATG | | | | | | | | | | | | | | | |
| NPM1 | c.863_864insCCTG | | | | | | | | | | | | | | | |
| NPM1 | c.863_864insTTTG | | | | | | | | | | | | | | | |
| NRAS | c.112 − | | | 1 | | | | | | 1 | | | | | | |
| | 8A > G | | | | | | | | | | | | | | | |
| NRAS | c.176C > G | | | 1 | | | | | | 1 | | | | | | |
| NRAS | c.181C > A | | | 1 | | | | | | 1 | | | | | | |
| NRAS | c.182A > G | | | 1 | | | | | | 1 | | | | | | |
| NRAS | c.225C > T | | | 1 | | | | | | 1 | | | | | | |
| NRAS | c.34G > A | | | 1 | | | | | | 1 | | | | | | |
| NRAS | c.35G > A | | | 1 | | | | | | 1 | | | | | | |
| NRAS | c.35G > C | | | 1 | | | | | | 1 | | | | | | |
| NRAS | c.35G > T | | | 1 | | | | | | 1 | | | | | | |
| NRAS | c.37G > C | | | 1 | | | | | | 1 | | | | | | |
| NRAS | c.37G > T | | | 1 | | | | | | 1 | | | | | | |
| NRAS | c.38G > A | | | 1 | | | | | | 1 | | | | | | |
| NRAS | c.38G > T | | | 1 | | | | | | 1 | | | | | | |
| PDGFRA | c.1654 − | | | | | | | | | | | | | | | |
| | 8A > G | | | | | | | | | | | | | | | |
| PML | c.121C > G | | | | | | | | | | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| NF1 | c.1308G > A | | | | | | | | | | | | | | |
| NF1 | c.1750A > T | | | | | | | | | | | | | | |
| NF1 | c.1870T > C | | | | | | | | | | | | | | |
| NF1 | c.2033dupC | | | | | | | | | | | | | | |
| NF1 | c.2193C > T | | | | | | | | | | | | | | |
| NF1 | c.2618G > A | | | | | | | | | | | | | | |
| NF1 | c.3198 − | | | | | | | | | | | | | | |
| | 2A > T | | | | | | | | | | | | | | |
| NF1 | c.3200A > T | | | | | | | | | | | | | | |
| NF1 | c.3250C > A | | | | | | | | | | | | | | |
| NF1 | c.3651T > G | | | | | | | | | | | | | | |
| NF1 | c.3686A > G | | | | | | | | | | | | | | |
| NF1 | c.3827G > A | | | | | | | | | | | | | | |
| NF1 | c.4269 + | | | | | | | | | | | | | | |
| | 22_4269 + | | | | | | | | | | | | | | |
| | 25delGCTA | | | | | | | | | | | | | | |
| NF1 | c.4553T > A | | | | | | | | | | | | | | |
| NF1 | c.480 − | | | | | | | | | | | | | | |
| | 7T > A | | | | | | | | | | | | | | |
| NF1 | c.499_502delTGTT | | | | | | | | | | | | | | |
| NF1 | c.5242C > T | | | | | | | | | | | | | | |
| NF1 | c.5843A > T | | | | | | | | | | | | | | |
| NF1 | c.6109A > G | | | | | | | | | | | | | | |
| NF1 | c.7806 + | | | | | | | | | | | | | | |
| | 3A > C | | | | | | | | | | | | | | |
| NF1 | c.7908 − | | | | | | | | | | | | | | |
| | 8delT | | | | | | | | | | | | | | |
| NPM1 | c.860_863dupTCTG | | | | | | | | | | | | | | |
| NPM1 | c.863_864insCATG | | | | | | | | | | | | | | |
| NPM1 | c.863_864insCCTG | | | | | | | | | | | | | | |
| NPM1 | c.863_864insTTTG | | | | | | | | | | | | | | |
| NRAS | c.112 − | | | | | 1 | | | | | | | | | |
| | 8A > G | | | | | | | | | | | | | | |
| NRAS | c.176C > G | 1 | | | | 1 | | | | | | | | | |
| NRAS | c.181C > A | 1 | | | | 1 | 1 | | | | | | | | |
| NRAS | c.182A > G | 1 | | | | 1 | | | | | | | | | |
| NRAS | c.225C > T | 1 | | | | 1 | | | | | | | | | |
| NRAS | c.34G > A | 1 | | | | 1 | | | | | | | | | |
| NRAS | c.35G > A | 1 | | | | 1 | 1 | | | | | | | | |
| NRAS | c.35G > C | 1 | | | | 1 | 1 | | | | | | | | |
| NRAS | c.35G > T | 1 | | | | 1 | 1 | | | | | | | | |
| NRAS | c.37G > C | 1 | | | | 1 | 1 | | | | | | | | |
| NRAS | c.37G > T | 1 | | | | 1 | 1 | | | | | | | | |
| NRAS | c.38G > A | 1 | | | | 1 | | | | | | | | | |
| NRAS | c.38G > T | 1 | | | | 1 | 1 | | | | | | | | |
| PDGFRA | c.1654 − | | | | 2 | | | | | | | | | | |
| | 8A > G | | | | | | | | | | | | | | |
| PML | c.121C > G | | 2 | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| NF1 | c.1308G > A | | | | | | | | | | | | | |
| NF1 | c.1750A > T | | | | | | | | | | | | | |
| NF1 | c.1870T > C | | | | | | | | | | | | | |
| NF1 | c.2033dupC | | | | | | | | | | | | | |
| NF1 | c.2193C > T | | | | | | | | | | | | | |
| NF1 | c.2618G > A | | | | | | | | | | | | | |
| NF1 | c.3198 − | | | | | | | | | | | | | |
| | 2A > T | | | | | | | | | | | | | |
| NF1 | c.3200A > T | | | | | | | | | | | | | |
| NF1 | c.3250C > A | | | | | | | | | | | | | |
| NF1 | c.3651T > G | | | | | | | | | | | | | |
| NF1 | c.3686A > G | | | | | | | | | | | | | |
| NF1 | c.3827G > A | | | | | | | | | | | | | |
| NF1 | c.4269 + | | | | | | | | | | | | | |
| | 22_4269 + | | | | | | | | | | | | | |
| | 25delGCTA | | | | | | | | | | | | | |
| NF1 | c.4553T > A | | | | | | | | | | | | | |
| NF1 | c.480 − | | | | | | | | | | | | | |
| | 7T > A | | | | | | | | | | | | | |
| NF1 | c.499_502delTGTT | | | | | | | | | | | | | |
| NF1 | c.5242C > T | | | | | | | | | | | | | |
| NF1 | c.5843A > T | | | | | | | | | | | | | |
| NF1 | c.6109A > G | | | | | | | | | | | | | |
| NF1 | c.7806 + | | | | | | | | | | | | | |
| | 3A > C | | | | | | | | | | | | | |
| NF1 | c.7908 − | | | | | | | | | | | | | |
| | 8delT | | | | | | | | | | | | | |
| NPM1 | c.860_863dupTCTG | | | | | | | | | | | | | |
| NPM1 | c.863_864insCATG | | | | | | | | | | | | | |
| NPM1 | c.863_864insCCTG | | | | | | | | | | | | | |
| NPM1 | c.863_864insTTTG | | | | | | | | | | | | | |
| NRAS | c.112 − | | | | | | | | | 2 | | | | |
| | 8A > G | | | | | | | | | | | | | |
| NRAS | c.176C > G | | | | | | | | | 4 | | | | |
| NRAS | c.181C > A | | | | | | | | | 5 | | | | |
| NRAS | c.182A > G | | | | | | | | | 4 | | | | |
| NRAS | c.225C > T | | | | | | | | | 3 | | | | |
| NRAS | c.34G > A | | | | | | | | | 4 | | | | |
| NRAS | c.35G > A | | | | | | | | | 5 | | | | |
| NRAS | c.35G > C | | | | | | | | | 5 | | | | |
| NRAS | c.35G > T | | | | | | | | | 5 | | | | |
| NRAS | c.37G > C | | | | | | | | | 5 | | | | |
| NRAS | c.37G > T | | | | | | | | | 5 | | | | |
| NRAS | c.38G > A | | | | | | | | | 4 | | | | |
| NRAS | c.38G > T | | | | | | | | | 5 | | | | |
| PDGFRA | c.1654 − | | | | | | | | | | | | | |
| | 8A > G | | | | | | | | | | | | | |
| PML | c.121C > G | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| NF1 | c.1308G > A | | | | | | | | | | | |
| NF1 | c.1750A > T | | | | | | | | | | | |
| NF1 | c.1870T > C | | | | | | | | | | | |
| NF1 | c.2033dupC | | | | | | | | | | | |
| NF1 | c.2193C > T | | | | | | | | | | | |
| NF1 | c.2618G > A | | | | | | | | | | | |
| NF1 | c.3198 − | | | | | | | | | | | |
| | 2A > T | | | | | | | | | | | |
| NF1 | c.3200A > T | | | | | | | | | | | |
| NF1 | c.3250C > A | | | | | | | | | | | |
| NF1 | c.3651T > G | | | | | | | | | | | |
| NF1 | c.3686A > G | | | | | | | | | | | |
| NF1 | c.3827G > A | | | | | | | | | | | |
| NF1 | c.4269 + | | | | | | | | | | | |
| | 22_4269 + | | | | | | | | | | | |
| | 25delGCTA | | | | | | | | | | | |
| NF1 | c.4553T > A | | | | | | | | | | | |
| NF1 | c.480 − | | | | | | | | | | | |
| | 7T > A | | | | | | | | | | | |
| NF1 | c.499_502delTGTT | | | | | | | | | | | |
| NF1 | c.5242C > T | | | | | | | | | | | |
| NF1 | c.5843A > T | | | | | | | | | | | |
| NF1 | c.6109A > G | | | | | | | | | | | |
| NF1 | c.7806 + | | | | | | | | | | | |
| | 3A > C | | | | | | | | | | | |
| NF1 | c.7908 − | | | | | | | | | | | |
| | 8delT | | | | | | | | | | | |
| NPM1 | c.860_863dupTCTG | | | | | | | | | | | |
| NPM1 | c.863_864insCATG | | | | | | | | | | | |
| NPM1 | c.863_864insCCTG | | | | | | | | | | | |
| NPM1 | c.863_864insTTTG | | | | | | | | | | | |
| NRAS | c.112 − | | | | | | | | | | | |
| | 8A > G | | | | | | | | | | | |
| NRAS | c.176C > G | | | | | | | | | | | |
| NRAS | c.181C > A | | | | | | | | | | | |
| NRAS | c.182A > G | | | | | | | | | | | |
| NRAS | c.225C > T | | | | | | | | | | | |
| NRAS | c.34G > A | | | | | | | | | | | |
| NRAS | c.35G > A | | | | | | | | | | | |
| NRAS | c.35G > C | | | | | | | | | | | |
| NRAS | c.35G > T | | | | | | | | | | | |
| NRAS | c.37G > C | | | | | | | | | | | |
| NRAS | c.37G > T | | | | | | | | | | | |
| NRAS | c.38G > A | | | | | | | | | | | |
| NRAS | c.38G > T | | | | | | | | | | | |
| PDGFRA | c.1654 − | | | | | | | | | | | |
| | 8A > G | | | | | | | | | | | |
| PML | c.121C > G | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| NF1 | c.1308G > A | | | | | | | | | | | | | |
| NF1 | c.1750A > T | | | | | | | | | | | | | |
| NF1 | c.1870T > C | | | | | | | | | | | | | |
| NF1 | c.2033dupC | | | | | | | | | | | | | |
| NF1 | c.2193C > T | | | | | | | | | | | | | |
| NF1 | c.2618G > A | | | | | | | | | | | | | |
| NF1 | c.3198 − | | | | | | | | | | | | | |
| | 2A > T | | | | | | | | | | | | | |
| NF1 | c.3200A > T | | | | | | | | | | | | | |
| NF1 | c.3250C > A | | | | | | | | | | | | | |
| NF1 | c.3651T > G | | | | | | | | | | | | | |
| NF1 | c.3686A > G | | | | | | | | | | | | | |
| NF1 | c.3827G > A | | | | | | | | | | | | | |
| NF1 | c.4269 + | | | | | | | | | | | | | |
| | 22_4269 + | | | | | | | | | | | | | |
| | 25delGCTA | | | | | | | | | | | | | |
| NF1 | c.4553T > A | | | | | | | | | | | | | |
| NF1 | c.480 − | | | | | | | | | | | | | |
| | 7T > A | | | | | | | | | | | | | |
| NF1 | c.499_502delTGTT | | | | | | | | | | | | | |
| NF1 | c.5242C > T | | | | | | | | | | | | | |
| NF1 | c.5843A > T | | | | | | | | | | | | | |
| NF1 | c.6109A > G | | | | | | | | | | | | | |
| NF1 | c.7806 + | | | | | | | | | | | | | |
| | 3A > C | | | | | | | | | | | | | |
| NF1 | c.7908 − | | | | | | | | | | | | | |
| | 8delT | | | | | | | | | | | | | |
| NPM1 | c.860_863dupTCTG | | | | | | | | | 1 | | | | |
| NPM1 | c.863_864insCATG | 1 | | | | | | | | 1 | | | | |
| NPM1 | c.863_864insCCTG | 1 | | | | | | | | 1 | | | | |
| NPM1 | c.863_864insTTTG | 1 | | | | | | | | 1 | | | | |
| NRAS | c.112 − | | | | | | | | | | | | | |
| | 8A > G | | | | | | | | | | | | | |
| NRAS | c.176C > G | | | | | | | | | | | | | |
| NRAS | c.181C > A | | | 1 | | 1 | 1 | | | | | | | |
| NRAS | c.182A > G | | | 1 | | 1 | 1 | | | | | | | |
| NRAS | c.225C > T | | | | | | | | | | | | | |
| NRAS | c.34G > A | | | 1 | | 1 | | | | | | | | |
| NRAS | c.35G > A | | | 1 | | 1 | | | | | | | | |
| NRAS | c.35G > C | | | 1 | | 1 | | | | | | | | |
| NRAS | c.35G > T | | | 1 | | 1 | | | | | | | | |
| NRAS | c.37G > C | | | 1 | | | | | | | | | | |
| NRAS | c.37G > T | | | 1 | | | | | | | | | | |
| NRAS | c.38G > A | | | 1 | | | | | | | | | | |
| NRAS | c.38G > T | | | 1 | | | | | | | | | | |
| PDGFRA | c.1654 − | | | | | | | 2 | | | | | | |
| | 8A > G | | | | | | | | | | | | | |
| PML | c.121C > G | | | | | | | | | | | | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| NF1 | c.1308G > A | | | | | | | | | | | |
| NF1 | c.1750A > T | | | | | | | | | | | |
| NF1 | c.1870T > C | | | | | | | | | | | |
| NF1 | c.2033dupC | | | | | | | | | | | |
| NF1 | c.2193C > T | | | | | | | | | | | |
| NF1 | c.2618G > A | | | | | | | | | | | |
| NF1 | c.3198 − | | | | | | | | | | | |
| | 2A > T | | | | | | | | | | | |
| NF1 | c.3200A > T | | | | | | | | | | | |
| NF1 | c.3250C > A | | | | | | | | | | | |
| NF1 | c.3651T > G | | | | | | | | | | | |
| NF1 | c.3686A > G | | | | | | | | | | | |
| NF1 | c.3827G > A | | | | | | | | | | | |
| NF1 | c.4269 + | | | | | | | | | | | |
| | 22_4269 + | | | | | | | | | | | |
| | 25delGCTA | | | | | | | | | | | |
| NF1 | c.4553T > A | | | | | | | | | | | |
| NF1 | c.480 − | | | | | | | | | | | |
| | 7T > A | | | | | | | | | | | |
| NF1 | c.499_502delTGTT | | | | | | | | | | | |
| NF1 | c.5242C > T | | | | | | | | | | | |
| NF1 | c.5843A > T | | | | | | | | | | | |
| NF1 | c.6109A > G | | | | | | | | | | | |
| NF1 | c.7806 + | | | | | | | | | | | |
| | 3A > C | | | | | | | | | | | |
| NF1 | c.7908 − | | | | | | | | | | | |
| | 8delT | | | | | | | | | | | |
| NPM1 | c.860_863dupTCTG | | | | | | | | | | | |
| NPM1 | c.863_864insCATG | | | | | | | | | | | |
| NPM1 | c.863_864insCCTG | | | | | | | | | | | |
| NPM1 | c.863_864insTTTG | | | | | | | | | | | |
| NRAS | c.112 − | | | | | | | | | | | |
| | 8A > G | | | | | | | | | | | |
| NRAS | c.176C > G | | | | | | | | | | | |
| NRAS | c.181C > A | | | | | | | | | | | |
| NRAS | c.182A > G | | | | | | | | | | | |
| NRAS | c.225C > T | | | | | | | | | | | |
| NRAS | c.34G > A | | | | | | | | | | | |
| NRAS | c.35G > A | | | | | | | | | | | |
| NRAS | c.35G > C | | | | | | | | | | | |
| NRAS | c.35G > T | | | | | | | | | | | |
| NRAS | c.37G > C | | | | | | | | | | | |
| NRAS | c.37G > T | | | | | | | | | | | |
| NRAS | c.38G > A | | | | | | | | | | | |
| NRAS | c.38G > T | | | | | | | | | | | |
| PDGFRA | c.1654 − | | | | | | | | | | | |
| | 8A > G | | | | | | | | | | | |
| PML | c.121C > G | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| NF1 | c.1308G > A | | | | | | | | | | | 1 | |
| NF1 | c.1750A > T | | | | | | | | | | | 1 | |
| NF1 | c.1870T > C | | | | | | | | | | | 1 | |
| NF1 | c.2033dupC | | | | | | | | | | | 1 | |
| NF1 | c.2193C > T | | | | | | | | | | | 1 | |
| NF1 | c.2618G > A | | | | | | | | | | | 1 | |
| NF1 | c.3198 − | | | | | | | | | | | 1 | |
| | 2A > T | | | | | | | | | | | | |
| NF1 | c.3200A > T | | | | | | | | | | | 1 | |
| NF1 | c.3250C > A | | | | | | | | | | | 1 | |
| NF1 | c.3651T > G | | | | | | | | | | | 1 | |
| NF1 | c.3686A > G | | | | | | | | | | | 1 | |
| NF1 | c.3827G > A | | | | | | | | | | | 1 | |
| NF1 | c.4269 + | | | | | | | | | | | 1 | |
| | 22_4269 + | | | | | | | | | | | | |
| | 25delGCTA | | | | | | | | | | | | |
| NF1 | c.4553T > A | | | | | | | | | | | 1 | |
| NF1 | c.480 − | | | | | | | | | | | 1 | |
| | 7T > A | | | | | | | | | | | | |
| NF1 | c.499_502delTGTT | | | | | | | | | | | 1 | |
| NF1 | c.5242C > T | | | | | | | | | | | 1 | |
| NF1 | c.5843A > T | | | | | | | | | | | 1 | |
| NF1 | c.6109A > G | | | | | | | | | | | 1 | |
| NF1 | c.7806 + | | | | | | | | | | | 1 | |
| | 3A > C | | | | | | | | | | | | |
| NF1 | c.7908 − | | | | | | | | | | | 1 | |
| | 8delT | | | | | | | | | | | | |
| NPM1 | c.860_863dupTCTG | | | | | | | | | | | | |
| NPM1 | c.863_864insCATG | | | | | | | | | | | | |
| NPM1 | c.863_864insCCTG | | | | | | | | | | | | |
| NPM1 | c.863_864insTTTG | | | | | | | | | | | | |
| NRAS | c.112 − | 1 | | | | | | | | | | | |
| | 8A > G | | | | | | | | | | | | |
| NRAS | c.176C > G | 1 | | | | | | | | | | | |
| NRAS | c.181C > A | 1 | | 1 | | | | | | | | | |
| NRAS | c.182A > G | 1 | | | | | | | | | | | |
| NRAS | c.225C > T | 1 | | | | | | | | | | | |
| NRAS | c.34G > A | 1 | | | | | | | | | | 1 | |
| NRAS | c.35G > A | 1 | | 1 | | | | | | | | 1 | |
| NRAS | c.35G > C | 1 | | 1 | | | | | | | | 1 | |
| NRAS | c.35G > T | 1 | | 1 | | | | | | | | 1 | |
| NRAS | c.37G > C | 1 | | | | | | | | | | | |
| NRAS | c.37G > T | 1 | | | | | | | | | | | |
| NRAS | c.38G > A | 1 | | | | | | | | | | | |
| NRAS | c.38G > T | 1 | | | | | | | | | | | |
| PDGFRA | c.1654 − | | | | | | | | | | 3 | | |
| | 8A > G | | | | | | | | | | | | |
| PML | c.121C > G | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | | | |
| | | lenalidomide | | rituximab | lenvatinib | lorlatinib | inhibitor | methotrexate | methylprednisolone | midostaurin | mitoxantrone |
| Gene | Variant | ✓ | X | ✓ | ✓ | X | ✓ | X | X | ✓ | X |
| NF1 | c.1308G > A | | | | | | | | | | |
| NF1 | c.1750A > T | | | | | | | | | | |
| NF1 | c.1870T > C | | | | | | | | | | |
| NF1 | c.2033dupC | | | | | | | | | | |
| NF1 | c.2193C > T | | | | | | | | | | |
| NF1 | c.2618G > A | | | | | | | | | | |
| NF1 | c.3198 − | | | | | | | | | | |
| | 2A > T | | | | | | | | | | |
| NF1 | c.3200A > T | | | | | | | | | | |
| NF1 | c.3250C > A | | | | | | | | | | |
| NF1 | c.3651T > G | | | | | | | | | | |
| NF1 | c.3686A > G | | | | | | | | | | |
| NF1 | c.3827G > A | | | | | | | | | | |
| NF1 | c.4269 + | | | | | | | | | | |
| | 22_4269 + | | | | | | | | | | |
| | 25delGCTA | | | | | | | | | | |
| NF1 | c.4553T > A | | | | | | | | | | |
| NF1 | c.480 − | | | | | | | | | | |
| | 7T > A | | | | | | | | | | |
| NF1 | c.499_502delTGTT | | | | | | | | | | |
| NF1 | c.5242C > T | | | | | | | | | | |
| NF1 | c.5843A > T | | | | | | | | | | |
| NF1 | c.6109A > G | | | | | | | | | | |
| NF1 | c.7806 + | | | | | | | | | | |
| | 3A > C | | | | | | | | | | |
| NF1 | c.7908 − | | | | | | | | | | |
| | 8delT | | | | | | | | | | |
| NPM1 | c.860_863dupTCTG | | | | | | | | | | |
| NPM1 | c.863_864insCATG | | | | | | | | | | |
| NPM1 | c.863_864insCCTG | | | | | | | | | | |
| NPM1 | c.863_864insTTTG | | | | | | | | | | |
| NRAS | c.112 − | | | | | | | | | | |
| | 8A > G | | | | | | | | | | |
| NRAS | c.176C > G | | | | | | | | | | |
| NRAS | c.181C > A | | | | | | | | | | |
| NRAS | c.182A > G | | | | | | | | | | |
| NRAS | c.225C > T | | | | | | | | | | |
| NRAS | c.34G > A | | | | | | | | | | |
| NRAS | c.35G > A | | | | | | | | | | |
| NRAS | c.35G > C | | | | | | | | | | |
| NRAS | c.35G > T | | | | | | | | | | |
| NRAS | c.37G > C | | | | | | | | | | |
| NRAS | c.37G > T | | | | | | | | | | |
| NRAS | c.38G > A | | | | | | | | | | |
| NRAS | c.38G > T | | | | | | | | | | |
| PDGFRA | c.1654 − | | | | 2 | | | | | 2 | |
| | 8A > G | | | | | | | | | | |
| PML | c.121C > G | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| NF1 | c.1308G > A | | | | | | | | | |
| NF1 | c.1750A > T | | | | | | | | | |
| NF1 | c.1870T > C | | | | | | | | | |
| NF1 | c.2033dupC | | | | | | | | | |
| NF1 | c.2193C > T | | | | | | | | | |
| NF1 | c.2618G > A | | | | | | | | | |
| NF1 | c.3198 − | | | | | | | | | |
| | 2A > T | | | | | | | | | |
| NF1 | c.3200A > T | | | | | | | | | |
| NF1 | c.3250C > A | | | | | | | | | |
| NF1 | c.3651T > G | | | | | | | | | |
| NF1 | c.3686A > G | | | | | | | | | |
| NF1 | c.3827G > A | | | | | | | | | |
| NF1 | c.4269 + | | | | | | | | | |
| | 22_4269 + | | | | | | | | | |
| | 25delGCTA | | | | | | | | | |
| NF1 | c.4553T > A | | | | | | | | | |
| NF1 | c.480 − | | | | | | | | | |
| | 7T > A | | | | | | | | | |
| NF1 | c.499_502delTGTT | | | | | | | | | |
| NF1 | c.5242C > T | | | | | | | | | |
| NF1 | c.5843A > T | | | | | | | | | |
| NF1 | c.6109A > G | | | | | | | | | |
| NF1 | c.7806 + | | | | | | | | | |
| | 3A > C | | | | | | | | | |
| NF1 | c.7908 − | | | | | | | | | |
| | 8delT | | | | | | | | | |
| NPM1 | c.860_863dupTCTG | | | | | | | | | |
| NPM1 | c.863_864insCATG | | | | | | | | | |
| NPM1 | c.863_864insCCTG | | | | | | | | | |
| NPM1 | c.863_864insTTTG | | | | | | | | | |
| NRAS | c.112 − | | | | | | | | | |
| | 8A > G | | | | | | | | | |
| NRAS | c.176C > G | | | | | | | | | |
| NRAS | c.181C > A | | | | | | | | | |
| NRAS | c.182A > G | | | | | | | | | |
| NRAS | c.225C > T | | | | | | | | | |
| NRAS | c.34G > A | | | | | | | | | |
| NRAS | c.35G > A | | | | | | | | | |
| NRAS | c.35G > C | | | | | | | | | |
| NRAS | c.35G > T | | | | | | | | | |
| NRAS | c.37G > C | | | | | | | | | |
| NRAS | c.37G > T | | | | | | | | | |
| NRAS | c.38G > A | | | | | | | | | |
| NRAS | c.38G > T | | | | | | | | | |
| PDGFRA | c.1654 − | 2 | | 2 | | | | 2 | | |
| | 8A > G | | | | | | | | | |
| PML | c.121C > G | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alpha-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| NF1 | c.1308G > A | | | | | | | | | | | | |
| NF1 | c.1750A > T | | | | | | | | | | | | |
| NF1 | c.1870T > C | | | | | | | | | | | | |
| NF1 | c.2033dupC | | | | | | | | | | | | |
| NF1 | c.2193C > T | | | | | | | | | | | | |
| NF1 | c.2618G > A | | | | | | | | | | | | |
| NF1 | c.3198 − | | | | | | | | | | | | |
| | 2A > T | | | | | | | | | | | | |
| NF1 | c.3200A > T | | | | | | | | | | | | |
| NF1 | c.3250C > A | | | | | | | | | | | | |
| NF1 | c.3651T > G | | | | | | | | | | | | |
| NF1 | c.3686A > G | | | | | | | | | | | | |
| NF1 | c.3827G > A | | | | | | | | | | | | |
| NF1 | c.4269 + | | | | | | | | | | | | |
| | 22_4269 + | | | | | | | | | | | | |
| | 25delGCTA | | | | | | | | | | | | |
| NF1 | c.4553T > A | | | | | | | | | | | | |
| NF1 | c.480 − | | | | | | | | | | | | |
| | 7T > A | | | | | | | | | | | | |
| NF1 | c.499_502delTGTT | | | | | | | | | | | | |
| NF1 | c.5242C > T | | | | | | | | | | | | |
| NF1 | c.5843A > T | | | | | | | | | | | | |
| NF1 | c.6109A > G | | | | | | | | | | | | |
| NF1 | c.7806 + | | | | | | | | | | | | |
| | 3A > C | | | | | | | | | | | | |
| NF1 | c.7908 − | | | | | | | | | | | | |
| | 8delT | | | | | | | | | | | | |
| NPM1 | c.860_863dupTCTG | | | | | | | | | | | | |
| NPM1 | c.863_864insCATG | | | | | | | | | | | | |
| NPM1 | c.863_864insCCTG | | | | | | | | | | | | |
| NPM1 | c.863_864insTTTG | | | | | | | | | | | | |
| NRAS | c.112 − | | 2 | | | | | | | | | | |
| | 8A > G | | | | | | | | | | | | |
| NRAS | c.176C > G | | 4 | | | | | | | | | | |
| NRAS | c.181C > A | | 5 | | | | | | | | | | |
| NRAS | c.182A > G | | 4 | | | | | | | | | | |
| NRAS | c.225C > T | | 2 | | | | | | | | | | |
| NRAS | c.34G > A | | 4 | | | | | | | | | | |
| NRAS | c.35G > A | | 4 | | | | | | | | | | |
| NRAS | c.35G > C | | 4 | | | | | | | | | | |
| NRAS | c.35G > T | | 4 | | | | | | | | | | |
| NRAS | c.37G > C | | 4 | | | | | | | | | | |
| NRAS | c.37G > T | | 4 | | | | | | | | | | |
| NRAS | c.38G > A | | 4 | | | | | | | | | | |
| NRAS | c.38G > T | | 4 | | | | | | | | | | |
| PDGFRA | c.1654 − | | | 2 | | | | | | 2 | | | |
| | 8A > G | | | | | | | | | | | | |
| PML | c.121C > G | | | | | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| NF1 | c.1308G > A | | | | | | | | | | | |
| NF1 | c.1750A > T | | | | | | | | | | | |
| NF1 | c.1870T > C | | | | | | | | | | | |
| NF1 | c.2033dupC | | | | | | | | | | | |
| NF1 | c.2193C > T | | | | | | | | | | | |
| NF1 | c.2618G > A | | | | | | | | | | | |
| NF1 | c.3198 − | | | | | | | | | | | |
| | 2A > T | | | | | | | | | | | |
| NF1 | c.3200A > T | | | | | | | | | | | |
| NF1 | c.3250C > A | | | | | | | | | | | |
| NF1 | c.3651T > G | | | | | | | | | | | |
| NF1 | c.3686A > G | | | | | | | | | | | |
| NF1 | c.3827G > A | | | | | | | | | | | |
| NF1 | c.4269 + | | | | | | | | | | | |
| | 22_4269 + | | | | | | | | | | | |
| | 25delGCTA | | | | | | | | | | | |
| NF1 | c.4553T > A | | | | | | | | | | | |
| NF1 | c.480 − | | | | | | | | | | | |
| | 7T > A | | | | | | | | | | | |
| NF1 | c.499_502delTGTT | | | | | | | | | | | |
| NF1 | c.5242C > T | | | | | | | | | | | |
| NF1 | c.5843A > T | | | | | | | | | | | |
| NF1 | c.6109A > G | | | | | | | | | | | |
| NF1 | c.7806 + | | | | | | | | | | | |
| | 3A > C | | | | | | | | | | | |
| NF1 | c.7908 − | | | | | | | | | | | |
| | 8delT | | | | | | | | | | | |
| NPM1 | c.860_863dupTCTG | | | | | | | | | | | |
| NPM1 | c.863_864insCATG | | | | | | | | | | | |
| NPM1 | c.863_864insCCTG | | | | | | | | | | | |
| NPM1 | c.863_864insTTTG | | | | | | | | | | | |
| NRAS | c.112 − | | 1 | 1 | | | | | | | | |
| | 8A > G | | | | | | | | | | | |
| NRAS | c.176C > G | | 1 | 1 | | | | | | | | |
| NRAS | c.181C > A | | 1 | 1 | | | | | | | | |
| NRAS | c.182A > G | | 1 | 1 | | | | | | | | |
| NRAS | c.225C > T | | 1 | 1 | | | | | | | | |
| NRAS | c.34G > A | | 1 | 1 | | | | | | | | |
| NRAS | c.35G > A | | 1 | 1 | | | | | | | | |
| NRAS | c.35G > C | | 1 | 1 | | | | | | | | |
| NRAS | c.35G > T | | 1 | 1 | | | | | | | | |
| NRAS | c.37G > C | | 1 | 1 | | | | | | | | |
| NRAS | c.37G > T | | 1 | 1 | | | | | | | | |
| NRAS | c.38G > A | | 1 | 1 | | | | | | | | |
| NRAS | c.38G > T | | 1 | 1 | | | | | | | | |
| PDGFRA | c.1654 − | | | 2 | | | | | | | | |
| | 8A > G | | | | | | | | | | | |
| PML | c.121C > G | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| NF1 | c.1308G > A | | | | | | | | | | | |
| NF1 | c.1750A > T | | | | | | | | | | | |
| NF1 | c.1870T > C | | | | | | | | | | | |
| NF1 | c.2033dupC | | | | | | | | | | | |
| NF1 | c.2193C > T | | | | | | | | | | | |
| NF1 | c.2618G > A | | | | | | | | | | | |
| NF1 | c.3198 − | | | | | | | | | | | |
| | 2A > T | | | | | | | | | | | |
| NF1 | c.3200A > T | | | | | | | | | | | |
| NF1 | c.3250C > A | | | | | | | | | | | |
| NF1 | c.3651T > G | | | | | | | | | | | |
| NF1 | c.3686A > G | | | | | | | | | | | |
| NF1 | c.3827G > A | | | | | | | | | | | |
| NF1 | c.4269 + | | | | | | | | | | | |
| | 22_4269 + | | | | | | | | | | | |
| | 25delGCTA | | | | | | | | | | | |
| NF1 | c.4553T > A | | | | | | | | | | | |
| NF1 | c.480 − | | | | | | | | | | | |
| | 7T > A | | | | | | | | | | | |
| NF1 | c.499_502delTGTT | | | | | | | | | | | |
| NF1 | c.5242C > T | | | | | | | | | | | |
| NF1 | c.5843A > T | | | | | | | | | | | |
| NF1 | c.6109A > G | | | | | | | | | | | |
| NF1 | c.7806 + | | | | | | | | | | | |
| | 3A > C | | | | | | | | | | | |
| NF1 | c.7908 − | | | | | | | | | | | |
| | 8delT | | | | | | | | | | | |
| NPM1 | c.860_863dupTCTG | | | | | | | | | | | |
| NPM1 | c.863_864insCATG | | | | | | | | | | | |
| NPM1 | c.863_864insCCTG | | | | | | | | | | | |
| NPM1 | c.863_864insTTTG | | | | | | | | | | | |
| NRAS | c.112 − | | | | | | 1 | | 1 | | | |
| | 8A > G | | | | | | | | | | | |
| NRAS | c.176C > G | | | | | | 1 | | 1 | | | |
| NRAS | c.181C > A | | | | | | 1 | | 1 | | | |
| NRAS | c.182A > G | | | | | | 1 | | 1 | | | |
| NRAS | c.225C > T | | | | | | 1 | | 1 | | | |
| NRAS | c.34G > A | | | | | | 1 | | 1 | | | |
| NRAS | c.35G > A | | | | | | 1 | | 1 | | | |
| NRAS | c.35G > C | | | | | | 1 | | 1 | | | |
| NRAS | c.35G > T | | | | | | 1 | | 1 | | | |
| NRAS | c.37G > C | | | | | | 1 | | 1 | | | |
| NRAS | c.37G > T | | | | | | 1 | | 1 | | | |
| NRAS | c.38G > A | | | | | | 1 | | 1 | | | |
| NRAS | c.38G > T | | | | | | 1 | | 1 | | | |
| PDGFRA | c.1654 − | 2 | | | | | | | | | | |
| | 8A > G | | | | | | | | | | | |
| PML | c.121C > G | | | | | | | | | | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| PML | c.1335A > G | | | | | | | | | | | | | | | |
| PML | c.1718G > A | | | | | | | | | | | | | | | |
| PML | c.−1C > T | | | | | | | | | | | | | | | |
| PML | c.2456G > C | | | | | | | | | | | | | | | |
| PML | c.2533G > A | | | | | | | | | | | | | | | |
| PTPN11 | c.1382C > G | | | | | | | | | | | | | | | |
| PTPN11 | c.1403C > T | | | | | | | | | | | | | | | |
| PTPN11 | c.1450G > A | | | | | | | | | | | | | | | |
| PTPN11 | c.1507G > A | | | | | | | | | | | | | | | |
| PTPN11 | c.181G > A | | | | | | | | | | | | | | | |
| PTPN11 | c.182A > C | | | | | | | | | | | | | | | |
| PTPN11 | c.214G > C | | | | | | | | | | | | | | | |
| PTPN11 | c.215C > T | | | | | | | | | | | | | | | |
| PTPN11 | c.226G > C | | | | | | | | | | | | | | | |
| PTPN11 | c.227A > G | | | | | | | | | | | | | | | |
| TET2 | c.1069delA | 1 | | | | | | | | | | | | | | |
| TET2 | c.1220delC | 1 | | | | | | | | | | | | | | |
| TET2 | c.1351dupA | 1 | | | | | | | | | | | | | | |
| TET2 | c.1496delC | 1 | | | | | | | | | | | | | | |
| TET2 | c.1608dupA | 1 | | | | | | | | | | | | | | |
| TET2 | c.1630C > T | 1 | | | | | | | | | | | | | | |
| TET2 | c.1648C > T | 1 | | | | | | | | | | | | | | |
| TET2 | c.1724C > T | 1 | | | | | | | | | | | | | | |
| TET2 | c.1754C > G | 1 | | | | | | | | | | | | | | |
| TET2 | c.1852C > T | 1 | | | | | | | | | | | | | | |
| TET2 | c.1903C > T | 1 | | | | | | | | | | | | | | |
| TET2 | c.1973_1974insAA | 1 | | | | | | | | | | | | | | |
| TET2 | c.2116C > T | 1 | | | | | | | | | | | | | | |
| TET2 | c.2146_2147delTC | 1 | | | | | | | | | | | | | | |
| TET2 | c.2185C > T | 1 | | | | | | | | | | | | | | |
| TET2 | c.2255_2261delATAAAGA | 1 | | | | | | | | | | | | | | |
| TET2 | c.2276delC | 1 | | | | | | | | | | | | | | |
| TET2 | c.2402delA | 1 | | | | | | | | | | | | | | |
| TET2 | c.2421G > A | 1 | | | | | | | | | | | | | | |
| TET2 | c.2596C > T | 1 | | | | | | | | | | | | | | |
| TET2 | c.2626C > T | 1 | | | | | | | | | | | | | | |
| TET2 | c.2646C > A | 1 | | | | | | | | | | | | | | |
| TET2 | c.2985dupA | 1 | | | | | | | | | | | | | | |
| TET2 | c.3016delG | 1 | | | | | | | | | | | | | | |
| TET2 | c.3095T > G | 1 | | | | | | | | | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| PML | c.1335A > G | | 2 | | | | | | | | | | | | |
| PML | c.1718G > A | | 2 | | | | | | | | | | | | |
| PML | c.−1C > T | | 2 | | | | | | | | | | | | |
| PML | c.2456G > C | | 2 | | | | | | | | | | | | |
| PML | c.2533G > A | | 2 | | | | | | | | | | | | |
| PTPN11 | c.1382C > G | | | | | | | | | | | | | | |
| PTPN11 | c.1403C > T | | | | | | | | | | | | | | |
| PTPN11 | c.1450G > A | | | | | | | | | | | | | | |
| PTPN11 | c.1507G > A | | | | | | | | | | | | | | |
| PTPN11 | c.181G > A | | | | | | | | | | | | | | |
| PTPN11 | c.182A > C | | | | | | | | | | | | | | |
| PTPN11 | c.214G > C | | | | | | | | | | | | | | |
| PTPN11 | c.215C > T | | | | | | | | | | | | | | |
| PTPN11 | c.226G > C | | | | | | | | | | | | | | |
| PTPN11 | c.227A > G | | | | | | | | | | | | | | |
| TET2 | c.1069delA | | | | | | | | | | | | | | |
| TET2 | c.1220delC | | | | | | | | | | | | | | |
| TET2 | c.1351dupA | | | | | | | | | | | | | | |
| TET2 | c.1496delC | | | | | | | | | | | | | | |
| TET2 | c.1608dupA | | | | | | | | | | | | | | |
| TET2 | c.1630C > T | | | | | | | | | | | | | | |
| TET2 | c.1648C > T | | | | | | | | | | | | | | |
| TET2 | c.1724C > T | | | | | | | | | | | | | | |
| TET2 | c.1754C > G | | | | | | | | | | | | | | |
| TET2 | c.1852C > T | | | | | | | | | | | | | | |
| TET2 | c.1903C > T | | | | | | | | | | | | | | |
| TET2 | c.1973_1974insAA | | | | | | | | | | | | | | |
| TET2 | c.2116C > T | | | | | | | | | | | | | | |
| TET2 | c.2146_2147delTC | | | | | | | | | | | | | | |
| TET2 | c.2185C > T | | | | | | | | | | | | | | |
| TET2 | c.2255_2261delATAAAGA | | | | | | | | | | | | | | |
| TET2 | c.2276delC | | | | | | | | | | | | | | |
| TET2 | c.2402delA | | | | | | | | | | | | | | |
| TET2 | c.2421G > A | | | | | | | | | | | | | | |
| TET2 | c.2596C > T | | | | | | | | | | | | | | |
| TET2 | c.2626C > T | | | | | | | | | | | | | | |
| TET2 | c.2646C > A | | | | | | | | | | | | | | |
| TET2 | c.2985dupA | | | | | | | | | | | | | | |
| TET2 | c.3016delG | | | | | | | | | | | | | | |
| TET2 | c.3095T > G | | | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| PML | c.1335A > G | | | | | | | | | | | | | |
| PML | c.1718G > A | | | | | | | | | | | | | |
| PML | c.−1C > T | | | | | | | | | | | | | |
| PML | c.2456G > C | | | | | | | | | | | | | |
| PML | c.2533G > A | | | | | | | | | | | | | |
| PTPN11 | c.1382C > G | | | | | | | | | | | | | |
| PTPN11 | c.1403C > T | | | | | | | | | | | | | |
| PTPN11 | c.1450G > A | | | | | | | | | | | | | |
| PTPN11 | c.1507G > A | | | | | | | | | | | | | |
| PTPN11 | c.181G > A | | | | | | | | | | | | | |
| PTPN11 | c.182A > C | | | | | | | | | | | | | |
| PTPN11 | c.214G > C | | | | | | | | | | | | | |
| PTPN11 | c.215C > T | | | | | | | | | | | | | |
| PTPN11 | c.226G > C | | | | | | | | | | | | | |
| PTPN11 | c.227A > G | | | | | | | | | | | | | |
| TET2 | c.1069delA | | | | | | | | | | | | | |
| TET2 | c.1220delC | | | | | | | | | | | | | |
| TET2 | c.1351dupA | | | | | | | | | | | | | |
| TET2 | c.1496delC | | | | | | | | | | | | | |
| TET2 | c.1608dupA | | | | | | | | | | | | | |
| TET2 | c.1630C > T | | | | | | | | | | | | | |
| TET2 | c.1648C > T | | | | | | | | | | | | | |
| TET2 | c.1724C > T | | | | | | | | | | | | | |
| TET2 | c.1754C > G | | | | | | | | | | | | | |
| TET2 | c.1852C > T | | | | | | | | | | | | | |
| TET2 | c.1903C > T | | | | | | | | | | | | | |
| TET2 | c.1973_1974insAA | | | | | | | | | | | | | |
| TET2 | c.2116C > T | | | | | | | | | | | | | |
| TET2 | c.2146_2147delTC | | | | | | | | | | | | | |
| TET2 | c.2185C > T | | | | | | | | | | | | | |
| TET2 | c.2255_2261delATAAAGA | | | | | | | | | | | | | |
| TET2 | c.2276delC | | | | | | | | | | | | | |
| TET2 | c.2402delA | | | | | | | | | | | | | |
| TET2 | c.2421G > A | | | | | | | | | | | | | |
| TET2 | c.2596C > T | | | | | | | | | | | | | |
| TET2 | c.2626C > T | | | | | | | | | | | | | |
| TET2 | c.2646C > A | | | | | | | | | | | | | |
| TET2 | c.2985dupA | | | | | | | | | | | | | |
| TET2 | c.3016delG | | | | | | | | | | | | | |
| TET2 | c.3095T > G | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| PML | c.1335A > G | | | | | | | | | | | |
| PML | c.1718G > A | | | | | | | | | | | |
| PML | c.−1C > T | | | | | | | | | | | |
| PML | c.2456G > C | | | | | | | | | | | |
| PML | c.2533G > A | | | | | | | | | | | |
| PTPN11 | c.1382C > G | | | | | | 1 | | | | | |
| PTPN11 | c.1403C > T | | | | | | 1 | | | | | |
| PTPN11 | c.1450G > A | | | | | | 1 | | | | | |
| PTPN11 | c.1507G > A | | | | | | 1 | | | | | |
| PTPN11 | c.181G > A | | | | | | 1 | | | | | |
| PTPN11 | c.182A > C | | | | | | 1 | | | | | |
| PTPN11 | c.214G > C | | | | | | 1 | | | | | |
| PTPN11 | c.215C > T | | | | | | 1 | | | | | |
| PTPN11 | c.226G > C | | | | | | 1 | | | | | |
| PTPN11 | c.227A > G | | | | | | 1 | | | | | |
| TET2 | c.1069delA | | | | | | | | | | | |
| TET2 | c.1220delC | | | | | | | | | | | |
| TET2 | c.1351dupA | | | | | | | | | | | |
| TET2 | c.1496delC | | | | | | | | | | | |
| TET2 | c.1608dupA | | | | | | | | | | | |
| TET2 | c.1630C > T | | | | | | | | | | | |
| TET2 | c.1648C > T | | | | | | | | | | | |
| TET2 | c.1724C > T | | | | | | | | | | | |
| TET2 | c.1754C > G | | | | | | | | | | | |
| TET2 | c.1852C > T | | | | | | | | | | | |
| TET2 | c.1903C > T | | | | | | | | | | | |
| TET2 | c.1973_1974insAA | | | | | | | | | | | |
| TET2 | c.2116C > T | | | | | | | | | | | |
| TET2 | c.2146_2147delTC | | | | | | | | | | | |
| TET2 | c.2185C > T | | | | | | | | | | | |
| TET2 | c.2255_2261delATAAAGA | | | | | | | | | | | |
| TET2 | c.2276delC | | | | | | | | | | | |
| TET2 | c.2402delA | | | | | | | | | | | |
| TET2 | c.2421G > A | | | | | | | | | | | |
| TET2 | c.2596C > T | | | | | | | | | | | |
| TET2 | c.2626C > T | | | | | | | | | | | |
| TET2 | c.2646C > A | | | | | | | | | | | |
| TET2 | c.2985dupA | | | | | | | | | | | |
| TET2 | c.3016delG | | | | | | | | | | | |
| TET2 | c.3095T > G | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| PML | c.1335A > G | | | | | | | | | | | | | |
| PML | c.1718G > A | | | | | | | | | | | | | |
| PML | c.−1C > T | | | | | | | | | | | | | |
| PML | c.2456G > C | | | | | | | | | | | | | |
| PML | c.2533G > A | | | | | | | | | | | | | |
| PTPN11 | c.1382C > G | | | | | | | | | | | | | |
| PTPN11 | c.1403C > T | | | | | | | | | | | | | |
| PTPN11 | c.1450G > A | | | | | | | | | | | | | |
| PTPN11 | c.1507G > A | | | | | | | | | | | | | |
| PTPN11 | c.181G > A | | | | | | | | | | | | | |
| PTPN11 | c.182A > C | | | | | | | | | | | | | |
| PTPN11 | c.214G > C | | | | | | | | | | | | | |
| PTPN11 | c.215C > T | | | | | | | | | | | | | |
| PTPN11 | c.226G > C | | | | | | | | | | | | | |
| PTPN11 | c.227A > G | | | | | | | | | | | | | |
| TET2 | c.1069delA | | | | | | | | | | | 1 | | |
| TET2 | c.1220delC | | | | | | | | | | | 1 | | |
| TET2 | c.1351dupA | | | | | | | | | | | 1 | | |
| TET2 | c.1496delC | | | | | | | | | | | 1 | | |
| TET2 | c.1608dupA | | | | | | | | | | | 1 | | |
| TET2 | c.1630C > T | | | | | | | | | | | 1 | | |
| TET2 | c.1648C > T | | | | | | | | | | | 1 | | |
| TET2 | c.1724C > T | | | | | | | | | | | 1 | | |
| TET2 | c.1754C > G | | | | | | | | | | | 1 | | |
| TET2 | c.1852C > T | | | | | | | | | | | 1 | | |
| TET2 | c.1903C > T | | | | | | | | | | | 1 | | |
| TET2 | c.1973_1974insAA | | | | | | | | | | | 1 | | |
| TET2 | c.2116C > T | | | | | | | | | | | 1 | | |
| TET2 | c.2146_2147delTC | | | | | | | | | | | 1 | | |
| TET2 | c.2185C > T | | | | | | | | | | | 1 | | |
| TET2 | c.2255_2261delATAAAGA | | | | | | | | | | | 1 | | |
| TET2 | c.2276delC | | | | | | | | | | | 1 | | |
| TET2 | c.2402delA | | | | | | | | | | | 1 | | |
| TET2 | c.2421G > A | | | | | | | | | | | 1 | | |
| TET2 | c.2596C > T | | | | | | | | | | | 1 | | |
| TET2 | c.2626C > T | | | | | | | | | | | 1 | | |
| TET2 | c.2646C > A | | | | | | | | | | | 1 | | |
| TET2 | c.2985dupA | | | | | | | | | | | 1 | | |
| TET2 | c.3016delG | | | | | | | | | | | 1 | | |
| TET2 | c.3095T > G | | | | | | | | | | | 1 | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| PML | c.1335A > G | | | | | | | | | | | |
| PML | c.1718G > A | | | | | | | | | | | |
| PML | c.−1C > T | | | | | | | | | | | |
| PML | c.2456G > C | | | | | | | | | | | |
| PML | c.2533G > A | | | | | | | | | | | |
| PTPN11 | c.1382C > G | | | | | | | | | | | |
| PTPN11 | c.1403C > T | | | | | | | | | | | |
| PTPN11 | c.1450G > A | | | | | | | | | | | |
| PTPN11 | c.1507G > A | | | | | | | | | | | |
| PTPN11 | c.181G > A | | | | | | | | | | | |
| PTPN11 | c.182A > C | | | | | | | | | | | |
| PTPN11 | c.214G > C | | | | | | | | | | | |
| PTPN11 | c.215C > T | | | | | | | | | | | |
| PTPN11 | c.226G > C | | | | | | | | | | | |
| PTPN11 | c.227A > G | | | | | | | | | | | |
| TET2 | c.1069delA | | | | | | | | | | | |
| TET2 | c.1220delC | | | | | | | | | | | |
| TET2 | c.1351dupA | | | | | | | | | | | |
| TET2 | c.1496delC | | | | | | | | | | | |
| TET2 | c.1608dupA | | | | | | | | | | | |
| TET2 | c.1630C > T | | | | | | | | | | | |
| TET2 | c.1648C > T | | | | | | | | | | | |
| TET2 | c.1724C > T | | | | | | | | | | | |
| TET2 | c.1754C > G | | | | | | | | | | | |
| TET2 | c.1852C > T | | | | | | | | | | | |
| TET2 | c.1903C > T | | | | | | | | | | | |
| TET2 | c.1973_1974insAA | | | | | | | | | | | |
| TET2 | c.2116C > T | | | | | | | | | | | |
| TET2 | c.2146_2147delTC | | | | | | | | | | | |
| TET2 | c.2185C > T | | | | | | | | | | | |
| TET2 | c.2255_2261delATAAAGA | | | | | | | | | | | |
| TET2 | c.2276delC | | | | | | | | | | | |
| TET2 | c.2402delA | | | | | | | | | | | |
| TET2 | c.2421G > A | | | | | | | | | | | |
| TET2 | c.2596C > T | | | | | | | | | | | |
| TET2 | c.2626C > T | | | | | | | | | | | |
| TET2 | c.2646C > A | | | | | | | | | | | |
| TET2 | c.2985dupA | | | | | | | | | | | |
| TET2 | c.3016delG | | | | | | | | | | | |
| TET2 | c.3095T > G | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| PML | c.1335A > G | | | | | | | | | | | | |
| PML | c.1718G > A | | | | | | | | | | | | |
| PML | c.−1C > T | | | | | | | | | | | | |
| PML | c.2456G > C | | | | | | | | | | | | |
| PML | c.2533G > A | | | | | | | | | | | | |
| PTPN11 | c.1382C > G | | | | | | | | | | | | |
| PTPN11 | c.1403C > T | | | | | | | | | | | | |
| PTPN11 | c.1450G > A | | | | | | | | | | | | |
| PTPN11 | c.1507G > A | | | | | | | | | | | | |
| PTPN11 | c.181G > A | | | | | | | | | | | | |
| PTPN11 | c.182A > C | | | | | | | | | | | | |
| PTPN11 | c.214G > C | | | | | | | | | | | | |
| PTPN11 | c.215C > T | | | | | | | | | | | | |
| PTPN11 | c.226G > C | | | | | | | | | | | | |
| PTPN11 | c.227A > G | | | | | | | | | | | | |
| TET2 | c.1069delA | | | | | | | | | | | | |
| TET2 | c.1220delC | | | | | | | | | | | | |
| TET2 | c.1351dupA | | | | | | | | | | | | |
| TET2 | c.1496delC | | | | | | | | | | | | |
| TET2 | c.1608dupA | | | | | | | | | | | | |
| TET2 | c.1630C > T | | | | | | | | | | | | |
| TET2 | c.1648C > T | | | | | | | | | | | | |
| TET2 | c.1724C > T | | | | | | | | | | | | |
| TET2 | c.1754C > G | | | | | | | | | | | | |
| TET2 | c.1852C > T | | | | | | | | | | | | |
| TET2 | c.1903C > T | | | | | | | | | | | | |
| TET2 | c.1973_1974insAA | | | | | | | | | | | | |
| TET2 | c.2116C > T | | | | | | | | | | | | |
| TET2 | c.2146_2147delTC | | | | | | | | | | | | |
| TET2 | c.2185C > T | | | | | | | | | | | | |
| TET2 | c.2255_2261delATAAAGA | | | | | | | | | | | | |
| TET2 | c.2276delC | | | | | | | | | | | | |
| TET2 | c.2402delA | | | | | | | | | | | | |
| TET2 | c.2421G > A | | | | | | | | | | | | |
| TET2 | c.2596C > T | | | | | | | | | | | | |
| TET2 | c.2626C > T | | | | | | | | | | | | |
| TET2 | c.2646C > A | | | | | | | | | | | | |
| TET2 | c.2985dupA | | | | | | | | | | | | |
| TET2 | c.3016delG | | | | | | | | | | | | |
| TET2 | c.3095T > G | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | | | |
| | | lenalidomide | | rituximab | lenvatinib | lorlatinib | inhibitor | methotrexate | methylprednisolone | midostaurin | mitoxantrone |
| Gene | Variant | ✓ | X | ✓ | ✓ | X | ✓ | X | X | ✓ | X |
| PML | c.1335A > G | | | | | | | | | | |
| PML | c.1718G > A | | | | | | | | | | |
| PML | c.−1C > T | | | | | | | | | | |
| PML | c.2456G > C | | | | | | | | | | |
| PML | c.2533G > A | | | | | | | | | | |
| PTPN11 | c.1382C > G | | | | | | | | | | |
| PTPN11 | c.1403C > T | | | | | | | | | | |
| PTPN11 | c.1450G > A | | | | | | | | | | |
| PTPN11 | c.1507G > A | | | | | | | | | | |
| PTPN11 | c.181G > A | | | | | | | | | | |
| PTPN11 | c.182A > C | | | | | | | | | | |
| PTPN11 | c.214G > C | | | | | | | | | | |
| PTPN11 | c.215C > T | | | | | | | | | | |
| PTPN11 | c.226G > C | | | | | | | | | | |
| PTPN11 | c.227A > G | | | | | | | | | | |
| TET2 | c.1069delA | | | | | | | | | | |
| TET2 | c.1220delC | | | | | | | | | | |
| TET2 | c.1351dupA | | | | | | | | | | |
| TET2 | c.1496delC | | | | | | | | | | |
| TET2 | c.1608dupA | | | | | | | | | | |
| TET2 | c.1630C > T | | | | | | | | | | |
| TET2 | c.1648C > T | | | | | | | | | | |
| TET2 | c.1724C > T | | | | | | | | | | |
| TET2 | c.1754C > G | | | | | | | | | | |
| TET2 | c.1852C > T | | | | | | | | | | |
| TET2 | c.1903C > T | | | | | | | | | | |
| TET2 | c.1973_1974insAA | | | | | | | | | | |
| TET2 | c.2116C > T | | | | | | | | | | |
| TET2 | c.2146_2147delTC | | | | | | | | | | |
| TET2 | c.2185C > T | | | | | | | | | | |
| TET2 | c.2255_2261delATAAAGA | | | | | | | | | | |
| TET2 | c.2276delC | | | | | | | | | | |
| TET2 | c.2402delA | | | | | | | | | | |
| TET2 | c.2421G > A | | | | | | | | | | |
| TET2 | c.2596C > T | | | | | | | | | | |
| TET2 | c.2626C > T | | | | | | | | | | |
| TET2 | c.2646C > A | | | | | | | | | | |
| TET2 | c.2985dupA | | | | | | | | | | |
| TET2 | c.3016delG | | | | | | | | | | |
| TET2 | c.3095T > G | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| PML | c.1335A > G | | | | | | | | | |
| PML | c.1718G > A | | | | | | | | | |
| PML | c.−1C > T | | | | | | | | | |
| PML | c.2456G > C | | | | | | | | | |
| PML | c.2533G > A | | | | | | | | | |
| PTPN11 | c.1382C > G | | | | | | | | | |
| PTPN11 | c.1403C > T | | | | | | | | | |
| PTPN11 | c.1450G > A | | | | | | | | | |
| PTPN11 | c.1507G > A | | | | | | | | | |
| PTPN11 | c.181G > A | | | | | | | | | |
| PTPN11 | c.182A > C | | | | | | | | | |
| PTPN11 | c.214G > C | | | | | | | | | |
| PTPN11 | c.215C > T | | | | | | | | | |
| PTPN11 | c.226G > C | | | | | | | | | |
| PTPN11 | c.227A > G | | | | | | | | | |
| TET2 | c.1069delA | | | | | | | | | |
| TET2 | c.1220delC | | | | | | | | | |
| TET2 | c.1351dupA | | | | | | | | | |
| TET2 | c.1496delC | | | | | | | | | |
| TET2 | c.1608dupA | | | | | | | | | |
| TET2 | c.1630C > T | | | | | | | | | |
| TET2 | c.1648C > T | | | | | | | | | |
| TET2 | c.1724C > T | | | | | | | | | |
| TET2 | c.1754C > G | | | | | | | | | |
| TET2 | c.1852C > T | | | | | | | | | |
| TET2 | c.1903C > T | | | | | | | | | |
| TET2 | c.1973_1974insAA | | | | | | | | | |
| TET2 | c.2116C > T | | | | | | | | | |
| TET2 | c.2146_2147delTC | | | | | | | | | |
| TET2 | c.2185C > T | | | | | | | | | |
| TET2 | c.2255_2261delATAAAGA | | | | | | | | | |
| TET2 | c.2276delC | | | | | | | | | |
| TET2 | c.2402delA | | | | | | | | | |
| TET2 | c.2421G > A | | | | | | | | | |
| TET2 | c.2596C > T | | | | | | | | | |
| TET2 | c.2626C > T | | | | | | | | | |
| TET2 | c.2646C > A | | | | | | | | | |
| TET2 | c.2985dupA | | | | | | | | | |
| TET2 | c.3016delG | | | | | | | | | |
| TET2 | c.3095T > G | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alpha-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| PML | c.1335A > G | | | | | | | | | | | | |
| PML | c.1718G > A | | | | | | | | | | | | |
| PML | c.−1C > T | | | | | | | | | | | | |
| PML | c.2456G > C | | | | | | | | | | | | |
| PML | c.2533G > A | | | | | | | | | | | | |
| PTPN11 | c.1382C > G | | | | | | | | | | | | |
| PTPN11 | c.1403C > T | | | | | | | | | | | | |
| PTPN11 | c.1450G > A | | | | | | | | | | | | |
| PTPN11 | c.1507G > A | | | | | | | | | | | | |
| PTPN11 | c.181G > A | | | | | | | | | | | | |
| PTPN11 | c.182A > C | | | | | | | | | | | | |
| PTPN11 | c.214G > C | | | | | | | | | | | | |
| PTPN11 | c.215C > T | | | | | | | | | | | | |
| PTPN11 | c.226G > C | | | | | | | | | | | | |
| PTPN11 | c.227A > G | | | | | | | | | | | | |
| TET2 | c.1069delA | | | | 1 | | | | | | | | |
| TET2 | c.1220delC | | | | 1 | | | | | | | | |
| TET2 | c.1351dupA | | | | 1 | | | | | | | | |
| TET2 | c.1496delC | | | | 1 | | | | | | | | |
| TET2 | c.1608dupA | | | | 1 | | | | | | | | |
| TET2 | c.1630C > T | | | | 1 | | | | | | | | |
| TET2 | c.1648C > T | | | | 1 | | | | | | | | |
| TET2 | c.1724C > T | | | | 1 | | | | | | | | |
| TET2 | c.1754C > G | | | | | | | | | | | | |
| TET2 | c.1852C > T | | | | 1 | | | | | | | | |
| TET2 | c.1903C > T | | | | | | | | | | | | |
| TET2 | c.1973_1974insAA | | | | 1 | | | | | | | | |
| TET2 | c.2116C > T | | | | 1 | | | | | | | | |
| TET2 | c.2146_2147delTC | | | | 1 | | | | | | | | |
| TET2 | c.2185C > T | | | | 1 | | | | | | | | |
| TET2 | c.2255_2261delATAAAGA | | | | 1 | | | | | | | | |
| TET2 | c.2276delC | | | | 1 | | | | | | | | |
| TET2 | c.2402delA | | | | | | | | | | | | |
| TET2 | c.2421G > A | | | | | | | | | | | | |
| TET2 | c.2596C > T | | | | 1 | | | | | | | | |
| TET2 | c.2626C > T | | | | | | | | | | | | |
| TET2 | c.2646C > A | | | | | | | | | | | | |
| TET2 | c.2985dupA | | | | 1 | | | | | | | | |
| TET2 | c.3016delG | | | | 1 | | | | | | | | |
| TET2 | c.3095T > G | | | | 1 | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| PML | c.1335A > G | | | | | | | | | | | |
| PML | c.1718G > A | | | | | | | | | | | |
| PML | c.−1C > T | | | | | | | | | | | |
| PML | c.2456G > C | | | | | | | | | | | |
| PML | c.2533G > A | | | | | | | | | | | |
| PTPN11 | c.1382C > G | | | | | | | | | | | |
| PTPN11 | c.1403C > T | | | | | | | | | | | |
| PTPN11 | c.1450G > A | | | | | | | | | | | |
| PTPN11 | c.1507G > A | | | | | | | | | | | |
| PTPN11 | c.181G > A | | | | | | | | | | | |
| PTPN11 | c.182A > C | | | | | | | | | | | |
| PTPN11 | c.214G > C | | | | | | | | | | | |
| PTPN11 | c.215C > T | | | | | | | | | | | |
| PTPN11 | c.226G > C | | | | | | | | | | | |
| PTPN11 | c.227A > G | | | | | | | | | | | |
| TET2 | c.1069delA | | | | | | | | | | | |
| TET2 | c.1220delC | | | | | | | | | | | |
| TET2 | c.1351dupA | | | | | | | | | | | |
| TET2 | c.1496delC | | | | | | | | | | | |
| TET2 | c.1608dupA | | | | | | | | | | | |
| TET2 | c.1630C > T | | | | | | | | | | | |
| TET2 | c.1648C > T | | | | | | | | | | | |
| TET2 | c.1724C > T | | | | | | | | | | | |
| TET2 | c.1754C > G | | | | | | | | | | | |
| TET2 | c.1852C > T | | | | | | | | | | | |
| TET2 | c.1903C > T | | | | | | | | | | | |
| TET2 | c.1973_1974insAA | | | | | | | | | | | |
| TET2 | c.2116C > T | | | | | | | | | | | |
| TET2 | c.2146_2147delTC | | | | | | | | | | | |
| TET2 | c.2185C > T | | | | | | | | | | | |
| TET2 | c.2255_2261delATAAAGA | | | | | | | | | | | |
| TET2 | c.2276delC | | | | | | | | | | | |
| TET2 | c.2402delA | | | | | | | | | | | |
| TET2 | c.2421G > A | | | | | | | | | | | |
| TET2 | c.2596C > T | | | | | | | | | | | |
| TET2 | c.2626C > T | | | | | | | | | | | |
| TET2 | c.2646C > A | | | | | | | | | | | |
| TET2 | c.2985dupA | | | | | | | | | | | |
| TET2 | c.3016delG | | | | | | | | | | | |
| TET2 | c.3095T > G | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| PML | c.1335A > G | | | | | | | | | | | |
| PML | c.1718G > A | | | | | | | | | | | |
| PML | c.−1C > T | | | | | | | | | | | |
| PML | c.2456G > C | | | | | | | | | | | |
| PML | c.2533G > A | | | | | | | | | | | |
| PTPN11 | c.1382C > G | | | | | | | | | | | |
| PTPN11 | c.1403C > T | | | | | | | | | | | |
| PTPN11 | c.1450G > A | | | | | | | | | | | |
| PTPN11 | c.1507G > A | | | | | | | | | | | |
| PTPN11 | c.181G > A | | | | | | | | | | | |
| PTPN11 | c.182A > C | | | | | | | | | | | |
| PTPN11 | c.214G > C | | | | | | | | | | | |
| PTPN11 | c.215C > T | | | | | | | | | | | |
| PTPN11 | c.226G > C | | | | | | | | | | | |
| PTPN11 | c.227A > G | | | | | | | | | | | |
| TET2 | c.1069delA | | | | | | | | | | | |
| TET2 | c.1220delC | | | | | | | | | | | |
| TET2 | c.1351dupA | | | | | | | | | | | |
| TET2 | c.1496delC | | | | | | | | | | | |
| TET2 | c.1608dupA | | | | | | | | | | | |
| TET2 | c.1630C > T | | | | | | | | | | | |
| TET2 | c.1648C > T | | | | | | | | | | | |
| TET2 | c.1724C > T | | | | | | | | | | | |
| TET2 | c.1754C > G | | | | | | | | | | | |
| TET2 | c.1852C > T | | | | | | | | | | | |
| TET2 | c.1903C > T | | | | | | | | | | | |
| TET2 | c.1973_1974insAA | | | | | | | | | | | |
| TET2 | c.2116C > T | | | | | | | | | | | |
| TET2 | c.2146_2147delTC | | | | | | | | | | | |
| TET2 | c.2185C > T | | | | | | | | | | | |
| TET2 | c.2255_2261delATAAAGA | | | | | | | | | | | |
| TET2 | c.2276delC | | | | | | | | | | | |
| TET2 | c.2402delA | | | | | | | | | | | |
| TET2 | c.2421G > A | | | | | | | | | | | |
| TET2 | c.2596C > T | | | | | | | | | | | |
| TET2 | c.2626C > T | | | | | | | | | | | |
| TET2 | c.2646C > A | | | | | | | | | | | |
| TET2 | c.2985dupA | | | | | | | | | | | |
| TET2 | c.3016delG | | | | | | | | | | | |
| TET2 | c.3095T > G | | | | | | | | | | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| TET2 | c.3172A > G | 1 | | | | | | | | | | | | | | |
| TET2 | c.3312delT | 1 | | | | | | | | | | | | | | |
| TET2 | c.3312dupT | 1 | | | | | | | | | | | | | | |
| TET2 | c.3344delC | 1 | | | | | | | | | | | | | | |
| TET2 | c.3380_3381delAA | 1 | | | | | | | | | | | | | | |
| TET2 | c.3383_3384delAT | 1 | | | | | | | | | | | | | | |
| TET2 | c.3481A > G | 1 | | | | | | | | | | | | | | |
| TET2 | c.3500 + | 1 | | | | | | | | | | | | | | |
| | 1G > A | | | | | | | | | | | | | | | |
| TET2 | c.3535dupA | 1 | | | | | | | | | | | | | | |
| TET2 | c.3546delT | 1 | | | | | | | | | | | | | | |
| TET2 | c.3555delA | 1 | | | | | | | | | | | | | | |
| TET2 | c.3578delG | 1 | | | | | | | | | | | | | | |
| TET2 | c.3583_3585dupATT | 1 | | | | | | | | | | | | | | |
| TET2 | c.3594 + | 1 | | | | | | | | | | | | | | |
| | 5G > A | | | | | | | | | | | | | | | |
| TET2 | c.3594G > T | 1 | | | | | | | | | | | | | | |
| TET2 | c.3618delA | 1 | | | | | | | | | | | | | | |
| TET2 | c.3626T > C | 1 | | | | | | | | | | | | | | |
| TET2 | c.3641G > A | 1 | | | | | | | | | | | | | | |
| TET2 | c.3646C > T | 1 | | | | | | | | | | | | | | |
| TET2 | c.3689T > G | 1 | | | | | | | | | | | | | | |
| TET2 | c.3732_3733delCT | 1 | | | | | | | | | | | | | | |
| TET2 | c.3734A > G | 1 | | | | | | | | | | | | | | |
| TET2 | c.3764dupA | 1 | | | | | | | | | | | | | | |
| TET2 | c.3782G > A | 1 | | | | | | | | | | | | | | |
| TET2 | c.3789T > G | 1 | | | | | | | | | | | | | | |
| TET2 | c.3804 − | 1 | | | | | | | | | | | | | | |
| | 2A > G | | | | | | | | | | | | | | | |
| TET2 | c.3813C > G | 1 | | | | | | | | | | | | | | |
| TET2 | c.3818G > T | 1 | | | | | | | | | | | | | | |
| TET2 | c.3849dupC | 1 | | | | | | | | | | | | | | |
| TET2 | c.3863G > A | 1 | | | | | | | | | | | | | | |
| TET2 | c.3863G > T | 1 | | | | | | | | | | | | | | |
| TET2 | c.3897G > C | 1 | | | | | | | | | | | | | | |
| TET2 | c.3899T > C | 1 | | | | | | | | | | | | | | |
| TET2 | c.3899T > G | 1 | | | | | | | | | | | | | | |
| TET2 | c.3967G > T | 1 | | | | | | | | | | | | | | |
| TET2 | c.3972delT | 1 | | | | | | | | | | | | | | |
| TET2 | c.4075C > T | 1 | | | | | | | | | | | | | | |
| TET2 | c.4081G > T | 1 | | | | | | | | | | | | | | |
| TET2 | c.4104C > A | 1 | | | | | | | | | | | | | | |
| TET2 | c.4150G > C | 1 | | | | | | | | | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| TET2 | c.3172A > G | | | | | | | | | | | | | | |
| TET2 | c.3312delT | | | | | | | | | | | | | | |
| TET2 | c.3312dupT | | | | | | | | | | | | | | |
| TET2 | c.3344delC | | | | | | | | | | | | | | |
| TET2 | c.3380_3381delAA | | | | | | | | | | | | | | |
| TET2 | c.3383_3384delAT | | | | | | | | | | | | | | |
| TET2 | c.3481A > G | | | | | | | | | | | | | | |
| TET2 | c.3500 + | | | | | | | | | | | | | | |
| | 1G > A | | | | | | | | | | | | | | |
| TET2 | c.3535dupA | | | | | | | | | | | | | | |
| TET2 | c.3546delT | | | | | | | | | | | | | | |
| TET2 | c.3555delA | | | | | | | | | | | | | | |
| TET2 | c.3578delG | | | | | | | | | | | | | | |
| TET2 | c.3583_3585dupATT | | | | | | | | | | | | | | |
| TET2 | c.3594 + | | | | | | | | | | | | | | |
| | 5G > A | | | | | | | | | | | | | | |
| TET2 | c.3594G > T | | | | | | | | | | | | | | |
| TET2 | c.3618delA | | | | | | | | | | | | | | |
| TET2 | c.3626T > C | | | | | | | | | | | | | | |
| TET2 | c.3641G > A | | | | | | | | | | | | | | |
| TET2 | c.3646C > T | | | | | | | | | | | | | | |
| TET2 | c.3689T > G | | | | | | | | | | | | | | |
| TET2 | c.3732_3733delCT | | | | | | | | | | | | | | |
| TET2 | c.3734A > G | | | | | | | | | | | | | | |
| TET2 | c.3764dupA | | | | | | | | | | | | | | |
| TET2 | c.3782G > A | | | | | | | | | | | | | | |
| TET2 | c.3789T > G | | | | | | | | | | | | | | |
| TET2 | c.3804 − | | | | | | | | | | | | | | |
| | 2A > G | | | | | | | | | | | | | | |
| TET2 | c.3813C > G | | | | | | | | | | | | | | |
| TET2 | c.3818G > T | | | | | | | | | | | | | | |
| TET2 | c.3849dupC | | | | | | | | | | | | | | |
| TET2 | c.3863G > A | | | | | | | | | | | | | | |
| TET2 | c.3863G > T | | | | | | | | | | | | | | |
| TET2 | c.3897G > C | | | | | | | | | | | | | | |
| TET2 | c.3899T > C | | | | | | | | | | | | | | |
| TET2 | c.3899T > G | | | | | | | | | | | | | | |
| TET2 | c.3967G > T | | | | | | | | | | | | | | |
| TET2 | c.3972delT | | | | | | | | | | | | | | |
| TET2 | c.4075C > T | | | | | | | | | | | | | | |
| TET2 | c.4081G > T | | | | | | | | | | | | | | |
| TET2 | c.4104C > A | | | | | | | | | | | | | | |
| TET2 | c.4150G > C | | | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| TET2 | c.3172A > G | | | | | | | | | | | | | |
| TET2 | c.3312delT | | | | | | | | | | | | | |
| TET2 | c.3312dupT | | | | | | | | | | | | | |
| TET2 | c.3344delC | | | | | | | | | | | | | |
| TET2 | c.3380_3381delAA | | | | | | | | | | | | | |
| TET2 | c.3383_3384delAT | | | | | | | | | | | | | |
| TET2 | c.3481A > G | | | | | | | | | | | | | |
| TET2 | c.3500 + | | | | | | | | | | | | | |
| | 1G > A | | | | | | | | | | | | | |
| TET2 | c.3535dupA | | | | | | | | | | | | | |
| TET2 | c.3546delT | | | | | | | | | | | | | |
| TET2 | c.3555delA | | | | | | | | | | | | | |
| TET2 | c.3578delG | | | | | | | | | | | | | |
| TET2 | c.3583_3585dupATT | | | | | | | | | | | | | |
| TET2 | c.3594 + | | | | | | | | | | | | | |
| | 5G > A | | | | | | | | | | | | | |
| TET2 | c.3594G > T | | | | | | | | | | | | | |
| TET2 | c.3618delA | | | | | | | | | | | | | |
| TET2 | c.3626T > C | | | | | | | | | | | | | |
| TET2 | c.3641G > A | | | | | | | | | | | | | |
| TET2 | c.3646C > T | | | | | | | | | | | | | |
| TET2 | c.3689T > G | | | | | | | | | | | | | |
| TET2 | c.3732_3733delCT | | | | | | | | | | | | | |
| TET2 | c.3734A > G | | | | | | | | | | | | | |
| TET2 | c.3764dupA | | | | | | | | | | | | | |
| TET2 | c.3782G > A | | | | | | | | | | | | | |
| TET2 | c.3789T > G | | | | | | | | | | | | | |
| TET2 | c.3804 − | | | | | | | | | | | | | |
| | 2A > G | | | | | | | | | | | | | |
| TET2 | c.3813C > G | | | | | | | | | | | | | |
| TET2 | c.3818G > T | | | | | | | | | | | | | |
| TET2 | c.3849dupC | | | | | | | | | | | | | |
| TET2 | c.3863G > A | | | | | | | | | | | | | |
| TET2 | c.3863G > T | | | | | | | | | | | | | |
| TET2 | c.3897G > C | | | | | | | | | | | | | |
| TET2 | c.3899T > C | | | | | | | | | | | | | |
| TET2 | c.3899T > G | | | | | | | | | | | | | |
| TET2 | c.3967G > T | | | | | | | | | | | | | |
| TET2 | c.3972delT | | | | | | | | | | | | | |
| TET2 | c.4075C > T | | | | | | | | | | | | | |
| TET2 | c.4081G > T | | | | | | | | | | | | | |
| TET2 | c.4104C > A | | | | | | | | | | | | | |
| TET2 | c.4150G > C | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| TET2 | c.3172A > G | | | | | | | | | | | |
| TET2 | c.3312delT | | | | | | | | | | | |
| TET2 | c.3312dupT | | | | | | | | | | | |
| TET2 | c.3344delC | | | | | | | | | | | |
| TET2 | c.3380_3381delAA | | | | | | | | | | | |
| TET2 | c.3383_3384delAT | | | | | | | | | | | |
| TET2 | c.3481A > G | | | | | | | | | | | |
| TET2 | c.3500 + | | | | | | | | | | | |
| | 1G > A | | | | | | | | | | | |
| TET2 | c.3535dupA | | | | | | | | | | | |
| TET2 | c.3546delT | | | | | | | | | | | |
| TET2 | c.3555delA | | | | | | | | | | | |
| TET2 | c.3578delG | | | | | | | | | | | |
| TET2 | c.3583_3585dupATT | | | | | | | | | | | |
| TET2 | c.3594 + | | | | | | | | | | | |
| | 5G > A | | | | | | | | | | | |
| TET2 | c.3594G > T | | | | | | | | | | | |
| TET2 | c.3618delA | | | | | | | | | | | |
| TET2 | c.3626T > C | | | | | | | | | | | |
| TET2 | c.3641G > A | | | | | | | | | | | |
| TET2 | c.3646C > T | | | | | | | | | | | |
| TET2 | c.3689T > G | | | | | | | | | | | |
| TET2 | c.3732_3733delCT | | | | | | | | | | | |
| TET2 | c.3734A > G | | | | | | | | | | | |
| TET2 | c.3764dupA | | | | | | | | | | | |
| TET2 | c.3782G > A | | | | | | | | | | | |
| TET2 | c.3789T > G | | | | | | | | | | | |
| TET2 | c.3804 − | | | | | | | | | | | |
| | 2A > G | | | | | | | | | | | |
| TET2 | c.3813C > G | | | | | | | | | | | |
| TET2 | c.3818G > T | | | | | | | | | | | |
| TET2 | c.3849dupC | | | | | | | | | | | |
| TET2 | c.3863G > A | | | | | | | | | | | |
| TET2 | c.3863G > T | | | | | | | | | | | |
| TET2 | c.3897G > C | | | | | | | | | | | |
| TET2 | c.3899T > C | | | | | | | | | | | |
| TET2 | c.3899T > G | | | | | | | | | | | |
| TET2 | c.3967G > T | | | | | | | | | | | |
| TET2 | c.3972delT | | | | | | | | | | | |
| TET2 | c.4075C > T | | | | | | | | | | | |
| TET2 | c.4081G > T | | | | | | | | | | | |
| TET2 | c.4104C > A | | | | | | | | | | | |
| TET2 | c.4150G > C | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| TET2 | c.3172A > G | | | | | | | | | | | 1 | | |
| TET2 | c.3312delT | | | | | | | | | | | 1 | | |
| TET2 | c.3312dupT | | | | | | | | | | | 1 | | |
| TET2 | c.3344delC | | | | | | | | | | | 1 | | |
| TET2 | c.3380_3381delAA | | | | | | | | | | | 1 | | |
| TET2 | c.3383_3384delAT | | | | | | | | | | | 1 | | |
| TET2 | c.3481A > G | | | | | | | | | | | 1 | | |
| TET2 | c.3500 + | | | | | | | | | | | 1 | | |
| | 1G > A | | | | | | | | | | | | | |
| TET2 | c.3535dupA | | | | | | | | | | | 1 | | |
| TET2 | c.3546delT | | | | | | | | | | | 1 | | |
| TET2 | c.3555delA | | | | | | | | | | | 1 | | |
| TET2 | c.3578delG | | | | | | | | | | | 1 | | |
| TET2 | c.3583_3585dupATT | | | | | | | | | | | 1 | | |
| TET2 | c.3594 + | | | | | | | | | | | 1 | | |
| | 5G > A | | | | | | | | | | | | | |
| TET2 | c.3594G > T | | | | | | | | | | | 1 | | |
| TET2 | c.3618delA | | | | | | | | | | | 1 | | |
| TET2 | c.3626T > C | | | | | | | | | | | 1 | | |
| TET2 | c.3641G > A | | | | | | | | | | | 1 | | |
| TET2 | c.3646C > T | | | | | | | | | | | 1 | | |
| TET2 | c.3689T > G | | | | | | | | | | | 1 | | |
| TET2 | c.3732_3733delCT | | | | | | | | | | | 1 | | |
| TET2 | c.3734A > G | | | | | | | | | | | 1 | | |
| TET2 | c.3764dupA | | | | | | | | | | | 1 | | |
| TET2 | c.3782G > A | | | | | | | | | | | 1 | | |
| TET2 | c.3789T > G | | | | | | | | | | | 1 | | |
| TET2 | c.3804 − | | | | | | | | | | | 1 | | |
| | 2A > G | | | | | | | | | | | | | |
| TET2 | c.3813C > G | | | | | | | | | | | 1 | | |
| TET2 | c.3818G > T | | | | | | | | | | | 1 | | |
| TET2 | c.3849dupC | | | | | | | | | | | 1 | | |
| TET2 | c.3863G > A | | | | | | | | | | | 1 | | |
| TET2 | c.3863G > T | | | | | | | | | | | 1 | | |
| TET2 | c.3897G > C | | | | | | | | | | | 1 | | |
| TET2 | c.3899T > C | | | | | | | | | | | 1 | | |
| TET2 | c.3899T > G | | | | | | | | | | | 1 | | |
| TET2 | c.3967G > T | | | | | | | | | | | 1 | | |
| TET2 | c.3972delT | | | | | | | | | | | 1 | | |
| TET2 | c.4075C > T | | | | | | | | | | | 1 | | |
| TET2 | c.4081G > T | | | | | | | | | | | 1 | | |
| TET2 | c.4104C > A | | | | | | | | | | | 1 | | |
| TET2 | c.4150G > C | | | | | | | | | | | 1 | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| TET2 | c.3172A > G | | | | | | | | | | | |
| TET2 | c.3312delT | | | | | | | | | | | |
| TET2 | c.3312dupT | | | | | | | | | | | |
| TET2 | c.3344delC | | | | | | | | | | | |
| TET2 | c.3380_3381delAA | | | | | | | | | | | |
| TET2 | c.3383_3384delAT | | | | | | | | | | | |
| TET2 | c.3481A > G | | | | | | | | | | | |
| TET2 | c.3500 + | | | | | | | | | | | |
| | 1G > A | | | | | | | | | | | |
| TET2 | c.3535dupA | | | | | | | | | | | |
| TET2 | c.3546delT | | | | | | | | | | | |
| TET2 | c.3555delA | | | | | | | | | | | |
| TET2 | c.3578delG | | | | | | | | | | | |
| TET2 | c.3583_3585dupATT | | | | | | | | | | | |
| TET2 | c.3594 + | | | | | | | | | | | |
| | 5G > A | | | | | | | | | | | |
| TET2 | c.3594G > T | | | | | | | | | | | |
| TET2 | c.3618delA | | | | | | | | | | | |
| TET2 | c.3626T > C | | | | | | | | | | | |
| TET2 | c.3641G > A | | | | | | | | | | | |
| TET2 | c.3646C > T | | | | | | | | | | | |
| TET2 | c.3689T > G | | | | | | | | | | | |
| TET2 | c.3732_3733delCT | | | | | | | | | | | |
| TET2 | c.3734A > G | | | | | | | | | | | |
| TET2 | c.3764dupA | | | | | | | | | | | |
| TET2 | c.3782G > A | | | | | | | | | | | |
| TET2 | c.3789T > G | | | | | | | | | | | |
| TET2 | c.3804 − | | | | | | | | | | | |
| | 2A > G | | | | | | | | | | | |
| TET2 | c.3813C > G | | | | | | | | | | | |
| TET2 | c.3818G > T | | | | | | | | | | | |
| TET2 | c.3849dupC | | | | | | | | | | | |
| TET2 | c.3863G > A | | | | | | | | | | | |
| TET2 | c.3863G > T | | | | | | | | | | | |
| TET2 | c.3897G > C | | | | | | | | | | | |
| TET2 | c.3899T > C | | | | | | | | | | | |
| TET2 | c.3899T > G | | | | | | | | | | | |
| TET2 | c.3967G > T | | | | | | | | | | | |
| TET2 | c.3972delT | | | | | | | | | | | |
| TET2 | c.4075C > T | | | | | | | | | | | |
| TET2 | c.4081G > T | | | | | | | | | | | |
| TET2 | c.4104C > A | | | | | | | | | | | |
| TET2 | c.4150G > C | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| TET2 | c.3172A > G | | | | | | | | | | | | |
| TET2 | c.3312delT | | | | | | | | | | | | |
| TET2 | c.3312dupT | | | | | | | | | | | | |
| TET2 | c.3344delC | | | | | | | | | | | | |
| TET2 | c.3380_3381delAA | | | | | | | | | | | | |
| TET2 | c.3383_3384delAT | | | | | | | | | | | | |
| TET2 | c.3481A > G | | | | | | | | | | | | |
| TET2 | c.3500 + | | | | | | | | | | | | |
| | 1G > A | | | | | | | | | | | | |
| TET2 | c.3535dupA | | | | | | | | | | | | |
| TET2 | c.3546delT | | | | | | | | | | | | |
| TET2 | c.3555delA | | | | | | | | | | | | |
| TET2 | c.3578delG | | | | | | | | | | | | |
| TET2 | c.3583_3585dupATT | | | | | | | | | | | | |
| TET2 | c.3594 + | | | | | | | | | | | | |
| | 5G > A | | | | | | | | | | | | |
| TET2 | c.3594G > T | | | | | | | | | | | | |
| TET2 | c.3618delA | | | | | | | | | | | | |
| TET2 | c.3626T > C | | | | | | | | | | | | |
| TET2 | c.3641G > A | | | | | | | | | | | | |
| TET2 | c.3646C > T | | | | | | | | | | | | |
| TET2 | c.3689T > G | | | | | | | | | | | | |
| TET2 | c.3732_3733delCT | | | | | | | | | | | | |
| TET2 | c.3734A > G | | | | | | | | | | | | |
| TET2 | c.3764dupA | | | | | | | | | | | | |
| TET2 | c.3782G > A | | | | | | | | | | | | |
| TET2 | c.3789T > G | | | | | | | | | | | | |
| TET2 | c.3804 − | | | | | | | | | | | | |
| | 2A > G | | | | | | | | | | | | |
| TET2 | c.3813C > G | | | | | | | | | | | | |
| TET2 | c.3818G > T | | | | | | | | | | | | |
| TET2 | c.3849dupC | | | | | | | | | | | | |
| TET2 | c.3863G > A | | | | | | | | | | | | |
| TET2 | c.3863G > T | | | | | | | | | | | | |
| TET2 | c.3897G > C | | | | | | | | | | | | |
| TET2 | c.3899T > C | | | | | | | | | | | | |
| TET2 | c.3899T > G | | | | | | | | | | | | |
| TET2 | c.3967G > T | | | | | | | | | | | | |
| TET2 | c.3972delT | | | | | | | | | | | | |
| TET2 | c.4075C > T | | | | | | | | | | | | |
| TET2 | c.4081G > T | | | | | | | | | | | | |
| TET2 | c.4104C > A | | | | | | | | | | | | |
| TET2 | c.4150G > C | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | methylpred- | | mitoxan- |
| | | lenalidomide | | rituximab | lenvatinib | lorlatinib | inhibitor | methotrexate | nisolone | midostaurin | trone |
| Gene | Variant | ✓ | X | ✓ | ✓ | X | ✓ | X | X | ✓ | X |
| TET2 | c.3172A > G | | | | | | | | | | |
| TET2 | c.3312delT | | | | | | | | | | |
| TET2 | c.3312dupT | | | | | | | | | | |
| TET2 | c.3344delC | | | | | | | | | | |
| TET2 | c.3380_3381delAA | | | | | | | | | | |
| TET2 | c.3383_3384delAT | | | | | | | | | | |
| TET2 | c.3481A > G | | | | | | | | | | |
| TET2 | c.3500 + | | | | | | | | | | |
| | 1G > A | | | | | | | | | | |
| TET2 | c.3535dupA | | | | | | | | | | |
| TET2 | c.3546delT | | | | | | | | | | |
| TET2 | c.3555delA | | | | | | | | | | |
| TET2 | c.3578delG | | | | | | | | | | |
| TET2 | c.3583_3585dupATT | | | | | | | | | | |
| TET2 | c.3594 + | | | | | | | | | | |
| | 5G > A | | | | | | | | | | |
| TET2 | c.3594G > T | | | | | | | | | | |
| TET2 | c.3618delA | | | | | | | | | | |
| TET2 | c.3626T > C | | | | | | | | | | |
| TET2 | c.3641G > A | | | | | | | | | | |
| TET2 | c.3646C > T | | | | | | | | | | |
| TET2 | c.3689T > G | | | | | | | | | | |
| TET2 | c.3732_3733delCT | | | | | | | | | | |
| TET2 | c.3734A > G | | | | | | | | | | |
| TET2 | c.3764dupA | | | | | | | | | | |
| TET2 | c.3782G > A | | | | | | | | | | |
| TET2 | c.3789T > G | | | | | | | | | | |
| TET2 | c.3804 − | | | | | | | | | | |
| | 2A > G | | | | | | | | | | |
| TET2 | c.3813C > G | | | | | | | | | | |
| TET2 | c.3818G > T | | | | | | | | | | |
| TET2 | c.3849dupC | | | | | | | | | | |
| TET2 | c.3863G > A | | | | | | | | | | |
| TET2 | c.3863G > T | | | | | | | | | | |
| TET2 | c.3897G > C | | | | | | | | | | |
| TET2 | c.3899T > C | | | | | | | | | | |
| TET2 | c.3899T > G | | | | | | | | | | |
| TET2 | c.3967G > T | | | | | | | | | | |
| TET2 | c.3972delT | | | | | | | | | | |
| TET2 | c.4075C > T | | | | | | | | | | |
| TET2 | c.4081G > T | | | | | | | | | | |
| TET2 | c.4104C > A | | | | | | | | | | |
| TET2 | c.4150G > C | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| TET2 | c.3172A > G | | | | | | | | | |
| TET2 | c.3312delT | | | | | | | | | |
| TET2 | c.3312dupT | | | | | | | | | |
| TET2 | c.3344delC | | | | | | | | | |
| TET2 | c.3380_3381delAA | | | | | | | | | |
| TET2 | c.3383_3384delAT | | | | | | | | | |
| TET2 | c.3481A > G | | | | | | | | | |
| TET2 | c.3500 + | | | | | | | | | |
| | 1G > A | | | | | | | | | |
| TET2 | c.3535dupA | | | | | | | | | |
| TET2 | c.3546delT | | | | | | | | | |
| TET2 | c.3555delA | | | | | | | | | |
| TET2 | c.3578delG | | | | | | | | | |
| TET2 | c.3583_3585dupATT | | | | | | | | | |
| TET2 | c.3594 + | | | | | | | | | |
| | 5G > A | | | | | | | | | |
| TET2 | c.3594G > T | | | | | | | | | |
| TET2 | c.3618delA | | | | | | | | | |
| TET2 | c.3626T > C | | | | | | | | | |
| TET2 | c.3641G > A | | | | | | | | | |
| TET2 | c.3646C > T | | | | | | | | | |
| TET2 | c.3689T > G | | | | | | | | | |
| TET2 | c.3732_3733delCT | | | | | | | | | |
| TET2 | c.3734A > G | | | | | | | | | |
| TET2 | c.3764dupA | | | | | | | | | |
| TET2 | c.3782G > A | | | | | | | | | |
| TET2 | c.3789T > G | | | | | | | | | |
| TET2 | c.3804 − | | | | | | | | | |
| | 2A > G | | | | | | | | | |
| TET2 | c.3813C > G | | | | | | | | | |
| TET2 | c.3818G > T | | | | | | | | | |
| TET2 | c.3849dupC | | | | | | | | | |
| TET2 | c.3863G > A | | | | | | | | | |
| TET2 | c.3863G > T | | | | | | | | | |
| TET2 | c.3897G > C | | | | | | | | | |
| TET2 | c.3899T > C | | | | | | | | | |
| TET2 | c.3899T > G | | | | | | | | | |
| TET2 | c.3967G > T | | | | | | | | | |
| TET2 | c.3972delT | | | | | | | | | |
| TET2 | c.4075C > T | | | | | | | | | |
| TET2 | c.4081G > T | | | | | | | | | |
| TET2 | c.4104C > A | | | | | | | | | |
| TET2 | c.4150G > C | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | | alpha-2a | pembrolizumab | pexidartinib | inhibitor | vinorelbine | ponatinib | | prednisolone | prednisone |
| Gene | Variant | ✓ | X | pazopanib | X | ✓ | X | X | X | ✓ | X | X | X |
| TET2 | c.3172A > G | | | | | | | | | | | | |
| TET2 | c.3312delT | | | | | | | | | | | | |
| TET2 | c.3312dupT | | | | | | | | | | | | |
| TET2 | c.3344delC | | | | 1 | | | | | | | | |
| TET2 | c.3380_3381delAA | | | | 1 | | | | | | | | |
| TET2 | c.3383_3384delAT | | | | 1 | | | | | | | | |
| TET2 | c.3481A > G | | | | | | | | | | | | |
| TET2 | c.3500 + | | | | 1 | | | | | | | | |
| | 1G > A | | | | | | | | | | | | |
| TET2 | c.3535dupA | | | | | | | | | | | | |
| TET2 | c.3546delT | | | | 1 | | | | | | | | |
| TET2 | c.3555delA | | | | 1 | | | | | | | | |
| TET2 | c.3578delG | | | | 1 | | | | | | | | |
| TET2 | c.3583_3585dupATT | | | | 1 | | | | | | | | |
| TET2 | c.3594 + | | | | | | | | | | | | |
| | 5G > A | | | | | | | | | | | | |
| TET2 | c.3594G > T | | | | 1 | | | | | | | | |
| TET2 | c.3618delA | | | | | | | | | | | | |
| TET2 | c.3626T > C | | | | 1 | | | | | | | | |
| TET2 | c.3641G > A | | | | | | | | | | | | |
| TET2 | c.3646C > T | | | | | | | | | | | | |
| TET2 | c.3689T > G | | | | 1 | | | | | | | | |
| TET2 | c.3732_3733delCT | | | | 1 | | | | | | | | |
| TET2 | c.3734A > G | | | | 1 | | | | | | | | |
| TET2 | c.3764dupA | | | | 1 | | | | | | | | |
| TET2 | c.3782G > A | | | | 1 | | | | | | | | |
| TET2 | c.3789T > G | | | | 1 | | | | | | | | |
| TET2 | c.3804 − | | | | | | | | | | | | |
| | 2A > G | | | | | | | | | | | | |
| TET2 | c.3813C > G | | | | 1 | | | | | | | | |
| TET2 | c.3818G > T | | | | 1 | | | | | | | | |
| TET2 | c.3849dupC | | | | 1 | | | | | | | | |
| TET2 | c.3863G > A | | | | 1 | | | | | | | | |
| TET2 | c.3863G > T | | | | 1 | | | | | | | | |
| TET2 | c.3897G > C | | | | 1 | | | | | | | | |
| TET2 | c.3899T > C | | | | | | | | | | | | |
| TET2 | c.3899T > G | | | | 1 | | | | | | | | |
| TET2 | c.3967G > T | | | | 1 | | | | | | | | |
| TET2 | c.3972delT | | | | 1 | | | | | | | | |
| TET2 | c.4075C > T | | | | 1 | | | | | | | | |
| TET2 | c.4081G > T | | | | | | | | | | | | |
| TET2 | c.4104C > A | | | | | | | | | | | | |
| TET2 | c.4150G > C | | | | 1 | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| TET2 | c.3172A > G | | | | | | | | | | | |
| TET2 | c.3312delT | | | | | | | | | | | |
| TET2 | c.3312dupT | | | | | | | | | | | |
| TET2 | c.3344delC | | | | | | | | | | | |
| TET2 | c.3380_3381delAA | | | | | | | | | | | |
| TET2 | c.3383_3384delAT | | | | | | | | | | | |
| TET2 | c.3481A > G | | | | | | | | | | | |
| TET2 | c.3500 + | | | | | | | | | | | |
| | 1G > A | | | | | | | | | | | |
| TET2 | c.3535dupA | | | | | | | | | | | |
| TET2 | c.3546delT | | | | | | | | | | | |
| TET2 | c.3555delA | | | | | | | | | | | |
| TET2 | c.3578delG | | | | | | | | | | | |
| TET2 | c.3583_3585dupATT | | | | | | | | | | | |
| TET2 | c.3594 + | | | | | | | | | | | |
| | 5G > A | | | | | | | | | | | |
| TET2 | c.3594G > T | | | | | | | | | | | |
| TET2 | c.3618delA | | | | | | | | | | | |
| TET2 | c.3626T > C | | | | | | | | | | | |
| TET2 | c.3641G > A | | | | | | | | | | | |
| TET2 | c.3646C > T | | | | | | | | | | | |
| TET2 | c.3689T > G | | | | | | | | | | | |
| TET2 | c.3732_3733delCT | | | | | | | | | | | |
| TET2 | c.3734A > G | | | | | | | | | | | |
| TET2 | c.3764dupA | | | | | | | | | | | |
| TET2 | c.3782G > A | | | | | | | | | | | |
| TET2 | c.3789T > G | | | | | | | | | | | |
| TET2 | c.3804 − | | | | | | | | | | | |
| | 2A > G | | | | | | | | | | | |
| TET2 | c.3813C > G | | | | | | | | | | | |
| TET2 | c.3818G > T | | | | | | | | | | | |
| TET2 | c.3849dupC | | | | | | | | | | | |
| TET2 | c.3863G > A | | | | | | | | | | | |
| TET2 | c.3863G > T | | | | | | | | | | | |
| TET2 | c.3897G > C | | | | | | | | | | | |
| TET2 | c.3899T > C | | | | | | | | | | | |
| TET2 | c.3899T > G | | | | | | | | | | | |
| TET2 | c.3967G > T | | | | | | | | | | | |
| TET2 | c.3972delT | | | | | | | | | | | |
| TET2 | c.4075C > T | | | | | | | | | | | |
| TET2 | c.4081G > T | | | | | | | | | | | |
| TET2 | c.4104C > A | | | | | | | | | | | |
| TET2 | c.4150G > C | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| TET2 | c.3172A > G | | | | | | | | | | | |
| TET2 | c.3312delT | | | | | | | | | | | |
| TET2 | c.3312dupT | | | | | | | | | | | |
| TET2 | c.3344delC | | | | | | | | | | | |
| TET2 | c.3380_3381delAA | | | | | | | | | | | |
| TET2 | c.3383_3384delAT | | | | | | | | | | | |
| TET2 | c.3481A > G | | | | | | | | | | | |
| TET2 | c.3500 + | | | | | | | | | | | |
| | 1G > A | | | | | | | | | | | |
| TET2 | c.3535dupA | | | | | | | | | | | |
| TET2 | c.3546delT | | | | | | | | | | | |
| TET2 | c.3555delA | | | | | | | | | | | |
| TET2 | c.3578delG | | | | | | | | | | | |
| TET2 | c.3583_3585dupATT | | | | | | | | | | | |
| TET2 | c.3594 + | | | | | | | | | | | |
| | 5G > A | | | | | | | | | | | |
| TET2 | c.3594G > T | | | | | | | | | | | |
| TET2 | c.3618delA | | | | | | | | | | | |
| TET2 | c.3626T > C | | | | | | | | | | | |
| TET2 | c.3641G > A | | | | | | | | | | | |
| TET2 | c.3646C > T | | | | | | | | | | | |
| TET2 | c.3689T > G | | | | | | | | | | | |
| TET2 | c.3732_3733delCT | | | | | | | | | | | |
| TET2 | c.3734A > G | | | | | | | | | | | |
| TET2 | c.3764dupA | | | | | | | | | | | |
| TET2 | c.3782G > A | | | | | | | | | | | |
| TET2 | c.3789T > G | | | | | | | | | | | |
| TET2 | c.3804 − | | | | | | | | | | | |
| | 2A > G | | | | | | | | | | | |
| TET2 | c.3813C > G | | | | | | | | | | | |
| TET2 | c.3818G > T | | | | | | | | | | | |
| TET2 | c.3849dupC | | | | | | | | | | | |
| TET2 | c.3863G > A | | | | | | | | | | | |
| TET2 | c.3863G > T | | | | | | | | | | | |
| TET2 | c.3897G > C | | | | | | | | | | | |
| TET2 | c.3899T > C | | | | | | | | | | | |
| TET2 | c.3899T > G | | | | | | | | | | | |
| TET2 | c.3967G > T | | | | | | | | | | | |
| TET2 | c.3972delT | | | | | | | | | | | |
| TET2 | c.4075C > T | | | | | | | | | | | |
| TET2 | c.4081G > T | | | | | | | | | | | |
| TET2 | c.4104C > A | | | | | | | | | | | |
| TET2 | c.4150G > C | | | | | | | | | | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| TET2 | c.4160A > T | 1 | | | | | | | | | | | | | | |
| TET2 | c.4173delC | 1 | | | | | | | | | | | | | | |
| TET2 | c.4210C > T | 1 | | | | | | | | | | | | | | |
| TET2 | c.4393C > T | 1 | | | | | | | | | | | | | | |
| TET2 | c.4528C > T | 1 | | | | | | | | | | | | | | |
| TET2 | c.4546C > T | 1 | | | | | | | | | | | | | | |
| TET2 | c.4618C > T | 1 | | | | | | | | | | | | | | |
| TET2 | c.4707C > A | 1 | | | | | | | | | | | | | | |
| TET2 | c.4774_4775delAC | 1 | | | | | | | | | | | | | | |
| TET2 | c.483dupA | 1 | | | | | | | | | | | | | | |
| TET2 | c.4879C > T | 1 | | | | | | | | | | | | | | |
| TET2 | c.4918delG | 1 | | | | | | | | | | | | | | |
| TET2 | c.4944delC | 1 | | | | | | | | | | | | | | |
| TET2 | c.4960C > T | 1 | | | | | | | | | | | | | | |
| TET2 | c.4965G > A | 1 | | | | | | | | | | | | | | |
| TET2 | c.5152G > T | 1 | | | | | | | | | | | | | | |
| TET2 | c.5157delG | 1 | | | | | | | | | | | | | | |
| TET2 | c.5345_5346dupTC | 1 | | | | | | | | | | | | | | |
| TET2 | c.5458delA | 1 | | | | | | | | | | | | | | |
| TET2 | c.5581G > C | 1 | | | | | | | | | | | | | | |
| TET2 | c.5581G > T | 1 | | | | | | | | | | | | | | |
| TET2 | c.5582G > A | 1 | | | | | | | | | | | | | | |
| TET2 | c.5669A > G | 1 | | | | | | | | | | | | | | |
| TET2 | c.5711A > G | 1 | | | | | | | | | | | | | | |
| TET2 | c.5711A > T | 1 | | | | | | | | | | | | | | |
| TET2 | c.5733delA | 1 | | | | | | | | | | | | | | |
| TET2 | c.5734C > T | 1 | | | | | | | | | | | | | | |
| TET2 | c.640delT | 1 | | | | | | | | | | | | | | |
| TET2 | c.651delC | 1 | | | | | | | | | | | | | | |
| TET2 | c.741_742delTC | 1 | | | | | | | | | | | | | | |
| TET2 | c.753delC | 1 | | | | | | | | | | | | | | |
| TET2 | c.756delT | 1 | | | | | | | | | | | | | | |
| TET2 | c.758delA | 1 | | | | | | | | | | | | | | |
| TET2 | c.817C > T | 1 | | | | | | | | | | | | | | |
| TP53 | c.1005delT | | | | | | | | | | | 1 | | 1 | | |
| TP53 | c.1014C > T | | | | | | | | | | | 1 | | 1 | | |
| TP53 | c.139C > T | | | | | | | | | | | 1 | | 1 | | 1 |
| TP53 | c.141G > A | | | | | | | | | | | 1 | | 1 | | |
| TP53 | c.28G > A | | | | | | | | | | | 1 | | 1 | | 1 |
| TP53 | c.329G > A | | | | | | | | | | | 1 | | 1 | | 1 |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| TET2 | c.4160A > T | | | | | | | | | | | | | | |
| TET2 | c.4173delC | | | | | | | | | | | | | | |
| TET2 | c.4210C > T | | | | | | | | | | | | | | |
| TET2 | c.4393C > T | | | | | | | | | | | | | | |
| TET2 | c.4528C > T | | | | | | | | | | | | | | |
| TET2 | c.4546C > T | | | | | | | | | | | | | | |
| TET2 | c.4618C > T | | | | | | | | | | | | | | |
| TET2 | c.4707C > A | | | | | | | | | | | | | | |
| TET2 | c.4774_4775delAC | | | | | | | | | | | | | | |
| TET2 | c.483dupA | | | | | | | | | | | | | | |
| TET2 | c.4879C > T | | | | | | | | | | | | | | |
| TET2 | c.4918delG | | | | | | | | | | | | | | |
| TET2 | c.4944delC | | | | | | | | | | | | | | |
| TET2 | c.4960C > T | | | | | | | | | | | | | | |
| TET2 | c.4965G > A | | | | | | | | | | | | | | |
| TET2 | c.5152G > T | | | | | | | | | | | | | | |
| TET2 | c.5157delG | | | | | | | | | | | | | | |
| TET2 | c.5345_5346dupTC | | | | | | | | | | | | | | |
| TET2 | c.5458delA | | | | | | | | | | | | | | |
| TET2 | c.5581G > C | | | | | | | | | | | | | | |
| TET2 | c.5581G > T | | | | | | | | | | | | | | |
| TET2 | c.5582G > A | | | | | | | | | | | | | | |
| TET2 | c.5669A > G | | | | | | | | | | | | | | |
| TET2 | c.5711A > G | | | | | | | | | | | | | | |
| TET2 | c.5711A > T | | | | | | | | | | | | | | |
| TET2 | c.5733delA | | | | | | | | | | | | | | |
| TET2 | c.5734C > T | | | | | | | | | | | | | | |
| TET2 | c.640delT | | | | | | | | | | | | | | |
| TET2 | c.651delC | | | | | | | | | | | | | | |
| TET2 | c.741_742delTC | | | | | | | | | | | | | | |
| TET2 | c.753delC | | | | | | | | | | | | | | |
| TET2 | c.756delT | | | | | | | | | | | | | | |
| TET2 | c.758delA | | | | | | | | | | | | | | |
| TET2 | c.817C > T | | | | | | | | | | | | | | |
| TP53 | c.1005delT | | | | | | | | | | | | | | |
| TP53 | c.1014C > T | | | | | | | | | | | | | | |
| TP53 | c.139C > T | | | | | | | | | | | | | | |
| TP53 | c.141G > A | | | | | | | | | | | | | | |
| TP53 | c.28G > A | | | | | | | | | | | | | | |
| TP53 | c.329G > A | | | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| TET2 | c.4160A > T | | | | | | | | | | | | | |
| TET2 | c.4173delC | | | | | | | | | | | | | |
| TET2 | c.4210C > T | | | | | | | | | | | | | |
| TET2 | c.4393C > T | | | | | | | | | | | | | |
| TET2 | c.4528C > T | | | | | | | | | | | | | |
| TET2 | c.4546C > T | | | | | | | | | | | | | |
| TET2 | c.4618C > T | | | | | | | | | | | | | |
| TET2 | c.4707C > A | | | | | | | | | | | | | |
| TET2 | c.4774_4775delAC | | | | | | | | | | | | | |
| TET2 | c.483dupA | | | | | | | | | | | | | |
| TET2 | c.4879C > T | | | | | | | | | | | | | |
| TET2 | c.4918delG | | | | | | | | | | | | | |
| TET2 | c.4944delC | | | | | | | | | | | | | |
| TET2 | c.4960C > T | | | | | | | | | | | | | |
| TET2 | c.4965G > A | | | | | | | | | | | | | |
| TET2 | c.5152G > T | | | | | | | | | | | | | |
| TET2 | c.5157delG | | | | | | | | | | | | | |
| TET2 | c.5345_5346dupTC | | | | | | | | | | | | | |
| TET2 | c.5458delA | | | | | | | | | | | | | |
| TET2 | c.5581G > C | | | | | | | | | | | | | |
| TET2 | c.5581G > T | | | | | | | | | | | | | |
| TET2 | c.5582G > A | | | | | | | | | | | | | |
| TET2 | c.5669A > G | | | | | | | | | | | | | |
| TET2 | c.5711A > G | | | | | | | | | | | | | |
| TET2 | c.5711A > T | | | | | | | | | | | | | |
| TET2 | c.5733delA | | | | | | | | | | | | | |
| TET2 | c.5734C > T | | | | | | | | | | | | | |
| TET2 | c.640delT | | | | | | | | | | | | | |
| TET2 | c.651delC | | | | | | | | | | | | | |
| TET2 | c.741_742delTC | | | | | | | | | | | | | |
| TET2 | c.753delC | | | | | | | | | | | | | |
| TET2 | c.756delT | | | | | | | | | | | | | |
| TET2 | c.758delA | | | | | | | | | | | | | |
| TET2 | c.817C > T | | | | | | | | | | | | | |
| TP53 | c.1005delT | | | | | | | | | | | | | |
| TP53 | c.1014C > T | | | | | | | | | | | | | |
| TP53 | c.139C > T | | | | | | | | | | | | | |
| TP53 | c.141G > A | | | | | | | | | | | | | |
| TP53 | c.28G > A | | | | | | | | | | | | | |
| TP53 | c.329G > A | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| TET2 | c.4160A > T | | | | | | | | | | | |
| TET2 | c.4173delC | | | | | | | | | | | |
| TET2 | c.4210C > T | | | | | | | | | | | |
| TET2 | c.4393C > T | | | | | | | | | | | |
| TET2 | c.4528C > T | | | | | | | | | | | |
| TET2 | c.4546C > T | | | | | | | | | | | |
| TET2 | c.4618C > T | | | | | | | | | | | |
| TET2 | c.4707C > A | | | | | | | | | | | |
| TET2 | c.4774_4775delAC | | | | | | | | | | | |
| TET2 | c.483dupA | | | | | | | | | | | |
| TET2 | c.4879C > T | | | | | | | | | | | |
| TET2 | c.4918delG | | | | | | | | | | | |
| TET2 | c.4944delC | | | | | | | | | | | |
| TET2 | c.4960C > T | | | | | | | | | | | |
| TET2 | c.4965G > A | | | | | | | | | | | |
| TET2 | c.5152G > T | | | | | | | | | | | |
| TET2 | c.5157delG | | | | | | | | | | | |
| TET2 | c.5345_5346dupTC | | | | | | | | | | | |
| TET2 | c.5458delA | | | | | | | | | | | |
| TET2 | c.5581G > C | | | | | | | | | | | |
| TET2 | c.5581G > T | | | | | | | | | | | |
| TET2 | c.5582G > A | | | | | | | | | | | |
| TET2 | c.5669A > G | | | | | | | | | | | |
| TET2 | c.5711A > G | | | | | | | | | | | |
| TET2 | c.5711A > T | | | | | | | | | | | |
| TET2 | c.5733delA | | | | | | | | | | | |
| TET2 | c.5734C > T | | | | | | | | | | | |
| TET2 | c.640delT | | | | | | | | | | | |
| TET2 | c.651delC | | | | | | | | | | | |
| TET2 | c.741_742delTC | | | | | | | | | | | |
| TET2 | c.753delC | | | | | | | | | | | |
| TET2 | c.756delT | | | | | | | | | | | |
| TET2 | c.758delA | | | | | | | | | | | |
| TET2 | c.817C > T | | | | | | | | | | | |
| TP53 | c.1005delT | | | | | | | | | 1 | | |
| TP53 | c.1014C > T | | | | | | | | | 1 | | |
| TP53 | c.139C > T | | | | | | | | | 1 | | |
| TP53 | c.141G > A | | | | | | | | | 1 | | |
| TP53 | c.28G > A | | | | | | | | | 1 | | |
| TP53 | c.329G > A | | | | | | | | | 1 | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| TET2 | c.4160A > T | | | | | | | | | | | 1 | | |
| TET2 | c.4173delC | | | | | | | | | | | 1 | | |
| TET2 | c.4210C > T | | | | | | | | | | | 1 | | |
| TET2 | c.4393C > T | | | | | | | | | | | 1 | | |
| TET2 | c.4528C > T | | | | | | | | | | | 1 | | |
| TET2 | c.4546C > T | | | | | | | | | | | 1 | | |
| TET2 | c.4618C > T | | | | | | | | | | | 1 | | |
| TET2 | c.4707C > A | | | | | | | | | | | 1 | | |
| TET2 | c.4774_4775delAC | | | | | | | | | | | 1 | | |
| TET2 | c.483dupA | | | | | | | | | | | 1 | | |
| TET2 | c.4879C > T | | | | | | | | | | | 1 | | |
| TET2 | c.4918delG | | | | | | | | | | | 1 | | |
| TET2 | c.4944delC | | | | | | | | | | | 1 | | |
| TET2 | c.4960C > T | | | | | | | | | | | 1 | | |
| TET2 | c.4965G > A | | | | | | | | | | | 1 | | |
| TET2 | c.5152G > T | | | | | | | | | | | 1 | | |
| TET2 | c.5157delG | | | | | | | | | | | 1 | | |
| TET2 | c.5345_5346dupTC | | | | | | | | | | | 1 | | |
| TET2 | c.5458delA | | | | | | | | | | | 1 | | |
| TET2 | c.5581G > C | | | | | | | | | | | 1 | | |
| TET2 | c.5581G > T | | | | | | | | | | | 1 | | |
| TET2 | c.5582G > A | | | | | | | | | | | 1 | | |
| TET2 | c.5669A > G | | | | | | | | | | | 1 | | |
| TET2 | c.5711A > G | | | | | | | | | | | 1 | | |
| TET2 | c.5711A > T | | | | | | | | | | | 1 | | |
| TET2 | c.5733delA | | | | | | | | | | | 1 | | |
| TET2 | c.5734C > T | | | | | | | | | | | 1 | | |
| TET2 | c.640delT | | | | | | | | | | | 1 | | |
| TET2 | c.651delC | | | | | | | | | | | 1 | | |
| TET2 | c.741_742delTC | | | | | | | | | | | 1 | | |
| TET2 | c.753delC | | | | | | | | | | | 1 | | |
| TET2 | c.756delT | | | | | | | | | | | 1 | | |
| TET2 | c.758delA | | | | | | | | | | | 1 | | |
| TET2 | c.817C > T | | | | | | | | | | | 1 | | |
| TP53 | c.1005delT | | | | | | | | | | | | | |
| TP53 | c.1014C > T | | | | | | | | | | | | | |
| TP53 | c.139C > T | | | | | | | | | | | | | |
| TP53 | c.141G > A | | | | | | | | | | | | | |
| TP53 | c.28G > A | | | | | | | | | | | | | |
| TP53 | c.329G > A | | | | | | | | | | | | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| TET2 | c.4160A > T | | | | | | | | | | | |
| TET2 | c.4173delC | | | | | | | | | | | |
| TET2 | c.4210C > T | | | | | | | | | | | |
| TET2 | c.4393C > T | | | | | | | | | | | |
| TET2 | c.4528C > T | | | | | | | | | | | |
| TET2 | c.4546C > T | | | | | | | | | | | |
| TET2 | c.4618C > T | | | | | | | | | | | |
| TET2 | c.4707C > A | | | | | | | | | | | |
| TET2 | c.4774_4775delAC | | | | | | | | | | | |
| TET2 | c.483dupA | | | | | | | | | | | |
| TET2 | c.4879C > T | | | | | | | | | | | |
| TET2 | c.4918delG | | | | | | | | | | | |
| TET2 | c.4944delC | | | | | | | | | | | |
| TET2 | c.4960C > T | | | | | | | | | | | |
| TET2 | c.4965G > A | | | | | | | | | | | |
| TET2 | c.5152G > T | | | | | | | | | | | |
| TET2 | c.5157delG | | | | | | | | | | | |
| TET2 | c.5345_5346dupTC | | | | | | | | | | | |
| TET2 | c.5458delA | | | | | | | | | | | |
| TET2 | c.5581G > C | | | | | | | | | | | |
| TET2 | c.5581G > T | | | | | | | | | | | |
| TET2 | c.5582G > A | | | | | | | | | | | |
| TET2 | c.5669A > G | | | | | | | | | | | |
| TET2 | c.5711A > G | | | | | | | | | | | |
| TET2 | c.5711A > T | | | | | | | | | | | |
| TET2 | c.5733delA | | | | | | | | | | | |
| TET2 | c.5734C > T | | | | | | | | | | | |
| TET2 | c.640delT | | | | | | | | | | | |
| TET2 | c.651delC | | | | | | | | | | | |
| TET2 | c.741_742delTC | | | | | | | | | | | |
| TET2 | c.753delC | | | | | | | | | | | |
| TET2 | c.756delT | | | | | | | | | | | |
| TET2 | c.758delA | | | | | | | | | | | |
| TET2 | c.817C > T | | | | | | | | | | | |
| TP53 | c.1005delT | | | | | | | | | | | |
| TP53 | c.1014C > T | | | | | | | | | | | |
| TP53 | c.139C > T | | | | | | | | | | | |
| TP53 | c.141G > A | | | | | | | | | | | |
| TP53 | c.28G > A | | | | | | | | | | | 1 |
| TP53 | c.329G > A | | | | | | | | | | | 1 |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| TET2 | c.4160A > T | | | | | | | | | | | | |
| TET2 | c.4173delC | | | | | | | | | | | | |
| TET2 | c.4210C > T | | | | | | | | | | | | |
| TET2 | c.4393C > T | | | | | | | | | | | | |
| TET2 | c.4528C > T | | | | | | | | | | | | |
| TET2 | c.4546C > T | | | | | | | | | | | | |
| TET2 | c.4618C > T | | | | | | | | | | | | |
| TET2 | c.4707C > A | | | | | | | | | | | | |
| TET2 | c.4774_4775delAC | | | | | | | | | | | | |
| TET2 | c.483dupA | | | | | | | | | | | | |
| TET2 | c.4879C > T | | | | | | | | | | | | |
| TET2 | c.4918delG | | | | | | | | | | | | |
| TET2 | c.4944delC | | | | | | | | | | | | |
| TET2 | c.4960C > T | | | | | | | | | | | | |
| TET2 | c.4965G > A | | | | | | | | | | | | |
| TET2 | c.5152G > T | | | | | | | | | | | | |
| TET2 | c.5157delG | | | | | | | | | | | | |
| TET2 | c.5345_5346dupTC | | | | | | | | | | | | |
| TET2 | c.5458delA | | | | | | | | | | | | |
| TET2 | c.5581G > C | | | | | | | | | | | | |
| TET2 | c.5581G > T | | | | | | | | | | | | |
| TET2 | c.5582G > A | | | | | | | | | | | | |
| TET2 | c.5669A > G | | | | | | | | | | | | |
| TET2 | c.5711A > G | | | | | | | | | | | | |
| TET2 | c.5711A > T | | | | | | | | | | | | |
| TET2 | c.5733delA | | | | | | | | | | | | |
| TET2 | c.5734C > T | | | | | | | | | | | | |
| TET2 | c.640delT | | | | | | | | | | | | |
| TET2 | c.651delC | | | | | | | | | | | | |
| TET2 | c.741_742delTC | | | | | | | | | | | | |
| TET2 | c.753delC | | | | | | | | | | | | |
| TET2 | c.756delT | | | | | | | | | | | | |
| TET2 | c.758delA | | | | | | | | | | | | |
| TET2 | c.817C > T | | | | | | | | | | | | |
| TP53 | c.1005delT | | | | | | 1 | 2 | 1 | 1 | | 1 | |
| TP53 | c.1014C > T | | | | | | 1 | 2 | 1 | 1 | | 1 | |
| TP53 | c.139C > T | | | | | | 1 | 1 | 1 | 2 | | 1 | |
| TP53 | c.141G > A | | | | | | 1 | 2 | 1 | 1 | | 1 | |
| TP53 | c.28G > A | | | | | | 1 | 1 | 1 | 2 | | 1 | |
| TP53 | c.329G > A | | | | | | 1 | 1 | 1 | 2 | | 1 | |

| | | | | | | | MAP | | | | |
|---|---|---|---|---|---|---|---|---|---|---|---|
| | | | | | | | kinase | | | | |
| | | | | lenalidomide/ | | | kinase | | methylpred- | | mitoxan- |
| | | lenalidomide | | rituximab | lenvatinib | lorlatinib | inhibitor | methotrexate | nisolone | midostaurin | trone |
| Gene | Variant | ✓ | X | ✓ | ✓ | X | ✓ | X | X | ✓ | X |
| TET2 | c.4160A > T | | | | | | | | | | |
| TET2 | c.4173delC | | | | | | | | | | |
| TET2 | c.4210C > T | | | | | | | | | | |
| TET2 | c.4393C > T | | | | | | | | | | |
| TET2 | c.4528C > T | | | | | | | | | | |
| TET2 | c.4546C > T | | | | | | | | | | |
| TET2 | c.4618C > T | | | | | | | | | | |
| TET2 | c.4707C > A | | | | | | | | | | |
| TET2 | c.4774_4775delAC | | | | | | | | | | |
| TET2 | c.483dupA | | | | | | | | | | |
| TET2 | c.4879C > T | | | | | | | | | | |
| TET2 | c.4918delG | | | | | | | | | | |
| TET2 | c.4944delC | | | | | | | | | | |
| TET2 | c.4960C > T | | | | | | | | | | |
| TET2 | c.4965G > A | | | | | | | | | | |
| TET2 | c.5152G > T | | | | | | | | | | |
| TET2 | c.5157delG | | | | | | | | | | |
| TET2 | c.5345_5346dupTC | | | | | | | | | | |
| TET2 | c.5458delA | | | | | | | | | | |
| TET2 | c.5581G > C | | | | | | | | | | |
| TET2 | c.5581G > T | | | | | | | | | | |
| TET2 | c.5582G > A | | | | | | | | | | |
| TET2 | c.5669A > G | | | | | | | | | | |
| TET2 | c.5711A > G | | | | | | | | | | |
| TET2 | c.5711A > T | | | | | | | | | | |
| TET2 | c.5733delA | | | | | | | | | | |
| TET2 | c.5734C > T | | | | | | | | | | |
| TET2 | c.640delT | | | | | | | | | | |
| TET2 | c.651delC | | | | | | | | | | |
| TET2 | c.741_742delTC | | | | | | | | | | |
| TET2 | c.753delC | | | | | | | | | | |
| TET2 | c.756delT | | | | | | | | | | |
| TET2 | c.758delA | | | | | | | | | | |
| TET2 | c.817C > T | | | | | | | | | | |
| TP53 | c.1005delT | 1 | 1 | 1 | | 1 | | | | | |
| TP53 | c.1014C > T | 1 | 1 | 1 | | 1 | | | | | |
| TP53 | c.139C > T | 1 | 1 | 1 | | 1 | | | | | |
| TP53 | c.141G > A | 1 | 1 | 1 | | 1 | | | | | |
| TP53 | c.28G > A | 1 | 1 | 1 | | 1 | | | | | |
| TP53 | c.329G > A | 1 | 1 | 1 | | 1 | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| TET2 | c.4160A > T | | | | | | | | | |
| TET2 | c.4173delC | | | | | | | | | |
| TET2 | c.4210C > T | | | | | | | | | |
| TET2 | c.4393C > T | | | | | | | | | |
| TET2 | c.4528C > T | | | | | | | | | |
| TET2 | c.4546C > T | | | | | | | | | |
| TET2 | c.4618C > T | | | | | | | | | |
| TET2 | c.4707C > A | | | | | | | | | |
| TET2 | c.4774_4775delAC | | | | | | | | | |
| TET2 | c.483dupA | | | | | | | | | |
| TET2 | c.4879C > T | | | | | | | | | |
| TET2 | c.4918delG | | | | | | | | | |
| TET2 | c.4944delC | | | | | | | | | |
| TET2 | c.4960C > T | | | | | | | | | |
| TET2 | c.4965G > A | | | | | | | | | |
| TET2 | c.5152G > T | | | | | | | | | |
| TET2 | c.5157delG | | | | | | | | | |
| TET2 | c.5345_5346dupTC | | | | | | | | | |
| TET2 | c.5458delA | | | | | | | | | |
| TET2 | c.5581G > C | | | | | | | | | |
| TET2 | c.5581G > T | | | | | | | | | |
| TET2 | c.5582G > A | | | | | | | | | |
| TET2 | c.5669A > G | | | | | | | | | |
| TET2 | c.5711A > G | | | | | | | | | |
| TET2 | c.5711A > T | | | | | | | | | |
| TET2 | c.5733delA | | | | | | | | | |
| TET2 | c.5734C > T | | | | | | | | | |
| TET2 | c.640delT | | | | | | | | | |
| TET2 | c.651delC | | | | | | | | | |
| TET2 | c.741_742delTC | | | | | | | | | |
| TET2 | c.753delC | | | | | | | | | |
| TET2 | c.756delT | | | | | | | | | |
| TET2 | c.758delA | | | | | | | | | |
| TET2 | c.817C > T | | | | | | | | | |
| TP53 | c.1005delT | | | | | 1 | 1 | | | |
| TP53 | c.1014C > T | | | | | 1 | 1 | | | |
| TP53 | c.139C > T | | | | | 1 | 1 | | | |
| TP53 | c.141G > A | | | | | 1 | 1 | | | |
| TP53 | c.28G > A | | | | | 1 | 1 | | | |
| TP53 | c.329G > A | | | | | 1 | 1 | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alpha-2a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| TET2 | c.4160A > T | | | | 1 | | | | | | | | |
| TET2 | c.4173delC | | | | 1 | | | | | | | | |
| TET2 | c.4210C > T | | | | | | | | | | | | |
| TET2 | c.4393C > T | | | | | | | | | | | | |
| TET2 | c.4528C > T | | | | | | | | | | | | |
| TET2 | c.4546C > T | | | | 1 | | | | | | | | |
| TET2 | c.4618C > T | | | | | | | | | | | | |
| TET2 | c.4707C > A | | | | 1 | | | | | | | | |
| TET2 | c.4774_4775delAC | | | | 1 | | | | | | | | |
| TET2 | c.483dupA | | | | | | | | | | | | |
| TET2 | c.4879C > T | | | | 1 | | | | | | | | |
| TET2 | c.4918delG | | | | 1 | | | | | | | | |
| TET2 | c.4944delC | | | | 1 | | | | | | | | |
| TET2 | c.4960C > T | | | | 1 | | | | | | | | |
| TET2 | c.4965G > A | | | | 1 | | | | | | | | |
| TET2 | c.5152G > T | | | | 1 | | | | | | | | |
| TET2 | c.5157delG | | | | 1 | | | | | | | | |
| TET2 | c.5345_5346dupTC | | | | | | | | | | | | |
| TET2 | c.5458delA | | | | | | | | | | | | |
| TET2 | c.5581G > C | | | | 1 | | | | | | | | |
| TET2 | c.5581G > T | | | | 1 | | | | | | | | |
| TET2 | c.5582G > A | | | | 1 | | | | | | | | |
| TET2 | c.5669A > G | | | | 1 | | | | | | | | |
| TET2 | c.5711A > G | | | | 1 | | | | | | | | |
| TET2 | c.5711A > T | | | | | | | | | | | | |
| TET2 | c.5733delA | | | | 1 | | | | | | | | |
| TET2 | c.5734C > T | | | | | | | | | | | | |
| TET2 | c.640delT | | | | | | | | | | | | |
| TET2 | c.651delC | | | | 1 | | | | | | | | |
| TET2 | c.741_742delTC | | | | 1 | | | | | | | | |
| TET2 | c.753delC | | | | | | | | | | | | |
| TET2 | c.756delT | | | | 1 | | | | | | | | |
| TET2 | c.758delA | | | | | | | | | | | | |
| TET2 | c.817C > T | | | | 1 | | | | | | | | |
| TP53 | c.1005delT | | | | | | | | | | | | |
| TP53 | c.1014C > T | | | | | | | | | | | | |
| TP53 | c.139C > T | | | | | | | | | | | | |
| TP53 | c.141G > A | | | | | | | | | | | | |
| TP53 | c.28G > A | | | | | | | | | | | | |
| TP53 | c.329G > A | | | | | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| TET2 | c.4160A > T | | | | | | | | | | | |
| TET2 | c.4173delC | | | | | | | | | | | |
| TET2 | c.4210C > T | | | | | | | | | | | |
| TET2 | c.4393C > T | | | | | | | | | | | |
| TET2 | c.4528C > T | | | | | | | | | | | |
| TET2 | c.4546C > T | | | | | | | | | | | |
| TET2 | c.4618C > T | | | | | | | | | | | |
| TET2 | c.4707C > A | | | | | | | | | | | |
| TET2 | c.4774_4775delAC | | | | | | | | | | | |
| TET2 | c.483dupA | | | | | | | | | | | |
| TET2 | c.4879C > T | | | | | | | | | | | |
| TET2 | c.4918delG | | | | | | | | | | | |
| TET2 | c.4944delC | | | | | | | | | | | |
| TET2 | c.4960C > T | | | | | | | | | | | |
| TET2 | c.4965G > A | | | | | | | | | | | |
| TET2 | c.5152G > T | | | | | | | | | | | |
| TET2 | c.5157delG | | | | | | | | | | | |
| TET2 | c.5345_5346dupTC | | | | | | | | | | | |
| TET2 | c.5458delA | | | | | | | | | | | |
| TET2 | c.5581G > C | | | | | | | | | | | |
| TET2 | c.5581G > T | | | | | | | | | | | |
| TET2 | c.5582G > A | | | | | | | | | | | |
| TET2 | c.5669A > G | | | | | | | | | | | |
| TET2 | c.5711A > G | | | | | | | | | | | |
| TET2 | c.5711A > T | | | | | | | | | | | |
| TET2 | c.5733delA | | | | | | | | | | | |
| TET2 | c.5734C > T | | | | | | | | | | | |
| TET2 | c.640delT | | | | | | | | | | | |
| TET2 | c.651delC | | | | | | | | | | | |
| TET2 | c.741_742delTC | | | | | | | | | | | |
| TET2 | c.753delC | | | | | | | | | | | |
| TET2 | c.756delT | | | | | | | | | | | |
| TET2 | c.758delA | | | | | | | | | | | |
| TET2 | c.817C > T | | | | | | | | | | | |
| TP53 | c.1005delT | | | | 1 | 2 | 1 | | | | | |
| TP53 | c.1014C > T | | | | 1 | 2 | 1 | | | | | |
| TP53 | c.139C > T | | | | 1 | 2 | 1 | | | | | |
| TP53 | c.141G > A | | | | 1 | 2 | 1 | | | | | |
| TP53 | c.28G > A | | | | 1 | 2 | 1 | | | | | |
| TP53 | c.329G > A | | | | 1 | 2 | 1 | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| TET2 | c.4160A > T | | ✓ | | | | | | | | | |
| TET2 | c.4173delC | | | | | | | | | | | |
| TET2 | c.4210C > T | | | | | | | | | | | |
| TET2 | c.4393C > T | | | | | | | | | | | |
| TET2 | c.4528C > T | | | | | | | | | | | |
| TET2 | c.4546C > T | | | | | | | | | | | |
| TET2 | c.4618C > T | | | | | | | | | | | |
| TET2 | c.4707C > A | | | | | | | | | | | |
| TET2 | c.4774_4775delAC | | | | | | | | | | | |
| TET2 | c.483dupA | | | | | | | | | | | |
| TET2 | c.4879C > T | | | | | | | | | | | |
| TET2 | c.4918delG | | | | | | | | | | | |
| TET2 | c.4944delC | | | | | | | | | | | |
| TET2 | c.4960C > T | | | | | | | | | | | |
| TET2 | c.4965G > A | | | | | | | | | | | |
| TET2 | c.5152G > T | | | | | | | | | | | |
| TET2 | c.5157delG | | | | | | | | | | | |
| TET2 | c.5345_5346dupTC | | | | | | | | | | | |
| TET2 | c.5458delA | | | | | | | | | | | |
| TET2 | c.5581G > C | | | | | | | | | | | |
| TET2 | c.5581G > T | | | | | | | | | | | |
| TET2 | c.5582G > A | | | | | | | | | | | |
| TET2 | c.5669A > G | | | | | | | | | | | |
| TET2 | c.5711A > G | | | | | | | | | | | |
| TET2 | c.5711A > T | | | | | | | | | | | |
| TET2 | c.5733delA | | | | | | | | | | | |
| TET2 | c.5734C > T | | | | | | | | | | | |
| TET2 | c.640delT | | | | | | | | | | | |
| TET2 | c.651delC | | | | | | | | | | | |
| TET2 | c.741_742delTC | | | | | | | | | | | |
| TET2 | c.753delC | | | | | | | | | | | |
| TET2 | c.756delT | | | | | | | | | | | |
| TET2 | c.758delA | | | | | | | | | | | |
| TET2 | c.817C > T | | | | | | | | | | | |
| TP53 | c.1005delT | | | | | | | | | 1 | | |
| TP53 | c.1014C > T | | | | | | | | | 1 | | |
| TP53 | c.139C > T | | | | | | | | | 1 | | |
| TP53 | c.141G > A | | | | | | | | | 1 | | |
| TP53 | c.28G > A | | | | | | | | | 1 | | |
| TP53 | c.329G > A | | | | | | | | | 1 | | |

| | | | | 5-fluorouracil/ | | | | | | | | | | | | anti- |
|---|---|---|---|---|---|---|---|---|---|---|---|---|---|---|---|---|
| | | | | irinotecan/ | | 5-fluorouracil/ | | | | | | | | | | CD20 |
| | | | 5-azacytidine/ | leucovorin/ | | leucovorin/ | | | | | | | | alemtuzumab/ | | antibody/ |
| | | 5-azacytidine | sorafenib | oxaliplatin | | oxaliplatin | | abiraterone | afatinib | aflibercept | alectinib | alemtuzumab | | rituximab | AMG 337 | idelalisib |
| Gene | Variant | ✓ | ✓ | ✓ | X | ✓ | X | X | X | ✓ | X | ✓ | X | ✓ | X | ✓ |
| TP53 | c.332T > C | | | | | | | | | | | 1 | | 1 | | 1 |
| TP53 | c.358A > G | | | | | | | | | | | 1 | | 1 | | 1 |
| TP53 | c.376 − | | | | | | | | | | | 1 | | 1 | | 1 |
| | 1G > A | | | | | | | | | | | | | | | |
| TP53 | c.395A > G | | | | | | | | | | | 1 | 1 | 1 | | 1 |
| TP53 | c.404G > A | | | | | | | | | | | 1 | | 1 | | 1 |
| TP53 | c.405C > A | | | | | | | | | | | 1 | | 1 | | 1 |
| TP53 | c.488A > G | | | | | | | | | | | 1 | | 1 | | 1 |
| TP53 | c.524G > A | | | | | | | 1 | | | | 1 | | 1 | | 1 |
| TP53 | c.527G > T | | | | | | | | | | | 1 | | 1 | | 1 |
| TP53 | c.542G > A | | | | | | | | | | | 1 | | 1 | | 1 |
| TP53 | c.559 + | | | | | | | | | | | 1 | | 1 | | 1 |
| | 1G > A | | | | | | | | | | | | | | | |
| TP53 | c.559 + | | | | | | | | | | | 1 | | 1 | | 1 |
| | 1G > C | | | | | | | | | | | | | | | |
| TP53 | c.584T > C | | | | | | | | | | | 1 | | 1 | | 1 |
| TP53 | c.614A > G | | | | | | | | | | | 1 | | 1 | | 1 |
| TP53 | c.637C > T | | | | | | | | | | | 1 | | 1 | | 1 |
| TP53 | c.646G > A | | | | | | | | | | | 1 | | 1 | | |
| TP53 | c.673 − | | | | | | | | | | | 1 | | 1 | | 1 |
| | 19G > A | | | | | | | | | | | | | | | |
| TP53 | c.695T > A | | | | | | | | | | | 1 | | 1 | | 1 |
| TP53 | c.701A > G | | | | | | | | | | | 1 | | 1 | | |
| TP53 | c.713G > A | | | | | | | | | | | 1 | | 1 | | 1 |
| TP53 | c.713G > C | | | | | | | | | | | 1 | | 1 | | 1 |
| TP53 | c.742C > T | | | | | | | | | | | 1 | | 1 | | 1 |
| TP53 | c.743G > A | | | | | | | | | | | 1 | | 1 | | 1 |
| TP53 | c.743G > T | | | | | | | | | | | 1 | | 1 | | 1 |
| TP53 | c.817C > T | | | | | | | | | | | 1 | | 1 | | 1 |
| TP53 | c.818G > A | | | | | | | | | | | 1 | | 1 | | |
| TP53 | c.844C > G | | | | | | | | | | | 1 | | 1 | | |
| TP53 | c.91G > A | | | | | | | | | | | 1 | | 1 | | |
| TP53 | c.994 − | | | | | | | | | | | 1 | | 1 | | |
| | 2A > G | | | | | | | | | | | | | | | |

| | | | | | | | | | | | | | BRAF | | |
|---|---|---|---|---|---|---|---|---|---|---|---|---|---|---|---|
| | | anti- | | | | | | | | | | | inhibitor/ | | |
| | | EGFR | arsenic | | | | | bevacizumab/ | bevacizumab/ | | | BRAF | MEK | | |
| | | antibody | trioxide | atezolizumab | axitinib | bevacizumab | | cetuximab | erlotinib | bosutinib | | inhibitor | inhibitor | brigatinib | cabazitaxel |
| Gene | Variant | X | ✓ | ✓ | ✓ | ✓ | X | X | X | ✓ | X | ✓ | ✓ | ✓ | X |
| TP53 | c.332T > C | | | | | | | | | | | | | | |
| TP53 | c.358A > G | | | | | | | | | | | | | | |
| TP53 | c.376 − | | | | | | | | | | | | | | |
| | 1G > A | | | | | | | | | | | | | | |
| TP53 | c.395A > G | | | | | | | | | | | | | | |
| TP53 | c.404G > A | | | | | | | | | | | | | | |
| TP53 | c.405C > A | | | | | | | | | | | | | | |
| TP53 | c.488A > G | | | | | | | | | | | | | | |
| TP53 | c.524G > A | | | | | | | | | | | | | | 1 |
| TP53 | c.527G > T | | | | | | | | | | | | | | |
| TP53 | c.542G > A | | | | | | | | | | | | | | |
| TP53 | c.559 + | | | | | | | | | | | | | | |
| | 1G > A | | | | | | | | | | | | | | |
| TP53 | c.559 + | | | | | | | | | | | | | | |
| | 1G > C | | | | | | | | | | | | | | |
| TP53 | c.584T > C | | | | | | | | | | | | | | |
| TP53 | c.614A > G | | | | | | | | | | | | | | |
| TP53 | c.637C > T | | | | | | | | | | | | | | |
| TP53 | c.646G > A | | | | | | | | | | | | | | |
| TP53 | c.673 − | | | | | | | | | | | | | | |
| | 19G > A | | | | | | | | | | | | | | |
| TP53 | c.695T > A | | | | | | | | | | | | | | |
| TP53 | c.701A > G | | | | | | | | | | | | | | |
| TP53 | c.713G > A | | | | | | | | | | | | | | |
| TP53 | c.713G > C | | | | | | | | | | | | | | |
| TP53 | c.742C > T | | | | | | | | | | | | | | |
| TP53 | c.743G > A | | | | | | | | | | | | | | |
| TP53 | c.743G > T | | | | | | | | | | | | | | |
| TP53 | c.817C > T | | | | | | | | | | | | | | |
| TP53 | c.818G > A | | | | | | | | | | | | | | |
| TP53 | c.844C > G | | | | | | | | | | | | | | |
| TP53 | c.91G > A | | | | | | | | | | | | | | |
| TP53 | c.994 − | | | | | | | | | | | | | | |
| | 2A > G | | | | | | | | | | | | | | |

| | | | carboplatin/ | carboplatin/ | carboplatin/ | carboplatin/ | | carboplatin/ | | | | cisplatin/ | cisplatin/ | cisplatin/ |
|---|---|---|---|---|---|---|---|---|---|---|---|---|---|---|
| | | cabozantinib | docetaxel | etoposide | gemcitabine | paclitaxel | | pemetrexed | cetuximab | | cisplatin | docetaxel | etoposide | gemcitabine |
| Gene | Variant | ✓ | ✓ | ✓ | ✓ | ✓ | X | ✓ | ✓ | X | X | ✓ | ✓ | ✓ |
| TP53 | c.332T > C | | | | | | | | | | | | | |
| TP53 | c.358A > G | | | | | | | | | | | | | |
| TP53 | c.376 − | | | | | | | | | | | | | |
| | 1G > A | | | | | | | | | | | | | |
| TP53 | c.395A > G | | | | | | | | | | | | | |
| TP53 | c.404G > A | | | | | | | | | | | | | |
| TP53 | c.405C > A | | | | | | | | | | | | | |
| TP53 | c.488A > G | | | | | | | | | | | | | |
| TP53 | c.524G > A | | | | | | | | | | | | | |
| TP53 | c.527G > T | | | | | | | | | | | | | |
| TP53 | c.542G > A | | | | | | | | | | | | | |
| TP53 | c.559 + | | | | | | | | | | | | | |
| | 1G > A | | | | | | | | | | | | | |
| TP53 | c.559 + | | | | | | | | | | | | | |
| | 1G > C | | | | | | | | | | | | | |
| TP53 | c.584T > C | | | | | | | | | | | | | |
| TP53 | c.614A > G | | | | | | | | | | | | | |
| TP53 | c.637C > T | | | | | | | | | | | | | |
| TP53 | c.646G > A | | | | | | | | | 1 | | | | |
| TP53 | c.673 − | | | | | | | | | | | | | |
| | 19G > A | | | | | | | | | | | | | |
| TP53 | c.695T > A | | | | | | | | | | | | | |
| TP53 | c.701A > G | | | | | | | | | 1 | | | | |
| TP53 | c.713G > A | | | | | | | | | | | | | |
| TP53 | c.713G > C | | | | | | | | | | | | | |
| TP53 | c.742C > T | | | | | | | | | | | | | |
| TP53 | c.743G > A | | | | | | | | | | | | | |
| TP53 | c.743G > T | | | | | | | | | | | | | |
| TP53 | c.817C > T | | | | | | | | | | | | | |
| TP53 | c.818G > A | | | | | | | | | | | | | |
| TP53 | c.844C > G | | | | | | | | | | | | | |
| TP53 | c.91G > A | | | | | | | | | | | | | |
| TP53 | c.994 − | | | | | | | | | | | | | |
| | 2A > G | | | | | | | | | | | | | |

| | | | | | | | | | | cyclophosphamide/ | | |
|---|---|---|---|---|---|---|---|---|---|---|---|---|
| | | | | | | | | | | doxorubicin/ | | |
| | | cisplatin/ | cisplatin/ | | | cobimetinib/ | | | | prednisone/ | cyclophosphamide/ | |
| | | paclitaxel | pemetrexed | clofarabine | cobimetinib | vemurafenib | copanlisib | crizotinib | cyclophosphamide | vincristine | fludarabine | cytarabine |
| Gene | Variant | ✓ | ✓ | X | ✓ | ✓ | ✓ | X | X | X | X | X |
| TP53 | c.332T > C | | | | | | | | | 1 | | |
| TP53 | c.358A > G | | | | | | | | | 1 | | |
| TP53 | c.376 − | | | | | | | | | 1 | | |
| | 1G > A | | | | | | | | | | | |
| TP53 | c.395A > G | | | | | | | | | 1 | | |
| TP53 | c.404G > A | | | | | | | | | 1 | | |
| TP53 | c.405C > A | | | | | | | | | 1 | | |
| TP53 | c.488A > G | | | | | | | | | 1 | | |
| TP53 | c.524G > A | | | | | | | | | 1 | 1 | |
| TP53 | c.527G > T | | | | | | | | | 1 | | |
| TP53 | c.542G > A | | | | | | | | | 1 | | |
| TP53 | c.559 + | | | | | | | | | 1 | | |
| | 1G > A | | | | | | | | | | | |
| TP53 | c.559 + | | | | | | | | | 1 | | |
| | 1G > C | | | | | | | | | | | |
| TP53 | c.584T > C | | | | | | | | | 1 | | |
| TP53 | c.614A > G | | | | | | | | | 1 | | |
| TP53 | c.637C > T | | | | | | | | | 1 | | |
| TP53 | c.646G > A | | | | | | | | | 1 | | |
| TP53 | c.673 − | | | | | | | | | 1 | | |
| | 19G > A | | | | | | | | | | | |
| TP53 | c.695T > A | | | | | | | | | 1 | | |
| TP53 | c.701A > G | | | | | | | | | 1 | | |
| TP53 | c.713G > A | | | | | | | | | 1 | | |
| TP53 | c.713G > C | | | | | | | | | 1 | | |
| TP53 | c.742C > T | | | | | | | | | 1 | | |
| TP53 | c.743G > A | | | | | | | | | 1 | | |
| TP53 | c.743G > T | | | | | | | | | 1 | | |
| TP53 | c.817C > T | | | 1 | | | | | 1 | 1 | | 1 |
| TP53 | c.818G > A | | | 1 | | | | | 1 | 1 | | 1 |
| TP53 | c.844C > G | | | | | | | | | 1 | | |
| TP53 | c.91G > A | | | | | | | | | 1 | | |
| TP53 | c.994 − | | | | | | | | | 1 | | |
| | 2A > G | | | | | | | | | | | |

| | | | | | | | dabrafenib/ | | | | | | | |
|---|---|---|---|---|---|---|---|---|---|---|---|---|---|---|
| | | cytarabine/ | | | dabrafenib/ | | trametinib/ | | | | | | decitabine/ | |
| | | daunorubicin | dabrafenib | | trametinib | | vemurafenib | dasatinib | | daunorubicin | | decitabine | sorafenib | dexamethasone |
| Gene | Variant | ✓ | ✓ | X | ✓ | X | X | ✓ | X | ✓ | X | ✓ | ✓ | X |
| TP53 | c.332T > C | | | | | | | | | | | | | |
| TP53 | c.358A > G | | | | | | | | | | | | | |
| TP53 | c.376 − | | | | | | | | | | | | | |
| | 1G > A | | | | | | | | | | | | | |
| TP53 | c.395A > G | | | | | | | | | | | | | |
| TP53 | c.404G > A | | | | | | | | | | | | | |
| TP53 | c.405C > A | | | | | | | | | | | | | |
| TP53 | c.488A > G | | | | | | | | | | | | | |
| TP53 | c.524G > A | | | | | | | | | | | | | |
| TP53 | c.527G > T | | | | | | | | | | | | | |
| TP53 | c.542G > A | | | | | | | | | | | | | |
| TP53 | c.559 + | | | | | | | | | | | | | |
| | 1G > A | | | | | | | | | | | | | |
| TP53 | c.559 + | | | | | | | | | | | | | |
| | 1G > C | | | | | | | | | | | | | |
| TP53 | c.584T > C | | | | | | | | | | | | | |
| TP53 | c.614A > G | | | | | | | | | | | | | |
| TP53 | c.637C > T | | | | | | | | | | | | | |
| TP53 | c.646G > A | | | | | | | | | | | | | |
| TP53 | c.673 − | | | | | | | | | | | | | |
| | 19G > A | | | | | | | | | | | | | |
| TP53 | c.695T > A | | | | | | | | | | | | | |
| TP53 | c.701A > G | | | | | | | | | | | | | |
| TP53 | c.713G > A | | | | | | | | | | | | | |
| TP53 | c.713G > C | | | | | | | | | | | | | |
| TP53 | c.742C > T | | | | | | | | | | | | | |
| TP53 | c.743G > A | | | | | | | | | | | | | |
| TP53 | c.743G > T | | | | | | | | | | | | | |
| TP53 | c.817C > T | | | | | | | | | | | | | 1 |
| TP53 | c.818G > A | | | | | | | | | | | | | 1 |
| TP53 | c.844C > G | | | | | | | | | | | | | |
| TP53 | c.91G > A | | | | | | | | | | | | | |
| TP53 | c.994 − | | | | | | | | | | | | | |
| | 2A > G | | | | | | | | | | | | | |

| | | | | | EGFR | | | | | | | |
|---|---|---|---|---|---|---|---|---|---|---|---|---|
| | | | | | tyrosine | | | | | | | |
| | | | docetaxel/ | doxo- | kinase | | | | | | | fludarabine |
| | | docetaxel | gemcitabine | rubicin | inhibitor | enasidenib | enzalutamide | erlotinib | | etoposide | filgrastim | phosphate |
| Gene | Variant | X | ✓ | X | X | ✓ | X | ✓ | X | X | ✓ | ✓ |
| TP53 | c.332T > C | | | | | | | | | | | |
| TP53 | c.358A > G | | | | | | | | | | | |
| TP53 | c.376 − | | | | | | | | | | | 1 |
| | 1G > A | | | | | | | | | | | |
| TP53 | c.395A > G | | | | | | | | | | | 1 |
| TP53 | c.404G > A | | | | | | | | | | | 1 |
| TP53 | c.405C > A | | | | | | | | | | | 1 |
| TP53 | c.488A > G | | | | | | | | | | | |
| TP53 | c.524G > A | 1 | | | | | 1 | | | | | |
| TP53 | c.527G > T | | | | | | | | | | | |
| TP53 | c.542G > A | | | | | | | | | | | |
| TP53 | c.559 + | | | | | | | | | | | |
| | 1G > A | | | | | | | | | | | |
| TP53 | c.559 + | | | | | | | | | | | 1 |
| | 1G > C | | | | | | | | | | | |
| TP53 | c.584T > C | | | | | | | | | | | 1 |
| TP53 | c.614A > G | | | | | | | | | | | 1 |
| TP53 | c.637C > T | | | | | | | | | | | |
| TP53 | c.646G > A | | | | | | | | | | | |
| TP53 | c.673 − | | | | | | | | | | | 1 |
| | 19G > A | | | | | | | | | | | |
| TP53 | c.695T > A | | | | | | | | | | | |
| TP53 | c.701A > G | | | | | | | | | | | |
| TP53 | c.713G > A | | | | | | | | | | | 1 |
| TP53 | c.713G > C | | | | | | | | | | | 1 |
| TP53 | c.742C > T | | | | | | | | | | | |
| TP53 | c.743G > A | | | | | | | | | | | 1 |
| TP53 | c.743G > T | | | | | | | | | | | 1 |
| TP53 | c.817C > T | | | 1 | | | | | | 1 | | |
| TP53 | c.818G > A | | | 1 | | | | | | 1 | | |
| TP53 | c.844C > G | | | | | | | | | | | |
| TP53 | c.91G > A | | | | | | | | | | | |
| TP53 | c.994 − | | | | | | | | | | | |
| | 2A > G | | | | | | | | | | | |

| | | | fluoropyrimidine/ | | | gemcitabine/ | | | idelalisib/ | idelalisib/ | | | ipilimumab/ |
|---|---|---|---|---|---|---|---|---|---|---|---|---|---|
| | | fluoropyrimidine | oxaliplatin | | gefitinib | vinorelbine | ibrutinib | idelalisib | ofatumumab | rituximab | imatinib | | nivolumab |
| Gene | Variant | ✓ | ✓ | X | X | ✓ | ✓ | ✓ | ✓ | ✓ | ✓ | X | ✓ |
| TP53 | c.332T > C | | | | | | 1 | 1 | 1 | 2 | | 1 | |
| TP53 | c.358A > G | | | | | | 1 | 1 | 1 | 2 | | 1 | |
| TP53 | c.376 − | | | | | | 1 | 1 | 1 | 2 | | 1 | |
| | 1G > A | | | | | | | | | | | | |
| TP53 | c.395A > G | | | | | | 1 | 1 | 1 | 2 | | 1 | |
| TP53 | c.404G > A | | | | | | 1 | 1 | 1 | 2 | | 1 | |
| TP53 | c.405C > A | | | | | | 1 | 1 | 1 | 2 | | 1 | |
| TP53 | c.488A > G | | | | | | 1 | 1 | 1 | 2 | | 1 | |
| TP53 | c.524G > A | | | | | | 1 | 1 | 1 | 2 | | 1 | |
| TP53 | c.527G > T | | | | | | 1 | 1 | 1 | 2 | | 1 | |
| TP53 | c.542G > A | | | | | | 1 | 1 | 1 | 2 | | 1 | |
| TP53 | c.559 + | | | | | | 1 | 1 | 1 | 2 | | 1 | |
| | 1G > A | | | | | | | | | | | | |
| TP53 | c.559 + | | | | | | 1 | 1 | 1 | 2 | | 1 | |
| | 1G > C | | | | | | | | | | | | |
| TP53 | c.584T > C | | | | | | 1 | 1 | 1 | 2 | | 1 | |
| TP53 | c.614A > G | | | | | | 1 | 1 | 1 | 2 | | 1 | |
| TP53 | c.637C > T | | | | | | 1 | 1 | 1 | 2 | | 1 | |
| TP53 | c.646G > A | | | | | | 1 | 2 | 1 | 1 | | 1 | |
| TP53 | c.673 − | | | | | | 1 | 1 | 1 | 2 | | 1 | |
| | 19G > A | | | | | | | | | | | | |
| TP53 | c.695T > A | | | | | | 1 | 1 | 1 | 2 | | 1 | |
| TP53 | c.701A > G | | | | | | 1 | 2 | 1 | 1 | | 1 | |
| TP53 | c.713G > A | | | | | | 1 | 1 | 1 | 2 | | 1 | |
| TP53 | c.713G > C | | | | | | 1 | 1 | 1 | 2 | | 1 | |
| TP53 | c.742C > T | | | | | | 1 | 1 | 1 | 2 | | 1 | |
| TP53 | c.743G > A | | | | | | 1 | 1 | 1 | 2 | | 1 | |
| TP53 | c.743G > T | | | | | | 1 | 1 | 1 | 2 | | 1 | |
| TP53 | c.817C > T | | | | | | 1 | 1 | 1 | 2 | | 1 | |
| TP53 | c.818G > A | | | | | | 1 | 2 | 1 | 1 | | 1 | |
| TP53 | c.844C > G | | | | | | 1 | 2 | 1 | 1 | | 1 | |
| TP53 | c.91G > A | | | | | | 1 | 2 | 1 | 1 | | 1 | |
| TP53 | c.994 − | | | | | | 1 | 2 | 1 | 1 | | 1 | |
| | 2A > G | | | | | | | | | | | | |

| | | | | | | | MAP | | | | |
|---|---|---|---|---|---|---|---|---|---|---|---|
| | | | | | | | kinase | | | | |
| | | | | lenalidomide/ | | | kinase | | methylpred- | | |
| | | lenalidomide | | rituximab | lenvatinib | lorlatinib | inhibitor | methotrexate | nisolone | midostaurin | mitoxantrone |
| Gene | Variant | ✓ | X | ✓ | ✓ | X | ✓ | X | X | ✓ | X |
| TP53 | c.332T > C | 1 | 1 | 1 | | 1 | | | | | |
| TP53 | c.358A > G | 1 | 1 | 1 | | 1 | | | | | |
| TP53 | c.376 − | 1 | 1 | 1 | | 1 | | | | | |
| | 1G > A | | | | | | | | | | |
| TP53 | c.395A > G | 1 | 1 | 1 | | 1 | | | | | |
| TP53 | c.404G > A | 1 | 1 | 1 | | 1 | | | | | |
| TP53 | c.405C > A | 1 | 1 | 1 | | 1 | | | | | |
| TP53 | c.488A > G | 1 | 1 | 1 | | 1 | | | | | |
| TP53 | c.524G > A | 1 | 1 | 1 | | 1 | | | | | |
| TP53 | c.527G > T | 1 | 1 | 1 | | 1 | | | | | |
| TP53 | c.542G > A | 1 | 1 | 1 | | 1 | | | | | |
| TP53 | c.559 + | 1 | 1 | 1 | | 1 | | | | | |
| | 1G > A | | | | | | | | | | |
| TP53 | c.559 + | 1 | 1 | 1 | | 1 | | | | | |
| | 1G > C | | | | | | | | | | |
| TP53 | c.584T > C | 1 | 1 | 1 | | 1 | | | | | |
| TP53 | c.614A > G | 1 | 1 | 1 | | 1 | | | | | |
| TP53 | c.637C > T | 1 | 1 | 1 | | 1 | | | | | |
| TP53 | c.646G > A | 1 | 1 | 1 | | 1 | | | | | |
| TP53 | c.673 − | 1 | 1 | 1 | | 1 | | | | | |
| | 19G > A | | | | | | | | | | |
| TP53 | c.695T > A | 1 | 1 | 1 | | 1 | | | | | |
| TP53 | c.701A > G | 1 | 1 | 1 | | 1 | | | | | |
| TP53 | c.713G > A | 1 | 1 | 1 | | 1 | | | | | |
| TP53 | c.713G > C | 1 | 1 | 1 | | 1 | | | | | |
| TP53 | c.742C > T | 1 | 1 | 1 | | 1 | | | | | |
| TP53 | c.743G > A | 1 | 1 | 1 | | 1 | | | | | |
| TP53 | c.743G > T | 1 | 1 | 1 | | 1 | | | | | |
| TP53 | c.817C > T | 1 | 1 | 1 | | 1 | | | | | 1 |
| TP53 | c.818G > A | 1 | 1 | 1 | | 1 | | | | | 1 |
| TP53 | c.844C > G | 1 | 1 | 1 | | 1 | | | | | |
| TP53 | c.91G > A | 1 | 1 | 1 | | 1 | | | | | |
| TP53 | c.994 − | 1 | 1 | 1 | | 1 | | | | | |
| | 2A > G | | | | | | | | | | |

| | | | | | | | | | omacetaxine | |
|---|---|---|---|---|---|---|---|---|---|---|
| | | nilotinib | | nintedanib | nivolumab | obinutuzumab | ofatumumab | olaratumab | mepesuccinate | osimertinib |
| Gene | Variant | ✓ | X | ✓ | ✓ | ✓ | ✓ | ✓ | ✓ | X |
| TP53 | c.332T > C | | | | | 1 | 1 | | | |
| TP53 | c.358A > G | | | | | 1 | 1 | | | |
| TP53 | c.376 − | | | | | 1 | 1 | | | |
| | 1G > A | | | | | | | | | |
| TP53 | c.395A > G | | | | | 1 | 1 | | | |
| TP53 | c.404G > A | | | | | 1 | 1 | | | |
| TP53 | c.405C > A | | | | | 1 | 1 | | | |
| TP53 | c.488A > G | | | | | 1 | 1 | | | |
| TP53 | c.524G > A | | | | | 1 | 1 | | | |
| TP53 | c.527G > T | | | | | 1 | 1 | | | |
| TP53 | c.542G > A | | | | | 1 | 1 | | | |
| TP53 | c.559 + | | | | | 1 | 1 | | | |
| | 1G > A | | | | | | | | | |
| TP53 | c.559 + | | | | | 1 | 1 | | | |
| | 1G > C | | | | | | | | | |
| TP53 | c.584T > C | | | | | 1 | 1 | | | |
| TP53 | c.614A > G | | | | | 1 | 1 | | | |
| TP53 | c.637C > T | | | | | 1 | 1 | | | |
| TP53 | c.646G > A | | | | | 1 | 1 | | | |
| TP53 | c.673 − | | | | | 1 | 1 | | | |
| | 19G > A | | | | | | | | | |
| TP53 | c.695T > A | | | | | 1 | 1 | | | |
| TP53 | c.701A > G | | | | | 1 | 1 | | | |
| TP53 | c.713G > A | | | | | 1 | 1 | | | |
| TP53 | c.713G > C | | | | | 1 | 1 | | | |
| TP53 | c.742C > T | | | | | 1 | 1 | | | |
| TP53 | c.743G > A | | | | | 1 | 1 | | | |
| TP53 | c.743G > T | | | | | 1 | 1 | | | |
| TP53 | c.817C > T | | | | | 1 | 1 | | | |
| TP53 | c.818G > A | | | | | 1 | 1 | | | |
| TP53 | c.844C > G | | | | | 1 | 1 | | | |
| TP53 | c.91G > A | | | | | 1 | 1 | | | |
| TP53 | c.994 − | | | | | 1 | 1 | | | |
| | 2A > G | | | | | | | | | |

| | | | | | | | | | platinum | | | | |
|---|---|---|---|---|---|---|---|---|---|---|---|---|---|
| | | | | | PEG- | | | PI 3- | chemotherapy | | | | |
| | | | | | interferon | | | kinase | regimen/ | | | | |
| | | panitumumab | | pazopanib | alfa 2-a | pembrolizumab | pexidartinib | inhibitor | vinorelbine | ponatinib | | prednisolone | prednisone |
| Gene | Variant | ✓ | X | ✓ | X | ✓ | X | X | X | ✓ | X | X | X |
| TP53 | c.332T > C | | | | | | | | | | | | |
| TP53 | c.358A > G | | | | | | | | | | | | |
| TP53 | c.376 − | | | | | | | | | | | | |
| | 1G > A | | | | | | | | | | | | |
| TP53 | c.395A > G | | | | | | | | | | | | |
| TP53 | c.404G > A | | | | | | | | | | | | |
| TP53 | c.405C > A | | | | | | | | | | | | |
| TP53 | c.488A > G | | | | | | | | | | | | |
| TP53 | c.524G > A | | | | | | | | | | | 1 | |
| TP53 | c.527G > T | | | | | | | | | | | | |
| TP53 | c.542G > A | | | | | | | | | | | | |
| TP53 | c.559 + | | | | | | | | | | | | |
| | 1G > A | | | | | | | | | | | | |
| TP53 | c.559 + | | | | | | | | | | | | |
| | 1G > C | | | | | | | | | | | | |
| TP53 | c.584T > C | | | | | | | | | | | | |
| TP53 | c.614A > G | | | | | | | | | | | | |
| TP53 | c.637C > T | | | | | | | | | | | | |
| TP53 | c.646G > A | | | | | | | | | | 1 | | |
| TP53 | c.673 − | | | | | | | | | | | | |
| | 19G > A | | | | | | | | | | | | |
| TP53 | c.695T > A | | | | | | | | | | | | |
| TP53 | c.701A > G | | | | | | | | | | | | |
| TP53 | c.713G > A | | | | | | | | | | | | |
| TP53 | c.713G > C | | | | | | | | | | | | |
| TP53 | c.742C > T | | | | | | | | | | | | |
| TP53 | c.743G > A | | | | | | | | | | | | |
| TP53 | c.743G > T | | | | | | | | | | | | |
| TP53 | c.817C > T | | | | | | | | | | | | |
| TP53 | c.818G > A | | | | | | | | | | | | |
| TP53 | c.844C > G | | | | | | | | | | | | |
| TP53 | c.91G > A | | | | | | | | | | | | |
| TP53 | c.994 − | | | | | | | | | | | | |
| | 2A > G | | | | | | | | | | | | |

| | | | | | | | rituximab/ | | | | | |
|---|---|---|---|---|---|---|---|---|---|---|---|---|
| | | quizartinib | ramucirumab | regorafenib | rituximab | | venetoclax | rociletinib | ruxolitinib | selumetinib | sorafenib | |
| Gene | Variant | X | ✓ | ✓ | ✓ | X | ✓ | X | ✓ | ✓ | ✓ | X |
| TP53 | c.332T > C | | | | 1 | 2 | 1 | | | | | |
| TP53 | c.358A > G | | | | 1 | 2 | 1 | | | | | |
| TP53 | c.376 − | | | | 1 | 2 | 1 | | | | | |
| | 1G > A | | | | | | | | | | | |
| TP53 | c.395A > G | | | | 1 | 2 | 1 | | | | | |
| TP53 | c.404G > A | | | | 1 | 2 | 1 | | | | | |
| TP53 | c.405C > A | | | | 1 | 2 | 1 | | | | | |
| TP53 | c.488A > G | | | | 1 | 2 | 1 | | | | | |
| TP53 | c.524G > A | | | | 1 | 2 | 1 | | | | | |
| TP53 | c.527G > T | | | | 1 | 2 | 1 | | | | | |
| TP53 | c.542G > A | | | | 1 | 2 | 1 | | | | | |
| TP53 | c.559 + | | | | 1 | 2 | 1 | | | | | |
| | 1G > A | | | | | | | | | | | |
| TP53 | c.559 + | | | | 1 | 2 | 1 | | | | | |
| | 1G > C | | | | | | | | | | | |
| TP53 | c.584T > C | | | | 1 | 2 | 1 | | | | | |
| TP53 | c.614A > G | | | | 1 | 2 | 1 | | | | | |
| TP53 | c.637C > T | | | | 1 | 2 | 1 | | | | | |
| TP53 | c.646G > A | | | | 1 | 2 | 1 | | | | | |
| TP53 | c.673 − | | | | 1 | 2 | 1 | | | | | |
| | 19G > A | | | | | | | | | | | |
| TP53 | c.695T > A | | | | 1 | 2 | 1 | | | | | |
| TP53 | c.701A > G | | | | 1 | 2 | 1 | | | | | |
| TP53 | c.713G > A | | | | 1 | 2 | 1 | | | | | |
| TP53 | c.713G > C | | | | 1 | 2 | 1 | | | | | |
| TP53 | c.742C > T | | | | 1 | 2 | 1 | | | | | |
| TP53 | c.743G > A | | | | 1 | 2 | 1 | | | | | |
| TP53 | c.743G > T | | | | 1 | 2 | 1 | | | | | |
| TP53 | c.817C > T | | | | 1 | 2 | 1 | | | | | |
| TP53 | c.818G > A | | | | 1 | 2 | 1 | | | | | |
| TP53 | c.844C > G | | | | 1 | 2 | 1 | | | | | |
| TP53 | c.91G > A | | | | 1 | 2 | 1 | | | | | |
| TP53 | c.994 − | | | | 1 | 2 | 1 | | | | | |
| | 2A > G | | | | | | | | | | | |

| | | sunitinib | | temozolomide | trametinib | tretinoin | trifluridine | vemurafenib | | venetoclax | vincristine | vinorelbine |
|---|---|---|---|---|---|---|---|---|---|---|---|---|
| Gene | Variant | ✓ | X | ✓ | ✓ | X | ✓ | ✓ | X | ✓ | X | X |
| TP53 | c.332T > C | | | | | | | | | 1 | | |
| TP53 | c.358A > G | | | | | | | | | 1 | | |
| TP53 | c.376 − | | | | | | | | | 1 | | |
| | 1G > A | | | | | | | | | | | |
| TP53 | c.395A > G | | | | | | | | | 1 | | |
| TP53 | c.404G > A | | | | | | | | | 1 | | |
| TP53 | c.405C > A | | | | | | | | | 1 | | |
| TP53 | c.488A > G | | | | | | | | | 1 | | |
| TP53 | c.524G > A | | | | | | | | | 1 | | |
| TP53 | c.527G > T | | | | | | | | | 1 | | |
| TP53 | c.542G > A | | | | | | | | | 1 | | |
| TP53 | c.559 + | | | | | | | | | 1 | | |
| | 1G > A | | | | | | | | | | | |
| TP53 | c.559 + | | | | | | | | | 1 | | |
| | 1G > C | | | | | | | | | | | |
| TP53 | c.584T > C | | | | | | | | | 1 | | |
| TP53 | c.614A > G | | | | | | | | | 1 | | |
| TP53 | c.637C > T | | | | | | | | | 1 | | |
| TP53 | c.646G > A | | | | | | | | | 1 | | |
| TP53 | c.673 − | | | | | | | | | 1 | | |
| | 19G > A | | | | | | | | | | | |
| TP53 | c.695T > A | | | | | | | | | 1 | | |
| TP53 | c.701A > G | | | | | | | | | 1 | | |
| TP53 | c.713G > A | | | | | | | | | 1 | | |
| TP53 | c.713G > C | | | | | | | | | 1 | | |
| TP53 | c.742C > T | | | | | | | | | 1 | | |
| TP53 | c.743G > A | | | | | | | | | 1 | | |
| TP53 | c.743G > T | | | | | | | | | 1 | | |
| TP53 | c.817C > T | | | | | | | | | 1 | 1 | |
| TP53 | c.818G > A | | | | | | | | | 1 | 1 | |
| TP53 | c.844C > G | | | | | | | | | 1 | | |
| TP53 | c.91G > A | | | | | | | | | 1 | | |
| TP53 | c.994 − | | | | | | | | | 1 | | |
| | 2A > G | | | | | | | | | | | |

✓ = sensitive;
X = resistant

TABLE 2

| | | 5-azacyti- | afati- | arsenic | axiti- | | | brigati- | cabozan- | carboplatin/ | | co- | cobimetinib/ | co- | | dabrafenib/ | dasati- |
|---|---|---|---|---|---|---|---|---|---|---|---|---|---|---|---|---|---|
| | | dine | nib | trioxide | nib | bosutinib | | nib | tinib | paclitaxel | cetuximab | bimetinib | vemurafenib | panlisib | dabrafenib | trametinib | nib |
| Gene | Variant | ✓ | X | ✓ | ✓ | ✓ | X | ✓ | ✓ | X | X | ✓ | ✓ | ✓ | ✓ | ✓ | ✓ |
| ABL1 | c.1270 + | | | | | 1 | | | | | | | | | | | 2 |
| | 42A > G | | | | | | | | | | | | | | | | |
| ABL1 | c.128G > A | | | | | 1 | | | | | | | | | | | 2 |
| ABL1 | c.1418G > A | | | | | 1 | | | | | | | | | | | 2 |
| ABL1 | c.1423 + | | | | | 1 | | | | | | | | | | | 2 |
| | 26C > G | | | | | | | | | | | | | | | | |
| ABL1 | c.1497A > G | | | | | 1 | | | | | | | | | | | 2 |
| ABL1 | c.−15_−14insC | | | | | 1 | | | | | | | | | | | 2 |
| ABL1 | c.1512C > T | | | | | 1 | | | | | | | | | | | 2 |
| ABL1 | c.1513 + | | | | | 1 | | | | | | | | | | | 2 |
| | 20_1513 + | | | | | | | | | | | | | | | | |
| | 21delGT | | | | | | | | | | | | | | | | |
| ABL1 | c.1513 + | | | | | 1 | | | | | | | | | | | 2 |
| | 21_1513 + | | | | | | | | | | | | | | | | |
| | 22insCACCCC | | | | | | | | | | | | | | | | |
| ABL1 | c.1596G > A | | | | | 1 | | | | | | | | | | | 2 |
| ABL1 | c.1674G > A | | | | | 1 | | | | | | | | | | | 2 |
| ABL1 | c.1677C > T | | | | | 1 | | | | | | | | | | | 2 |
| ABL1 | c.1678 + | | | | | 1 | | | | | | | | | | | 2 |
| | 14T > C | | | | | | | | | | | | | | | | |
| ABL1 | c.1679 − | | | | | 1 | | | | | | | | | | | 2 |
| | 11T > C | | | | | | | | | | | | | | | | |
| ABL1 | c.1701C > T | | | | | 1 | | | | | | | | | | | 2 |
| ABL1 | c.1826_1828delAGA | | | | | 1 | | | | | | | | | | | 2 |
| ABL1 | c.1854C > T | | | | | 1 | | | | | | | | | | | 2 |
| ABL1 | c.1869G > A | | | | | 1 | | | | | | | | | | | 2 |
| ABL1 | c.1899C > T | | | | | 1 | | | | | | | | | | | 2 |
| ABL1 | c.2040C > T | | | | | 1 | | | | | | | | | | | 2 |
| ABL1 | c.2116G > A | | | | | 1 | | | | | | | | | | | 2 |
| ABL1 | c.2117G > T | | | | | 1 | | | | | | | | | | | 2 |
| ABL1 | c.2187G > A | | | | | 1 | | | | | | | | | | | 2 |
| ABL1 | c.2342C > T | | | | | 1 | | | | | | | | | | | 2 |
| ABL1 | c.2352C > G | | | | | 1 | | | | | | | | | | | 2 |
| ABL1 | c.2429C > T | | | | | 1 | | | | | | | | | | | 2 |
| ABL1 | c.2470G > A | | | | | 1 | | | | | | | | | | | 2 |
| ABL1 | c.249T > G | | | | | 1 | | | | | | | | | | | 2 |
| ABL1 | c.254 − | | | | | 1 | | | | | | | | | | | 2 |
| | 50G > A | | | | | | | | | | | | | | | | |
| ABL1 | c.2703C > G | | | | | 1 | | | | | | | | | | | 2 |
| ABL1 | c.2782C > T | | | | | 1 | | | | | | | | | | | 2 |
| ABL1 | c.2882C > T | | | | | 1 | | | | | | | | | | | 2 |
| ABL1 | c.2883G > A | | | | | 1 | | | | | | | | | | | 2 |
| ABL1 | c.2904C > T | | | | | 1 | | | | | | | | | | | 2 |
| ABL1 | c.2915C > T | | | | | 1 | | | | | | | | | | | 2 |
| ABL1 | c.2916G > A | | | | | 1 | | | | | | | | | | | 2 |
| ABL1 | c.2940C > T | | | | | 1 | | | | | | | | | | | 2 |
| ABL1 | c.2958A > G | | | | | 1 | | | | | | | | | | | 2 |
| ABL1 | c.2970G > A | | | | | 1 | | | | | | | | | | | 2 |
| ABL1 | c.3000C > T | | | | | 1 | | | | | | | | | | | 2 |
| ABL1 | c.3043A > C | | | | | 1 | | | | | | | | | | | 2 |
| ABL1 | c.3072C > T | | | | | 1 | | | | | | | | | | | 2 |
| ABL1 | c.−30T > C | | | | | 1 | | | | | | | | | | | 2 |
| ABL1 | c.3324A > G | | | | | 1 | | | | | | | | | | | 2 |
| ABL1 | c.3344G > C | | | | | 1 | | | | | | | | | | | 2 |
| ABL1 | c.501C > T | | | | | 1 | | | | | | | | | | | 2 |
| ABL1 | c.−53C > G | | | | | 1 | | | | | | | | | | | 2 |
| ABL1 | c.549 + | | | | | 1 | | | | | | | | | | | 2 |
| | 47A > G | | | | | | | | | | | | | | | | |
| ABL1 | c.−588A > T | | | | | 1 | | | | | | | | | | | 2 |
| ABL1 | c.−70C > A | | | | | 1 | | | | | | | | | | | 2 |
| ABL1 | c.720G > A | | | | | 1 | | | | | | | | | | | 2 |
| ABL1 | c.740A > G | | | | | 1 | | | | | | | | | | | 2 |
| ABL1 | c.763G > A | | | | | 1 | | | | | | | | | | | 1 |
| ABL1 | c.764A > T | | | | | 1 | | | | | | | | | | | 1 |
| ABL1 | c.−79G > A | | | | | 1 | | | | | | | | | | | 2 |
| ABL1 | c.822 + | | | | | 1 | | | | | | | | | | | 2 |
| | 16C > G | | | | | | | | | | | | | | | | |
| ABL1 | c.843A > G | | | | | 1 | | | | | | | | | | | 2 |
| ABL1 | c.908 − | | | | | 1 | | | | | | | | | | | 2 |
| | 31C > A | | | | | | | | | | | | | | | | |
| ABL1 | c.908 − | | | | | 1 | | | | | | | | | | | 2 |
| | 45T > C | | | | | | | | | | | | | | | | |
| ABL1 | c.944C > T | | | | | | 1 | | | | | | | | | | |
| ABL1 | c.991A > G | | | | | 1 | | | | | | | | | | | 2 |
| ASXL1 | c.*22A > G | | | | | | | | | | | | | | | | |
| ASXL1 | c.1158G > T | | | | | | | | | | | | | | | | |
| ASXL1 | c.1210C > T | | | | | | | | | | | | | | | | |
| ASXL1 | c.1331C > T | | | | | | | | | | | | | | | | |
| ASXL1 | c.140 + | | | | | | | | | | | | | | | | |
| | 26A > G | | | | | | | | | | | | | | | | |
| ASXL1 | c.1429G > C | | | | | | | | | | | | | | | | |
| ASXL1 | c.143 + | | | | | | | | | | | | | | | | |
| | 50C > T | | | | | | | | | | | | | | | | |
| ASXL1 | c.1465C > G | | | | | | | | | | | | | | | | |
| ASXL1 | c.1520T > A | | | | | | | | | | | | | | | | |
| ASXL1 | c.1749G > A | | | | | | | | | | | | | | | | |
| ASXL1 | c.1895_1896delinsAA | | | | | | | | | | | | | | | | |
| ASXL1 | c.1898A > G | | | | | | | | | | | | | | | | |
| ASXL1 | c.1923C > T | | | | | | | | | | | | | | | | |
| ASXL1 | c.1926_1927delAG | | | | | | | | | | | | | | | | |
| ASXL1 | c.1926delA | | | | | | | | | | | | | | | | |
| ASXL1 | c.1935dupT | | | | | | | | | | | | | | | | |
| ASXL1 | c.1954G > A | | | | | | | | | | | | | | | | |
| ASXL1 | c.1965C > T | | | | | | | | | | | | | | | | |
| ASXL1 | c.2059T > C | | | | | | | | | | | | | | | | |
| ASXL1 | c.2078_2103del26 | | | | | | | | | | | | | | | | |
| ASXL1 | c.2129delG | | | | | | | | | | | | | | | | |
| ASXL1 | c.2250C > T | | | | | | | | | | | | | | | | |
| ASXL1 | c.2251G > A | | | | | | | | | | | | | | | | |
| ASXL1 | c.2395G > T | | | | | | | | | | | | | | | | |
| ASXL1 | c.2416_2417dupAC | | | | | | | | | | | | | | | | |
| ASXL1 | c.2444T > C | | | | | | | | | | | | | | | | |
| ASXL1 | c.2461delG | | | | | | | | | | | | | | | | |
| ASXL1 | c.2473A > T | | | | | | | | | | | | | | | | |
| ASXL1 | c.2513A > G | | | | | | | | | | | | | | | | |
| ASXL1 | c.252 + | | | | | | | | | | | | | | | | |
| | 11delT | | | | | | | | | | | | | | | | |
| ASXL1 | c.252 + | | | | | | | | | | | | | | | | |
| | 11T > C | | | | | | | | | | | | | | | | |
| ASXL1 | c.2929C > T | | | | | | | | | | | | | | | | |
| ASXL1 | c.2957A > G | | | | | | | | | | | | | | | | |
| ASXL1 | c.2985C > T | | | | | | | | | | | | | | | | |
| ASXL1 | c.3029C > T | | | | | | | | | | | | | | | | |
| ASXL1 | c.3098A > T | | | | | | | | | | | | | | | | |
| ASXL1 | c.3384C > T | | | | | | | | | | | | | | | | |
| ASXL1 | c.3498C > G | | | | | | | | | | | | | | | | |
| ASXL1 | c.3503G > C | | | | | | | | | | | | | | | | |
| ASXL1 | c.3513G > A | | | | | | | | | | | | | | | | |
| ASXL1 | c.3519G > A | | | | | | | | | | | | | | | | |
| ASXL1 | c.3662C > A | | | | | | | | | | | | | | | | |
| ASXL1 | c.3692C > T | | | | | | | | | | | | | | | | |
| ASXL1 | c.373 + | | | | | | | | | | | | | | | | |
| | 16T > G | | | | | | | | | | | | | | | | |
| ASXL1 | c.3739C > T | | | | | | | | | | | | | | | | |
| ASXL1 | c.3745A > G | | | | | | | | | | | | | | | | |
| ASXL1 | c.374 − | | | | | | | | | | | | | | | | |
| | 9C > T | | | | | | | | | | | | | | | | |
| ASXL1 | c.3759T > C | | | | | | | | | | | | | | | | |
| ASXL1 | c.3889G > A | | | | | | | | | | | | | | | | |
| ASXL1 | c.3973C > T | | | | | | | | | | | | | | | | |
| ASXL1 | c.4098C > T | | | | | | | | | | | | | | | | |
| ASXL1 | c.4189G > A | | | | | | | | | | | | | | | | |
| ASXL1 | c.4470G > T | | | | | | | | | | | | | | | | |
| ASXL1 | c.472 − | | | | | | | | | | | | | | | | |
| | 22T > C | | | | | | | | | | | | | | | | |
| ASXL1 | c.540C > T | | | | | | | | | | | | | | | | |
| ASXL1 | c.57 + | | | | | | | | | | | | | | | | |
| | 19G > A | | | | | | | | | | | | | | | | |
| ASXL1 | c.57 + | | | | | | | | | | | | | | | | |
| | 49dupG | | | | | | | | | | | | | | | | |
| ASXL1 | c.57 + | | | | | | | | | | | | | | | | |
| | 52C > T | | | | | | | | | | | | | | | | |
| ASXL1 | c.582C > T | | | | | | | | | | | | | | | | |
| ASXL1 | c.58 − | | | | | | | | | | | | | | | | |
| | 49A > C | | | | | | | | | | | | | | | | |
| ASXL1 | c.719 − | | | | | | | | | | | | | | | | |
| | 100C > T | | | | | | | | | | | | | | | | |
| ASXL1 | c.993T > C | | | | | | | | | | | | | | | | |
| BRAF | c.1068A > G | | | | | | | | | | 1 | | | | 1 | | |
| BRAF | c.1140 + | | | | | | | | | | 1 | | | | 1 | | |
| | 45A > G | | | | | | | | | | | | | | | | |
| BRAF | c.1141 − | | | | | | | | | | 1 | | | | 1 | | |
| | 19C > T | | | | | | | | | | | | | | | | |
| BRAF | c.1150A > G | | | | | | | | | | 1 | | | | 1 | | |
| BRAF | c.1227A > G | | | | | | | | | | 1 | | | | 1 | | |
| BRAF | c.1315 − | | | | | | | | | | 1 | | | | 1 | | |
| | 18T > G | | | | | | | | | | | | | | | | |
| BRAF | c.1383A > G | | | | | | | | | | 1 | | | | 1 | | |
| BRAF | c.1405G > C | | | | | | | | | | | | | | 1 | | |
| BRAF | c.1517 + | | | | | | | | | | 1 | | | | 1 | | |
| | 20T > C | | | | | | | | | | | | | | | | |
| BRAF | c.1517 + | | | | | | | | | | 1 | | | | 1 | | |
| | 35G > C | | | | | | | | | | | | | | | | |
| BRAF | c.1517 + | | | | | | | | | | 1 | | | | 1 | | |
| | 46T > C | | | | | | | | | | | | | | | | |
| BRAF | c.1518 − | | | | | | | | | | 1 | | | | 1 | | |
| | 48C > T | | | | | | | | | | | | | | | | |
| BRAF | c.1694 + | | | | | | | | | | 1 | | | | 1 | | |
| | 14G > A | | | | | | | | | | | | | | | | |
| BRAF | c.1742A > G | | | | | | | | | | 1 | | | | 1 | | |
| BRAF | c.1781A > G | | | | | | | | | | 1 | | | | 1 | | |
| BRAF | c.1782T > A | | | | | | | | | | | | | | | | |
| BRAF | c.1799T > A | | | | | | | | | | 2 | 2 | 1 | | 3 | 1 | |
| BRAF | c.1929A > G | | | | | | | | | | 1 | | | | 1 | | |
| BRAF | c.1992 + | | | | | | | | | | 1 | | | | 1 | | |
| | 16G > A | | | | | | | | | | | | | | | | |
| BRAF | c.1992 + | | | | | | | | | | 1 | | | | 1 | | |
| | 16G > C | | | | | | | | | | | | | | | | |
| BRAF | c.1992 + | | | | | | | | | | 1 | | | | 1 | | |
| | 1847G > C | | | | | | | | | | | | | | | | |
| BRAF | c.1992 + | | | | | | | | | | 1 | | | | 1 | | |
| | 40A > G | | | | | | | | | | | | | | | | |
| BRAF | c.2127 + | | | | | | | | | | 1 | | | | 1 | | |
| | 3A > G | | | | | | | | | | | | | | | | |
| BRAF | c.2128 − | | | | | | | | | | 1 | | | | 1 | | |
| | 16C > T | | | | | | | | | | | | | | | | |
| BRAF | c.2128 − | | | | | | | | | | 1 | | | | 1 | | |
| | 17dupT | | | | | | | | | | | | | | | | |
| BRAF | c.2128 − | | | | | | | | | | 1 | | | | 1 | | |
| | 26T > C | | | | | | | | | | | | | | | | |
| BRAF | c.2128 − | | | | | | | | | | 1 | | | | 1 | | |
| | 27_2128 − | | | | | | | | | | | | | | | | |
| | 18del10 | | | | | | | | | | | | | | | | |
| BRAF | c.2128 − | | | | | | | | | | 1 | | | | 1 | | |
| | 27_2128 − | | | | | | | | | | | | | | | | |
| | 19delCTTTTTTTT | | | | | | | | | | | | | | | | |
| BRAF | c.2128 − | | | | | | | | | | 1 | | | | 1 | | |
| | 28dupT | | | | | | | | | | | | | | | | |
| BRAF | c.2128 − | | | | | | | | | | 1 | | | | 1 | | |
| | 29_2128 − | | | | | | | | | | | | | | | | |
| | 28dupTT | | | | | | | | | | | | | | | | |
| BRAF | c.2128 − | | | | | | | | | | 1 | | | | 1 | | |
| | 30_2128 − | | | | | | | | | | | | | | | | |
| | 28delTTT | | | | | | | | | | | | | | | | |
| BRAF | c.2128 − | | | | | | | | | | 1 | | | | 1 | | |
| | 44_2128 − | | | | | | | | | | | | | | | | |
| | 42delCTT | | | | | | | | | | | | | | | | |
| BRAF | c.2128 − | | | | | | | | | | 1 | | | | 1 | | |
| | 50T > C | | | | | | | | | | | | | | | | |
| BRAF | c.2128 − | | | | | | | | | | 1 | | | | 1 | | |
| | 54_2128 − | | | | | | | | | | | | | | | | |
| | 51dupCTTT | | | | | | | | | | | | | | | | |
| BRAF | c.2128 − | | | | | | | | | | 1 | | | | 1 | | |
| | 5delT | | | | | | | | | | | | | | | | |
| BRAF | c.2235A > G | | | | | | | | | | 1 | | | | 1 | | |
| BRAF | c.240 + | | | | | | | | | | 1 | | | | 1 | | |
| | 46delA | | | | | | | | | | | | | | | | |
| BRAF | c.36G > A | | | | | | | | | | 1 | | | | 1 | | |
| BRAF | c.609 − | | | | | | | | | | 1 | | | | 1 | | |
| | 290delT | | | | | | | | | | | | | | | | |
| BRAF | c.609 − | | | | | | | | | | 1 | | | | 1 | | |
| | 291_609 − | | | | | | | | | | | | | | | | |
| | 290delTT | | | | | | | | | | | | | | | | |
| BRAF | c.609 − | | | | | | | | | | 1 | | | | 1 | | |
| | 292_609 − | | | | | | | | | | | | | | | | |
| | 290delTTT | | | | | | | | | | | | | | | | |
| BRAF | c.609 − | | | | | | | | | | 1 | | | | 1 | | |
| | 293_609 − | | | | | | | | | | | | | | | | |
| | 290delTTTT | | | | | | | | | | | | | | | | |
| BRAF | c.609 − | | | | | | | | | | 1 | | | | 1 | | |
| | 299_609 − | | | | | | | | | | | | | | | | |
| | 290del10 | | | | | | | | | | | | | | | | |
| BRAF | c.609 − | | | | | | | | | | 1 | | | | 1 | | |
| | 300_609 − | | | | | | | | | | | | | | | | |
| | 290del11 | | | | | | | | | | | | | | | | |
| BRAF | c.708C > T | | | | | | | | | | 1 | | | | 1 | | |
| BRAF | c.711 + | | | | | | | | | | 1 | | | | 1 | | |
| | 24delT | | | | | | | | | | | | | | | | |
| BRAF | c.712 − | | | | | | | | | | 1 | | | | 1 | | |
| | 18T > C | | | | | | | | | | | | | | | | |
| BRAF | c.712 − | | | | | | | | | | 1 | | | | 1 | | |
| | 40T > A | | | | | | | | | | | | | | | | |
| BRAF | c.980 + | | | | | | | | | | 1 | | | | 1 | | |
| | 27G > A | | | | | | | | | | | | | | | | |
| BRAF | c.981 − | | | | | | | | | | 1 | | | | 1 | | |
| | 20C > T | | | | | | | | | | | | | | | | |
| CSF3R | c.1809C > T | | | | | | | | | | | | | | | | |
| CSF3R | c.1853C > T | | | | | | | | | | | | | | | | |
| CSF3R | c.2041 − | | | | | | | | | | | | | | | | |
| | 8C > T | | | | | | | | | | | | | | | | |
| DNMT3A | c.1014 + | | | | | | | | | | | | | | | | |
| | 34G > A | | | | | | | | | | | | | | | | |
| DNMT3A | c.−10C > T | | | | | | | | | | | | | | | | |
| DNMT3A | c.1122 + | | | | | | | | | | | | | | | | |
| | 7G > A | | | | | | | | | | | | | | | | |
| DNMT3A | c.1140G > A | | | | | | | | | | | | | | | | |
| DNMT3A | c.1170C > T | | | | | | | | | | | | | | | | |
| DNMT3A | c.1204C > T | | | | | | | | | | | | | | | | |
| DNMT3A | c.120G > A | | | | | | | | | | | | | | | | |
| DNMT3A | c.1266G > A | | | | | | | | | | | | | | | | |
| DNMT3A | c.1279 + | | | | | | | | | | | | | | | | |
| | 18G > A | | | | | | | | | | | | | | | | |
| DNMT3A | c.1279 + | | | | | | | | | | | | | | | | |
| | 38G > A | | | | | | | | | | | | | | | | |
| DNMT3A | c.1280 − | | | | | | | | | | | | | | | | |
| | 46delT | | | | | | | | | | | | | | | | |
| DNMT3A | c.1280 − | | | | | | | | | | | | | | | | |
| | 7C > T | | | | | | | | | | | | | | | | |
| DNMT3A | c.1430 − | | | | | | | | | | | | | | | | |
| | 13C > T | | | | | | | | | | | | | | | | |
| DNMT3A | c.1439T > G | | | | | | | | | | | | | | | | |
| DNMT3A | c.1448T > G | | | | | | | | | | | | | | | | |
| DNMT3A | c.1502A > G | | | | | | | | | | | | | | | | |
| DNMT3A | c.1554 + | | | | | | | | | | | | | | | | |
| | 48G > A | | | | | | | | | | | | | | | | |
| DNMT3A | c.1555 − | | | | | | | | | | | | | | | | |
| | 49G > A | | | | | | | | | | | | | | | | |
| DNMT3A | c.1668 − | | | | | | | | | | | | | | | | |
| | 46C > T | | | | | | | | | | | | | | | | |
| DNMT3A | c.−178 + | | | | | | | | | | | | | | | | |
| | 13112G > C | | | | | | | | | | | | | | | | |
| DNMT3A | c.−178 + | | | | | | | | | | | | | | | | |
| | 13219G > A | | | | | | | | | | | | | | | | |
| DNMT3A | c.1852 − | | | | | | | | | | | | | | | | |
| | 37C > A | | | | | | | | | | | | | | | | |
| DNMT3A | c.1937 − | | | | | | | | | | | | | | | | |
| | 5T > G | | | | | | | | | | | | | | | | |
| DNMT3A | c.2173 + | | | | | | | | | | | | | | | | |
| | 26C > T | | | | | | | | | | | | | | | | |
| DNMT3A | c.2173 + | | | | | | | | | | | | | | | | |
| | 45G > A | | | | | | | | | | | | | | | | |
| DNMT3A | c.2206C > T | | | | | | | | | | | | | | | | |
| DNMT3A | c.2261T > C | | | | | | | | | | | | | | | | |
| DNMT3A | c.2323 − | | | | | | | | | | | | | | | | |
| | 243G > C | | | | | | | | | | | | | | | | |
| DNMT3A | c.2446C > T | | | | | | | | | | | | | | | | |
| DNMT3A | c.2597 + | | | | | | | | | | | | | | | | |
| | 30G > A | | | | | | | | | | | | | | | | |
| DNMT3A | c.2597 + | | | | | | | | | | | | | | | | |
| | 36G > A | | | | | | | | | | | | | | | | |
| DNMT3A | c.2652G > A | | | | | | | | | | | | | | | | |
| DNMT3A | c.2676A > G | | | | | | | | | | | | | | | | |
| DNMT3A | c.2688A > G | | | | | | | | | | | | | | | | |
| DNMT3A | c.2706delC | | | | | | | | | | | | | | | | |
| DNMT3A | c.2732G > A | | | | | | | | | | | | | | | | |
| DNMT3A | c.27C > T | | | | | | | | | | | | | | | | |
| DNMT3A | c.448 + | | | | | | | | | | | | | | | | |
| | 23A > T | | | | | | | | | | | | | | | | |
| DNMT3A | c.639 + | | | | | | | | | | | | | | | | |
| | 6G > A | | | | | | | | | | | | | | | | |
| DNMT3A | c.640 − | | | | | | | | | | | | | | | | |
| | 1439G > A | | | | | | | | | | | | | | | | |
| DNMT3A | c.640 − | | | | | | | | | | | | | | | | |
| | 3916G > T | | | | | | | | | | | | | | | | |
| DNMT3A | c.640 − | | | | | | | | | | | | | | | | |
| | 49delG | | | | | | | | | | | | | | | | |
| DNMT3A | c.−6G > A | | | | | | | | | | | | | | | | |
| DNMT3A | c.72 + | | | | | | | | | | | | | | | | |
| | 10G > A | | | | | | | | | | | | | | | | |
| DNMT3A | c.73 − | | | | | | | | | | | | | | | | |
| | 43T > C | | | | | | | | | | | | | | | | |
| DNMT3A | c.759C > T | | | | | | | | | | | | | | | | |
| DNMT3A | c.801C > T | | | | | | | | | | | | | | | | |
| DNMT3A | c.855 + | | | | | | | | | | | | | | | | |
| | 16A > C | | | | | | | | | | | | | | | | |
| DNMT3A | c.89A > C | | | | | | | | | | | | | | | | |
| DNMT3A | c.939G > A | | | | | | | | | | | | | | | | |
| DNMT3A | c.998delA | | | | | | | | | | | | | | | | |
| EZH2 | c.*20C > A | | | | | | | | | | | | | | | | |
| EZH2 | c.*21delC | | | | | | | | | | | | | | | | |
| EZH2 | c.1000 − | | | | | | | | | | | | | | | | |
| | 47A > G | | | | | | | | | | | | | | | | |
| EZH2 | c.1017T > C | | | | | | | | | | | | | | | | |
| EZH2 | c.118 − | | | | | | | | | | | | | | | | |
| | 4delT | | | | | | | | | | | | | | | | |
| EZH2 | c.118 − | | | | | | | | | | | | | | | | |
| | 4dupT | | | | | | | | | | | | | | | | |
| EZH2 | c.118 − | | | | | | | | | | | | | | | | |
| | 5_118 − | | | | | | | | | | | | | | | | |
| | 4delTT | | | | | | | | | | | | | | | | |
| EZH2 | c.1240 + | | | | | | | | | | | | | | | | |
| | 35T > C | | | | | | | | | | | | | | | | |
| EZH2 | c.1240 + | | | | | | | | | | | | | | | | |
| | 48G > A | | | | | | | | | | | | | | | | |
| EZH2 | c.1240 + | | | | | | | | | | | | | | | | |
| | 9C > A | | | | | | | | | | | | | | | | |
| EZH2 | c.1281A > G | | | | | | | | | | | | | | | | |
| EZH2 | c.1392G > A | | | | | | | | | | | | | | | | |
| EZH2 | c.1410 + | | | | | | | | | | | | | | | | |
| | 25_1410 + | | | | | | | | | | | | | | | | |
| | 33delTTAAAAGAG | | | | | | | | | | | | | | | | |
| EZH2 | c.1452A > G | | | | | | | | | | | | | | | | |
| EZH2 | c.1459G > A | | | | | | | | | | | | | | | | |
| EZH2 | c.1505 + | | | | | | | | | | | | | | | | |
| | 12A > G | | | | | | | | | | | | | | | | |
| EZH2 | c.1505 + | | | | | | | | | | | | | | | | |
| | 20C > G | | | | | | | | | | | | | | | | |
| EZH2 | c.1719C > A | | | | | | | | | | | | | | | | |
| EZH2 | c.1731G > A | | | | | | | | | | | | | | | | |
| EZH2 | c.1852 − | | | | | | | | | | | | | | | | |
| | 21T > C | | | | | | | | | | | | | | | | |
| EZH2 | c.1852 − | | | | | | | | | | | | | | | | |
| | 6C > T | | | | | | | | | | | | | | | | |
| EZH2 | c.1852 − | | | | | | | | | | | | | | | | |
| | 9A > G | | | | | | | | | | | | | | | | |
| EZH2 | c.1865C > T | | | | | | | | | | | | | | | | |
| EZH2 | c.1948 − | | | | | | | | | | | | | | | | |
| | 29dupA | | | | | | | | | | | | | | | | |
| EZH2 | c.1967C > T | | | | | | | | | | | | | | | | |
| EZH2 | c.2028T > C | | | | | | | | | | | | | | | | |
| EZH2 | c.2035G > A | | | | | | | | | | | | | | | | |
| EZH2 | c.2068C > T | | | | | | | | | | | | | | | | |
| EZH2 | c.2110 + | | | | | | | | | | | | | | | | |
| | 39A > C | | | | | | | | | | | | | | | | |
| EZH2 | c.2110 + | | | | | | | | | | | | | | | | |
| | 6T > G | | | | | | | | | | | | | | | | |
| EZH2 | c.2111 − | | | | | | | | | | | | | | | | |
| | 38G > A | | | | | | | | | | | | | | | | |
| EZH2 | c.2111 − | | | | | | | | | | | | | | | | |
| | 46C > T | | | | | | | | | | | | | | | | |
| EZH2 | c.2196 − | | | | | | | | | | | | | | | | |
| | 20T > C | | | | | | | | | | | | | | | | |
| EZH2 | c.246 + | | | | | | | | | | | | | | | | |
| | 37T > C | | | | | | | | | | | | | | | | |
| EZH2 | c.247 − | | | | | | | | | | | | | | | | |
| | 10dupT | | | | | | | | | | | | | | | | |
| EZH2 | c.363 + | | | | | | | | | | | | | | | | |
| | 43T > C | | | | | | | | | | | | | | | | |
| EZH2 | c.396T > C | | | | | | | | | | | | | | | | |
| EZH2 | c.434T > C | | | | | | | | | | | | | | | | |
| EZH2 | c.484 + | | | | | | | | | | | | | | | | |
| | 13C > T | | | | | | | | | | | | | | | | |
| EZH2 | c.485 − | | | | | | | | | | | | | | | | |
| | 7G > A | | | | | | | | | | | | | | | | |
| EZH2 | c.538C > T | | | | | | | | | | | | | | | | |
| EZH2 | c.553G > C | | | | | | | | | | | | | | | | |
| EZH2 | c.625 + | | | | | | | | | | | | | | | | |
| | 25T > C | | | | | | | | | | | | | | | | |
| EZH2 | c.625 + | | | | | | | | | | | | | | | | |
| | 26G > T | | | | | | | | | | | | | | | | |
| EZH2 | c.625 + | | | | | | | | | | | | | | | | |
| | 5G > A | | | | | | | | | | | | | | | | |
| EZH2 | c.626 − | | | | | | | | | | | | | | | | |
| | 39A > G | | | | | | | | | | | | | | | | |
| EZH2 | c.728 + | | | | | | | | | | | | | | | | |
| | 19G > C | | | | | | | | | | | | | | | | |
| EZH2 | c.72dupG | | | | | | | | | | | | | | | | |
| FLT3 | c.1713G > A | | | | | | | 1 | 1 | | | | | | | | |
| FLT3 | c.1715A > G | | | | | | | 1 | 1 | | | | | | | | |
| FLT3 | c.1782_1805dup24 | | | | | | | 1 | 1 | | | | | | | | |
| FLT3 | c.1788_1789ins15 | | | | | | | | 1 | | | | | | | | |
| FLT3 | c.1795_1796ins21 | | | | | | | 1 | 1 | | | | | | | | |
| FLT3 | c.2503G > A | | | | | | | 1 | 1 | | | | | | | | |
| FLT3 | c.2503G > C | | | | | | | 1 | 1 | | | | | | | | |
| FLT3 | c.2503G > T | | | | | | | | | | | | | | | | |
| FLT3 | c.2508_2510delCAT | | | | | | | 1 | 1 | | | | | | | | |
| FLT3 | c.2522A > T | | | | | | | | | | | | | | | | |
| FLT3 | c.2524T > G | | | | | | | | | | | | | | | | |
| FLT3 | c.2525A > G | | | | | | | 1 | 1 | | | | | | | | |
| FLT3 | c.FLT3_ITD | | | | | | | 1 | 1 | | | | | | | | |
| IDH2 | c.407A > G | | | | | | | | | | | | | | | | |
| IDH2 | c.419G > A | | | | | | | | | | | | | | | | |
| IDH2 | c.515G > A | | | | | | | | | | | | | | | | |
| JAK2 | c.*4A > C | | | | | | | | | | | | | | | | |
| JAK2 | c.*52A > G | | | | | | | | | | | | | | | | |
| JAK2 | c.1009A > G | | | | | | | | | | | | | | | | |
| JAK2 | c.1174G > A | | | | | | | | | | | | | | | | |
| JAK2 | c.1177C > G | | | | | | | | | | | | | | | | |
| JAK2 | c.1284T > C | | | | | | | | | | | | | | | | |
| JAK2 | c.1323C > T | | | | | | | | | | | | | | | | |
| JAK2 | c.1326 + | | | | | | | | | | | | | | | | |
| | 25G > C | | | | | | | | | | | | | | | | |
| JAK2 | c.1439G > T | | | | | | | | | | | | | | | | |
| JAK2 | c.1458T > G | | | | | | | | | | | | | | | | |
| JAK2 | c.1514 − | | | | | | | | | | | | | | | | |
| | 42C > T | | | | | | | | | | | | | | | | |
| JAK2 | c.1542G > A | | | | | | | | | | | | | | | | |
| JAK2 | c.1622_1627delGAAATG | | | | | | | | | | | | | | | | |
| JAK2 | c.1624_1629delAATGAA | | | | | | | | | | | | | | | | |
| JAK2 | c.1641 + | | | | | | | | | | | | | | | | |
| | 6T > C | | | | | | | | | | | | | | | | |
| JAK2 | c.1710C > T | | | | | | | | | | | | | | | | |
| JAK2 | c.1777 − | | | | | | | | | | | | | | | | |
| | 7delT | | | | | | | | | | | | | | | | |
| JAK2 | c.1777 − | | | | | | | | | | | | | | | | |
| | 7dupT | | | | | | | | | | | | | | | | |
| JAK2 | c.1849G > T | | | | | | | | | | | | | | | | |
| JAK2 | c.1860C > T | | | | | | | | | | | | | | | | |
| JAK2 | c.1864 + | | | | | | | | | | | | | | | | |
| | 14C > T | | | | | | | | | | | | | | | | |
| JAK2 | c.1929T > C | | | | | | | | | | | | | | | | |
| JAK2 | c.1992 + | | | | | | | | | | | | | | | | |
| | 10A > G | | | | | | | | | | | | | | | | |
| JAK2 | c.1992 + | | | | | | | | | | | | | | | | |
| | 21A > T | | | | | | | | | | | | | | | | |
| JAK2 | c.1992 + | | | | | | | | | | | | | | | | |
| | 35T > C | | | | | | | | | | | | | | | | |
| JAK2 | c.1993 − | | | | | | | | | | | | | | | | |
| | 15G > A | | | | | | | | | | | | | | | | |
| JAK2 | c.1993 − | | | | | | | | | | | | | | | | |
| | 15G > T | | | | | | | | | | | | | | | | |
| JAK2 | c.1993 − | | | | | | | | | | | | | | | | |
| | 16C > T | | | | | | | | | | | | | | | | |
| JAK2 | c.2132 − | | | | | | | | | | | | | | | | |
| | 44A > G | | | | | | | | | | | | | | | | |
| JAK2 | c.227 − | | | | | | | | | | | | | | | | |
| | 38T > C | | | | | | | | | | | | | | | | |
| JAK2 | c.227 − | | | | | | | | | | | | | | | | |
| | 7T > C | | | | | | | | | | | | | | | | |
| JAK2 | c.2283 + | | | | | | | | | | | | | | | | |
| | 50A > T | | | | | | | | | | | | | | | | |
| JAK2 | c.2434 + | | | | | | | | | | | | | | | | |
| | 18C > A | | | | | | | | | | | | | | | | |
| JAK2 | c.2434 + | | | | | | | | | | | | | | | | |
| | 35T > C | | | | | | | | | | | | | | | | |
| JAK2 | c.2490G > A | | | | | | | | | | | | | | | | |
| JAK2 | c.2571 + | | | | | | | | | | | | | | | | |
| | 49A > T | | | | | | | | | | | | | | | | |
| JAK2 | c.2577T > C | | | | | | | | | | | | | | | | |
| JAK2 | c.2586T > C | | | | | | | | | | | | | | | | |
| JAK2 | c.2762 − | | | | | | | | | | | | | | | | |
| | 10_2762 − | | | | | | | | | | | | | | | | |
| | 9delAT | | | | | | | | | | | | | | | | |
| JAK2 | c.2850C > T | | | | | | | | | | | | | | | | |
| JAK2 | c.2886 + | | | | | | | | | | | | | | | | |
| | 42T > C | | | | | | | | | | | | | | | | |
| JAK2 | c.2931G > A | | | | | | | | | | | | | | | | |
| JAK2 | c.2958C > T | | | | | | | | | | | | | | | | |
| JAK2 | c.3059 + | | | | | | | | | | | | | | | | |
| | 23A > T | | | | | | | | | | | | | | | | |
| JAK2 | c.3059 + | | | | | | | | | | | | | | | | |
| | 44C > A | | | | | | | | | | | | | | | | |
| JAK2 | c.3188G > A | | | | | | | | | | | | | | | | |
| JAK2 | c.3252T > C | | | | | | | | | | | | | | | | |
| JAK2 | c.3262T > C | | | | | | | | | | | | | | | | |
| JAK2 | c.3288T > A | | | | | | | | | | | | | | | | |
| JAK2 | c.3291 + | | | | | | | | | | | | | | | | |
| | 14T > C | | | | | | | | | | | | | | | | |
| JAK2 | c.3291 + | | | | | | | | | | | | | | | | |
| | 16delT | | | | | | | | | | | | | | | | |
| JAK2 | c.3291 + | | | | | | | | | | | | | | | | |
| | 51T > C | | | | | | | | | | | | | | | | |
| JAK2 | c.3291 + | | | | | | | | | | | | | | | | |
| | 8T > C | | | | | | | | | | | | | | | | |
| JAK2 | c.3323A > G | | | | | | | | | | | | | | | | |
| JAK2 | c.380G > A | | | | | | | | | | | | | | | | |
| JAK2 | c.489C > T | | | | | | | | | | | | | | | | |
| JAK2 | c.525G > A | | | | | | | | | | | | | | | | |
| JAK2 | c.579C > T | | | | | | | | | | | | | | | | |
| JAK2 | c.614 + | | | | | | | | | | | | | | | | |
| | 33T > C | | | | | | | | | | | | | | | | |
| JAK2 | c.615 − | | | | | | | | | | | | | | | | |
| | 24C > T | | | | | | | | | | | | | | | | |
| JAK2 | c.731A > G | | | | | | | | | | | | | | | | |
| JAK2 | c.936 + | | | | | | | | | | | | | | | | |
| | 13G > A | | | | | | | | | | | | | | | | |
| KIT | c.1854G > A | | | | | | | | 1 | | | | | | | | 2 |
| KIT | c.2447A > T | | | | 1 | | | | 1 | | | | | | | | 2 |
| KRAS | c.*512T > C | | 1 | | | | | | | | 1 | | | | | | |
| KRAS | c.*550C > T | | 1 | | | | | | | | 1 | | | | | | |
| KRAS | c.111 + | | 1 | | | | | | | | 1 | | | | | | |
| | 46T > G | | | | | | | | | | | | | | | | |
| KRAS | c.173C > T | | 1 | | | | | | | | 1 | | | | | | |
| KRAS | c.179G > A | | 1 | | | | | | | | 1 | | | | | | |
| KRAS | c.182A > C | | | | | | | | | | 1 | | | | | | |
| KRAS | c.182A > G | | | | | | | | | | 1 | | | | | | |
| KRAS | c.183A > T | | | | | | | | | | 1 | | | | | | |
| KRAS | c.34G > A | | | | | | | | | 1 | 1 | | | | | | |
| KRAS | c.350A > G | | 1 | | | | | | | | 2 | | | | | | |
| KRAS | c.35G > A | | | | | | | | | | 1 | | | | | | |
| KRAS | c.35G > C | | | | | | | | | | 1 | | | | | | |
| KRAS | c.35G > T | | | | | | | | | | 1 | | | | | | |
| KRAS | c.38G > A | | | | | | | | | | 1 | | | | | | |
| KRAS | c.451 − | | 1 | | | | | | | | 1 | | | | | | |
| | 18A > G | | | | | | | | | | | | | | | | |
| KRAS | c.451 − | | 1 | | | | | | | | 1 | | | | | | |
| | 5494A > G | | | | | | | | | | | | | | | | |
| KRAS | c.451 − | | 1 | | | | | | | | 1 | | | | | | |
| | 5521G > A | | | | | | | | | | | | | | | | |
| KRAS | c.451 − | | 1 | | | | | | | | 1 | | | | | | |
| | 5617G > A | | | | | | | | | | | | | | | | |
| KRAS | c.451 − | | 1 | | | | | | | | 1 | | | | | | |
| | 9G > A | | | | | | | | | | | | | | | | |
| KRAS | c.519T > C | | 1 | | | | | | | | 1 | | | | | | |
| NF1 | c.*29A > G | | | | | | | | | | | | | | | | |
| NF1 | c.*4T > C | | | | | | | | | | | | | | | | |
| NF1 | c.1185 + | | | | | | | | | | | | | | | | |
| | 30T > C | | | | | | | | | | | | | | | | |
| NF1 | c.1186 − | | | | | | | | | | | | | | | | |
| | 13delT | | | | | | | | | | | | | | | | |
| NF1 | c.1186 − | | | | | | | | | | | | | | | | |
| | 13dupT | | | | | | | | | | | | | | | | |
| NF1 | c.1261 − | | | | | | | | | | | | | | | | |
| | 45G > A | | | | | | | | | | | | | | | | |
| NF1 | c.−126G > A | | | | | | | | | | | | | | | | |
| NF1 | c.1308G > A | | | | | | | | | | | | | | | | |
| NF1 | c.1393 − | | | | | | | | | | | | | | | | |
| | 31G > A | | | | | | | | | | | | | | | | |
| NF1 | c.1393 − | | | | | | | | | | | | | | | | |
| | 32T > C | | | | | | | | | | | | | | | | |
| NF1 | c.1528 − | | | | | | | | | | | | | | | | |
| | 29dupT | | | | | | | | | | | | | | | | |
| NF1 | c.1641 + | | | | | | | | | | | | | | | | |
| | 39T > C | | | | | | | | | | | | | | | | |
| NF1 | c.168C > T | | | | | | | | | | | | | | | | |
| NF1 | c.1721 + | | | | | | | | | | | | | | | | |
| | 72C > T | | | | | | | | | | | | | | | | |
| NF1 | c.1750A > T | | | | | | | | | | | | | | | | |
| NF1 | c.1845 + | | | | | | | | | | | | | | | | |
| | 13A > T | | | | | | | | | | | | | | | | |
| NF1 | c.1845 + | | | | | | | | | | | | | | | | |
| | 45G > A | | | | | | | | | | | | | | | | |
| NF1 | c.1845 + | | | | | | | | | | | | | | | | |
| | 45G > T | | | | | | | | | | | | | | | | |
| NF1 | c.1846 − | | | | | | | | | | | | | | | | |
| | 12A > T | | | | | | | | | | | | | | | | |
| NF1 | c.1846 − | | | | | | | | | | | | | | | | |
| | 39C > T | | | | | | | | | | | | | | | | |
| NF1 | c.1994C > T | | | | | | | | | | | | | | | | |
| NF1 | c.2022C > T | | | | | | | | | | | | | | | | |
| NF1 | c.2034G > A | | | | | | | | | | | | | | | | |
| NF1 | c.204 + | | | | | | | | | | | | | | | | |
| | 51G > C | | | | | | | | | | | | | | | | |
| NF1 | c.2224G > T | | | | | | | | | | | | | | | | |
| NF1 | c.2252 − | | | | | | | | | | | | | | | | |
| | 31A > T | | | | | | | | | | | | | | | | |
| NF1 | c.2252 − | | | | | | | | | | | | | | | | |
| | 34T > C | | | | | | | | | | | | | | | | |
| NF1 | c.2252 − | | | | | | | | | | | | | | | | |
| | 46C > T | | | | | | | | | | | | | | | | |
| NF1 | c.−22G > C | | | | | | | | | | | | | | | | |
| NF1 | c.2544G > A | | | | | | | | | | | | | | | | |
| NF1 | c.2553C > T | | | | | | | | | | | | | | | | |
| NF1 | c.2851 − | | | | | | | | | | | | | | | | |
| | 16T > C | | | | | | | | | | | | | | | | |
| NF1 | c.288 + | | | | | | | | | | | | | | | | |
| | 41G > A | | | | | | | | | | | | | | | | |
| NF1 | c.2921A > G | | | | | | | | | | | | | | | | |
| NF1 | c.2985G > C | | | | | | | | | | | | | | | | |
| NF1 | c.3114 − | | | | | | | | | | | | | | | | |
| | 7T > C | | | | | | | | | | | | | | | | |
| NF1 | c.3197 + | | | | | | | | | | | | | | | | |
| | 10G > A | | | | | | | | | | | | | | | | |
| NF1 | c.3197 + | | | | | | | | | | | | | | | | |
| | 17C > T | | | | | | | | | | | | | | | | |
| NF1 | c.3197 + | | | | | | | | | | | | | | | | |
| | 23G > A | | | | | | | | | | | | | | | | |
| NF1 | c.3197 + | | | | | | | | | | | | | | | | |
| | 47G > A | | | | | | | | | | | | | | | | |
| NF1 | c.3198 − | | | | | | | | | | | | | | | | |
| | 11_3198 − | | | | | | | | | | | | | | | | |
| | 4delTTTTTTTT | | | | | | | | | | | | | | | | |
| NF1 | c.3198 − | | | | | | | | | | | | | | | | |
| | 3dupC | | | | | | | | | | | | | | | | |
| NF1 | c.3198 − | | | | | | | | | | | | | | | | |
| | 4dupT | | | | | | | | | | | | | | | | |
| NF1 | c.3198 − | | | | | | | | | | | | | | | | |
| | 4T > C | | | | | | | | | | | | | | | | |
| NF1 | c.3198 − | | | | | | | | | | | | | | | | |
| | 5_3198 − | | | | | | | | | | | | | | | | |
| | 4dupTT | | | | | | | | | | | | | | | | |
| NF1 | c.3198 − | | | | | | | | | | | | | | | | |
| | 6_3198 − | | | | | | | | | | | | | | | | |
| | 4dupTTT | | | | | | | | | | | | | | | | |
| NF1 | c.3198 − | | | | | | | | | | | | | | | | |
| | 7_3198 − | | | | | | | | | | | | | | | | |
| | 4dupTTTT | | | | | | | | | | | | | | | | |
| NF1 | c.3270A > C | | | | | | | | | | | | | | | | |
| NF1 | c.340C > T | | | | | | | | | | | | | | | | |
| NF1 | c.3496 + | | | | | | | | | | | | | | | | |
| | 19T > C | | | | | | | | | | | | | | | | |
| NF1 | c.3496 + | | | | | | | | | | | | | | | | |
| | 33C > A | | | | | | | | | | | | | | | | |
| NF1 | c.3496 + | | | | | | | | | | | | | | | | |
| | 43A > C | | | | | | | | | | | | | | | | |
| NF1 | c.3498C > T | | | | | | | | | | | | | | | | |
| NF1 | c.3686A > G | | | | | | | | | | | | | | | | |
| NF1 | c.369C > G | | | | | | | | | | | | | | | | |
| NF1 | c.3709 − | | | | | | | | | | | | | | | | |
| | 47A > G | | | | | | | | | | | | | | | | |
| NF1 | c.3867C > T | | | | | | | | | | | | | | | | |
| NF1 | c.3870 + | | | | | | | | | | | | | | | | |
| | 25A > G | | | | | | | | | | | | | | | | |
| NF1 | c.3870 + | | | | | | | | | | | | | | | | |
| | 50A > G | | | | | | | | | | | | | | | | |
| NF1 | c.3871 − | | | | | | | | | | | | | | | | |
| | 31A > G | | | | | | | | | | | | | | | | |
| NF1 | c.3974 + | | | | | | | | | | | | | | | | |
| | 36_3974 + | | | | | | | | | | | | | | | | |
| | 37dupGT | | | | | | | | | | | | | | | | |
| NF1 | c.3974 + | | | | | | | | | | | | | | | | |
| | 36G > T | | | | | | | | | | | | | | | | |
| NF1 | c.3974 + | | | | | | | | | | | | | | | | |
| | 37_3974 + | | | | | | | | | | | | | | | | |
| | 38insG | | | | | | | | | | | | | | | | |
| NF1 | c.3974 + | | | | | | | | | | | | | | | | |
| | 38T > G | | | | | | | | | | | | | | | | |
| NF1 | c.3974 + | | | | | | | | | | | | | | | | |
| | 40T > G | | | | | | | | | | | | | | | | |
| NF1 | c.3974 + | | | | | | | | | | | | | | | | |
| | 43_3974 + | | | | | | | | | | | | | | | | |
| | 44insG | | | | | | | | | | | | | | | | |
| NF1 | c.3974 + | | | | | | | | | | | | | | | | |
| | 46T > G | | | | | | | | | | | | | | | | |
| NF1 | c.3974 + | | | | | | | | | | | | | | | | |
| | 48_3974 + | | | | | | | | | | | | | | | | |
| | 49delTT | | | | | | | | | | | | | | | | |
| NF1 | c.3974 + | | | | | | | | | | | | | | | | |
| | 48_3974 + | | | | | | | | | | | | | | | | |
| | 49dupTT | | | | | | | | | | | | | | | | |
| NF1 | c.3974 + | | | | | | | | | | | | | | | | |
| | 49delT | | | | | | | | | | | | | | | | |
| NF1 | c.3974 + | | | | | | | | | | | | | | | | |
| | 49dupT | | | | | | | | | | | | | | | | |
| NF1 | c.4110 + | | | | | | | | | | | | | | | | |
| | 3780C > T | | | | | | | | | | | | | | | | |
| NF1 | c.4111 − | | | | | | | | | | | | | | | | |
| | 48_4111 − | | | | | | | | | | | | | | | | |
| | 23dup26 | | | | | | | | | | | | | | | | |
| NF1 | c.4182T > C | | | | | | | | | | | | | | | | |
| NF1 | c.4206A > G | | | | | | | | | | | | | | | | |
| NF1 | c.4368 − | | | | | | | | | | | | | | | | |
| | 46G > C | | | | | | | | | | | | | | | | |
| NF1 | c.4514 + | | | | | | | | | | | | | | | | |
| | 12C > T | | | | | | | | | | | | | | | | |
| NF1 | c.4514 + | | | | | | | | | | | | | | | | |
| | 35C > G | | | | | | | | | | | | | | | | |
| NF1 | c.4515 − | | | | | | | | | | | | | | | | |
| | 39A > G | | | | | | | | | | | | | | | | |
| NF1 | c.4772 + | | | | | | | | | | | | | | | | |
| | 29689A > G | | | | | | | | | | | | | | | | |
| NF1 | c.4772 + | | | | | | | | | | | | | | | | |
| | 29883T > G | | | | | | | | | | | | | | | | |
| NF1 | c.4772 + | | | | | | | | | | | | | | | | |
| | 36T > C | | | | | | | | | | | | | | | | |
| NF1 | c.4773 − | | | | | | | | | | | | | | | | |
| | 17877_4773 − | | | | | | | | | | | | | | | | |
| | 17876delAG | | | | | | | | | | | | | | | | |
| NF1 | c.4773 − | | | | | | | | | | | | | | | | |
| | 20174A > C | | | | | | | | | | | | | | | | |
| NF1 | c.4773 − | | | | | | | | | | | | | | | | |
| | 20184delG | | | | | | | | | | | | | | | | |
| NF1 | c.4773 − | | | | | | | | | | | | | | | | |
| | 20194G > A | | | | | | | | | | | | | | | | |
| NF1 | c.4773 − | | | | | | | | | | | | | | | | |
| | 20367C > T | | | | | | | | | | | | | | | | |
| NF1 | c.4773 − | | | | | | | | | | | | | | | | |
| | 20371T > A | | | | | | | | | | | | | | | | |
| NF1 | c.4773 − | | | | | | | | | | | | | | | | |
| | 20399G > A | | | | | | | | | | | | | | | | |
| NF1 | c.4773 − | | | | | | | | | | | | | | | | |
| | 20486T > C | | | | | | | | | | | | | | | | |
| NF1 | c.4773 − | | | | | | | | | | | | | | | | |
| | 20567T > C | | | | | | | | | | | | | | | | |
| NF1 | c.4773 − | | | | | | | | | | | | | | | | |
| | 20708T > C | | | | | | | | | | | | | | | | |
| NF1 | c.4773 − | | | | | | | | | | | | | | | | |
| | 20889A > T | | | | | | | | | | | | | | | | |
| NF1 | c.4773 − | | | | | | | | | | | | | | | | |
| | 20968C > A | | | | | | | | | | | | | | | | |
| NF1 | c.4773 − | | | | | | | | | | | | | | | | |
| | 21063C > G | | | | | | | | | | | | | | | | |
| NF1 | c.4773 − | | | | | | | | | | | | | | | | |
| | 21229G > C | | | | | | | | | | | | | | | | |
| NF1 | c.4773 − | | | | | | | | | | | | | | | | |
| | 21352T > C | | | | | | | | | | | | | | | | |
| NF1 | c.4773 − | | | | | | | | | | | | | | | | |
| | 21440A > G | | | | | | | | | | | | | | | | |
| NF1 | c.4773 − | | | | | | | | | | | | | | | | |
| | 29479G > A | | | | | | | | | | | | | | | | |
| NF1 | c.4773 − | | | | | | | | | | | | | | | | |
| | 29550C > T | | | | | | | | | | | | | | | | |
| NF1 | c.4773 − | | | | | | | | | | | | | | | | |
| | 30170A > G | | | | | | | | | | | | | | | | |
| NF1 | c.4773 − | | | | | | | | | | | | | | | | |
| | 5353G > A | | | | | | | | | | | | | | | | |
| NF1 | c.4773 − | | | | | | | | | | | | | | | | |
| | 5368C > T | | | | | | | | | | | | | | | | |
| NF1 | c.4773 − | | | | | | | | | | | | | | | | |
| | 5461delA | | | | | | | | | | | | | | | | |
| NF1 | c.4773 − | | | | | | | | | | | | | | | | |
| | 5461dupA | | | | | | | | | | | | | | | | |
| NF1 | c.4773 − | | | | | | | | | | | | | | | | |
| | 5462_4773 − | | | | | | | | | | | | | | | | |
| | 5461delAA | | | | | | | | | | | | | | | | |
| NF1 | c.4773 − | | | | | | | | | | | | | | | | |
| | 5463_4773 − | | | | | | | | | | | | | | | | |
| | 5461delAAA | | | | | | | | | | | | | | | | |
| NF1 | c.4773 − | | | | | | | | | | | | | | | | |
| | 5475A > T | | | | | | | | | | | | | | | | |
| NF1 | c.4773 − | | | | | | | | | | | | | | | | |
| | 5476A > T | | | | | | | | | | | | | | | | |
| NF1 | c.4773 − | | | | | | | | | | | | | | | | |
| | 5477dupT | | | | | | | | | | | | | | | | |
| NF1 | c.4773 − | | | | | | | | | | | | | | | | |
| | 6806G > A | | | | | | | | | | | | | | | | |
| NF1 | c.4773 − | | | | | | | | | | | | | | | | |
| | 6844T > C | | | | | | | | | | | | | | | | |
| NF1 | c.4773 − | | | | | | | | | | | | | | | | |
| | 6867C > G | | | | | | | | | | | | | | | | |
| NF1 | c.4773 − | | | | | | | | | | | | | | | | |
| | 6962G > A | | | | | | | | | | | | | | | | |
| NF1 | c.4773 − | | | | | | | | | | | | | | | | |
| | 7106G > T | | | | | | | | | | | | | | | | |
| NF1 | c.4773 − | | | | | | | | | | | | | | | | |
| | 7179C > T | | | | | | | | | | | | | | | | |
| NF1 | c.4773 − | | | | | | | | | | | | | | | | |
| | 7300G > T | | | | | | | | | | | | | | | | |
| NF1 | c.4773 − | | | | | | | | | | | | | | | | |
| | 7333T > C | | | | | | | | | | | | | | | | |
| NF1 | c.4773 − | | | | | | | | | | | | | | | | |
| | 7365C > T | | | | | | | | | | | | | | | | |
| NF1 | c.4866G > A | | | | | | | | | | | | | | | | |
| NF1 | c.5172G > A | | | | | | | | | | | | | | | | |
| NF1 | c.5205 + | | | | | | | | | | | | | | | | |
| | 23T > C | | | | | | | | | | | | | | | | |
| NF1 | c.528T > A | | | | | | | | | | | | | | | | |
| NF1 | c.5546 + | | | | | | | | | | | | | | | | |
| | 19T > A | | | | | | | | | | | | | | | | |
| NF1 | c.5750 − | | | | | | | | | | | | | | | | |
| | 168delT | | | | | | | | | | | | | | | | |
| NF1 | c.5750 − | | | | | | | | | | | | | | | | |
| | 43A > G | | | | | | | | | | | | | | | | |
| NF1 | c.5750 − | | | | | | | | | | | | | | | | |
| | 75C > T | | | | | | | | | | | | | | | | |
| NF1 | c.5820G > A | | | | | | | | | | | | | | | | |
| NF1 | c.60 + | | | | | | | | | | | | | | | | |
| | 16C > A | | | | | | | | | | | | | | | | |
| NF1 | c.6084 + | | | | | | | | | | | | | | | | |
| | 11T > C | | | | | | | | | | | | | | | | |
| NF1 | c.6084 + | | | | | | | | | | | | | | | | |
| | 8C > G | | | | | | | | | | | | | | | | |
| NF1 | c.6085 − | | | | | | | | | | | | | | | | |
| | 17delA | | | | | | | | | | | | | | | | |
| NF1 | c.6085 − | | | | | | | | | | | | | | | | |
| | 17dupA | | | | | | | | | | | | | | | | |
| NF1 | c.6085 − | | | | | | | | | | | | | | | | |
| | 18_6085 − | | | | | | | | | | | | | | | | |
| | 17dupAA | | | | | | | | | | | | | | | | |
| NF1 | c.6085 − | | | | | | | | | | | | | | | | |
| | 19_6085 − | | | | | | | | | | | | | | | | |
| | 17dupAAA | | | | | | | | | | | | | | | | |
| NF1 | c.6085 − | | | | | | | | | | | | | | | | |
| | 27A > T | | | | | | | | | | | | | | | | |
| NF1 | c.6085 − | | | | | | | | | | | | | | | | |
| | 28delT | | | | | | | | | | | | | | | | |
| NF1 | c.6085 − | | | | | | | | | | | | | | | | |
| | 28dupT | | | | | | | | | | | | | | | | |
| NF1 | c.6085 − | | | | | | | | | | | | | | | | |
| | 28T > A | | | | | | | | | | | | | | | | |
| NF1 | c.6085 − | | | | | | | | | | | | | | | | |
| | 29_6085 − | | | | | | | | | | | | | | | | |
| | 28delTT | | | | | | | | | | | | | | | | |
| NF1 | c.6085 − | | | | | | | | | | | | | | | | |
| | 29_6085 − | | | | | | | | | | | | | | | | |
| | 28insA | | | | | | | | | | | | | | | | |
| NF1 | c.6085 − | | | | | | | | | | | | | | | | |
| | 29T > A | | | | | | | | | | | | | | | | |
| NF1 | c.6085 − | | | | | | | | | | | | | | | | |
| | 30_6085 − | | | | | | | | | | | | | | | | |
| | 28delTTT | | | | | | | | | | | | | | | | |
| NF1 | c.6085 − | | | | | | | | | | | | | | | | |
| | 30T > A | | | | | | | | | | | | | | | | |
| NF1 | c.61 − | | | | | | | | | | | | | | | | |
| | 13C > T | | | | | | | | | | | | | | | | |
| NF1 | c.61 − | | | | | | | | | | | | | | | | |
| | 14delT | | | | | | | | | | | | | | | | |
| NF1 | c.61 − | | | | | | | | | | | | | | | | |
| | 14dupT | | | | | | | | | | | | | | | | |
| NF1 | c.61 − | | | | | | | | | | | | | | | | |
| | 4delT | | | | | | | | | | | | | | | | |
| NF1 | c.61 − | | | | | | | | | | | | | | | | |
| | 4dupT | | | | | | | | | | | | | | | | |
| NF1 | c.6330C > T | | | | | | | | | | | | | | | | |
| NF1 | c.6364 + | | | | | | | | | | | | | | | | |
| | 32A > G | | | | | | | | | | | | | | | | |
| NF1 | c.655 − | | | | | | | | | | | | | | | | |
| | 22A > C | | | | | | | | | | | | | | | | |
| NF1 | c.655 − | | | | | | | | | | | | | | | | |
| | 29T > C | | | | | | | | | | | | | | | | |
| NF1 | c.655 − | | | | | | | | | | | | | | | | |
| | 35A > C | | | | | | | | | | | | | | | | |
| NF1 | c.6579 + | | | | | | | | | | | | | | | | |
| | 45T > A | | | | | | | | | | | | | | | | |
| NF1 | c.6580 − | | | | | | | | | | | | | | | | |
| | 19A > G | | | | | | | | | | | | | | | | |
| NF1 | c.6858 + | | | | | | | | | | | | | | | | |
| | 40A > G | | | | | | | | | | | | | | | | |
| NF1 | c.6859 − | | | | | | | | | | | | | | | | |
| | 14_6859 − | | | | | | | | | | | | | | | | |
| | 13delAT | | | | | | | | | | | | | | | | |
| NF1 | c.7000 − | | | | | | | | | | | | | | | | |
| | 22A > G | | | | | | | | | | | | | | | | |
| NF1 | c.702G > A | | | | | | | | | | | | | | | | |
| NF1 | c.7126 + | | | | | | | | | | | | | | | | |
| | 37C > G | | | | | | | | | | | | | | | | |
| NF1 | c.7127 − | | | | | | | | | | | | | | | | |
| | 11_7127 − | | | | | | | | | | | | | | | | |
| | 8delGTTT | | | | | | | | | | | | | | | | |
| NF1 | c.7127 − | | | | | | | | | | | | | | | | |
| | 11_7127 − | | | | | | | | | | | | | | | | |
| | 8dupGTTT | | | | | | | | | | | | | | | | |
| NF1 | c.7127 − | | | | | | | | | | | | | | | | |
| | 19_7127 − | | | | | | | | | | | | | | | | |
| | 8del12 | | | | | | | | | | | | | | | | |
| NF1 | c.7258 + | | | | | | | | | | | | | | | | |
| | 43A > G | | | | | | | | | | | | | | | | |
| NF1 | c.7259 − | | | | | | | | | | | | | | | | |
| | 17C > T | | | | | | | | | | | | | | | | |
| NF1 | c.730 + | | | | | | | | | | | | | | | | |
| | 15G > T | | | | | | | | | | | | | | | | |
| NF1 | c.730 + | | | | | | | | | | | | | | | | |
| | 30_730 + | | | | | | | | | | | | | | | | |
| | 32dupTTT | | | | | | | | | | | | | | | | |
| NF1 | c.730 + | | | | | | | | | | | | | | | | |
| | 31_730 + | | | | | | | | | | | | | | | | |
| | 32delTT | | | | | | | | | | | | | | | | |
| NF1 | c.730 + | | | | | | | | | | | | | | | | |
| | 31_730 + | | | | | | | | | | | | | | | | |
| | 32dupTT | | | | | | | | | | | | | | | | |
| NF1 | c.730 + | | | | | | | | | | | | | | | | |
| | 32delT | | | | | | | | | | | | | | | | |
| NF1 | c.730 + | | | | | | | | | | | | | | | | |
| | 32dupT | | | | | | | | | | | | | | | | |
| NF1 | c.730 + | | | | | | | | | | | | | | | | |
| | 33G > T | | | | | | | | | | | | | | | | |
| NF1 | c.730 + | | | | | | | | | | | | | | | | |
| | 35C > T | | | | | | | | | | | | | | | | |
| NF1 | c.7305A > G | | | | | | | | | | | | | | | | |
| NF1 | c.7347T > C | | | | | | | | | | | | | | | | |
| NF1 | c.7394 + | | | | | | | | | | | | | | | | |
| | 21A > G | | | | | | | | | | | | | | | | |
| NF1 | c.7395 − | | | | | | | | | | | | | | | | |
| | 29G > A | | | | | | | | | | | | | | | | |
| NF1 | c.7398A > G | | | | | | | | | | | | | | | | |
| NF1 | c.7468G > C | | | | | | | | | | | | | | | | |
| NF1 | c.7552 + | | | | | | | | | | | | | | | | |
| | 24delA | | | | | | | | | | | | | | | | |
| NF1 | c.7675 + | | | | | | | | | | | | | | | | |
| | 46G > A | | | | | | | | | | | | | | | | |
| NF1 | c.7676 − | | | | | | | | | | | | | | | | |
| | 33G > A | | | | | | | | | | | | | | | | |
| NF1 | c.7692C > T | | | | | | | | | | | | | | | | |
| NF1 | c.7807 − | | | | | | | | | | | | | | | | |
| | 13dupT | | | | | | | | | | | | | | | | |
| NF1 | c.7908 − | | | | | | | | | | | | | | | | |
| | 19G > T | | | | | | | | | | | | | | | | |
| NF1 | c.7908 − | | | | | | | | | | | | | | | | |
| | 8delT | | | | | | | | | | | | | | | | |
| NF1 | c.8050 + | | | | | | | | | | | | | | | | |
| | 20G > A | | | | | | | | | | | | | | | | |
| NF1 | c.8050 + | | | | | | | | | | | | | | | | |
| | 22delA | | | | | | | | | | | | | | | | |
| NF1 | c.8050 + | | | | | | | | | | | | | | | | |
| | 49C > T | | | | | | | | | | | | | | | | |
| NF1 | c.8051 − | | | | | | | | | | | | | | | | |
| | 50A > G | | | | | | | | | | | | | | | | |
| NF1 | c.8070G > T | | | | | | | | | | | | | | | | |
| NF1 | c.8088G > A | | | | | | | | | | | | | | | | |
| NF1 | c.8098 − | | | | | | | | | | | | | | | | |
| | 34delA | | | | | | | | | | | | | | | | |
| NF1 | c.8098 − | | | | | | | | | | | | | | | | |
| | 45A > C | | | | | | | | | | | | | | | | |
| NF1 | c.846G > A | | | | | | | | | | | | | | | | |
| NF1 | c.889 − | | | | | | | | | | | | | | | | |
| | 25_889 − | | | | | | | | | | | | | | | | |
| | 21delTTATC | | | | | | | | | | | | | | | | |
| NRAS | c.176C > G | | | | | | | | | | 1 | | | | | | |
| NRAS | c.181C > A | | | | | | | | | | 1 | | | | | | |
| NRAS | c.34G > A | | | | | | | | | | 1 | | | | | | |
| NRAS | c.35G > A | | | | | | | | | | 1 | | | | | | |
| NRAS | c.35G > T | | | | | | | | | | 1 | | | | | | |
| NRAS | c.38G > A | | | | | | | | | | 1 | | | | | | |
| NRAS | c.38G > T | | | | | | | | | | 1 | | | | | | |
| PDGFRA | c.2021C > T | | | | 1 | | | | | | | | | | | | 2 |
| PML | c.647C > T | | | 2 | | | | | | | | | | | | | |
| PTPN11 | c.1028G > A | | | | | | | | | | | | | 1 | | | |
| PTPN11 | c.1052G > A | | | | | | | | | | | | | 1 | | | |
| PTPN11 | c.1093 − | | | | | | | | | | | | | 1 | | | |
| | 9C > A | | | | | | | | | | | | | | | | |
| PTPN11 | c.1221A > G | | | | | | | | | | | | | 1 | | | |
| PTPN11 | c.1379 + | | | | | | | | | | | | | 1 | | | |
| | 20C > T | | | | | | | | | | | | | | | | |
| PTPN11 | c.1380 − | | | | | | | | | | | | | 1 | | | |
| | 16G > T | | | | | | | | | | | | | | | | |
| PTPN11 | c.14 + | | | | | | | | | | | | | 1 | | | |
| | 25G > C | | | | | | | | | | | | | | | | |
| PTPN11 | c.14 + | | | | | | | | | | | | | 1 | | | |
| | 42C > T | | | | | | | | | | | | | | | | |
| PTPN11 | c.1447 + | | | | | | | | | | | | | 1 | | | |
| | 26G > A | | | | | | | | | | | | | | | | |
| PTPN11 | c.1448 − | | | | | | | | | | | | | 1 | | | |
| | 38G > C | | | | | | | | | | | | | | | | |
| PTPN11 | c.1507G > A | | | | | | | | | | | | | 1 | | | |
| PTPN11 | c.182A > C | | | | | | | | | | | | | 1 | | | |
| PTPN11 | c.215C > T | | | | | | | | | | | | | 1 | | | |
| PTPN11 | c.226G > C | | | | | | | | | | | | | 1 | | | |
| PTPN11 | c.227A > G | | | | | | | | | | | | | 1 | | | |
| PTPN11 | c.255C > T | | | | | | | | | | | | | 1 | | | |
| PTPN11 | c.332 + | | | | | | | | | | | | | 1 | | | |
| | 17T > G | | | | | | | | | | | | | | | | |
| PTPN11 | c.525 + | | | | | | | | | | | | | 1 | | | |
| | 12G > C | | | | | | | | | | | | | | | | |
| PTPN11 | c.525 + | | | | | | | | | | | | | 1 | | | |
| | 34A > G | | | | | | | | | | | | | | | | |
| PTPN11 | c.526 − | | | | | | | | | | | | | 1 | | | |
| | 17T > C | | | | | | | | | | | | | | | | |
| PTPN11 | c.526 − | | | | | | | | | | | | | 1 | | | |
| | 33_526 − | | | | | | | | | | | | | | | | |
| | 31delCAA | | | | | | | | | | | | | | | | |
| PTPN11 | c.558G > T | | | | | | | | | | | | | 1 | | | |
| PTPN11 | c.616T > C | | | | | | | | | | | | | 1 | | | |
| PTPN11 | c.642 + | | | | | | | | | | | | | 1 | | | |
| | 138C > T | | | | | | | | | | | | | | | | |
| PTPN11 | c.642 + | | | | | | | | | | | | | 1 | | | |
| | 157_642 + | | | | | | | | | | | | | | | | |
| | 159delTTT | | | | | | | | | | | | | | | | |
| PTPN11 | c.642 + | | | | | | | | | | | | | 1 | | | |
| | 158_642 + | | | | | | | | | | | | | | | | |
| | 159delTT | | | | | | | | | | | | | | | | |
| PTPN11 | c.642 + | | | | | | | | | | | | | 1 | | | |
| | 158_642 + | | | | | | | | | | | | | | | | |
| | 159dupTT | | | | | | | | | | | | | | | | |
| PTPN11 | c.642 + | | | | | | | | | | | | | 1 | | | |
| | 159delT | | | | | | | | | | | | | | | | |
| PTPN11 | c.642 + | | | | | | | | | | | | | 1 | | | |
| | 159dupT | | | | | | | | | | | | | | | | |
| PTPN11 | c.854 − | | | | | | | | | | | | | 1 | | | |
| | 21C > T | | | | | | | | | | | | | | | | |
| PTPN11 | c.854 − | | | | | | | | | | | | | 1 | | | |
| | 30T > C | | | | | | | | | | | | | | | | |
| PTPN11 | c.951G > A | | | | | | | | | | | | | 1 | | | |
| TET2 | c.100C > T | | | | | | | | | | | | | | | | |
| TET2 | c.1064G > A | | | | | | | | | | | | | | | | |
| TET2 | c.1069delA | | | | | | | | | | | | | | | | |
| TET2 | c.1088C > T | | | | | | | | | | | | | | | | |
| TET2 | c.1297_1299delGAA | | | | | | | | | | | | | | | | |
| TET2 | c.1395A > G | | | | | | | | | | | | | | | | |
| TET2 | c.1630C > T | | | | | | | | | | | | | | | | |
| TET2 | c.1884G > A | | | | | | | | | | | | | | | | |
| TET2 | c.2039G > T | | | | | | | | | | | | | | | | |
| TET2 | c.2116C > T | | | | | | | | | | | | | | | | |
| TET2 | c.2185C > T | | | | | | | | | | | | | | | | |
| TET2 | c.2299A > G | | | | | | | | | | | | | | | | |
| TET2 | c.22C > G | | | | | | | | | | | | | | | | |
| TET2 | c.2429A > G | | | | | | | | | | | | | | | | |
| TET2 | c.2596C > T | | | | | | | | | | | | | | | | |
| TET2 | c.2599T > C | | | | | | | | | | | | | | | | |
| TET2 | c.2732C > A | | | | | | | | | | | | | | | | |
| TET2 | c.2771A > G | | | | | | | | | | | | | | | | |
| TET2 | c.3090G > A | | | | | | | | | | | | | | | | |
| TET2 | c.3095T > G | | | | | | | | | | | | | | | | |
| TET2 | c.3116C > T | | | | | | | | | | | | | | | | |
| TET2 | c.3117G > A | | | | | | | | | | | | | | | | |
| TET2 | c.−31C > T | | | | | | | | | | | | | | | | |
| TET2 | c.3251A > C | | | | | | | | | | | | | | | | |
| TET2 | c.3380_3381delAA | | | | | | | | | | | | | | | | |
| TET2 | c.3409 + | | | | | | | | | | | | | | | | |
| | 18A > G | | | | | | | | | | | | | | | | |
| TET2 | c.3409 + | | | | | | | | | | | | | | | | |
| | 37A > C | | | | | | | | | | | | | | | | |
| TET2 | c.3409 + | | | | | | | | | | | | | | | | |
| | 42G > T | | | | | | | | | | | | | | | | |
| TET2 | c.3409 + | | | | | | | | | | | | | | | | |
| | 70G > A | | | | | | | | | | | | | | | | |
| TET2 | c.3595 − | | | | | | | | | | | | | | | | |
| | 4G > A | | | | | | | | | | | | | | | | |
| TET2 | c.37A > C | | | | | | | | | | | | | | | | |
| TET2 | c.3803 + | | | | | | | | | | | | | | | | |
| | 45G > A | | | | | | | | | | | | | | | | |
| TET2 | c.3804 − | | | | | | | | | | | | | | | | |
| | 34C > A | | | | | | | | | | | | | | | | |
| TET2 | c.3863G > A | | | | | | | | | | | | | | | | |
| TET2 | c.3899T > G | | | | | | | | | | | | | | | | |
| TET2 | c.3954 + | | | | | | | | | | | | | | | | |
| | 26C > A | | | | | | | | | | | | | | | | |
| TET2 | c.4045 − | | | | | | | | | | | | | | | | |
| | 27G > A | | | | | | | | | | | | | | | | |
| TET2 | c.4045 − | | | | | | | | | | | | | | | | |
| | 35C > A | | | | | | | | | | | | | | | | |
| TET2 | c.4075C > T | 1 | | | | | | | | | | | | | | | |
| TET2 | c.4140T > C | | | | | | | | | | | | | | | | |
| TET2 | c.4182 + | | | | | | | | | | | | | | | | |
| | 9G > A | | | | | | | | | | | | | | | | |
| TET2 | c.434G > A | | | | | | | | | | | | | | | | |
| TET2 | c.5103G > A | | | | | | | | | | | | | | | | |
| TET2 | c.5162T > G | | | | | | | | | | | | | | | | |
| TET2 | c.5167C > T | | | | | | | | | | | | | | | | |
| TET2 | c.521C > A | | | | | | | | | | | | | | | | |
| TET2 | c.5284A > G | | | | | | | | | | | | | | | | |
| TET2 | c.5333A > G | | | | | | | | | | | | | | | | |
| TET2 | c.5352C > T | | | | | | | | | | | | | | | | |
| TET2 | c.606C > T | | | | | | | | | | | | | | | | |
| TET2 | c.652G > A | | | | | | | | | | | | | | | | |
| TET2 | c.86C > G | | | | | | | | | | | | | | | | |
| TET2 | c.−93G > A | | | | | | | | | | | | | | | | |
| TET2 | c.972A > G | | | | | | | | | | | | | | | | |

| | | | | EGFR | | | | | | | | | | | | | |
|---|---|---|---|---|---|---|---|---|---|---|---|---|---|---|---|---|---|
| | | | | tyrosine | | | | | | | | | | | | | |
| | | | | kinase | | | | | | | | | | | | | |
| | | dasatinib | decitabine | inhibitor | enasidenib | erlotinib | filgrastim | gefitinib | imatinib | | lenvatinib | midostaurin | nilotinib | | nintedanib | nivolumab | olaratumab |
| Gene | Variant | X | ✓ | X | ✓ | X | ✓ | X | ✓ | X | ✓ | ✓ | ✓ | X | ✓ | ✓ | ✓ |
| ABL1 | c.1270 + | | | | | | | | 3 | 1 | | | 1 | | | | |
| | 42A > G | | | | | | | | | | | | | | | | |
| ABL1 | c.128G > A | | | | | | | | 3 | 1 | | | 1 | | | | |
| ABL1 | c.1418G > A | | | | | | | | 3 | 1 | | | 1 | | | | |
| ABL1 | c.1423 + | | | | | | | | 3 | 1 | | | 1 | | | | |
| | 26C > G | | | | | | | | | | | | | | | | |
| ABL1 | c.1497A > G | | | | | | | | 3 | 1 | | | 1 | | | | |
| ABL1 | c.−15_−14insC | | | | | | | | 3 | 1 | | | 1 | | | | |
| ABL1 | c.1512C > T | | | | | | | | 3 | 1 | | | 1 | | | | |
| ABL1 | c.1513 + | | | | | | | | 3 | 1 | | | 1 | | | | |
| | 20_1513 + | | | | | | | | | | | | | | | | |
| | 21delGT | | | | | | | | | | | | | | | | |
| ABL1 | c.1513 + | | | | | | | | 3 | 1 | | | 1 | | | | |
| | 21_1513 + | | | | | | | | | | | | | | | | |
| | 22insCACCCC | | | | | | | | | | | | | | | | |
| ABL1 | c.1596G > A | | | | | | | | 3 | 1 | | | 1 | | | | |
| ABL1 | c.1674G > A | | | | | | | | 3 | 1 | | | 1 | | | | |
| ABL1 | c.1677C > T | | | | | | | | 3 | 1 | | | 1 | | | | |
| ABL1 | c.1678 + | | | | | | | | 3 | 1 | | | 1 | | | | |
| | 14T > C | | | | | | | | | | | | | | | | |
| ABL1 | c.1679 − | | | | | | | | 3 | 1 | | | 1 | | | | |
| | 11T > C | | | | | | | | | | | | | | | | |
| ABL1 | c.1701C > T | | | | | | | | 3 | 1 | | | 1 | | | | |
| ABL1 | c.1826_1828delAGA | | | | | | | | 3 | 1 | | | 1 | | | | |
| ABL1 | c.1854C > T | | | | | | | | 3 | 1 | | | 1 | | | | |
| ABL1 | c.1869G > A | | | | | | | | 3 | 1 | | | 1 | | | | |
| ABL1 | c.1899C > T | | | | | | | | 3 | 1 | | | 1 | | | | |
| ABL1 | c.2040C > T | | | | | | | | 3 | 1 | | | 1 | | | | |
| ABL1 | c.2116G > A | | | | | | | | 3 | 1 | | | 1 | | | | |
| ABL1 | c.2117G > T | | | | | | | | 3 | 1 | | | 1 | | | | |
| ABL1 | c.2187G > A | | | | | | | | 3 | 1 | | | 1 | | | | |
| ABL1 | c.2342C > T | | | | | | | | 3 | 1 | | | 1 | | | | |
| ABL1 | c.2352C > G | | | | | | | | 3 | 1 | | | 1 | | | | |
| ABL1 | c.2429C > T | | | | | | | | 3 | 1 | | | 1 | | | | |
| ABL1 | c.2470G > A | | | | | | | | 3 | 1 | | | 1 | | | | |
| ABL1 | c.249T > G | | | | | | | | 3 | 1 | | | 1 | | | | |
| ABL1 | c.254 − | | | | | | | | 3 | 1 | | | 1 | | | | |
| | 50G > A | | | | | | | | | | | | | | | | |
| ABL1 | c.2703C > G | | | | | | | | 3 | 1 | | | 1 | | | | |
| ABL1 | c.2782C > T | | | | | | | | 3 | 1 | | | 1 | | | | |
| ABL1 | c.2882C > T | | | | | | | | 3 | 1 | | | 1 | | | | |
| ABL1 | c.2883G > A | | | | | | | | 3 | 1 | | | 1 | | | | |
| ABL1 | c.2904C > T | | | | | | | | 3 | 1 | | | 1 | | | | |
| ABL1 | c.2915C > T | | | | | | | | 3 | 1 | | | 1 | | | | |
| ABL1 | c.2916G > A | | | | | | | | 3 | 1 | | | 1 | | | | |
| ABL1 | c.2940C > T | | | | | | | | 3 | 1 | | | 1 | | | | |
| ABL1 | c.2958A > G | | | | | | | | 3 | 1 | | | 1 | | | | |
| ABL1 | c.2970G > A | | | | | | | | 3 | 1 | | | 1 | | | | |
| ABL1 | c.3000C > T | | | | | | | | 3 | 1 | | | 1 | | | | |
| ABL1 | c.3043A > C | | | | | | | | 3 | 1 | | | 1 | | | | |
| ABL1 | c.3072C > T | | | | | | | | 3 | 1 | | | 1 | | | | |
| ABL1 | c.−30T > C | | | | | | | | 3 | 1 | | | 1 | | | | |
| ABL1 | c.3324A > G | | | | | | | | 3 | 1 | | | 1 | | | | |
| ABL1 | c.3344G > C | | | | | | | | 3 | 1 | | | 1 | | | | |
| ABL1 | c.501C > T | | | | | | | | 3 | 1 | | | 1 | | | | |
| ABL1 | c.−53C > G | | | | | | | | 3 | 1 | | | 1 | | | | |
| ABL1 | c.549 + | | | | | | | | 3 | 1 | | | 1 | | | | |
| | 47A > G | | | | | | | | | | | | | | | | |
| ABL1 | c.−588A > T | | | | | | | | 3 | 1 | | | 1 | | | | |
| ABL1 | c.−70C > A | | | | | | | | 3 | 1 | | | 1 | | | | |
| ABL1 | c.720G > A | | | | | | | | 3 | 1 | | | 1 | | | | |
| ABL1 | c.740A > G | | | | | | | | 3 | 1 | | | 1 | | | | |
| ABL1 | c.763G > A | | | | | | | | 1 | | | | 1 | | | | |
| ABL1 | c.764A > T | | | | | | | | 1 | | | | 1 | | | | |
| ABL1 | c.−79G > A | | | | | | | | 3 | 1 | | | 1 | | | | |
| ABL1 | c.822 + | | | | | | | | 3 | 1 | | | 1 | | | | |
| | 16C > G | | | | | | | | | | | | | | | | |
| ABL1 | c.843A > G | | | | | | | | 3 | 1 | | | 1 | | | | |
| ABL1 | c.908 − | | | | | | | | 3 | 1 | | | 1 | | | | |
| | 31C > A | | | | | | | | | | | | | | | | |
| ABL1 | c.908 − | | | | | | | | 3 | 1 | | | 1 | | | | |
| | 45T > C | | | | | | | | | | | | | | | | |
| ABL1 | c.944C > T | 1 | | | | | | | | 1 | | | | 1 | | | |
| ABL1 | c.991A > G | | | | | | | | 3 | 1 | | | 1 | | | | |
| ASXL1 | c.*22A > G | | | | | | | | | | | | | | | | |
| ASXL1 | c.1158G > T | | | | | | | | | | | | | | | | |
| ASXL1 | c.1210C > T | | | | | | | | | | | | | | | | |
| ASXL1 | c.1331C > T | | | | | | | | | | | | | | | | |
| ASXL1 | c.140 + | | | | | | | | | | | | | | | | |
| | 26A > G | | | | | | | | | | | | | | | | |
| ASXL1 | c.1429G > C | | | | | | | | | | | | | | | | |
| ASXL1 | c.143 + | | | | | | | | | | | | | | | | |
| | 50C > T | | | | | | | | | | | | | | | | |
| ASXL1 | c.1465C > G | | | | | | | | | | | | | | | | |
| ASXL1 | c.1520T > A | | | | | | | | | | | | | | | | |
| ASXL1 | c.1749G > A | | | | | | | | | | | | | | | | |
| ASXL1 | c.1895_1896delinsAA | | | | | | | | | | | | | | | | |
| ASXL1 | c.1898A > G | | | | | | | | | | | | | | | | |
| ASXL1 | c.1923C > T | | | | | | | | | | | | | | | | |
| ASXL1 | c.1926_1927delAG | | | | | | | | | | | | | | | | |
| ASXL1 | c.1926delA | | | | | | | | | | | | | | | | |
| ASXL1 | c.1935dupT | | | | | | | | | | | | | | | | |
| ASXL1 | c.1954G > A | | | | | | | | | | | | | | | | |
| ASXL1 | c.1965C > T | | | | | | | | | | | | | | | | |
| ASXL1 | c.2059T > C | | | | | | | | | | | | | | | | |
| ASXL1 | c.2078_2103del26 | | | | | | | | | | | | | | | | |
| ASXL1 | c.2129delG | | | | | | | | | | | | | | | | |
| ASXL1 | c.2250C > T | | | | | | | | | | | | | | | | |
| ASXL1 | c.2251G > A | | | | | | | | | | | | | | | | |
| ASXL1 | c.2395G > T | | | | | | | | | | | | | | | | |
| ASXL1 | c.2416_2417dupAC | | | | | | | | | | | | | | | | |
| ASXL1 | c.2444T > C | | | | | | | | | | | | | | | | |
| ASXL1 | c.2461delG | | | | | | | | | | | | | | | | |
| ASXL1 | c.2473A > T | | | | | | | | | | | | | | | | |
| ASXL1 | c.2513A > G | | | | | | | | | | | | | | | | |
| ASXL1 | c.252 + | | | | | | | | | | | | | | | | |
| | 11delT | | | | | | | | | | | | | | | | |
| ASXL1 | c.252 + | | | | | | | | | | | | | | | | |
| | 11T > C | | | | | | | | | | | | | | | | |
| ASXL1 | c.2929C > T | | | | | | | | | | | | | | | | |
| ASXL1 | c.2957A > G | | | | | | | | | | | | | | | | |
| ASXL1 | c.2985C > T | | | | | | | | | | | | | | | | |
| ASXL1 | c.3029C > T | | | | | | | | | | | | | | | | |
| ASXL1 | c.3098A > T | | | | | | | | | | | | | | | | |
| ASXL1 | c.3384C > T | | | | | | | | | | | | | | | | |
| ASXL1 | c.3498C > G | | | | | | | | | | | | | | | | |
| ASXL1 | c.3503G > C | | | | | | | | | | | | | | | | |
| ASXL1 | c.3513G > A | | | | | | | | | | | | | | | | |
| ASXL1 | c.3519G > A | | | | | | | | | | | | | | | | |
| ASXL1 | c.3662C > A | | | | | | | | | | | | | | | | |
| ASXL1 | c.3692C > T | | | | | | | | | | | | | | | | |
| ASXL1 | c.373 + | | | | | | | | | | | | | | | | |
| | 16T > G | | | | | | | | | | | | | | | | |
| ASXL1 | c.3739C > T | | | | | | | | | | | | | | | | |
| ASXL1 | c.3745A > G | | | | | | | | | | | | | | | | |
| ASXL1 | c.374 − | | | | | | | | | | | | | | | | |
| | 9C > T | | | | | | | | | | | | | | | | |
| ASXL1 | c.3759T > C | | | | | | | | | | | | | | | | |
| ASXL1 | c.3889G > A | | | | | | | | | | | | | | | | |
| ASXL1 | c.3973C > T | | | | | | | | | | | | | | | | |
| ASXL1 | c.4098C > T | | | | | | | | | | | | | | | | |
| ASXL1 | c.4189G > A | | | | | | | | | | | | | | | | |
| ASXL1 | c.4470G > T | | | | | | | | | | | | | | | | |
| ASXL1 | c.472 − | | | | | | | | | | | | | | | | |
| | 22T > C | | | | | | | | | | | | | | | | |
| ASXL1 | c.540C > T | | | | | | | | | | | | | | | | |
| ASXL1 | c.57 + | | | | | | | | | | | | | | | | |
| | 19G > A | | | | | | | | | | | | | | | | |
| ASXL1 | c.57 + | | | | | | | | | | | | | | | | |
| | 49dupG | | | | | | | | | | | | | | | | |
| ASXL1 | c.57 + | | | | | | | | | | | | | | | | |
| | 52C > T | | | | | | | | | | | | | | | | |
| ASXL1 | c.582C > T | | | | | | | | | | | | | | | | |
| ASXL1 | c.58 − | | | | | | | | | | | | | | | | |
| | 49A > C | | | | | | | | | | | | | | | | |
| ASXL1 | c.719 − | | | | | | | | | | | | | | | | |
| | 100C > T | | | | | | | | | | | | | | | | |
| ASXL1 | c.993T > C | | | | | | | | | | | | | | | | |
| BRAF | c.1068A > G | | | | | | | | | 1 | | | | | | | |
| BRAF | c.1140 + | | | | | | | | | 1 | | | | | | | |
| | 45A > G | | | | | | | | | | | | | | | | |
| BRAF | c.1141 − | | | | | | | | | 1 | | | | | | | |
| | 19C > T | | | | | | | | | | | | | | | | |
| BRAF | c.1150A > G | | | | | | | | | 1 | | | | | | | |
| BRAF | c.1227A > G | | | | | | | | | 1 | | | | | | | |
| BRAF | c.1315 − | | | | | | | | | 1 | | | | | | | |
| | 18T > G | | | | | | | | | | | | | | | | |
| BRAF | c.1383A > G | | | | | | | | | 1 | | | | | | | |
| BRAF | c.1405G > C | | | | | | | | | | | | | | | | |
| BRAF | c.1517 + | | | | | | | | | 1 | | | | | | | |
| | 20T > C | | | | | | | | | | | | | | | | |
| BRAF | c.1517 + | | | | | | | | | 1 | | | | | | | |
| | 35G > C | | | | | | | | | | | | | | | | |
| BRAF | c.1517 + | | | | | | | | | 1 | | | | | | | |
| | 46T > C | | | | | | | | | | | | | | | | |
| BRAF | c.1518 − | | | | | | | | | 1 | | | | | | | |
| | 48C > T | | | | | | | | | | | | | | | | |
| BRAF | c.1694 + | | | | | | | | | 1 | | | | | | | |
| | 14G > A | | | | | | | | | | | | | | | | |
| BRAF | c.1742A > G | | | | | | | | | 1 | | | | | | | |
| BRAF | c.1781A > G | | | | | | | | | 1 | | | | | | | |
| BRAF | c.1782T > A | | | | | | | | | | | | | | | | |
| BRAF | c.1799T > A | | | | | | | | | 2 | | | | | | 2 | |
| BRAF | c.1929A > G | | | | | | | | | 1 | | | | | | | |
| BRAF | c.1992 + | | | | | | | | | 1 | | | | | | | |
| | 16G > A | | | | | | | | | | | | | | | | |
| BRAF | c.1992 + | | | | | | | | | 1 | | | | | | | |
| | 16G > C | | | | | | | | | | | | | | | | |
| BRAF | c.1992 + | | | | | | | | | 1 | | | | | | | |
| | 1847G > C | | | | | | | | | | | | | | | | |
| BRAF | c.1992 + | | | | | | | | | 1 | | | | | | | |
| | 40A > G | | | | | | | | | | | | | | | | |
| BRAF | c.2127 + | | | | | | | | | 1 | | | | | | | |
| | 3A > G | | | | | | | | | | | | | | | | |
| BRAF | c.2128 − | | | | | | | | | 1 | | | | | | | |
| | 16C > T | | | | | | | | | | | | | | | | |
| BRAF | c.2128 − | | | | | | | | | 1 | | | | | | | |
| | 17dupT | | | | | | | | | | | | | | | | |
| BRAF | c.2128 − | | | | | | | | | 1 | | | | | | | |
| | 26T > C | | | | | | | | | | | | | | | | |
| BRAF | c.2128 − | | | | | | | | | 1 | | | | | | | |
| | 27_2128 − | | | | | | | | | | | | | | | | |
| | 18del10 | | | | | | | | | | | | | | | | |
| BRAF | c.2128 − | | | | | | | | | 1 | | | | | | | |
| | 27_2128 − | | | | | | | | | | | | | | | | |
| | 19delCTTTTTTTT | | | | | | | | | | | | | | | | |
| BRAF | c.2128 − | | | | | | | | | 1 | | | | | | | |
| | 28dupT | | | | | | | | | | | | | | | | |
| BRAF | c.2128 − | | | | | | | | | 1 | | | | | | | |
| | 29_2128 − | | | | | | | | | | | | | | | | |
| | 28dupTT | | | | | | | | | | | | | | | | |
| BRAF | c.2128 − | | | | | | | | | 1 | | | | | | | |
| | 30_2128 − | | | | | | | | | | | | | | | | |
| | 28delTTT | | | | | | | | | | | | | | | | |
| BRAF | c.2128 − | | | | | | | | | 1 | | | | | | | |
| | 44_2128 − | | | | | | | | | | | | | | | | |
| | 42delCTT | | | | | | | | | | | | | | | | |
| BRAF | c.2128 − | | | | | | | | | 1 | | | | | | | |
| | 50T > C | | | | | | | | | | | | | | | | |
| BRAF | c.2128 − | | | | | | | | | 1 | | | | | | | |
| | 54_2128 − | | | | | | | | | | | | | | | | |
| | 51dupCTTT | | | | | | | | | | | | | | | | |
| BRAF | c.2128 − | | | | | | | | | 1 | | | | | | | |
| | 5delT | | | | | | | | | | | | | | | | |
| BRAF | c.2235A > G | | | | | | | | | 1 | | | | | | | |
| BRAF | c.240 + | | | | | | | | | 1 | | | | | | | |
| | 46delA | | | | | | | | | | | | | | | | |
| BRAF | c.36G > A | | | | | | | | | 1 | | | | | | | |
| BRAF | c.609 − | | | | | | | | | 1 | | | | | | | |
| | 290delT | | | | | | | | | | | | | | | | |
| BRAF | c.609 − | | | | | | | | | 1 | | | | | | | |
| | 291_609 − | | | | | | | | | | | | | | | | |
| | 290delTT | | | | | | | | | | | | | | | | |
| BRAF | c.609 − | | | | | | | | | 1 | | | | | | | |
| | 292_609 − | | | | | | | | | | | | | | | | |
| | 290delTTT | | | | | | | | | | | | | | | | |
| BRAF | c.609 − | | | | | | | | | 1 | | | | | | | |
| | 293_609 − | | | | | | | | | | | | | | | | |
| | 290delTTTT | | | | | | | | | | | | | | | | |
| BRAF | c.609 − | | | | | | | | | 1 | | | | | | | |
| | 299_609 − | | | | | | | | | | | | | | | | |
| | 290del10 | | | | | | | | | | | | | | | | |
| BRAF | c.609 − | | | | | | | | | 1 | | | | | | | |
| | 300_609 − | | | | | | | | | | | | | | | | |
| | 290del11 | | | | | | | | | | | | | | | | |
| BRAF | c.708C > T | | | | | | | | | 1 | | | | | | | |
| BRAF | c.711 + | | | | | | | | | 1 | | | | | | | |
| | 24delT | | | | | | | | | | | | | | | | |
| BRAF | c.712 − | | | | | | | | | 1 | | | | | | | |
| | 18T > C | | | | | | | | | | | | | | | | |
| BRAF | c.712 − | | | | | | | | | 1 | | | | | | | |
| | 40T > A | | | | | | | | | | | | | | | | |
| BRAF | c.980 + | | | | | | | | | 1 | | | | | | | |
| | 27G > A | | | | | | | | | | | | | | | | |
| BRAF | c.981 − | | | | | | | | | 1 | | | | | | | |
| | 20C > T | | | | | | | | | | | | | | | | |
| CSF3R | c.1809C > T | | | | | | 1 | | | | | | | | | | |
| CSF3R | c.1853C > T | | | | | | 1 | | | | | | | | | | |
| CSF3R | c.2041 − | | | | | | 1 | | | | | | | | | | |
| | 8C > T | | | | | | | | | | | | | | | | |
| DNMT3A | c.1014 + | | 3 | | | | | | | | | | | | | | |
| | 34G > A | | | | | | | | | | | | | | | | |
| DNMT3A | c.−10C > T | | 3 | | | | | | | | | | | | | | |
| DNMT3A | c.1122 + | | 3 | | | | | | | | | | | | | | |
| | 7G > A | | | | | | | | | | | | | | | | |
| DNMT3A | c.1140G > A | | 3 | | | | | | | | | | | | | | |
| DNMT3A | c.1170C > T | | 3 | | | | | | | | | | | | | | |
| DNMT3A | c.1204C > T | | 3 | | | | | | | | | | | | | | |
| DNMT3A | c.120G > A | | 3 | | | | | | | | | | | | | | |
| DNMT3A | c.1266G > A | | 3 | | | | | | | | | | | | | | |
| DNMT3A | c.1279 + | | 3 | | | | | | | | | | | | | | |
| | 18G > A | | | | | | | | | | | | | | | | |
| DNMT3A | c.1279 + | | 3 | | | | | | | | | | | | | | |
| | 38G > A | | | | | | | | | | | | | | | | |
| DNMT3A | c.1280 − | | 3 | | | | | | | | | | | | | | |
| | 46delT | | | | | | | | | | | | | | | | |
| DNMT3A | c.1280 − | | 3 | | | | | | | | | | | | | | |
| | 7C > T | | | | | | | | | | | | | | | | |
| DNMT3A | c.1430 − | | 3 | | | | | | | | | | | | | | |
| | 13C > T | | | | | | | | | | | | | | | | |
| DNMT3A | c.1439T > G | | 3 | | | | | | | | | | | | | | |
| DNMT3A | c.1448T > G | | 3 | | | | | | | | | | | | | | |
| DNMT3A | c.1502A > G | | 3 | | | | | | | | | | | | | | |
| DNMT3A | c.1554 + | | 3 | | | | | | | | | | | | | | |
| | 48G > A | | | | | | | | | | | | | | | | |
| DNMT3A | c.1555 − | | 3 | | | | | | | | | | | | | | |
| | 49G > A | | | | | | | | | | | | | | | | |
| DNMT3A | c.1668 − | | 3 | | | | | | | | | | | | | | |
| | 46C > T | | | | | | | | | | | | | | | | |
| DNMT3A | c.−178 + | | 3 | | | | | | | | | | | | | | |
| | 13112G > C | | | | | | | | | | | | | | | | |
| DNMT3A | c.−178 + | | 3 | | | | | | | | | | | | | | |
| | 13219G > A | | | | | | | | | | | | | | | | |
| DNMT3A | c.1852 − | | 3 | | | | | | | | | | | | | | |
| | 37C > A | | | | | | | | | | | | | | | | |
| DNMT3A | c.1937 − | | 3 | | | | | | | | | | | | | | |
| | 5T > G | | | | | | | | | | | | | | | | |
| DNMT3A | c.2173 + | | 3 | | | | | | | | | | | | | | |
| | 26C > T | | | | | | | | | | | | | | | | |
| DNMT3A | c.2173 + | | 3 | | | | | | | | | | | | | | |
| | 45G > A | | | | | | | | | | | | | | | | |
| DNMT3A | c.2206C > T | | 1 | | | | | | | | | | | | | | |
| DNMT3A | c.2261T > C | | 3 | | | | | | | | | | | | | | |
| DNMT3A | c.2323 − | | 3 | | | | | | | | | | | | | | |
| | 243G > C | | | | | | | | | | | | | | | | |
| DNMT3A | c.2446C > T | | 1 | | | | | | | | | | | | | | |
| DNMT3A | c.2597 + | | 3 | | | | | | | | | | | | | | |
| | 30G > A | | | | | | | | | | | | | | | | |
| DNMT3A | c.2597 + | | 3 | | | | | | | | | | | | | | |
| | 36G > A | | | | | | | | | | | | | | | | |
| DNMT3A | c.2652G > A | | 3 | | | | | | | | | | | | | | |
| DNMT3A | c.2676A > G | | 3 | | | | | | | | | | | | | | |
| DNMT3A | c.2688A > G | | 3 | | | | | | | | | | | | | | |
| DNMT3A | c.2706delC | | 2 | | | | | | | | | | | | | | |
| DNMT3A | c.2732G > A | | 3 | | | | | | | | | | | | | | |
| DNMT3A | c.27C > T | | 3 | | | | | | | | | | | | | | |
| DNMT3A | c.448 + | | 3 | | | | | | | | | | | | | | |
| | 23A > T | | | | | | | | | | | | | | | | |
| DNMT3A | c.639 + | | 3 | | | | | | | | | | | | | | |
| | 6G > A | | | | | | | | | | | | | | | | |
| DNMT3A | c.640 − | | 3 | | | | | | | | | | | | | | |
| | 1439G > A | | | | | | | | | | | | | | | | |
| DNMT3A | c.640 − | | 3 | | | | | | | | | | | | | | |
| | 3916G > T | | | | | | | | | | | | | | | | |
| DNMT3A | c.640 − | | 3 | | | | | | | | | | | | | | |
| | 49delG | | | | | | | | | | | | | | | | |
| DNMT3A | c.−6G > A | | 3 | | | | | | | | | | | | | | |
| DNMT3A | c.72 + | | 3 | | | | | | | | | | | | | | |
| | 10G > A | | | | | | | | | | | | | | | | |
| DNMT3A | c.73 − | | 3 | | | | | | | | | | | | | | |
| | 43T > C | | | | | | | | | | | | | | | | |
| DNMT3A | c.759C > T | | 3 | | | | | | | | | | | | | | |
| DNMT3A | c.801C > T | | 3 | | | | | | | | | | | | | | |
| DNMT3A | c.855 + | | 3 | | | | | | | | | | | | | | |
| | 16A > C | | | | | | | | | | | | | | | | |
| DNMT3A | c.89A > C | | 3 | | | | | | | | | | | | | | |
| DNMT3A | c.939G > A | | 1 | | | | | | | | | | | | | | |
| DNMT3A | c.998delA | | 3 | | | | | | | | | | | | | | |
| EZH2 | c.*20C > A | | | | | | | | | | | | | | | | |
| EZH2 | c.*21delC | | | | | | | | | | | | | | | | |
| EZH2 | c.1000 − | | | | | | | | | | | | | | | | |
| | 47A > G | | | | | | | | | | | | | | | | |
| EZH2 | c.1017T > C | | | | | | | | | | | | | | | | |
| EZH2 | c.118 − | | | | | | | | | | | | | | | | |
| | 4delT | | | | | | | | | | | | | | | | |
| EZH2 | c.118 − | | | | | | | | | | | | | | | | |
| | 4dupT | | | | | | | | | | | | | | | | |
| EZH2 | c.118 − | | | | | | | | | | | | | | | | |
| | 5_118 − | | | | | | | | | | | | | | | | |
| | 4delTT | | | | | | | | | | | | | | | | |
| EZH2 | c.1240 + | | | | | | | | | | | | | | | | |
| | 35T > C | | | | | | | | | | | | | | | | |
| EZH2 | c.1240 + | | | | | | | | | | | | | | | | |
| | 48G > A | | | | | | | | | | | | | | | | |
| EZH2 | c.1240 + | | | | | | | | | | | | | | | | |
| | 9C > A | | | | | | | | | | | | | | | | |
| EZH2 | c.1281A > G | | | | | | | | | | | | | | | | |
| EZH2 | c.1392G > A | | | | | | | | | | | | | | | | |
| EZH2 | c.1410 + | | | | | | | | | | | | | | | | |
| | 25_1410 + | | | | | | | | | | | | | | | | |
| | 33delTTAAAAGAG | | | | | | | | | | | | | | | | |
| EZH2 | c.1452A > G | | | | | | | | | | | | | | | | |
| EZH2 | c.1459G > A | | | | | | | | | | | | | | | | |
| EZH2 | c.1505 + | | | | | | | | | | | | | | | | |
| | 12A > G | | | | | | | | | | | | | | | | |
| EZH2 | c.1505 + | | | | | | | | | | | | | | | | |
| | 20C > G | | | | | | | | | | | | | | | | |
| EZH2 | c.1719C > A | | | | | | | | | | | | | | | | |
| EZH2 | c.1731G > A | | | | | | | | | | | | | | | | |
| EZH2 | c.1852 − | | | | | | | | | | | | | | | | |
| | 21T > C | | | | | | | | | | | | | | | | |
| EZH2 | c.1852 − | | | | | | | | | | | | | | | | |
| | 6C > T | | | | | | | | | | | | | | | | |
| EZH2 | c.1852 − | | | | | | | | | | | | | | | | |
| | 9A > G | | | | | | | | | | | | | | | | |
| EZH2 | c.1865C > T | | | | | | | | | | | | | | | | |
| EZH2 | c.1948 − | | | | | | | | | | | | | | | | |
| | 29dupA | | | | | | | | | | | | | | | | |
| EZH2 | c.1967C > T | | | | | | | | | | | | | | | | |
| EZH2 | c.2028T > C | | | | | | | | | | | | | | | | |
| EZH2 | c.2035G > A | | | | | | | | | | | | | | | | |
| EZH2 | c.2068C > T | | | | | | | | | | | | | | | | |
| EZH2 | c.2110 + | | | | | | | | | | | | | | | | |
| | 39A > C | | | | | | | | | | | | | | | | |
| EZH2 | c.2110 + | | | | | | | | | | | | | | | | |
| | 6T > G | | | | | | | | | | | | | | | | |
| EZH2 | c.2111 − | | | | | | | | | | | | | | | | |
| | 38G > A | | | | | | | | | | | | | | | | |
| EZH2 | c.2111 − | | | | | | | | | | | | | | | | |
| | 46C > T | | | | | | | | | | | | | | | | |
| EZH2 | c.2196 − | | | | | | | | | | | | | | | | |
| | 20T > C | | | | | | | | | | | | | | | | |
| EZH2 | c.246 + | | | | | | | | | | | | | | | | |
| | 37T > C | | | | | | | | | | | | | | | | |
| EZH2 | c.247 − | | | | | | | | | | | | | | | | |
| | 10dupT | | | | | | | | | | | | | | | | |
| EZH2 | c.363 + | | | | | | | | | | | | | | | | |
| | 43T > C | | | | | | | | | | | | | | | | |
| EZH2 | c.396T > C | | | | | | | | | | | | | | | | |
| EZH2 | c.434T > C | | | | | | | | | | | | | | | | |
| EZH2 | c.484 + | | | | | | | | | | | | | | | | |
| | 13C > T | | | | | | | | | | | | | | | | |
| EZH2 | c.485 − | | | | | | | | | | | | | | | | |
| | 7G > A | | | | | | | | | | | | | | | | |
| EZH2 | c.538C > T | | | | | | | | | | | | | | | | |
| EZH2 | c.553G > C | | | | | | | | | | | | | | | | |
| EZH2 | c.625 + | | | | | | | | | | | | | | | | |
| | 25T > C | | | | | | | | | | | | | | | | |
| EZH2 | c.625 + | | | | | | | | | | | | | | | | |
| | 26G > T | | | | | | | | | | | | | | | | |
| EZH2 | c.625 + | | | | | | | | | | | | | | | | |
| | 5G > A | | | | | | | | | | | | | | | | |
| EZH2 | c.626 − | | | | | | | | | | | | | | | | |
| | 39A > G | | | | | | | | | | | | | | | | |
| EZH2 | c.728 + | | | | | | | | | | | | | | | | |
| | 19G > C | | | | | | | | | | | | | | | | |
| EZH2 | c.72dupG | | | | | | | | | | | | | | | | |
| FLT3 | c.1713G > A | | | | | | | | | | | | | | 1 | | |
| FLT3 | c.1715A > G | | | | | | | | | | | 1 | | | 1 | | |
| FLT3 | c.1782_1805dup24 | | | | | | | | | | | 30 | | | 1 | | |
| FLT3 | c.1788_1789ins15 | | | | | | | | | | | 30 | | | 1 | | |
| FLT3 | c.1795_1796ins21 | | | | | | | | | | | 30 | | | 1 | | |
| FLT3 | c.2503G > A | | | | | | | | | | | 30 | | | 1 | | |
| FLT3 | c.2503G > C | | | | | | | | | | | 30 | | | 1 | | |
| FLT3 | c.2503G > T | | | | | | | | | | | 1 | | | | | |
| FLT3 | c.2508_2510delCAT | | | | | | | | | | | 1 | | | 1 | | |
| FLT3 | c.2522A > T | | | | | | | | | | | 1 | | | | | |
| FLT3 | c.2524T > G | | | | | | | | | | | 1 | | | | | |
| FLT3 | c.2525A > G | | | | | | | | | | | 30 | | | 1 | | |
| FLT3 | c.FLT3_ITD | | | | | | | | | | | 30 | | | 1 | | |
| IDH2 | c.407A > G | | | | 30 | | | | | | | | | | | | |
| IDH2 | c.419G > A | | | | 33 | | | | | | | | | | | | |
| IDH2 | c.515G > A | | | | 60 | | | | | | | | | | | | |
| JAK2 | c.*4A > C | | | | | | | | | | | | | | | | |
| JAK2 | c.*52A > G | | | | | | | | | | | | | | | | |
| JAK2 | c.1009A > G | | | | | | | | | | | | | | | | |
| JAK2 | C.1174G > A | | | | | | | | | | | | | | | | |
| JAK2 | c.1177C > G | | | | | | | | | | | | | | | | |
| JAK2 | c.1284T > C | | | | | | | | | | | | | | | | |
| JAK2 | c.1323C > T | | | | | | | | | | | | | | | | |
| JAK2 | c.1326 + | | | | | | | | | | | | | | | | |
| | 25G > C | | | | | | | | | | | | | | | | |
| JAK2 | c.1439G > T | | | | | | | | | | | | | | | | |
| JAK2 | c.1458T > G | | | | | | | | | | | | | | | | |
| JAK2 | c.1514 − | | | | | | | | | | | | | | | | |
| | 42C > T | | | | | | | | | | | | | | | | |
| JAK2 | c.1542G > A | | | | | | | | | | | | | | | | |
| JAK2 | c.1622_1627delGAAATG | | | | | | | | | | | | | | | | |
| JAK2 | c.1624_1629delAATGAA | | | | | | | | | | | | | | | | |
| JAK2 | c.1641 + | | | | | | | | | | | | | | | | |
| | 6T > C | | | | | | | | | | | | | | | | |
| JAK2 | c.1710C > T | | | | | | | | | | | | | | | | |
| JAK2 | c.1777 − | | | | | | | | | | | | | | | | |
| | 7delT | | | | | | | | | | | | | | | | |
| JAK2 | c.1777 − | | | | | | | | | | | | | | | | |
| | 7dupT | | | | | | | | | | | | | | | | |
| JAK2 | c.1849G > T | | | | | | | | | | | | | | | | |
| JAK2 | c.1860C > T | | | | | | | | | | | | | | | | |
| JAK2 | c.1864 + | | | | | | | | | | | | | | | | |
| | 14C > T | | | | | | | | | | | | | | | | |
| JAK2 | c.1929T > C | | | | | | | | | | | | | | | | |
| JAK2 | c.1992 + | | | | | | | | | | | | | | | | |
| | 10A > G | | | | | | | | | | | | | | | | |
| JAK2 | c.1992 + | | | | | | | | | | | | | | | | |
| | 21A > T | | | | | | | | | | | | | | | | |
| JAK2 | c.1992 + | | | | | | | | | | | | | | | | |
| | 35T > C | | | | | | | | | | | | | | | | |
| JAK2 | c.1993 − | | | | | | | | | | | | | | | | |
| | 15G > A | | | | | | | | | | | | | | | | |
| JAK2 | c.1993 − | | | | | | | | | | | | | | | | |
| | 15G > T | | | | | | | | | | | | | | | | |
| JAK2 | c.1993 − | | | | | | | | | | | | | | | | |
| | 16C > T | | | | | | | | | | | | | | | | |
| JAK2 | c.2132 − | | | | | | | | | | | | | | | | |
| | 44A > G | | | | | | | | | | | | | | | | |
| JAK2 | c.227 − | | | | | | | | | | | | | | | | |
| | 38T > C | | | | | | | | | | | | | | | | |
| JAK2 | c.227 − | | | | | | | | | | | | | | | | |
| | 7T > C | | | | | | | | | | | | | | | | |
| JAK2 | c.2283 + | | | | | | | | | | | | | | | | |
| | 50A > T | | | | | | | | | | | | | | | | |
| JAK2 | c.2434 + | | | | | | | | | | | | | | | | |
| | 18C > A | | | | | | | | | | | | | | | | |
| JAK2 | c.2434 + | | | | | | | | | | | | | | | | |
| | 35T > C | | | | | | | | | | | | | | | | |
| JAK2 | c.2490G > A | | | | | | | | | | | | | | | | |
| JAK2 | c.2571 + | | | | | | | | | | | | | | | | |
| | 49A > T | | | | | | | | | | | | | | | | |
| JAK2 | c.2577T > C | | | | | | | | | | | | | | | | |
| JAK2 | c.2586T > C | | | | | | | | | | | | | | | | |
| JAK2 | c.2762 − | | | | | | | | | | | | | | | | |
| | 10_2762 − | | | | | | | | | | | | | | | | |
| | 9delAT | | | | | | | | | | | | | | | | |
| JAK2 | c.2850C > T | | | | | | | | | | | | | | | | |
| JAK2 | c.2886 + | | | | | | | | | | | | | | | | |
| | 42T > C | | | | | | | | | | | | | | | | |
| JAK2 | c.2931G > A | | | | | | | | | | | | | | | | |
| JAK2 | c.2958C > T | | | | | | | | | | | | | | | | |
| JAK2 | c.3059 + | | | | | | | | | | | | | | | | |
| | 23A > T | | | | | | | | | | | | | | | | |
| JAK2 | c.3059 + | | | | | | | | | | | | | | | | |
| | 44C > A | | | | | | | | | | | | | | | | |
| JAK2 | c.3188G > A | | | | | | | | | | | | | | | | |
| JAK2 | c.3252T > C | | | | | | | | | | | | | | | | |
| JAK2 | c.3262T > C | | | | | | | | | | | | | | | | |
| JAK2 | c.3288T > A | | | | | | | | | | | | | | | | |
| JAK2 | c.3291 + | | | | | | | | | | | | | | | | |
| | 14T > C | | | | | | | | | | | | | | | | |
| JAK2 | c.3291 + | | | | | | | | | | | | | | | | |
| | 16delT | | | | | | | | | | | | | | | | |
| JAK2 | c.3291 + | | | | | | | | | | | | | | | | |
| | 51T > C | | | | | | | | | | | | | | | | |
| JAK2 | c.3291 + | | | | | | | | | | | | | | | | |
| | 8T > C | | | | | | | | | | | | | | | | |
| JAK2 | c.3323A > G | | | | | | | | | | | | | | | | |
| JAK2 | c.380G > A | | | | | | | | | | | | | | | | |
| JAK2 | c.489C > T | | | | | | | | | | | | | | | | |
| JAK2 | c.525G > A | | | | | | | | | | | | | | | | |
| JAK2 | c.579C > T | | | | | | | | | | | | | | | | |
| JAK2 | c.614 + | | | | | | | | | | | | | | | | |
| | 33T > C | | | | | | | | | | | | | | | | |
| JAK2 | c.615 − | | | | | | | | | | | | | | | | |
| | 24C > T | | | | | | | | | | | | | | | | |
| JAK2 | c.731A > G | | | | | | | | | | | | | | | | |
| JAK2 | c.936 + | | | | | | | | | | | | | | | | |
| | 13G > A | | | | | | | | | | | | | | | | |
| KIT | c.1854G > A | | | | | | | | | 8 | 1 | 30 | 1 | | | | |
| KIT | c.2447A > T | | | | | | | | | 1 | 1 | 1 | 1 | | | | |
| KRAS | c.*512T > C | | | 1 | | 1 | | 1 | | | | | | | | | |
| KRAS | c.*550C > T | | | 1 | | 1 | | 1 | | | | | | | | | |
| KRAS | c.111 + | | | 1 | | 1 | | 1 | | | | | | | | | |
| | 46T > G | | | | | | | | | | | | | | | | |
| KRAS | c.173C > T | | | 1 | | 1 | | 1 | | | | | | | | | |
| KRAS | c.179G > A | | | 1 | | 1 | | 1 | | | | | | | | | |
| KRAS | c.182A > C | | | | | | | | | | | | | | | | |
| KRAS | c.182A > G | | | | | | | | | | | | | | | | |
| KRAS | c.183A > T | | | | | | | | | | | | | | | | |
| KRAS | c.34G > A | | | | | | | | | | | | | | | | |
| KRAS | c.350A > G | | | 1 | | 1 | | 1 | | | | | | | | | |
| KRAS | c.35G > A | | | | | | | | | | | | | | | | |
| KRAS | c.35G > C | | | | | | | | | | | | | | | | |
| KRAS | c.35G > T | | | | | | | | | | | | | | | | |
| KRAS | c.38G > A | | | | | | | | | | | | | | | | |
| KRAS | c.451 − | | | 1 | | 1 | | 1 | | | | | | | | | |
| | 18A > G | | | | | | | | | | | | | | | | |
| KRAS | c.451 − | | | 1 | | 1 | | 1 | | | | | | | | | |
| | 5494A > G | | | | | | | | | | | | | | | | |
| KRAS | c.451 − | | | 1 | | 1 | | 1 | | | | | | | | | |
| | 5521G > A | | | | | | | | | | | | | | | | |
| KRAS | c.451 − | | | 1 | | 1 | | 1 | | | | | | | | | |
| | 5617G > A | | | | | | | | | | | | | | | | |
| KRAS | c.451 − | | | 1 | | 1 | | 1 | | | | | | | | | |
| | 9G > A | | | | | | | | | | | | | | | | |
| KRAS | c.519T > C | | | 1 | | 1 | | 1 | | | | | | | | | |
| NF1 | c.*29A > G | | | | | | | | | 1 | | | | | | | |
| NF1 | c.*4T > C | | | | | | | | | 1 | | | | | | | |
| NF1 | c.1185 + | | | | | | | | | 1 | | | | | | | |
| | 30T > C | | | | | | | | | | | | | | | | |
| NF1 | c.1186 − | | | | | | | | | 1 | | | | | | | |
| | 13delT | | | | | | | | | | | | | | | | |
| NF1 | c.1186 − | | | | | | | | | 1 | | | | | | | |
| | 13dupT | | | | | | | | | | | | | | | | |
| NF1 | c.1261 − | | | | | | | | | 1 | | | | | | | |
| | 45G > A | | | | | | | | | | | | | | | | |
| NF1 | c.−126G > A | | | | | | | | | 1 | | | | | | | |
| NF1 | c.1308G > A | | | | | | | | | 1 | | | | | | | |
| NF1 | c.1393 − | | | | | | | | | 1 | | | | | | | |
| | 31G > A | | | | | | | | | | | | | | | | |
| NF1 | c.1393 − | | | | | | | | | 1 | | | | | | | |
| | 32T > C | | | | | | | | | | | | | | | | |
| NF1 | c.1528 − | | | | | | | | | 1 | | | | | | | |
| | 29dupT | | | | | | | | | | | | | | | | |
| NF1 | c.1641 + | | | | | | | | | 1 | | | | | | | |
| | 39T > C | | | | | | | | | | | | | | | | |
| NF1 | c.168C > T | | | | | | | | | 1 | | | | | | | |
| NF1 | c.1721 + | | | | | | | | | 1 | | | | | | | |
| | 72C > T | | | | | | | | | | | | | | | | |
| NF1 | c.1750A > T | | | | | | | | | 1 | | | | | | | |
| NF1 | c.1845 + | | | | | | | | | 1 | | | | | | | |
| | 13A > T | | | | | | | | | | | | | | | | |
| NF1 | c.1845 + | | | | | | | | | 1 | | | | | | | |
| | 45G > A | | | | | | | | | | | | | | | | |
| NF1 | c.1845 + | | | | | | | | | 1 | | | | | | | |
| | 45G > T | | | | | | | | | | | | | | | | |
| NF1 | c.1846 − | | | | | | | | | 1 | | | | | | | |
| | 12A > T | | | | | | | | | | | | | | | | |
| NF1 | c.1846 − | | | | | | | | | 1 | | | | | | | |
| | 39C > T | | | | | | | | | | | | | | | | |
| NF1 | c.1994C > T | | | | | | | | | 1 | | | | | | | |
| NF1 | c.2022C > T | | | | | | | | | 1 | | | | | | | |
| NF1 | c.2034G > A | | | | | | | | | 1 | | | | | | | |
| NF1 | c.204 + | | | | | | | | | 1 | | | | | | | |
| | 51G > C | | | | | | | | | | | | | | | | |
| NF1 | c.2224G > T | | | | | | | | | 1 | | | | | | | |
| NF1 | c.2252 − | | | | | | | | | 1 | | | | | | | |
| | 31A > T | | | | | | | | | | | | | | | | |
| NF1 | c.2252 − | | | | | | | | | 1 | | | | | | | |
| | 34T > C | | | | | | | | | | | | | | | | |
| NF1 | c.2252 − | | | | | | | | | 1 | | | | | | | |
| | 46C > T | | | | | | | | | | | | | | | | |
| NF1 | c.−22G > C | | | | | | | | | 1 | | | | | | | |
| NF1 | c.2544G > A | | | | | | | | | 1 | | | | | | | |
| NF1 | c.2553C > T | | | | | | | | | 1 | | | | | | | |
| NF1 | c.2851 − | | | | | | | | | 1 | | | | | | | |
| | 16T > C | | | | | | | | | | | | | | | | |
| NF1 | c.288 + | | | | | | | | | 1 | | | | | | | |
| | 41G > A | | | | | | | | | | | | | | | | |
| NF1 | c.2921A > G | | | | | | | | | 1 | | | | | | | |
| NF1 | c.2985G > C | | | | | | | | | 1 | | | | | | | |
| NF1 | c.3114 − | | | | | | | | | 1 | | | | | | | |
| | 7T > C | | | | | | | | | | | | | | | | |
| NF1 | c.3197 + | | | | | | | | | 1 | | | | | | | |
| | 10G > A | | | | | | | | | | | | | | | | |
| NF1 | c.3197 + | | | | | | | | | 1 | | | | | | | |
| | 17C > T | | | | | | | | | | | | | | | | |
| NF1 | c.3197 + | | | | | | | | | 1 | | | | | | | |
| | 23G > A | | | | | | | | | | | | | | | | |
| NF1 | c.3197 + | | | | | | | | | 1 | | | | | | | |
| | 47G > A | | | | | | | | | | | | | | | | |
| NF1 | c.3198 − | | | | | | | | | 1 | | | | | | | |
| | 11_3198 − | | | | | | | | | | | | | | | | |
| | 4delTTTTTTTT | | | | | | | | | | | | | | | | |
| NF1 | c.3198 − | | | | | | | | | 1 | | | | | | | |
| | 3dupC | | | | | | | | | | | | | | | | |
| NF1 | c.3198 − | | | | | | | | | 1 | | | | | | | |
| | 4dupT | | | | | | | | | | | | | | | | |
| NF1 | c.3198 − | | | | | | | | | 1 | | | | | | | |
| | 4T > C | | | | | | | | | | | | | | | | |
| NF1 | c.3198 − | | | | | | | | | 1 | | | | | | | |
| | 5_3198 − | | | | | | | | | | | | | | | | |
| | 4dupTT | | | | | | | | | | | | | | | | |
| NF1 | c.3198 − | | | | | | | | | 1 | | | | | | | |
| | 6_3198 − | | | | | | | | | | | | | | | | |
| | 4dupTTT | | | | | | | | | | | | | | | | |
| NF1 | c.3198 − | | | | | | | | | 1 | | | | | | | |
| | 7_3198 − | | | | | | | | | | | | | | | | |
| | 4dupTTTT | | | | | | | | | | | | | | | | |
| NF1 | c.3270A > C | | | | | | | | | 1 | | | | | | | |
| NF1 | c.340C > T | | | | | | | | | 1 | | | | | | | |
| NF1 | c.3496 + | | | | | | | | | 1 | | | | | | | |
| | 19T > C | | | | | | | | | | | | | | | | |
| NF1 | c.3496 + | | | | | | | | | 1 | | | | | | | |
| | 33C > A | | | | | | | | | | | | | | | | |
| NF1 | c.3496 + | | | | | | | | | 1 | | | | | | | |
| | 43A > C | | | | | | | | | | | | | | | | |
| NF1 | c.3498C > T | | | | | | | | | 1 | | | | | | | |
| NF1 | c.3686A > G | | | | | | | | | 1 | | | | | | | |
| NF1 | c.369C > G | | | | | | | | | 1 | | | | | | | |
| NF1 | c.3709 − | | | | | | | | | 1 | | | | | | | |
| | 47A > G | | | | | | | | | | | | | | | | |
| NF1 | c.3867C > T | | | | | | | | | 1 | | | | | | | |
| NF1 | c.3870 + | | | | | | | | | 1 | | | | | | | |
| | 25A > G | | | | | | | | | | | | | | | | |
| NF1 | c.3870 + | | | | | | | | | 1 | | | | | | | |
| | 50A > G | | | | | | | | | | | | | | | | |
| NF1 | c.3871 − | | | | | | | | | 1 | | | | | | | |
| | 31A > G | | | | | | | | | | | | | | | | |
| NF1 | c.3974 + | | | | | | | | | 1 | | | | | | | |
| | 36_3974 + | | | | | | | | | | | | | | | | |
| | 37dupGT | | | | | | | | | | | | | | | | |
| NF1 | c.3974 + | | | | | | | | | 1 | | | | | | | |
| | 36G > T | | | | | | | | | | | | | | | | |
| NF1 | c.3974 + | | | | | | | | | 1 | | | | | | | |
| | 37_3974 + | | | | | | | | | | | | | | | | |
| | 38insG | | | | | | | | | | | | | | | | |
| NF1 | c.3974 + | | | | | | | | | 1 | | | | | | | |
| | 38T > G | | | | | | | | | | | | | | | | |
| NF1 | c.3974 + | | | | | | | | | 1 | | | | | | | |
| | 40T > G | | | | | | | | | | | | | | | | |
| NF1 | c.3974 + | | | | | | | | | 1 | | | | | | | |
| | 43_3974 + | | | | | | | | | | | | | | | | |
| | 44insG | | | | | | | | | | | | | | | | |
| NF1 | c.3974 + | | | | | | | | | 1 | | | | | | | |
| | 46T > G | | | | | | | | | | | | | | | | |
| NF1 | c.3974 + | | | | | | | | | 1 | | | | | | | |
| | 48_3974 + | | | | | | | | | | | | | | | | |
| | 49delTT | | | | | | | | | | | | | | | | |
| NF1 | c.3974 + | | | | | | | | | 1 | | | | | | | |
| | 48_3974 + | | | | | | | | | | | | | | | | |
| | 49dupTT | | | | | | | | | | | | | | | | |
| NF1 | c.3974 + | | | | | | | | | 1 | | | | | | | |
| | 49delT | | | | | | | | | | | | | | | | |
| NF1 | c.3974 + | | | | | | | | | 1 | | | | | | | |
| | 49dupT | | | | | | | | | | | | | | | | |
| NF1 | c.4110 + | | | | | | | | | 1 | | | | | | | |
| | 3780C > T | | | | | | | | | | | | | | | | |
| NF1 | c.4111 − | | | | | | | | | 1 | | | | | | | |
| | 48_4111 − | | | | | | | | | | | | | | | | |
| | 23dup26 | | | | | | | | | | | | | | | | |
| NF1 | c.4182T > C | | | | | | | | | 1 | | | | | | | |
| NF1 | c.4206A > G | | | | | | | | | 1 | | | | | | | |
| NF1 | c.4368 − | | | | | | | | | 1 | | | | | | | |
| | 46G > C | | | | | | | | | | | | | | | | |
| NF1 | c.4514 + | | | | | | | | | 1 | | | | | | | |
| | 12C > T | | | | | | | | | | | | | | | | |
| NF1 | c.4514 + | | | | | | | | | 1 | | | | | | | |
| | 35C > G | | | | | | | | | | | | | | | | |
| NF1 | c.4515 − | | | | | | | | | 1 | | | | | | | |
| | 39A > G | | | | | | | | | | | | | | | | |
| NF1 | c.4772 + | | | | | | | | | 1 | | | | | | | |
| | 29689A > G | | | | | | | | | | | | | | | | |
| NF1 | c.4772 + | | | | | | | | | 1 | | | | | | | |
| | 29883T > G | | | | | | | | | | | | | | | | |
| NF1 | c.4772 + | | | | | | | | | 1 | | | | | | | |
| | 36T > C | | | | | | | | | | | | | | | | |
| NF1 | c.4773 − | | | | | | | | | 1 | | | | | | | |
| | 17877_4773 − | | | | | | | | | | | | | | | | |
| | 17876delAG | | | | | | | | | | | | | | | | |
| NF1 | c.4773 − | | | | | | | | | 1 | | | | | | | |
| | 20174A > C | | | | | | | | | | | | | | | | |
| NF1 | c.4773 − | | | | | | | | | 1 | | | | | | | |
| | 20184delG | | | | | | | | | | | | | | | | |
| NF1 | c.4773 − | | | | | | | | | 1 | | | | | | | |
| | 20194G > A | | | | | | | | | | | | | | | | |
| NF1 | c.4773 − | | | | | | | | | 1 | | | | | | | |
| | 20367C > T | | | | | | | | | | | | | | | | |
| NF1 | c.4773 − | | | | | | | | | 1 | | | | | | | |
| | 20371T > A | | | | | | | | | | | | | | | | |
| NF1 | c.4773 − | | | | | | | | | 1 | | | | | | | |
| | 20399G > A | | | | | | | | | | | | | | | | |
| NF1 | c.4773 − | | | | | | | | | 1 | | | | | | | |
| | 20486T > C | | | | | | | | | | | | | | | | |
| NF1 | c.4773 − | | | | | | | | | 1 | | | | | | | |
| | 20567T > C | | | | | | | | | | | | | | | | |
| NF1 | c.4773 − | | | | | | | | | 1 | | | | | | | |
| | 20708T > C | | | | | | | | | | | | | | | | |
| NF1 | c.4773 − | | | | | | | | | 1 | | | | | | | |
| | 20889A > T | | | | | | | | | | | | | | | | |
| NF1 | c.4773 − | | | | | | | | | 1 | | | | | | | |
| | 20968C > A | | | | | | | | | | | | | | | | |
| NF1 | c.4773 − | | | | | | | | | 1 | | | | | | | |
| | 21063C > G | | | | | | | | | | | | | | | | |
| NF1 | c.4773 − | | | | | | | | | 1 | | | | | | | |
| | 21229G > C | | | | | | | | | | | | | | | | |
| NF1 | c.4773 − | | | | | | | | | 1 | | | | | | | |
| | 21352T > C | | | | | | | | | | | | | | | | |
| NF1 | c.4773 − | | | | | | | | | 1 | | | | | | | |
| | 21440A > G | | | | | | | | | | | | | | | | |
| NF1 | c.4773 − | | | | | | | | | 1 | | | | | | | |
| | 29479G > A | | | | | | | | | | | | | | | | |
| NF1 | c.4773 − | | | | | | | | | 1 | | | | | | | |
| | 29550C > T | | | | | | | | | | | | | | | | |
| NF1 | c.4773 − | | | | | | | | | 1 | | | | | | | |
| | 30170A > G | | | | | | | | | | | | | | | | |
| NF1 | c.4773 − | | | | | | | | | 1 | | | | | | | |
| | 5353G > A | | | | | | | | | | | | | | | | |
| NF1 | c.4773 − | | | | | | | | | 1 | | | | | | | |
| | 5368C > T | | | | | | | | | | | | | | | | |
| NF1 | c.4773 − | | | | | | | | | 1 | | | | | | | |
| | 5461delA | | | | | | | | | | | | | | | | |
| NF1 | c.4773 − | | | | | | | | | 1 | | | | | | | |
| | 5461dupA | | | | | | | | | | | | | | | | |
| NF1 | c.4773 − | | | | | | | | | 1 | | | | | | | |
| | 5462_4773 − | | | | | | | | | | | | | | | | |
| | 5461delAA | | | | | | | | | | | | | | | | |
| NF1 | c.4773 − | | | | | | | | | 1 | | | | | | | |
| | 5463_4773 − | | | | | | | | | | | | | | | | |
| | 5461delAAA | | | | | | | | | | | | | | | | |
| NF1 | c.4773 − | | | | | | | | | 1 | | | | | | | |
| | 5475A > T | | | | | | | | | | | | | | | | |
| NF1 | c.4773 − | | | | | | | | | 1 | | | | | | | |
| | 5476A > T | | | | | | | | | | | | | | | | |
| NF1 | c.4773 − | | | | | | | | | 1 | | | | | | | |
| | 5477dupT | | | | | | | | | | | | | | | | |
| NF1 | c.4773 − | | | | | | | | | 1 | | | | | | | |
| | 6806G > A | | | | | | | | | | | | | | | | |
| NF1 | c.4773 − | | | | | | | | | 1 | | | | | | | |
| | 6844T > C | | | | | | | | | | | | | | | | |
| NF1 | c.4773 − | | | | | | | | | 1 | | | | | | | |
| | 6867C > G | | | | | | | | | | | | | | | | |
| NF1 | c.4773 − | | | | | | | | | 1 | | | | | | | |
| | 6962G > A | | | | | | | | | | | | | | | | |
| NF1 | c.4773 − | | | | | | | | | 1 | | | | | | | |
| | 7106G > T | | | | | | | | | | | | | | | | |
| NF1 | c.4773 − | | | | | | | | | 1 | | | | | | | |
| | 7179C > T | | | | | | | | | | | | | | | | |
| NF1 | c.4773 − | | | | | | | | | 1 | | | | | | | |
| | 7300G > T | | | | | | | | | | | | | | | | |
| NF1 | c.4773 − | | | | | | | | | 1 | | | | | | | |
| | 7333T > C | | | | | | | | | | | | | | | | |
| NF1 | c.4773 − | | | | | | | | | 1 | | | | | | | |
| | 7365C > T | | | | | | | | | | | | | | | | |
| NF1 | c.4866G > A | | | | | | | | | 1 | | | | | | | |
| NF1 | c.5172G > A | | | | | | | | | 1 | | | | | | | |
| NF1 | c.5205 + | | | | | | | | | 1 | | | | | | | |
| | 23T > C | | | | | | | | | | | | | | | | |
| NF1 | c.528T > A | | | | | | | | | 1 | | | | | | | |
| NF1 | c.5546 + | | | | | | | | | 1 | | | | | | | |
| | 19T > A | | | | | | | | | | | | | | | | |
| NF1 | c.5750 − | | | | | | | | | 1 | | | | | | | |
| | 168delT | | | | | | | | | | | | | | | | |
| NF1 | c.5750 − | | | | | | | | | 1 | | | | | | | |
| | 43A > G | | | | | | | | | | | | | | | | |
| NF1 | c.5750 − | | | | | | | | | 1 | | | | | | | |
| | 75C > T | | | | | | | | | | | | | | | | |
| NF1 | c.5820G > A | | | | | | | | | 1 | | | | | | | |
| NF1 | c.60 + | | | | | | | | | 1 | | | | | | | |
| | 16C > A | | | | | | | | | | | | | | | | |
| NF1 | c.6084 + | | | | | | | | | 1 | | | | | | | |
| | 11T > C | | | | | | | | | | | | | | | | |
| NF1 | c.6084 + | | | | | | | | | 1 | | | | | | | |
| | 8C > G | | | | | | | | | | | | | | | | |
| NF1 | c.6085 − | | | | | | | | | 1 | | | | | | | |
| | 17delA | | | | | | | | | | | | | | | | |
| NF1 | c.6085 − | | | | | | | | | 1 | | | | | | | |
| | 17dupA | | | | | | | | | | | | | | | | |
| NF1 | c.6085 − | | | | | | | | | 1 | | | | | | | |
| | 18_6085 − | | | | | | | | | | | | | | | | |
| | 17dupAA | | | | | | | | | | | | | | | | |
| NF1 | c.6085 − | | | | | | | | | 1 | | | | | | | |
| | 19_6085 − | | | | | | | | | | | | | | | | |
| | 17dupAAA | | | | | | | | | | | | | | | | |
| NF1 | c.6085 − | | | | | | | | | 1 | | | | | | | |
| | 27A > T | | | | | | | | | | | | | | | | |
| NF1 | c.6085 − | | | | | | | | | 1 | | | | | | | |
| | 28delT | | | | | | | | | | | | | | | | |
| NF1 | c.6085 − | | | | | | | | | 1 | | | | | | | |
| | 28dupT | | | | | | | | | | | | | | | | |
| NF1 | c.6085 − | | | | | | | | | 1 | | | | | | | |
| | 28T > A | | | | | | | | | | | | | | | | |
| NF1 | c.6085 − | | | | | | | | | 1 | | | | | | | |
| | 29_6085 − | | | | | | | | | | | | | | | | |
| | 28delTT | | | | | | | | | | | | | | | | |
| NF1 | c.6085 − | | | | | | | | | 1 | | | | | | | |
| | 29_6085 − | | | | | | | | | | | | | | | | |
| | 28insA | | | | | | | | | | | | | | | | |
| NF1 | c.6085 − | | | | | | | | | 1 | | | | | | | |
| | 29T > A | | | | | | | | | | | | | | | | |
| NF1 | c.6085 − | | | | | | | | | 1 | | | | | | | |
| | 30_6085 − | | | | | | | | | | | | | | | | |
| | 28delTTT | | | | | | | | | | | | | | | | |
| NF1 | c.6085 − | | | | | | | | | 1 | | | | | | | |
| | 30T > A | | | | | | | | | | | | | | | | |
| NF1 | c.61 − | | | | | | | | | 1 | | | | | | | |
| | 13C > T | | | | | | | | | | | | | | | | |
| NF1 | c.61 − | | | | | | | | | 1 | | | | | | | |
| | 14delT | | | | | | | | | | | | | | | | |
| NF1 | c.61 − | | | | | | | | | 1 | | | | | | | |
| | 14dupT | | | | | | | | | | | | | | | | |
| NF1 | c.61 − | | | | | | | | | 1 | | | | | | | |
| | 4delT | | | | | | | | | | | | | | | | |
| NF1 | c.61 − | | | | | | | | | 1 | | | | | | | |
| | 4dupT | | | | | | | | | | | | | | | | |
| NF1 | c.6330C > T | | | | | | | | | 1 | | | | | | | |
| NF1 | c.6364 + | | | | | | | | | 1 | | | | | | | |
| | 32A > G | | | | | | | | | | | | | | | | |
| NF1 | c.655 − | | | | | | | | | 1 | | | | | | | |
| | 22A > C | | | | | | | | | | | | | | | | |
| NF1 | c.655 − | | | | | | | | | 1 | | | | | | | |
| | 29T > C | | | | | | | | | | | | | | | | |
| NF1 | c.655 − | | | | | | | | | 1 | | | | | | | |
| | 35A > C | | | | | | | | | | | | | | | | |
| NF1 | c.6579 + | | | | | | | | | 1 | | | | | | | |
| | 45T > A | | | | | | | | | | | | | | | | |
| NF1 | c.6580 − | | | | | | | | | 1 | | | | | | | |
| | 19A > G | | | | | | | | | | | | | | | | |
| NF1 | c.6858 + | | | | | | | | | 1 | | | | | | | |
| | 40A > G | | | | | | | | | | | | | | | | |
| NF1 | c.6859 − | | | | | | | | | 1 | | | | | | | |
| | 14_6859 − | | | | | | | | | | | | | | | | |
| | 13delAT | | | | | | | | | | | | | | | | |
| NF1 | c.7000 − | | | | | | | | | 1 | | | | | | | |
| | 22A > G | | | | | | | | | | | | | | | | |
| NF1 | c.702G > A | | | | | | | | | 1 | | | | | | | |
| NF1 | c.7126 + | | | | | | | | | 1 | | | | | | | |
| | 37C > G | | | | | | | | | | | | | | | | |
| NF1 | c.7127 − | | | | | | | | | 1 | | | | | | | |
| | 11_7127 − | | | | | | | | | | | | | | | | |
| | 8delGTTT | | | | | | | | | | | | | | | | |
| NF1 | c.7127 − | | | | | | | | | 1 | | | | | | | |
| | 11_7127 − | | | | | | | | | | | | | | | | |
| | 8dupGTTT | | | | | | | | | | | | | | | | |
| NF1 | c.7127 − | | | | | | | | | 1 | | | | | | | |
| | 19_7127 − | | | | | | | | | | | | | | | | |
| | 8del12 | | | | | | | | | | | | | | | | |
| NF1 | c.7258 + | | | | | | | | | 1 | | | | | | | |
| | 43A > G | | | | | | | | | | | | | | | | |
| NF1 | c.7259 − | | | | | | | | | 1 | | | | | | | |
| | 17C > T | | | | | | | | | | | | | | | | |
| NF1 | c.730 + | | | | | | | | | 1 | | | | | | | |
| | 15G > T | | | | | | | | | | | | | | | | |
| NF1 | c.730 + | | | | | | | | | 1 | | | | | | | |
| | 30_730 + | | | | | | | | | | | | | | | | |
| | 32dupTTT | | | | | | | | | | | | | | | | |
| NF1 | c.730 + | | | | | | | | | 1 | | | | | | | |
| | 31_730 + | | | | | | | | | | | | | | | | |
| | 32delTT | | | | | | | | | | | | | | | | |
| NF1 | c.730 + | | | | | | | | | 1 | | | | | | | |
| | 31_730 + | | | | | | | | | | | | | | | | |
| | 32dupTT | | | | | | | | | | | | | | | | |
| NF1 | c.730 + | | | | | | | | | 1 | | | | | | | |
| | 32delT | | | | | | | | | | | | | | | | |
| NF1 | c.730 + | | | | | | | | | 1 | | | | | | | |
| | 32dupT | | | | | | | | | | | | | | | | |
| NF1 | c.730 + | | | | | | | | | 1 | | | | | | | |
| | 33G > T | | | | | | | | | | | | | | | | |
| NF1 | c.730 + | | | | | | | | | 1 | | | | | | | |
| | 35C > T | | | | | | | | | | | | | | | | |
| NF1 | c.7305A > G | | | | | | | | | 1 | | | | | | | |
| NF1 | c.7347T > C | | | | | | | | | 1 | | | | | | | |
| NF1 | c.7394 + | | | | | | | | | 1 | | | | | | | |
| | 21A > G | | | | | | | | | | | | | | | | |
| NF1 | c.7395 − | | | | | | | | | 1 | | | | | | | |
| | 29G > A | | | | | | | | | | | | | | | | |
| NF1 | c.7398A > G | | | | | | | | | 1 | | | | | | | |
| NF1 | c.7468G > C | | | | | | | | | 1 | | | | | | | |
| NF1 | c.7552 + | | | | | | | | | 1 | | | | | | | |
| | 24delA | | | | | | | | | | | | | | | | |
| NF1 | c.7675 + | | | | | | | | | 1 | | | | | | | |
| | 46G > A | | | | | | | | | | | | | | | | |
| NF1 | c.7676 − | | | | | | | | | 1 | | | | | | | |
| | 33G > A | | | | | | | | | | | | | | | | |
| NF1 | c.7692C > T | | | | | | | | | 1 | | | | | | | |
| NF1 | c.7807 − | | | | | | | | | 1 | | | | | | | |
| | 13dupT | | | | | | | | | | | | | | | | |
| NF1 | c.7908 − | | | | | | | | | 1 | | | | | | | |
| | 19G > T | | | | | | | | | | | | | | | | |
| NF1 | c.7908 − | | | | | | | | | 1 | | | | | | | |
| | 8delT | | | | | | | | | | | | | | | | |
| NF1 | c.8050 + | | | | | | | | | 1 | | | | | | | |
| | 20G > A | | | | | | | | | | | | | | | | |
| NF1 | c.8050 + | | | | | | | | | 1 | | | | | | | |
| | 22delA | | | | | | | | | | | | | | | | |
| NF1 | c.8050 + | | | | | | | | | 1 | | | | | | | |
| | 49C > T | | | | | | | | | | | | | | | | |
| NF1 | c.8051 − | | | | | | | | | 1 | | | | | | | |
| | 50A > G | | | | | | | | | | | | | | | | |
| NF1 | c.8070G > T | | | | | | | | | 1 | | | | | | | |
| NF1 | c.8088G > A | | | | | | | | | 1 | | | | | | | |
| NF1 | c.8098 − | | | | | | | | | 1 | | | | | | | |
| | 34delA | | | | | | | | | | | | | | | | |
| NF1 | c.8098 − | | | | | | | | | 1 | | | | | | | |
| | 45A > C | | | | | | | | | | | | | | | | |
| NF1 | c.846G > A | | | | | | | | | 1 | | | | | | | |
| NF1 | c.889 − | | | | | | | | | 1 | | | | | | | |
| | 25_889 − | | | | | | | | | | | | | | | | |
| | 21delTTATC | | | | | | | | | | | | | | | | |
| NRAS | c.176C > G | | | | | | | | | | | | | | | | |
| NRAS | c.181C > A | | | | | | | | | | | | | | | | |
| NRAS | c.34G > A | | | | | | | | | | | | | | | | |
| NRAS | c.35G > A | | | | | | | | | | | | | | | | |
| NRAS | c.35G > T | | | | | | | | | | | | | | | | |
| NRAS | c.38G > A | | | | | | | | | | | | | | | | |
| NRAS | c.38G > T | | | | | | | | | | | | | | | | |
| PDGFRA | c.2021C > T | | | | | | | | 8 | | 1 | 30 | 1 | | 1 | | 1 |
| PML | c.647C > T | | | | | | | | | | | | | | | | |
| PTPN11 | c.1028G > A | | | | | | | | | | | | | | | | |
| PTPN11 | c.1052G > A | | | | | | | | | | | | | | | | |
| PTPN11 | c.1093 − | | | | | | | | | | | | | | | | |
| | 9C > A | | | | | | | | | | | | | | | | |
| PTPN11 | c.1221A > G | | | | | | | | | | | | | | | | |
| PTPN11 | c.1379 + | | | | | | | | | | | | | | | | |
| | 20C > T | | | | | | | | | | | | | | | | |
| PTPN11 | c.1380 − | | | | | | | | | | | | | | | | |
| | 16G > T | | | | | | | | | | | | | | | | |
| PTPN11 | c.14 + | | | | | | | | | | | | | | | | |
| | 25G > C | | | | | | | | | | | | | | | | |
| PTPN11 | c.14 + | | | | | | | | | | | | | | | | |
| | 42C > T | | | | | | | | | | | | | | | | |
| PTPN11 | c.1447 + | | | | | | | | | | | | | | | | |
| | 26G > A | | | | | | | | | | | | | | | | |
| PTPN11 | c.1448 − | | | | | | | | | | | | | | | | |
| | 38G > C | | | | | | | | | | | | | | | | |
| PTPN11 | c.1507G > A | | | | | | | | | | | | | | | | |
| PTPN11 | c.182A > C | | | | | | | | | | | | | | | | |
| PTPN11 | c.215C > T | | | | | | | | | | | | | | | | |
| PTPN11 | c.226G > C | | | | | | | | | | | | | | | | |
| PTPN11 | c.227A > G | | | | | | | | | | | | | | | | |
| PTPN11 | c.255C > T | | | | | | | | | | | | | | | | |
| PTPN11 | c.332 + | | | | | | | | | | | | | | | | |
| | 17T > G | | | | | | | | | | | | | | | | |
| PTPN11 | c.525 + | | | | | | | | | | | | | | | | |
| | 12G > C | | | | | | | | | | | | | | | | |
| PTPN11 | c.525 + | | | | | | | | | | | | | | | | |
| | 34A > G | | | | | | | | | | | | | | | | |
| PTPN11 | c.526 − | | | | | | | | | | | | | | | | |
| | 17T > C | | | | | | | | | | | | | | | | |
| PTPN11 | c.526 − | | | | | | | | | | | | | | | | |
| | 33_526 − | | | | | | | | | | | | | | | | |
| | 31delCAA | | | | | | | | | | | | | | | | |
| PTPN11 | c.558G > T | | | | | | | | | | | | | | | | |
| PTPN11 | c.616T > C | | | | | | | | | | | | | | | | |
| PTPN11 | c.642 + | | | | | | | | | | | | | | | | |
| | 138C > T | | | | | | | | | | | | | | | | |
| PTPN11 | c.642 + | | | | | | | | | | | | | | | | |
| | 157_642 + | | | | | | | | | | | | | | | | |
| | 159delTTT | | | | | | | | | | | | | | | | |
| PTPN11 | c.642 + | | | | | | | | | | | | | | | | |
| | 158_642 + | | | | | | | | | | | | | | | | |
| | 159delTT | | | | | | | | | | | | | | | | |
| PTPN11 | c.642 + | | | | | | | | | | | | | | | | |
| | 158_642 + | | | | | | | | | | | | | | | | |
| | 159dupTT | | | | | | | | | | | | | | | | |
| PTPN11 | c.642 + | | | | | | | | | | | | | | | | |
| | 159delT | | | | | | | | | | | | | | | | |
| PTPN11 | c.642 + | | | | | | | | | | | | | | | | |
| | 159dupT | | | | | | | | | | | | | | | | |
| PTPN11 | c.854 − | | | | | | | | | | | | | | | | |
| | 21C > T | | | | | | | | | | | | | | | | |
| PTPN11 | c.854 − | | | | | | | | | | | | | | | | |
| | 30T > C | | | | | | | | | | | | | | | | |
| PTPN11 | c.951G > A | | | | | | | | | | | | | | | | |
| TET2 | c.100C > T | | | | | | | | | | | | | | | | |
| TET2 | c.1064G > A | | | | | | | | | | | | | | | | |
| TET2 | c.1069delA | | | | | | | | | | | | | | | | |
| TET2 | c.1088C > T | | | | | | | | | | | | | | | | |
| TET2 | c.1297_1299delGAA | | | | | | | | | | | | | | | | |
| TET2 | c.1395A > G | | | | | | | | | | | | | | | | |
| TET2 | c.1630C > T | | | | | | | | | | | | | | | | |
| TET2 | c.1884G > A | | | | | | | | | | | | | | | | |
| TET2 | c.2039G > T | | | | | | | | | | | | | | | | |
| TET2 | c.2116C > T | | | | | | | | | | | | | | | | |
| TET2 | c.2185C > T | | | | | | | | | | | | | | | | |
| TET2 | c.2299A > G | | | | | | | | | | | | | | | | |
| TET2 | c.22C > G | | | | | | | | | | | | | | | | |
| TET2 | c.2429A > G | | | | | | | | | | | | | | | | |
| TET2 | c.2596C > T | | | | | | | | | | | | | | | | |
| TET2 | c.2599T > C | | | | | | | | | | | | | | | | |
| TET2 | c.2732C > A | | | | | | | | | | | | | | | | |
| TET2 | c.2771A > G | | | | | | | | | | | | | | | | |
| TET2 | c.3090G > A | | | | | | | | | | | | | | | | |
| TET2 | c.3095T > G | | | | | | | | | | | | | | | | |
| TET2 | c.3116C > T | | | | | | | | | | | | | | | | |
| TET2 | c.3117G > A | | | | | | | | | | | | | | | | |
| TET2 | c.−31C > T | | | | | | | | | | | | | | | | |
| TET2 | c.3251A > C | | | | | | | | | | | | | | | | |
| TET2 | c.3380_3381delAA | | | | | | | | | | | | | | | | |
| TET2 | c.3409 + | | | | | | | | | | | | | | | | |
| | 18A > G | | | | | | | | | | | | | | | | |
| TET2 | c.3409 + | | | | | | | | | | | | | | | | |
| | 37A > C | | | | | | | | | | | | | | | | |
| TET2 | c.3409 + | | | | | | | | | | | | | | | | |
| | 42G > T | | | | | | | | | | | | | | | | |
| TET2 | c.3409 + | | | | | | | | | | | | | | | | |
| | 70G > A | | | | | | | | | | | | | | | | |
| TET2 | c.3595 − | | | | | | | | | | | | | | | | |
| | 4G > A | | | | | | | | | | | | | | | | |
| TET2 | c.37A > C | | | | | | | | | | | | | | | | |
| TET2 | c.3803 + | | | | | | | | | | | | | | | | |
| | 45G > A | | | | | | | | | | | | | | | | |
| TET2 | c.3804 − | | | | | | | | | | | | | | | | |
| | 34C > A | | | | | | | | | | | | | | | | |
| TET2 | c.3863G > A | | | | | | | | | | | | | | | | |
| TET2 | c.3899T > G | | | | | | | | | | | | | | | | |
| TET2 | c.3954 + | | | | | | | | | | | | | | | | |
| | 26C > A | | | | | | | | | | | | | | | | |
| TET2 | c.4045 − | | | | | | | | | | | | | | | | |
| | 27G > A | | | | | | | | | | | | | | | | |
| TET2 | c.4045 − | | | | | | | | | | | | | | | | |
| | 35C > A | | | | | | | | | | | | | | | | |
| TET2 | c.4075C > T | | 1 | | | | | | | | | | | | | | |
| TET2 | c.4140T > C | | | | | | | | | | | | | | | | |
| TET2 | c.4182 + | | | | | | | | | | | | | | | | |
| | 9G > A | | | | | | | | | | | | | | | | |
| TET2 | c.434G > A | | | | | | | | | | | | | | | | |
| TET2 | c.5103G > A | | | | | | | | | | | | | | | | |
| TET2 | c.5162T > G | | | | | | | | | | | | | | | | |
| TET2 | c.5167C > T | | | | | | | | | | | | | | | | |
| TET2 | c.521C > A | | | | | | | | | | | | | | | | |
| TET2 | c.5284A > G | | | | | | | | | | | | | | | | |
| TET2 | c.5333A > G | | | | | | | | | | | | | | | | |
| TET2 | c.5352C > T | | | | | | | | | | | | | | | | |
| TET2 | c.606C > T | | | | | | | | | | | | | | | | |
| TET2 | c.652G > A | | | | | | | | | | | | | | | | |
| TET2 | c.86C > G | | | | | | | | | | | | | | | | |
| TET2 | c.−93G > A | | | | | | | | | | | | | | | | |
| TET2 | c.972A > G | | | | | | | | | | | | | | | | |

| | | | | PEG- | platinum | | | | | | | | |
|---|---|---|---|---|---|---|---|---|---|---|---|---|---|
| | | | | interferon | chemotherapy | | | | | | | | |
| | | | | alfa- | regimen/ | | | | | | | | |
| | | panitumumab | pazopanib | 2a | vinorelbine | ponatinib | regorafenib | ruxolitinib | sorafenib | sunitinib | temozolomide | trametinib | vemurafenib |
| Gene | Variant | X | ✓ | X | X | ✓ | ✓ | ✓ | ✓ | ✓ | ✓ | ✓ | ✓ |
| ABL1 | c.1270 + | | | | | 2 | 1 | | | | 1 | | |
| | 42A > G | | | | | | | | | | | | |
| ABL1 | c.128G > A | | | | | 2 | 1 | | | | 1 | | |
| ABL1 | c.1418G > A | | | | | 2 | 1 | | | | 1 | | |
| ABL1 | c.1423 + | | | | | 2 | 1 | | | | 1 | | |
| | 26C > G | | | | | | | | | | | | |
| ABL1 | c.1497A > G | | | | | 2 | 1 | | | | 1 | | |
| ABL1 | c.−15_−14insC | | | | | 2 | 1 | | | | 1 | | |
| ABL1 | c.1512C > T | | | | | 2 | 1 | | | | 1 | | |
| ABL1 | c.1513 + | | | | | 2 | 1 | | | | 1 | | |
| | 20_1513 + | | | | | | | | | | | | |
| | 21delGT | | | | | | | | | | | | |
| ABL1 | c.1513 + | | | | | 2 | 1 | | | | 1 | | |
| | 21_1513 + | | | | | | | | | | | | |
| | 22insCACCCC | | | | | | | | | | | | |
| ABL1 | c.1596G > A | | | | | 2 | 1 | | | | 1 | | |
| ABL1 | c.1674G > A | | | | | 2 | 1 | | | | 1 | | |
| ABL1 | c.1677C > T | | | | | 2 | 1 | | | | 1 | | |
| ABL1 | c.1678 + | | | | | 2 | 1 | | | | 1 | | |
| | 14T > C | | | | | | | | | | | | |
| ABL1 | c.1679 − | | | | | 2 | 1 | | | | 1 | | |
| | 11T > C | | | | | | | | | | | | |
| ABL1 | c.1701C > T | | | | | 2 | 1 | | | | 1 | | |
| ABL1 | c.1826_1828delAGA | | | | | 2 | 1 | | | | 1 | | |
| ABL1 | c.1854C > T | | | | | 2 | 1 | | | | 1 | | |
| ABL1 | c.1869G > A | | | | | 2 | 1 | | | | 1 | | |
| ABL1 | c.1899C > T | | | | | 2 | 1 | | | | 1 | | |
| ABL1 | c.2040C > T | | | | | 2 | 1 | | | | 1 | | |
| ABL1 | c.2116G > A | | | | | 2 | 1 | | | | 1 | | |
| ABL1 | c.2117G > T | | | | | 2 | 1 | | | | 1 | | |
| ABL1 | c.2187G > A | | | | | 2 | 1 | | | | 1 | | |
| ABL1 | c.2342C > T | | | | | 2 | 1 | | | | 1 | | |
| ABL1 | c.2352C > G | | | | | 2 | 1 | | | | 1 | | |
| ABL1 | c.2429C > T | | | | | 2 | 1 | | | | 1 | | |
| ABL1 | c.2470G > A | | | | | 2 | 1 | | | | 1 | | |
| ABL1 | c.249T > G | | | | | 2 | 1 | | | | 1 | | |
| ABL1 | c.254 − | | | | | 2 | 1 | | | | 1 | | |
| | 50G > A | | | | | | | | | | | | |
| ABL1 | c.2703C > G | | | | | 2 | 1 | | | | 1 | | |
| ABL1 | c.2782C > T | | | | | 2 | 1 | | | | 1 | | |
| ABL1 | c.2882C > T | | | | | 2 | 1 | | | | 1 | | |
| ABL1 | c.2883G > A | | | | | 2 | 1 | | | | 1 | | |
| ABL1 | c.2904C > T | | | | | 2 | 1 | | | | 1 | | |
| ABL1 | c.2915C > T | | | | | 2 | 1 | | | | 1 | | |
| ABL1 | c.2916G > A | | | | | 2 | 1 | | | | 1 | | |
| ABL1 | c.2940C > T | | | | | 2 | 1 | | | | 1 | | |
| ABL1 | c.2958A > G | | | | | 2 | 1 | | | | 1 | | |
| ABL1 | c.2970G > A | | | | | 2 | 1 | | | | 1 | | |
| ABL1 | c.3000C > T | | | | | 2 | 1 | | | | 1 | | |
| ABL1 | c.3043A > C | | | | | 2 | 1 | | | | 1 | | |
| ABL1 | c.3072C > T | | | | | 2 | 1 | | | | 1 | | |
| ABL1 | c.−30T > C | | | | | 2 | 1 | | | | 1 | | |
| ABL1 | c.3324A > G | | | | | 2 | 1 | | | | 1 | | |
| ABL1 | c.3344G > C | | | | | 2 | 1 | | | | 1 | | |
| ABL1 | c.501C > T | | | | | 2 | 1 | | | | 1 | | |
| ABL1 | c.−53C > G | | | | | 2 | 1 | | | | 1 | | |
| ABL1 | c.549 + | | | | | 2 | 1 | | | | 1 | | |
| | 47A > G | | | | | | | | | | | | |
| ABL1 | c.−588A > T | | | | | 2 | 1 | | | | 1 | | |
| ABL1 | c.−70C > A | | | | | 2 | 1 | | | | 1 | | |
| ABL1 | c.720G > A | | | | | 2 | 1 | | | | 1 | | |
| ABL1 | c.740A > G | | | | | 2 | 1 | | | | 1 | | |
| ABL1 | c.763G > A | | | | | 1 | 1 | | | | 1 | | |
| ABL1 | c.764A > T | | | | | 1 | 1 | | | | 1 | | |
| ABL1 | c.−79G > A | | | | | 2 | 1 | | | | 1 | | |
| ABL1 | c.822 + | | | | | 2 | 1 | | | | 1 | | |
| | 16C > G | | | | | | | | | | | | |
| ABL1 | c.843A > G | | | | | 2 | 1 | | | | 1 | | |
| ABL1 | c.908 − | | | | | 2 | 1 | | | | 1 | | |
| | 31C > A | | | | | | | | | | | | |
| ABL1 | c.908 − | | | | | 2 | 1 | | | | 1 | | |
| | 45T > C | | | | | | | | | | | | |
| ABL1 | c.944C > T | | | | | 5 | 1 | | | | 1 | | |
| ABL1 | c.991A > G | | | | | 2 | 1 | | | | 1 | | |
| ASXL1 | c.*22A > G | | | 1 | | | | | | | | | |
| ASXL1 | c.1158G > T | | | 1 | | | | | | | | | |
| ASXL1 | c.1210C > T | | | 1 | | | | | | | | | |
| ASXL1 | c.1331C > T | | | 1 | | | | | | | | | |
| ASXL1 | c.140 + | | | 1 | | | | | | | | | |
| | 26A > G | | | | | | | | | | | | |
| ASXL1 | c.1429G > C | | | 1 | | | | | | | | | |
| ASXL1 | c.143 + | | | 1 | | | | | | | | | |
| | 50C > T | | | | | | | | | | | | |
| ASXL1 | c.1465C > G | | | 1 | | | | | | | | | |
| ASXL1 | c.1520T > A | | | 1 | | | | | | | | | |
| ASXL1 | c.1749G > A | | | 1 | | | | | | | | | |
| ASXL1 | c.1895_1896delinsAA | | | 1 | | | | | | | | | |
| ASXL1 | c.1898A > G | | | 1 | | | | | | | | | |
| ASXL1 | c.1923C > T | | | 1 | | | | | | | | | |
| ASXL1 | c.1926_1927delAG | | | 1 | | | | | | | | | |
| ASXL1 | c.1926delA | | | 1 | | | | | | | | | |
| ASXL1 | c.1935dupT | | | 1 | | | | | | | | | |
| ASXL1 | c.1954G > A | | | 1 | | | | | | | | | |
| ASXL1 | c.1965C > T | | | 1 | | | | | | | | | |
| ASXL1 | c.2059T > C | | | 1 | | | | | | | | | |
| ASXL1 | c.2078_2103del26 | | | 1 | | | | | | | | | |
| ASXL1 | c.2129delG | | | 1 | | | | | | | | | |
| ASXL1 | c.2250C > T | | | 1 | | | | | | | | | |
| ASXL1 | c.2251G > A | | | 1 | | | | | | | | | |
| ASXL1 | c.2395G > T | | | 1 | | | | | | | | | |
| ASXL1 | c.2416_2417dupAC | | | 1 | | | | | | | | | |
| ASXL1 | c.2444T > C | | | 1 | | | | | | | | | |
| ASXL1 | c.2461delG | | | 1 | | | | | | | | | |
| ASXL1 | c.2473A > T | | | 1 | | | | | | | | | |
| ASXL1 | c.2513A > G | | | 1 | | | | | | | | | |
| ASXL1 | c.252 + | | | 1 | | | | | | | | | |
| | 11delT | | | | | | | | | | | | |
| ASXL1 | c.252 + | | | 1 | | | | | | | | | |
| | 11T > C | | | | | | | | | | | | |
| ASXL1 | c.2929C > T | | | 1 | | | | | | | | | |
| ASXL1 | c.2957A > G | | | 1 | | | | | | | | | |
| ASXL1 | c.2985C > T | | | 1 | | | | | | | | | |
| ASXL1 | c.3029C > T | | | 1 | | | | | | | | | |
| ASXL1 | c.3098A > T | | | 1 | | | | | | | | | |
| ASXL1 | c.3384C > T | | | 1 | | | | | | | | | |
| ASXL1 | c.3498C > G | | | 1 | | | | | | | | | |
| ASXL1 | c.3503G > C | | | 1 | | | | | | | | | |
| ASXL1 | c.3513G > A | | | 1 | | | | | | | | | |
| ASXL1 | c.3519G > A | | | 1 | | | | | | | | | |
| ASXL1 | c.3662C > A | | | 1 | | | | | | | | | |
| ASXL1 | c.3692C > T | | | 1 | | | | | | | | | |
| ASXL1 | c.373 + | | | 1 | | | | | | | | | |
| | 16T > G | | | | | | | | | | | | |
| ASXL1 | c.3739C > T | | | 1 | | | | | | | | | |
| ASXL1 | c.3745A > G | | | 1 | | | | | | | | | |
| ASXL1 | c.374 − | | | 1 | | | | | | | | | |
| | 9C > T | | | | | | | | | | | | |
| ASXL1 | c.3759T > C | | | 1 | | | | | | | | | |
| ASXL1 | c.3889G > A | | | 1 | | | | | | | | | |
| ASXL1 | c.3973C > T | | | 1 | | | | | | | | | |
| ASXL1 | c.4098C > T | | | 1 | | | | | | | | | |
| ASXL1 | c.4189G > A | | | 1 | | | | | | | | | |
| ASXL1 | c.4470G > T | | | 1 | | | | | | | | | |
| ASXL1 | c.472 − | | | 1 | | | | | | | | | |
| | 22T > C | | | | | | | | | | | | |
| ASXL1 | c.540C > T | | | 1 | | | | | | | | | |
| ASXL1 | c.57 + | | | 1 | | | | | | | | | |
| | 19G > A | | | | | | | | | | | | |
| ASXL1 | c.57 + | | | 1 | | | | | | | | | |
| | 49dupG | | | | | | | | | | | | |
| ASXL1 | c.57 + | | | 1 | | | | | | | | | |
| | 52C > T | | | | | | | | | | | | |
| ASXL1 | c.582C > T | | | 1 | | | | | | | | | |
| ASXL1 | c.58 − | | | 1 | | | | | | | | | |
| | 49A > C | | | | | | | | | | | | |
| ASXL1 | c.719 − | | | 1 | | | | | | | | | |
| | 100C > T | | | | | | | | | | | | |
| ASXL1 | c.993T > C | | | 1 | | | | | | | | | |
| BRAF | c.1068A > G | | | | | | 1 | | 1 | | | | 1 |
| BRAF | c.1140 + | | | | | | 1 | | 1 | | | | 1 |
| | 45A > G | | | | | | | | | | | | |
| BRAF | c.1141 − | | | | | | 1 | | 1 | | | | 1 |
| | 19C > T | | | | | | | | | | | | |
| BRAF | c.1150A > G | | | | | | 1 | | 1 | | | | 1 |
| BRAF | c.1227A > G | | | | | | 1 | | 1 | | | | 1 |
| BRAF | c.1315 − | | | | | | 1 | | 1 | | | | 1 |
| | 18T > G | | | | | | | | | | | | |
| BRAF | c.1383A > G | | | | | | 1 | | 1 | | | | 1 |
| BRAF | c.1405G > C | | | | | | 1 | | 1 | | | | 1 |
| BRAF | c.1517 + | | | | | | 1 | | 1 | | | | 1 |
| | 20T > C | | | | | | | | | | | | |
| BRAF | c.1517 + | | | | | | 1 | | 1 | | | | 1 |
| | 35G > C | | | | | | | | | | | | |
| BRAF | c.1517 + | | | | | | 1 | | 1 | | | | 1 |
| | 46T > C | | | | | | | | | | | | |
| BRAF | c.1518 − | | | | | | 1 | | 1 | | | | 1 |
| | 48C > T | | | | | | | | | | | | |
| BRAF | c.1694 + | | | | | | 1 | | 1 | | | | 1 |
| | 14G > A | | | | | | | | | | | | |
| BRAF | c.1742A > G | | | | | | 1 | | 1 | | | | 1 |
| BRAF | c.1781A > G | | | | | | 1 | | 1 | | | | 1 |
| BRAF | c.1782T > A | | | | | | 1 | | 1 | | | | |
| BRAF | c.1799T > A | 1 | | | | | 3 | | 3 | | | 2 | 3 |
| BRAF | c.1929A > G | | | | | | 1 | | 1 | | | | 1 |
| BRAF | c.1992 + | | | | | | 1 | | 1 | | | | 1 |
| | 16G > A | | | | | | | | | | | | |
| BRAF | c.1992 + | | | | | | 1 | | 1 | | | | 1 |
| | 16G > C | | | | | | | | | | | | |
| BRAF | c.1992 + | | | | | | 1 | | 1 | | | | 1 |
| | 1847G > C | | | | | | | | | | | | |
| BRAF | c.1992 + | | | | | | 1 | | 1 | | | | 1 |
| | 40A > G | | | | | | | | | | | | |
| BRAF | c.2127 + | | | | | | 1 | | 1 | | | | 1 |
| | 3A > G | | | | | | | | | | | | |
| BRAF | c.2128 − | | | | | | 1 | | 1 | | | | 1 |
| | 16C > T | | | | | | | | | | | | |
| BRAF | c.2128 − | | | | | | 1 | | 1 | | | | 1 |
| | 17dupT | | | | | | | | | | | | |
| BRAF | c.2128 − | | | | | | 1 | | 1 | | | | 1 |
| | 26T > C | | | | | | | | | | | | |
| BRAF | c.2128 − | | | | | | 1 | | 1 | | | | 1 |
| | 27_2128 − | | | | | | | | | | | | |
| | 18del10 | | | | | | | | | | | | |
| BRAF | c.2128 − | | | | | | 1 | | 1 | | | | 1 |
| | 27_2128 − | | | | | | | | | | | | |
| | 19delCTTTTTTTT | | | | | | | | | | | | |
| BRAF | c.2128 − | | | | | | 1 | | 1 | | | | 1 |
| | 28dupT | | | | | | | | | | | | |
| BRAF | c.2128 − | | | | | | 1 | | 1 | | | | 1 |
| | 29_2128 − | | | | | | | | | | | | |
| | 28dupTT | | | | | | | | | | | | |
| BRAF | c.2128 − | | | | | | 1 | | 1 | | | | 1 |
| | 30_2128 − | | | | | | | | | | | | |
| | 28delTTT | | | | | | | | | | | | |
| BRAF | c.2128 − | | | | | | 1 | | 1 | | | | 1 |
| | 44_2128 − | | | | | | | | | | | | |
| | 42delCTT | | | | | | | | | | | | |
| BRAF | c.2128 − | | | | | | 1 | | 1 | | | | 1 |
| | 50T > C | | | | | | | | | | | | |
| BRAF | c.2128 − | | | | | | 1 | | 1 | | | | 1 |
| | 54_2128 − | | | | | | | | | | | | |
| | 51dupCTTT | | | | | | | | | | | | |
| BRAF | c.2128 − | | | | | | 1 | | 1 | | | | 1 |
| | 5delT | | | | | | | | | | | | |
| BRAF | c.2235A > G | | | | | | 1 | | 1 | | | | 1 |
| BRAF | c.240 + | | | | | | 1 | | 1 | | | | 1 |
| | 46delA | | | | | | | | | | | | |
| BRAF | c.36G > A | | | | | | 1 | | 1 | | | | 1 |
| BRAF | c.609 − | | | | | | 1 | | 1 | | | | 1 |
| | 290delT | | | | | | | | | | | | |
| BRAF | c.609 − | | | | | | 1 | | 1 | | | | 1 |
| | 291_609 − | | | | | | | | | | | | |
| | 290delTT | | | | | | | | | | | | |
| BRAF | c.609 − | | | | | | 1 | | 1 | | | | 1 |
| | 292_609 − | | | | | | | | | | | | |
| | 290delTTT | | | | | | | | | | | | |
| BRAF | c.609 − | | | | | | 1 | | 1 | | | | 1 |
| | 293_609 − | | | | | | | | | | | | |
| | 290delTTTT | | | | | | | | | | | | |
| BRAF | c.609 − | | | | | | 1 | | 1 | | | | 1 |
| | 299_609 − | | | | | | | | | | | | |
| | 290del10 | | | | | | | | | | | | |
| BRAF | c.609 − | | | | | | 1 | | 1 | | | | 1 |
| | 300_609 − | | | | | | | | | | | | |
| | 290del11 | | | | | | | | | | | | |
| BRAF | c.708C > T | | | | | | 1 | | 1 | | | | 1 |
| BRAF | c.711 + | | | | | | 1 | | 1 | | | | 1 |
| | 24delT | | | | | | | | | | | | |
| BRAF | c.712 − | | | | | | 1 | | 1 | | | | 1 |
| | 18T > C | | | | | | | | | | | | |
| BRAF | c.712 − | | | | | | 1 | | 1 | | | | 1 |
| | 40T > A | | | | | | | | | | | | |
| BRAF | c.980 + | | | | | | 1 | | 1 | | | | 1 |
| | 27G > A | | | | | | | | | | | | |
| BRAF | c.981 − | | | | | | 1 | | 1 | | | | 1 |
| | 20C > T | | | | | | | | | | | | |
| CSF3R | c.1809C > T | | | | | | | | | | | | |
| CSF3R | c.1853C > T | | | | | | | | | | | | |
| CSF3R | c.2041 − | | | | | | | | | | | | |
| | 8C > T | | | | | | | | | | | | |
| DNMT3A | c.1014 + | | | 1 | | | | | | | | | |
| | 34G > A | | | | | | | | | | | | |
| DNMT3A | c.−10C > T | | | 1 | | | | | | | | | |
| DNMT3A | c.1122 + | | | 1 | | | | | | | | | |
| | 7G > A | | | | | | | | | | | | |
| DNMT3A | c.1140G > A | | | 1 | | | | | | | | | |
| DNMT3A | c.1170C > T | | | 1 | | | | | | | | | |
| DNMT3A | c.1204C > T | | | 1 | | | | | | | | | |
| DNMT3A | c.120G > A | | | 1 | | | | | | | | | |
| DNMT3A | c.1266G > A | | | 1 | | | | | | | | | |
| DNMT3A | c.1279 + | | | 1 | | | | | | | | | |
| | 18G > A | | | | | | | | | | | | |
| DNMT3A | c.1279 + | | | 1 | | | | | | | | | |
| | 38G > A | | | | | | | | | | | | |
| DNMT3A | c.1280 − | | | 1 | | | | | | | | | |
| | 46delT | | | | | | | | | | | | |
| DNMT3A | c.1280 − | | | 1 | | | | | | | | | |
| | 7C > T | | | | | | | | | | | | |
| DNMT3A | c.1430 − | | | 1 | | | | | | | | | |
| | 13C > T | | | | | | | | | | | | |
| DNMT3A | c.1439T > G | | | 1 | | | | | | | | | |
| DNMT3A | c.1448T > G | | | 1 | | | | | | | | | |
| DNMT3A | c.1502A > G | | | 1 | | | | | | | | | |
| DNMT3A | c.1554 + | | | 1 | | | | | | | | | |
| | 48G > A | | | | | | | | | | | | |
| DNMT3A | c.1555 − | | | 1 | | | | | | | | | |
| | 49G > A | | | | | | | | | | | | |
| DNMT3A | c.1668 − | | | 1 | | | | | | | | | |
| | 46C > T | | | | | | | | | | | | |
| DNMT3A | c.−178 + | | | 1 | | | | | | | | | |
| | 13112G > C | | | | | | | | | | | | |
| DNMT3A | c.−178 + | | | 1 | | | | | | | | | |
| | 13219G > A | | | | | | | | | | | | |
| DNMT3A | c.1852 − | | | 1 | | | | | | | | | |
| | 37C > A | | | | | | | | | | | | |
| DNMT3A | c.1937 − | | | 1 | | | | | | | | | |
| | 5T > G | | | | | | | | | | | | |
| DNMT3A | c.2173 + | | | 1 | | | | | | | | | |
| | 26C > T | | | | | | | | | | | | |
| DNMT3A | c.2173 + | | | 1 | | | | | | | | | |
| | 45G > A | | | | | | | | | | | | |
| DNMT3A | c.2206C > T | | | | | | | | | | | | |
| DNMT3A | c.2261T > C | | | 1 | | | | | | | | | |
| DNMT3A | c.2323 − | | | 1 | | | | | | | | | |
| | 243G > C | | | | | | | | | | | | |
| DNMT3A | c.2446C > T | | | | | | | | | | | | |
| DNMT3A | c.2597 + | | | 1 | | | | | | | | | |
| | 30G > A | | | | | | | | | | | | |
| DNMT3A | c.2597 + | | | 1 | | | | | | | | | |
| | 36G > A | | | | | | | | | | | | |
| DNMT3A | c.2652G > A | | | 1 | | | | | | | | | |
| DNMT3A | c.2676A > G | | | 1 | | | | | | | | | |
| DNMT3A | c.2688A > G | | | 1 | | | | | | | | | |
| DNMT3A | c.2706delC | | | | | | | | | | | | |
| DNMT3A | c.2732G > A | | | 1 | | | | | | | | | |
| DNMT3A | c.27C > T | | | 1 | | | | | | | | | |
| DNMT3A | c.448 + | | | 1 | | | | | | | | | |
| | 23A > T | | | | | | | | | | | | |
| DNMT3A | c.639 + | | | 1 | | | | | | | | | |
| | 6G > A | | | | | | | | | | | | |
| DNMT3A | c.640 − | | | 1 | | | | | | | | | |
| | 1439G > A | | | | | | | | | | | | |
| DNMT3A | c.640 − | | | 1 | | | | | | | | | |
| | 3916G > T | | | | | | | | | | | | |
| DNMT3A | c.640 − | | | 1 | | | | | | | | | |
| | 49delG | | | | | | | | | | | | |
| DNMT3A | c.−6G > A | | | 1 | | | | | | | | | |
| DNMT3A | c.72 + | | | 1 | | | | | | | | | |
| | 10G > A | | | | | | | | | | | | |
| DNMT3A | c.73 − | | | 1 | | | | | | | | | |
| | 43T > C | | | | | | | | | | | | |
| DNMT3A | c.759C > T | | | 1 | | | | | | | | | |
| DNMT3A | c.801C > T | | | 1 | | | | | | | | | |
| DNMT3A | c.855 + | | | 1 | | | | | | | | | |
| | 16A > C | | | | | | | | | | | | |
| DNMT3A | c.89A > C | | | 1 | | | | | | | | | |
| DNMT3A | c.939G > A | | | | | | | | | | | | |
| DNMT3A | c.998delA | | | 1 | | | | | | | | | |
| EZH2 | c.*20C > A | | | 1 | | | | | | | | | |
| EZH2 | c.*21delC | | | 1 | | | | | | | | | |
| EZH2 | c.1000 − | | | 1 | | | | | | | | | |
| | 47A > G | | | | | | | | | | | | |
| EZH2 | c.1017T > C | | | 1 | | | | | | | | | |
| EZH2 | c.118 − | | | 1 | | | | | | | | | |
| | 4delT | | | | | | | | | | | | |
| EZH2 | c.118 − | | | 1 | | | | | | | | | |
| | 4dupT | | | | | | | | | | | | |
| EZH2 | c.118 − | | | 1 | | | | | | | | | |
| | 5_118 − | | | | | | | | | | | | |
| | 4delTT | | | | | | | | | | | | |
| EZH2 | c.1240 + | | | 1 | | | | | | | | | |
| | 35T > C | | | | | | | | | | | | |
| EZH2 | c.1240 + | | | 1 | | | | | | | | | |
| | 48G > A | | | | | | | | | | | | |
| EZH2 | c.1240 + | | | 1 | | | | | | | | | |
| | 9C > A | | | | | | | | | | | | |
| EZH2 | c.1281A > G | | | 1 | | | | | | | | | |
| EZH2 | c.1392G > A | | | 1 | | | | | | | | | |
| EZH2 | c.1410 + | | | 1 | | | | | | | | | |
| | 25_1410 + | | | | | | | | | | | | |
| | 33delTTAAAAGAG | | | | | | | | | | | | |
| EZH2 | c.1452A > G | | | 1 | | | | | | | | | |
| EZH2 | c.1459G > A | | | 1 | | | | | | | | | |
| EZH2 | c.1505 + | | | 1 | | | | | | | | | |
| | 12A > G | | | | | | | | | | | | |
| EZH2 | c.1505 + | | | 1 | | | | | | | | | |
| | 20C > G | | | | | | | | | | | | |
| EZH2 | c.1719C > A | | | 1 | | | | | | | | | |
| EZH2 | c.1731G > A | | | 1 | | | | | | | | | |
| EZH2 | c.1852 − | | | 1 | | | | | | | | | |
| | 21T > C | | | | | | | | | | | | |
| EZH2 | c.1852 − | | | 1 | | | | | | | | | |
| | 6C > T | | | | | | | | | | | | |
| EZH2 | c.1852 − | | | 1 | | | | | | | | | |
| | 9A > G | | | | | | | | | | | | |
| EZH2 | c.1865C > T | | | 1 | | | | | | | | | |
| EZH2 | c.1948 − | | | 1 | | | | | | | | | |
| | 29dupA | | | | | | | | | | | | |
| EZH2 | c.1967C > T | | | 1 | | | | | | | | | |
| EZH2 | c.2028T > C | | | 1 | | | | | | | | | |
| EZH2 | c.2035G > A | | | 1 | | | | | | | | | |
| EZH2 | c.2068C > T | | | 1 | | | | | | | | | |
| EZH2 | c.2110 + | | | 1 | | | | | | | | | |
| | 39A > C | | | | | | | | | | | | |
| EZH2 | c.2110 + | | | 1 | | | | | | | | | |
| | 6T > G | | | | | | | | | | | | |
| EZH2 | c.2111 − | | | 1 | | | | | | | | | |
| | 38G > A | | | | | | | | | | | | |
| EZH2 | c.2111 − | | | 1 | | | | | | | | | |
| | 46C > T | | | | | | | | | | | | |
| EZH2 | c.2196 − | | | 1 | | | | | | | | | |
| | 20T > C | | | | | | | | | | | | |
| EZH2 | c.246 + | | | 1 | | | | | | | | | |
| | 37T > C | | | | | | | | | | | | |
| EZH2 | c.247 − | | | 1 | | | | | | | | | |
| | 10dupT | | | | | | | | | | | | |
| EZH2 | c.363 + | | | 1 | | | | | | | | | |
| | 43T > C | | | | | | | | | | | | |
| EZH2 | c.396T > C | | | 1 | | | | | | | | | |
| EZH2 | c.434T > C | | | 1 | | | | | | | | | |
| EZH2 | c.484 + | | | 1 | | | | | | | | | |
| | 13C > T | | | | | | | | | | | | |
| EZH2 | c.485 − | | | 1 | | | | | | | | | |
| | 7G > A | | | | | | | | | | | | |
| EZH2 | c.538C > T | | | 1 | | | | | | | | | |
| EZH2 | c.553G > C | | | 1 | | | | | | | | | |
| EZH2 | c.625 + | | | 1 | | | | | | | | | |
| | 25T > C | | | | | | | | | | | | |
| EZH2 | c.625 + | | | 1 | | | | | | | | | |
| | 26G > T | | | | | | | | | | | | |
| EZH2 | c.625 + | | | 1 | | | | | | | | | |
| | 5G > A | | | | | | | | | | | | |
| EZH2 | c.626 − | | | 1 | | | | | | | | | |
| | 39A > G | | | | | | | | | | | | |
| EZH2 | c.728 + | | | 1 | | | | | | | | | |
| | 19G > C | | | | | | | | | | | | |
| EZH2 | c.72dupG | | | 1 | | | | | | | | | |
| FLT3 | c.1713G > A | | | | | 2 | | | 1 | 1 | | | |
| FLT3 | c.1715A > G | | | | | 1 | | | 1 | 1 | | | |
| FLT3 | c.1782_1805dup24 | | | | | 2 | | | 1 | 1 | | | |
| FLT3 | c.1788_1789ins15 | | | | | 2 | | | 1 | | | | |
| FLT3 | c.1795_1796ins21 | | | | | 2 | | | 1 | 1 | | | |
| FLT3 | c.2503G > A | | | | | 2 | | | 1 | 1 | | | |
| FLT3 | c.2503G > C | | | | | 2 | | | 1 | 1 | | | |
| FLT3 | c.2503G > T | | | | | | | | | | | | |
| FLT3 | c.2508_2510delCAT | | | | | 1 | | | 1 | 1 | | | |
| FLT3 | c.2522A > T | | | | | | | | | | | | |
| FLT3 | c.2524T > G | | | | | | | | | | | | |
| FLT3 | c.2525A > G | | | | | 2 | | | 1 | 1 | | | |
| FLT3 | c.FLT3_ITD | | | | | 2 | | | 1 | 1 | | | |
| IDH2 | c.407A > G | | | | | | | | | | | | |
| IDH2 | c.419G > A | | | | | | | | | | | | |
| IDH2 | c.515G > A | | | | | | | | | | | | |
| JAK2 | c.*4A > C | | | | | | | 1 | | | | | |
| JAK2 | c.*52A > G | | | | | | | 1 | | | | | |
| JAK2 | c.1009A > G | | | | | | | 1 | | | | | |
| JAK2 | C.1174G > A | | | | | | | 1 | | | | | |
| JAK2 | c.1177C > G | | | | | | | 1 | | | | | |
| JAK2 | c.1284T > C | | | | | | | 1 | | | | | |
| JAK2 | c.1323C > T | | | | | | | 1 | | | | | |
| JAK2 | c.1326 + | | | | | | | 1 | | | | | |
| | 25G > C | | | | | | | | | | | | |
| JAK2 | c.1439G > T | | | | | | | 1 | | | | | |
| JAK2 | c.1458T > G | | | | | | | 1 | | | | | |
| JAK2 | c.1514 − | | | | | | | 1 | | | | | |
| | 42C > T | | | | | | | | | | | | |
| JAK2 | c.1542G > A | | | | | | | 1 | | | | | |
| JAK2 | c.1622_1627delGAAATG | | | | | | | 3 | | | | | |
| JAK2 | c.1624_1629delAATGAA | | | | | | | 1 | | | | | |
| JAK2 | c.1641 + | | | | | | | 1 | | | | | |
| | 6T > C | | | | | | | | | | | | |
| JAK2 | c.1710C > T | | | | | | | 1 | | | | | |
| JAK2 | c.1777 − | | | | | | | 1 | | | | | |
| | 7delT | | | | | | | | | | | | |
| JAK2 | c.1777 − | | | | | | | 1 | | | | | |
| | 7dupT | | | | | | | | | | | | |
| JAK2 | c.1849G > T | | | | | | | 6 | | | | | |
| JAK2 | c.1860C > T | | | | | | | 1 | | | | | |
| JAK2 | c.1864 + | | | | | | | 1 | | | | | |
| | 14C > T | | | | | | | | | | | | |
| JAK2 | c.1929T > C | | | | | | | 1 | | | | | |
| JAK2 | c.1992 + | | | | | | | 1 | | | | | |
| | 10A > G | | | | | | | | | | | | |
| JAK2 | c.1992 + | | | | | | | 1 | | | | | |
| | 21A > T | | | | | | | | | | | | |
| JAK2 | c.1992 + | | | | | | | 1 | | | | | |
| | 35T > C | | | | | | | | | | | | |
| JAK2 | c.1993 − | | | | | | | 1 | | | | | |
| | 15G > A | | | | | | | | | | | | |
| JAK2 | c.1993 − | | | | | | | 1 | | | | | |
| | 15G > T | | | | | | | | | | | | |
| JAK2 | c.1993 − | | | | | | | 1 | | | | | |
| | 16C > T | | | | | | | | | | | | |
| JAK2 | c.2132 − | | | | | | | 1 | | | | | |
| | 44A > G | | | | | | | | | | | | |
| JAK2 | c.227 − | | | | | | | 1 | | | | | |
| | 38T > C | | | | | | | | | | | | |
| JAK2 | c.227 − | | | | | | | 1 | | | | | |
| | 7T > C | | | | | | | | | | | | |
| JAK2 | c.2283 + | | | | | | | 1 | | | | | |
| | 50A > T | | | | | | | | | | | | |
| JAK2 | c.2434 + | | | | | | | 1 | | | | | |
| | 18C > A | | | | | | | | | | | | |
| JAK2 | c.2434 + | | | | | | | 1 | | | | | |
| | 35T > C | | | | | | | | | | | | |
| JAK2 | c.2490G > A | | | | | | | 1 | | | | | |
| JAK2 | c.2571 + | | | | | | | 1 | | | | | |
| | 49A > T | | | | | | | | | | | | |
| JAK2 | c.2577T > C | | | | | | | 1 | | | | | |
| JAK2 | c.2586T > C | | | | | | | 1 | | | | | |
| JAK2 | c.2762 − | | | | | | | 1 | | | | | |
| | 10_2762 − | | | | | | | | | | | | |
| | 9delAT | | | | | | | | | | | | |
| JAK2 | c.2850C > T | | | | | | | 1 | | | | | |
| JAK2 | c.2886 + | | | | | | | 1 | | | | | |
| | 42T > C | | | | | | | | | | | | |
| JAK2 | c.2931G > A | | | | | | | 1 | | | | | |
| JAK2 | c.2958C > T | | | | | | | 1 | | | | | |
| JAK2 | c.3059 + | | | | | | | 1 | | | | | |
| | 23A > T | | | | | | | | | | | | |
| JAK2 | c.3059 + | | | | | | | 1 | | | | | |
| | 44C > A | | | | | | | | | | | | |
| JAK2 | c.3188G > A | | | | | | | 1 | | | | | |
| JAK2 | c.3252T > C | | | | | | | 1 | | | | | |
| JAK2 | c.3262T > C | | | | | | | 1 | | | | | |
| JAK2 | c.3288T > A | | | | | | | 1 | | | | | |
| JAK2 | c.3291 + | | | | | | | 1 | | | | | |
| | 14T > C | | | | | | | | | | | | |
| JAK2 | c.3291 + | | | | | | | 1 | | | | | |
| | 16delT | | | | | | | | | | | | |
| JAK2 | c.3291 + | | | | | | | 1 | | | | | |
| | 51T > C | | | | | | | | | | | | |
| JAK2 | c.3291 + | | | | | | | 1 | | | | | |
| | 8T > C | | | | | | | | | | | | |
| JAK2 | c.3323A > G | | | | | | | 1 | | | | | |
| JAK2 | c.380G > A | | | | | | | 1 | | | | | |
| JAK2 | c.489C > T | | | | | | | 1 | | | | | |
| JAK2 | c.525G > A | | | | | | | 1 | | | | | |
| JAK2 | c.579C > T | | | | | | | 1 | | | | | |
| JAK2 | c.614 + | | | | | | | 1 | | | | | |
| | 33T > C | | | | | | | | | | | | |
| JAK2 | c.615 − | | | | | | | 1 | | | | | |
| | 24C > T | | | | | | | | | | | | |
| JAK2 | c.731A > G | | | | | | | 1 | | | | | |
| JAK2 | c.936 + | | | | | | | 1 | | | | | |
| | 13G > A | | | | | | | | | | | | |
| KIT | c.1854G > A | | 1 | | | 2 | 1 | | 1 | 1 | | | |
| KIT | c.2447A > T | | 1 | | | 3 | 1 | | 1 | 1 | | | |
| KRAS | c.*512T > C | 1 | | | 1 | | | | | | | | |
| KRAS | c.*550C > T | 1 | | | 1 | | | | | | | | |
| KRAS | c.111 + | 1 | | | 1 | | | | | | | | |
| | 46T > G | | | | | | | | | | | | |
| KRAS | c.173C > T | 1 | | | 1 | | | | | | | | |
| KRAS | c.179G > A | 1 | | | 1 | | | | | | | | |
| KRAS | c.182A > C | 1 | | | | | | | | | | | |
| KRAS | c.182A > G | 1 | | | | | | | | | | | |
| KRAS | c.183A > T | 1 | | | | | | | | | | | |
| KRAS | c.34G > A | 1 | | | | | | | | | | | |
| KRAS | c.350A > G | 2 | | | 1 | | | | | | | | |
| KRAS | c.35G > A | 1 | | | | | | | | | | | |
| KRAS | c.35G > C | 1 | | | | | | | | | | | |
| KRAS | c.35G > T | 1 | | | | | | | | | | | |
| KRAS | c.38G > A | 1 | | | | | | | | | | | |
| KRAS | c.451 − | 1 | | | 1 | | | | | | | | |
| | 18A > G | | | | | | | | | | | | |
| KRAS | c.451 − | 1 | | | 1 | | | | | | | | |
| | 5494A > G | | | | | | | | | | | | |
| KRAS | c.451 − | 1 | | | 1 | | | | | | | | |
| | 5521G > A | | | | | | | | | | | | |
| KRAS | c.451 − | 1 | | | 1 | | | | | | | | |
| | 5617G > A | | | | | | | | | | | | |
| KRAS | c.451 − | 1 | | | 1 | | | | | | | | |
| | 9G > A | | | | | | | | | | | | |
| KRAS | c.519T > C | 1 | | | 1 | | | | | | | | |
| NF1 | c.*29A > G | | | | | | | | | | | | |
| NF1 | c.*4T > C | | | | | | | | | | | | |
| NF1 | c.1185 + | | | | | | | | | | | | |
| | 30T > C | | | | | | | | | | | | |
| NF1 | c.1186 − | | | | | | | | | | | | |
| | 13delT | | | | | | | | | | | | |
| NF1 | c.1186 − | | | | | | | | | | | | |
| | 13dupT | | | | | | | | | | | | |
| NF1 | c.1261 − | | | | | | | | | | | | |
| | 45G > A | | | | | | | | | | | | |
| NF1 | c.−126G > A | | | | | | | | | | | | |
| NF1 | c.1308G > A | | | | | | | | | | | | |
| NF1 | c.1393 − | | | | | | | | | | | | |
| | 31G > A | | | | | | | | | | | | |
| NF1 | c.1393 − | | | | | | | | | | | | |
| | 32T > C | | | | | | | | | | | | |
| NF1 | c.1528 − | | | | | | | | | | | | |
| | 29dupT | | | | | | | | | | | | |
| NF1 | c.1641 + | | | | | | | | | | | | |
| | 39T > C | | | | | | | | | | | | |
| NF1 | c.168C > T | | | | | | | | | | | | |
| NF1 | c.1721 + | | | | | | | | | | | | |
| | 72C > T | | | | | | | | | | | | |
| NF1 | c.1750A > T | | | | | | | | | | | | |
| NF1 | c.1845 + | | | | | | | | | | | | |
| | 13A > T | | | | | | | | | | | | |
| NF1 | c.1845 + | | | | | | | | | | | | |
| | 45G > A | | | | | | | | | | | | |
| NF1 | c.1845 + | | | | | | | | | | | | |
| | 45G > T | | | | | | | | | | | | |
| NF1 | c.1846 − | | | | | | | | | | | | |
| | 12A > T | | | | | | | | | | | | |
| NF1 | c.1846 − | | | | | | | | | | | | |
| | 39C > T | | | | | | | | | | | | |
| NF1 | c.1994C > T | | | | | | | | | | | | |
| NF1 | c.2022C > T | | | | | | | | | | | | |
| NF1 | c.2034G > A | | | | | | | | | | | | |
| NF1 | c.204 + | | | | | | | | | | | | |
| | 51G > C | | | | | | | | | | | | |
| NF1 | c.2224G > T | | | | | | | | | | | | |
| NF1 | c.2252 − | | | | | | | | | | | | |
| | 31A > T | | | | | | | | | | | | |
| NF1 | c.2252 − | | | | | | | | | | | | |
| | 34T > C | | | | | | | | | | | | |
| NF1 | c.2252 − | | | | | | | | | | | | |
| | 46C > T | | | | | | | | | | | | |
| NF1 | c.−22G > C | | | | | | | | | | | | |
| NF1 | c.2544G > A | | | | | | | | | | | | |
| NF1 | c.2553C > T | | | | | | | | | | | | |
| NF1 | c.2851 − | | | | | | | | | | | | |
| | 16T > C | | | | | | | | | | | | |
| NF1 | c.288 + | | | | | | | | | | | | |
| | 41G > A | | | | | | | | | | | | |
| NF1 | c.2921A > G | | | | | | | | | | | | |
| NF1 | c.2985G > C | | | | | | | | | | | | |
| NF1 | c.3114 − | | | | | | | | | | | | |
| | 7T > C | | | | | | | | | | | | |
| NF1 | c.3197 + | | | | | | | | | | | | |
| | 10G > A | | | | | | | | | | | | |
| NF1 | c.3197 + | | | | | | | | | | | | |
| | 17C > T | | | | | | | | | | | | |
| NF1 | c.3197 + | | | | | | | | | | | | |
| | 23G > A | | | | | | | | | | | | |
| NF1 | c.3197 + | | | | | | | | | | | | |
| | 47G > A | | | | | | | | | | | | |
| NF1 | c.3198 − | | | | | | | | | | | | |
| | 11_3198 − | | | | | | | | | | | | |
| | 4delTTTTTTTT | | | | | | | | | | | | |
| NF1 | c.3198 − | | | | | | | | | | | | |
| | 3dupC | | | | | | | | | | | | |
| NF1 | c.3198 − | | | | | | | | | | | | |
| | 4dupT | | | | | | | | | | | | |
| NF1 | c.3198 − | | | | | | | | | | | | |
| | 4T > C | | | | | | | | | | | | |
| NF1 | c.3198 − | | | | | | | | | | | | |
| | 5_3198 − | | | | | | | | | | | | |
| | 4dupTT | | | | | | | | | | | | |
| NF1 | c.3198 − | | | | | | | | | | | | |
| | 6_3198 − | | | | | | | | | | | | |
| | 4dupTTT | | | | | | | | | | | | |
| NF1 | c.3198 − | | | | | | | | | | | | |
| | 7_3198 − | | | | | | | | | | | | |
| | 4dupTTTT | | | | | | | | | | | | |
| NF1 | c.3270A > C | | | | | | | | | | | | |
| NF1 | c.340C > T | | | | | | | | | | | | |
| NF1 | c.3496 + | | | | | | | | | | | | |
| | 19T > C | | | | | | | | | | | | |
| NF1 | c.3496 + | | | | | | | | | | | | |
| | 33C > A | | | | | | | | | | | | |
| NF1 | c.3496 + | | | | | | | | | | | | |
| | 43A > C | | | | | | | | | | | | |
| NF1 | c.3498C > T | | | | | | | | | | | | |
| NF1 | c.3686A > G | | | | | | | | | | | | |
| NF1 | c.369C > G | | | | | | | | | | | | |
| NF1 | c.3709 − | | | | | | | | | | | | |
| | 47A > G | | | | | | | | | | | | |
| NF1 | c.3867C > T | | | | | | | | | | | | |
| NF1 | c.3870 + | | | | | | | | | | | | |
| | 25A > G | | | | | | | | | | | | |
| NF1 | c.3870 + | | | | | | | | | | | | |
| | 50A > G | | | | | | | | | | | | |
| NF1 | c.3871 − | | | | | | | | | | | | |
| | 31A > G | | | | | | | | | | | | |
| NF1 | c.3974 + | | | | | | | | | | | | |
| | 36_3974 + | | | | | | | | | | | | |
| | 37dupGT | | | | | | | | | | | | |
| NF1 | c.3974 + | | | | | | | | | | | | |
| | 36G > T | | | | | | | | | | | | |
| NF1 | c.3974 + | | | | | | | | | | | | |
| | 37_3974 + | | | | | | | | | | | | |
| | 38insG | | | | | | | | | | | | |
| NF1 | c.3974 + | | | | | | | | | | | | |
| | 38T > G | | | | | | | | | | | | |
| NF1 | c.3974 + | | | | | | | | | | | | |
| | 40T > G | | | | | | | | | | | | |
| NF1 | c.3974 + | | | | | | | | | | | | |
| | 43_3974 + | | | | | | | | | | | | |
| | 44insG | | | | | | | | | | | | |
| NF1 | c.3974 + | | | | | | | | | | | | |
| | 46T > G | | | | | | | | | | | | |
| NF1 | c.3974 + | | | | | | | | | | | | |
| | 48_3974 + | | | | | | | | | | | | |
| | 49delTT | | | | | | | | | | | | |
| NF1 | c.3974 + | | | | | | | | | | | | |
| | 48_3974 + | | | | | | | | | | | | |
| | 49dupTT | | | | | | | | | | | | |
| NF1 | c.3974 + | | | | | | | | | | | | |
| | 49delT | | | | | | | | | | | | |
| NF1 | c.3974 + | | | | | | | | | | | | |
| | 49dupT | | | | | | | | | | | | |
| NF1 | c.4110 + | | | | | | | | | | | | |
| | 3780C > T | | | | | | | | | | | | |
| NF1 | c.4111 − | | | | | | | | | | | | |
| | 48_4111 − | | | | | | | | | | | | |
| | 23dup26 | | | | | | | | | | | | |
| NF1 | c.4182T > C | | | | | | | | | | | | |
| NF1 | c.4206A > G | | | | | | | | | | | | |
| NF1 | c.4368 − | | | | | | | | | | | | |
| | 46G > C | | | | | | | | | | | | |
| NF1 | c.4514 + | | | | | | | | | | | | |
| | 12C > T | | | | | | | | | | | | |
| NF1 | c.4514 + | | | | | | | | | | | | |
| | 35C > G | | | | | | | | | | | | |
| NF1 | c.4515 − | | | | | | | | | | | | |
| | 39A > G | | | | | | | | | | | | |
| NF1 | c.4772 + | | | | | | | | | | | | |
| | 29689A > G | | | | | | | | | | | | |
| NF1 | c.4772 + | | | | | | | | | | | | |
| | 29883T > G | | | | | | | | | | | | |
| NF1 | c.4772 + | | | | | | | | | | | | |
| | 36T > C | | | | | | | | | | | | |
| NF1 | c.4773 − | | | | | | | | | | | | |
| | 17877_4773 − | | | | | | | | | | | | |
| | 17876delAG | | | | | | | | | | | | |
| NF1 | c.4773 − | | | | | | | | | | | | |
| | 20174A > C | | | | | | | | | | | | |
| NF1 | c.4773 − | | | | | | | | | | | | |
| | 20184delG | | | | | | | | | | | | |
| NF1 | c.4773 − | | | | | | | | | | | | |
| | 20194G > A | | | | | | | | | | | | |
| NF1 | c.4773 − | | | | | | | | | | | | |
| | 20367C > T | | | | | | | | | | | | |
| NF1 | c.4773 − | | | | | | | | | | | | |
| | 20371T > A | | | | | | | | | | | | |
| NF1 | c.4773 − | | | | | | | | | | | | |
| | 20399G > A | | | | | | | | | | | | |
| NF1 | c.4773 − | | | | | | | | | | | | |
| | 20486T > C | | | | | | | | | | | | |
| NF1 | c.4773 − | | | | | | | | | | | | |
| | 20567T > C | | | | | | | | | | | | |
| NF1 | c.4773 − | | | | | | | | | | | | |
| | 20708T > C | | | | | | | | | | | | |
| NF1 | c.4773 − | | | | | | | | | | | | |
| | 20889A > T | | | | | | | | | | | | |
| NF1 | c.4773 − | | | | | | | | | | | | |
| | 20968C > A | | | | | | | | | | | | |
| NF1 | c.4773 − | | | | | | | | | | | | |
| | 21063C > G | | | | | | | | | | | | |
| NF1 | c.4773 − | | | | | | | | | | | | |
| | 21229G > C | | | | | | | | | | | | |
| NF1 | c.4773 − | | | | | | | | | | | | |
| | 21352T > C | | | | | | | | | | | | |
| NF1 | c.4773 − | | | | | | | | | | | | |
| | 21440A > G | | | | | | | | | | | | |
| NF1 | c.4773 − | | | | | | | | | | | | |
| | 29479G > A | | | | | | | | | | | | |
| NF1 | c.4773 − | | | | | | | | | | | | |
| | 29550C > T | | | | | | | | | | | | |
| NF1 | c.4773 − | | | | | | | | | | | | |
| | 30170A > G | | | | | | | | | | | | |
| NF1 | c.4773 − | | | | | | | | | | | | |
| | 5353G > A | | | | | | | | | | | | |
| NF1 | c.4773 − | | | | | | | | | | | | |
| | 5368C > T | | | | | | | | | | | | |
| NF1 | c.4773 − | | | | | | | | | | | | |
| | 5461delA | | | | | | | | | | | | |
| NF1 | c.4773 − | | | | | | | | | | | | |
| | 5461dupA | | | | | | | | | | | | |
| NF1 | c.4773 − | | | | | | | | | | | | |
| | 5462_4773 − | | | | | | | | | | | | |
| | 5461delAA | | | | | | | | | | | | |
| NF1 | c.4773 − | | | | | | | | | | | | |
| | 5463_4773 − | | | | | | | | | | | | |
| | 5461delAAA | | | | | | | | | | | | |
| NF1 | c.4773 − | | | | | | | | | | | | |
| | 5475A > T | | | | | | | | | | | | |
| NF1 | c.4773 − | | | | | | | | | | | | |
| | 5476A > T | | | | | | | | | | | | |
| NF1 | c.4773 − | | | | | | | | | | | | |
| | 5477dupT | | | | | | | | | | | | |
| NF1 | c.4773 − | | | | | | | | | | | | |
| | 6806G > A | | | | | | | | | | | | |
| NF1 | c.4773 − | | | | | | | | | | | | |
| | 6844T > C | | | | | | | | | | | | |
| NF1 | c.4773 − | | | | | | | | | | | | |
| | 6867C > G | | | | | | | | | | | | |
| NF1 | c.4773 − | | | | | | | | | | | | |
| | 6962G > A | | | | | | | | | | | | |
| NF1 | c.4773 − | | | | | | | | | | | | |
| | 7106G > T | | | | | | | | | | | | |
| NF1 | c.4773 − | | | | | | | | | | | | |
| | 7179C > T | | | | | | | | | | | | |
| NF1 | c.4773 − | | | | | | | | | | | | |
| | 7300G > T | | | | | | | | | | | | |
| NF1 | c.4773 − | | | | | | | | | | | | |
| | 7333T > C | | | | | | | | | | | | |
| NF1 | c.4773 − | | | | | | | | | | | | |
| | 7365C > T | | | | | | | | | | | | |
| NF1 | c.4866G > A | | | | | | | | | | | | |
| NF1 | c.5172G > A | | | | | | | | | | | | |
| NF1 | c.5205 + | | | | | | | | | | | | |
| | 23T > C | | | | | | | | | | | | |
| NF1 | c.528T > A | | | | | | | | | | | | |
| NF1 | c.5546 + | | | | | | | | | | | | |
| | 19T > A | | | | | | | | | | | | |
| NF1 | c.5750 − | | | | | | | | | | | | |
| | 168delT | | | | | | | | | | | | |
| NF1 | c.5750 − | | | | | | | | | | | | |
| | 43A > G | | | | | | | | | | | | |
| NF1 | c.5750 − | | | | | | | | | | | | |
| | 75C > T | | | | | | | | | | | | |
| NF1 | c.5820G > A | | | | | | | | | | | | |
| NF1 | c.60 + | | | | | | | | | | | | |
| | 16C > A | | | | | | | | | | | | |
| NF1 | c.6084 + | | | | | | | | | | | | |
| | 11T > C | | | | | | | | | | | | |
| NF1 | c.6084 + | | | | | | | | | | | | |
| | 8C > G | | | | | | | | | | | | |
| NF1 | c.6085 − | | | | | | | | | | | | |
| | 17delA | | | | | | | | | | | | |
| NF1 | c.6085 − | | | | | | | | | | | | |
| | 17dupA | | | | | | | | | | | | |
| NF1 | c.6085 − | | | | | | | | | | | | |
| | 18_6085 − | | | | | | | | | | | | |
| | 17dupAA | | | | | | | | | | | | |
| NF1 | c.6085 − | | | | | | | | | | | | |
| | 19_6085 − | | | | | | | | | | | | |
| | 17dupAAA | | | | | | | | | | | | |
| NF1 | c.6085 − | | | | | | | | | | | | |
| | 27A > T | | | | | | | | | | | | |
| NF1 | c.6085 − | | | | | | | | | | | | |
| | 28delT | | | | | | | | | | | | |
| NF1 | c.6085 − | | | | | | | | | | | | |
| | 28dupT | | | | | | | | | | | | |
| NF1 | c.6085 − | | | | | | | | | | | | |
| | 28T > A | | | | | | | | | | | | |
| NF1 | c.6085 − | | | | | | | | | | | | |
| | 29_6085 − | | | | | | | | | | | | |
| | 28delTT | | | | | | | | | | | | |
| NF1 | c.6085 − | | | | | | | | | | | | |
| | 29_6085 − | | | | | | | | | | | | |
| | 28insA | | | | | | | | | | | | |
| NF1 | c.6085 − | | | | | | | | | | | | |
| | 29T > A | | | | | | | | | | | | |
| NF1 | c.6085 − | | | | | | | | | | | | |
| | 30_6085 − | | | | | | | | | | | | |
| | 28delTTT | | | | | | | | | | | | |
| NF1 | c.6085 − | | | | | | | | | | | | |
| | 30T > A | | | | | | | | | | | | |
| NF1 | c.61 − | | | | | | | | | | | | |
| | 13C > T | | | | | | | | | | | | |
| NF1 | c.61 − | | | | | | | | | | | | |
| | 14delT | | | | | | | | | | | | |
| NF1 | c.61 − | | | | | | | | | | | | |
| | 14dupT | | | | | | | | | | | | |
| NF1 | c.61 − | | | | | | | | | | | | |
| | 4delT | | | | | | | | | | | | |
| NF1 | c.61 − | | | | | | | | | | | | |
| | 4dupT | | | | | | | | | | | | |
| NF1 | c.6330C > T | | | | | | | | | | | | |
| NF1 | c.6364 + | | | | | | | | | | | | |
| | 32A > G | | | | | | | | | | | | |
| NF1 | c.655 − | | | | | | | | | | | | |
| | 22A > C | | | | | | | | | | | | |
| NF1 | c.655 − | | | | | | | | | | | | |
| | 29T > C | | | | | | | | | | | | |
| NF1 | c.655 − | | | | | | | | | | | | |
| | 35A > C | | | | | | | | | | | | |
| NF1 | c.6579 + | | | | | | | | | | | | |
| | 45T > A | | | | | | | | | | | | |
| NF1 | c.6580 − | | | | | | | | | | | | |
| | 19A > G | | | | | | | | | | | | |
| NF1 | c.6858 + | | | | | | | | | | | | |
| | 40A > G | | | | | | | | | | | | |
| NF1 | c.6859 − | | | | | | | | | | | | |
| | 14_6859 − | | | | | | | | | | | | |
| | 13delAT | | | | | | | | | | | | |
| NF1 | c.7000 − | | | | | | | | | | | | |
| | 22A > G | | | | | | | | | | | | |
| NF1 | c.702G > A | | | | | | | | | | | | |
| NF1 | c.7126 + | | | | | | | | | | | | |
| | 37C > G | | | | | | | | | | | | |
| NF1 | c.7127 − | | | | | | | | | | | | |
| | 11_7127 − | | | | | | | | | | | | |
| | 8delGTTT | | | | | | | | | | | | |
| NF1 | c.7127 − | | | | | | | | | | | | |
| | 11_7127 − | | | | | | | | | | | | |
| | 8dupGTTT | | | | | | | | | | | | |
| NF1 | c.7127 − | | | | | | | | | | | | |
| | 19_7127 − | | | | | | | | | | | | |
| | 8del12 | | | | | | | | | | | | |
| NF1 | c.7258 + | | | | | | | | | | | | |
| | 43A > G | | | | | | | | | | | | |
| NF1 | c.7259 − | | | | | | | | | | | | |
| | 17C > T | | | | | | | | | | | | |
| NF1 | c.730 + | | | | | | | | | | | | |
| | 15G > T | | | | | | | | | | | | |
| NF1 | c.730 + | | | | | | | | | | | | |
| | 30_730 + | | | | | | | | | | | | |
| | 32dupTTT | | | | | | | | | | | | |
| NF1 | c.730 + | | | | | | | | | | | | |
| | 31_730 + | | | | | | | | | | | | |
| | 32delTT | | | | | | | | | | | | |
| NF1 | c.730 + | | | | | | | | | | | | |
| | 31_730 + | | | | | | | | | | | | |
| | 32dupTT | | | | | | | | | | | | |
| NF1 | c.730 + | | | | | | | | | | | | |
| | 32delT | | | | | | | | | | | | |
| NF1 | c.730 + | | | | | | | | | | | | |
| | 32dupT | | | | | | | | | | | | |
| NF1 | c.730 + | | | | | | | | | | | | |
| | 33G > T | | | | | | | | | | | | |
| NF1 | c.730 + | | | | | | | | | | | | |
| | 35C > T | | | | | | | | | | | | |
| NF1 | c.7305A > G | | | | | | | | | | | | |
| NF1 | c.7347T > C | | | | | | | | | | | | |
| NF1 | c.7394 + | | | | | | | | | | | | |
| | 21A > G | | | | | | | | | | | | |
| NF1 | c.7395 − | | | | | | | | | | | | |
| | 29G > A | | | | | | | | | | | | |
| NF1 | c.7398A > G | | | | | | | | | | | | |
| NF1 | c.7468G > C | | | | | | | | | | | | |
| NF1 | c.7552 + | | | | | | | | | | | | |
| | 24delA | | | | | | | | | | | | |
| NF1 | c.7675 + | | | | | | | | | | | | |
| | 46G > A | | | | | | | | | | | | |
| NF1 | c.7676 − | | | | | | | | | | | | |
| | 33G > A | | | | | | | | | | | | |
| NF1 | c.7692C > T | | | | | | | | | | | | |
| NF1 | c.7807 − | | | | | | | | | | | | |
| | 13dupT | | | | | | | | | | | | |
| NF1 | c.7908 − | | | | | | | | | | | | |
| | 19G > T | | | | | | | | | | | | |
| NF1 | c.7908 − | | | | | | | | | | | | |
| | 8delT | | | | | | | | | | | | |
| NF1 | c.8050 + | | | | | | | | | | | | |
| | 20G > A | | | | | | | | | | | | |
| NF1 | c.8050 + | | | | | | | | | | | | |
| | 22delA | | | | | | | | | | | | |
| NF1 | c.8050 + | | | | | | | | | | | | |
| | 49C > T | | | | | | | | | | | | |
| NF1 | c.8051 − | | | | | | | | | | | | |
| | 50A > G | | | | | | | | | | | | |
| NF1 | c.8070G > T | | | | | | | | | | | | |
| NF1 | c.8088G > A | | | | | | | | | | | | |
| NF1 | c.8098 − | | | | | | | | | | | | |
| | 34delA | | | | | | | | | | | | |
| NF1 | c.8098 − | | | | | | | | | | | | |
| | 45A > C | | | | | | | | | | | | |
| NF1 | c.846G > A | | | | | | | | | | | | |
| NF1 | c.889 − | | | | | | | | | | | | |
| | 25_889 − | | | | | | | | | | | | |
| | 21delTTATC | | | | | | | | | | | | |
| NRAS | c.176C > G | 1 | | | | | | | | | | | |
| NRAS | c.181C > A | 1 | | | | | | | | | | | |
| NRAS | c.34G > A | 1 | | | | | | | | | | | |
| NRAS | c.35G > A | 1 | | | | | | | | | | | |
| NRAS | c.35G > T | 1 | | | | | | | | | | | |
| NRAS | c.38G > A | 1 | | | | | | | | | | | |
| NRAS | c.38G > T | 1 | | | | | | | | | | | |
| PDGFRA | c.2021C > T | | 1 | | | 2 | 1 | | | 1 | | | |
| PML | c.647C > T | | | | | | | | | | | | |
| PTPN11 | c.1028G > A | | | | | | | | | | | | |
| PTPN11 | c.1052G > A | | | | | | | | | | | | |
| PTPN11 | c.1093 − | | | | | | | | | | | | |
| | 9C > A | | | | | | | | | | | | |
| PTPN11 | c.1221A > G | | | | | | | | | | | | |
| PTPN11 | c.1379 + | | | | | | | | | | | | |
| | 20C > T | | | | | | | | | | | | |
| PTPN11 | c.1380 − | | | | | | | | | | | | |
| | 16G > T | | | | | | | | | | | | |
| PTPN11 | c.14 + | | | | | | | | | | | | |
| | 25G > C | | | | | | | | | | | | |
| PTPN11 | c.14 + | | | | | | | | | | | | |
| | 42C > T | | | | | | | | | | | | |
| PTPN11 | c.1447 + | | | | | | | | | | | | |
| | 26G > A | | | | | | | | | | | | |
| PTPN11 | c.1448 − | | | | | | | | | | | | |
| | 38G > C | | | | | | | | | | | | |
| PTPN11 | c.1507G > A | | | | | | | | | | | | |
| PTPN11 | c.182A > C | | | | | | | | | | | | |
| PTPN11 | c.215C > T | | | | | | | | | | | | |
| PTPN11 | c.226G > C | | | | | | | | | | | | |
| PTPN11 | c.227A > G | | | | | | | | | | | | |
| PTPN11 | c.255C > T | | | | | | | | | | | | |
| PTPN11 | c.332 + | | | | | | | | | | | | |
| | 17T > G | | | | | | | | | | | | |
| PTPN11 | c.525 + | | | | | | | | | | | | |
| | 12G > C | | | | | | | | | | | | |
| PTPN11 | c.525 + | | | | | | | | | | | | |
| | 34A > G | | | | | | | | | | | | |
| PTPN11 | c.526 − | | | | | | | | | | | | |
| | 17T > C | | | | | | | | | | | | |
| PTPN11 | c.526 − | | | | | | | | | | | | |
| | 33_526 − | | | | | | | | | | | | |
| | 31delCAA | | | | | | | | | | | | |
| PTPN11 | c.558G > T | | | | | | | | | | | | |
| PTPN11 | c.616T > C | | | | | | | | | | | | |
| PTPN11 | c.642 + | | | | | | | | | | | | |
| | 138C > T | | | | | | | | | | | | |
| PTPN11 | c.642 + | | | | | | | | | | | | |
| | 157_642 + | | | | | | | | | | | | |
| | 159delTTT | | | | | | | | | | | | |
| PTPN11 | c.642 + | | | | | | | | | | | | |
| | 158_642 + | | | | | | | | | | | | |
| | 159delTT | | | | | | | | | | | | |
| PTPN11 | c.642 + | | | | | | | | | | | | |
| | 158_642 + | | | | | | | | | | | | |
| | 159dupTT | | | | | | | | | | | | |
| PTPN11 | c.642 + | | | | | | | | | | | | |
| | 159delT | | | | | | | | | | | | |
| PTPN11 | c.642 + | | | | | | | | | | | | |
| | 159dupT | | | | | | | | | | | | |
| PTPN11 | c.854 − | | | | | | | | | | | | |
| | 21C > T | | | | | | | | | | | | |
| PTPN11 | c.854 − | | | | | | | | | | | | |
| | 30T > C | | | | | | | | | | | | |
| PTPN11 | c.951G > A | | | | | | | | | | | | |
| TET2 | c.100C > T | | | 1 | | | | | | | | | |
| TET2 | c.1064G > A | | | 1 | | | | | | | | | |
| TET2 | c.1069delA | | | 1 | | | | | | | | | |
| TET2 | c.1088C > T | | | 1 | | | | | | | | | |
| TET2 | c.1297_1299delGAA | | | 1 | | | | | | | | | |
| TET2 | c.1395A > G | | | 1 | | | | | | | | | |
| TET2 | c.1630C > T | | | 1 | | | | | | | | | |
| TET2 | c.1884G > A | | | 1 | | | | | | | | | |
| TET2 | c.2039G > T | | | 1 | | | | | | | | | |
| TET2 | c.2116C > T | | | 1 | | | | | | | | | |
| TET2 | c.2185C > T | | | 1 | | | | | | | | | |
| TET2 | c.2299A > G | | | 1 | | | | | | | | | |
| TET2 | c.22C > G | | | 1 | | | | | | | | | |
| TET2 | c.2429A > G | | | 1 | | | | | | | | | |
| TET2 | c.2596C > T | | | 1 | | | | | | | | | |
| TET2 | c.2599T > C | | | 1 | | | | | | | | | |
| TET2 | c.2732C > A | | | 1 | | | | | | | | | |
| TET2 | c.2771A > G | | | 1 | | | | | | | | | |
| TET2 | c.3090G > A | | | 1 | | | | | | | | | |
| TET2 | c.3095T > G | | | 1 | | | | | | | | | |
| TET2 | c.3116C > T | | | 1 | | | | | | | | | |
| TET2 | c.3117G > A | | | 1 | | | | | | | | | |
| TET2 | c.−31C > T | | | 1 | | | | | | | | | |
| TET2 | c.3251A > C | | | 1 | | | | | | | | | |
| TET2 | c.3380_3381delAA | | | 1 | | | | | | | | | |
| TET2 | c.3409 + | | | 1 | | | | | | | | | |
| | 18A > G | | | | | | | | | | | | |
| TET2 | c.3409 + | | | 1 | | | | | | | | | |
| | 37A > C | | | | | | | | | | | | |
| TET2 | c.3409 + | | | 1 | | | | | | | | | |
| | 42G > T | | | | | | | | | | | | |
| TET2 | c.3409 + | | | 1 | | | | | | | | | |
| | 70G > A | | | | | | | | | | | | |
| TET2 | c.3595 − | | | 1 | | | | | | | | | |
| | 4G > A | | | | | | | | | | | | |
| TET2 | c.37A > C | | | 1 | | | | | | | | | |
| TET2 | c.3803 + | | | 1 | | | | | | | | | |
| | 45G > A | | | | | | | | | | | | |
| TET2 | c.3804 − | | | 1 | | | | | | | | | |
| | 34C > A | | | | | | | | | | | | |
| TET2 | c.3863G > A | | | 1 | | | | | | | | | |
| TET2 | c.3899T > G | | | 1 | | | | | | | | | |
| TET2 | c.3954 + | | | 1 | | | | | | | | | |
| | 26C > A | | | | | | | | | | | | |
| TET2 | c.4045 − | | | 1 | | | | | | | | | |
| | 27G > A | | | | | | | | | | | | |
| TET2 | c.4045 − | | | 1 | | | | | | | | | |
| | 35C > A | | | | | | | | | | | | |
| TET2 | c.4075C > T | | | 1 | | | | | | | | | |
| TET2 | c.4140T > C | | | 1 | | | | | | | | | |
| TET2 | c.4182 + | | | 1 | | | | | | | | | |
| | 9G > A | | | | | | | | | | | | |
| TET2 | c.434G > A | | | 1 | | | | | | | | | |
| TET2 | c.5103G > A | | | 1 | | | | | | | | | |
| TET2 | c.5162T > G | | | 1 | | | | | | | | | |
| TET2 | c.5167C > T | | | 1 | | | | | | | | | |
| TET2 | c.521C > A | | | 1 | | | | | | | | | |
| TET2 | c.5284A > G | | | 1 | | | | | | | | | |
| TET2 | c.5333A > G | | | 1 | | | | | | | | | |
| TET2 | c.5352C > T | | | 1 | | | | | | | | | |
| TET2 | c.606C > T | | | 1 | | | | | | | | | |
| TET2 | c.652G > A | | | 1 | | | | | | | | | |
| TET2 | c.86C > G | | | 1 | | | | | | | | | |
| TET2 | c.−93G > A | | | 1 | | | | | | | | | |
| TET2 | c.972A > G | | | 1 | | | | | | | | | |

✓ = sensitive;
X = resistant

### Example 3—Embodiments

Embodiments of the present invention include:

A1. An in vitro method for assessing the functional effect of a somatic variation in a target sequence comprising:

(a) obtaining a biological sample from a subject;

(b) performing a genotyping assay on the biological sample to identify a variant of unknown significance (VUS) at a target sequence;

(c) generating a population of cells containing the nucleotide modification at the target sequence; and

(d) determining if the population of cells containing the nucleotide modification exhibits at least one different functional characteristic as compared to a population of cells not containing the nucleotide modification.

A2. The method of any of the previous or subsequent embodiments, wherein the target sequence is within a gene associated with chemosensitivity.
A3. The method of any of the previous or subsequent embodiments, wherein the functional characteristic is chemosensitivity.
A4. The method of any of the previous or subsequent embodiments, wherein the biological sample is cell-free nucleic acid, a solid tissue biopsy, a liquid biopsy, blood, bone marrow, urine, lymph, another bodily fluid, or a tissue sample.
A5. The method of any of the previous or subsequent embodiments, wherein the biological sample includes genetic material from a cancerous cell.
A6. The method of any of the previous or subsequent embodiments, wherein generating a population of cells containing the nucleotide modification at the target sequence comprises:

(a) providing a repair oligonucleotide, wherein the repair oligonucleotide comprises the sequence of the variant of unknown significance;

(b) providing a Cas9 guide RNA (gRNA) that individually recognize a portion of the gene recognized by the repair oligonucleotide;

(c) co-transfecting a population of cells with (i) an expression system capable of expressing Cas9 and the guide RNA, and (ii) the repair oligonucleotide and guide RNA, wherein the expression system is capable of introducing the oligonucleotide having the nucleotide modification into the target sequence in the population of cells; and

(d) confirming the presence of cells containing the nucleotide modification at the target sequence.

A7. The method of any of the previous or subsequent embodiments, wherein generating a population of cells containing the nucleotide modification at the target sequence comprises expanding a cell line derived from the biological sample taken from the subject.
A8. The method of any of the previous or subsequent embodiments, further comprising treating the subject based on the at least one different functional characteristic exhibited by the population of cells containing the nucleotide modification.
A9. The method of any of the previous or subsequent embodiments, wherein at least some of the plurality of nucleotide variants have been assessed and correlated with an effect on a function of the target sequence.
B1. A method of treating a subject comprising:

(a) obtaining a biological sample from the subject;

(b) performing a genotyping assay on the biological sample to identify a variant of unknown significance (VUS) in a target sequence;

(c) providing a database correlating a VUS in the target sequence with chemosensitivity; and

(d) determining, based on the VUS detected, and the correlation with the database whether a treatment option should be performed.

B2. The method of embodiment B1 wherein the identification of a VUS is performed by any of the previous or subsequent embodiments.
C1. An in vitro method for assessing the functional effect of a genetic variant in a target sequence comprising:

introducing a plurality of nucleotide modifications, each comprising an individual variant of unknown significance, at a plurality of sites in a target sequence; and

determining for each of the plurality of variants of unknown significance, whether the nucleotide change is associated with a change in a functional characteristic for the target sequence.

C2. The method of any of the previous or subsequent embodiments, further comprising generating a database of the plurality of variants of unknown significance.
C3. The method of any of the previous or subsequent embodiments, wherein the plurality of variants of unknown significance are generated using saturation genome editing.
C4. The method of any of the previous or subsequent embodiments, further comprising

(a) providing a plurality of a repair oligonucleotides, each comprising a portion of the target sequence and each individually containing a nucleotide modification at a different position of the target sequence;

(b) providing a library of Cas9 guide RNAs (gRNAs) that individually recognize a portion of the target sequence recognized by at least some the plurality of repair oligonucleotides;

(c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence;

(d) confirming the presence of cells containing at least one of the nucleotide modifications from the plurality of repair oligonucleotides in the population of cells; and

(e) determining if the cells containing the at least one nucleotide modification exhibit at least one different functional characteristic as compared to cells not containing the nucleotide modification.

C5. The method of any of the previous or subsequent embodiments, further comprising:

obtaining a biological sample from a first subject; and

predicting the effect of the variant of unknown significance in the subject.

C6. The method of any of the previous or subsequent embodiments, wherein the functional characteristic is chemosensitivity.
C7. The method of any of the previous or subsequent embodiments, wherein the variant of unknown significance was a previously identified mutation in a biological sample from a second subject who is different than the first subject.
C8. The method of any of the previous or subsequent embodiments, wherein the biological sample is cell-free nucleic acid, a solid tissue biopsy, a liquid biopsy, blood, urine, lymph, another bodily fluid, or a tissue sample.
C9. The method of any of the previous or subsequent embodiments, wherein the biological sample includes genetic material from a cancerous cell.
C10. The method of any of the previous or subsequent embodiments, wherein at least some of the plurality of nucleotide variants have been assessed and correlated with an effect on a function of the target sequence.
D1. An in vitro method for assessing the impact of a variant of unknown significance in a target sequence on chemosensitivity, the method comprising:

(a) providing a plurality of a repair oligonucleotides, each comprising a portion of the target sequence and each individually containing a nucleotide modification corresponding to a VUS at a different position of the target sequence;

(b) providing a library of Cas9 guide RNAs (gRNAs) that individually recognize a portion of the target sequence recognized by a defined group of the repair oligonucleotides;

(c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence;

(d) confirming the presence of cells containing at least one of the nucleotide modifications from the plurality of repair oligonucleotides in the population of cells; and

(e) determining if the cells containing at least one of the nucleotide modifications exhibit different chemosensitivity than cells not containing the nucleotide modification.

D2. The method of embodiment D1, wherein the VUS is identified using any of the previous and/or subsequent embodiments.
E1. A method of determining a treatment option for a subject comprising:

(a) obtaining a biological sample from the subject;

(b) performing a genotyping assay on the biological sample to identify a variant of unknown significance (VUS) in a target sequence;

(c) providing a database correlating variants in the target sequence with a diagnosis; and

(d) determining a treatment option for the subject based on the variant detected and the correlation with the database.

E2. The method of embodiment E1, wherein the VUS is identified using any of the previous and/or subsequent embodiments.
F1. A composition comprising library of cells comprising a defined set of variants of unknown significance (VUS) for a target sequence.
F2. The composition of F1, wherein the library of cells is made by the method of any one of the previous or subsequent embodiments, and comprising a plurality of nucleotide variants at known positions in the target sequence.
F3. The composition of any of the previous or subsequent embodiments, wherein at least some of the plurality of nucleotide variants have been assessed for an effect on a function of the target sequence.
G1. A system comprising a database comprising a compilation of a plurality of nucleotide variants of unknown significance (VUS) at known positions in the target sequence.
G2. The system of any of the previous or subsequent embodiments, made by a method of any of the previous or subsequent embodiments.
G3. The system of any of the previous or subsequent embodiments, wherein at least some of the plurality of nucleotide variants have been assessed for an effect on a function of the target sequence.
G4. The system of any of the previous or subsequent embodiments further comprising a computer.
G5. The system of any of the previous or subsequent embodiments further comprising a computer-implemented instructions.
H1. A composition comprising library of cells for assessing the functional effect of a somatic variation in a target sequence comprising:

one or more populations of cells each containing a nucleotide modification at a target sequence, wherein the nucleotide modification exhibits at least one different functional characteristic as compared to a population of cells not containing the nucleotide modification.

H2. The composition of any of the previous or subsequent embodiments, wherein the library is generated by:

(a) providing a plurality of a repair oligonucleotides, each comprising a portion of the target sequence and each individually containing a nucleotide modification at a different position of the target sequence;

(b) providing a library of Cas9 guide RNAs (gRNAs) that individually recognize a portion of the target sequence recognized by a defined group of the repair oligonucleotides;

(c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence;

(d) confirming the presence of cells containing at least one of the nucleotide modifications from the plurality of repair oligonucleotides in the population of cells; and

(e) determining if the cells containing at least one of the nucleotide modifications exhibit different a different functional characteristic than cells not containing the nucleotide modification.

H3. The composition of any of the previous or subsequent embodiments, wherein the functional characteristic is chemosensitivity.
H4. The composition of any of the previous or subsequent embodiments, wherein at least some of the plurality of nucleotide variants have been assessed and correlated with an effect on a function of the target sequence.
I1. A system for performing any of the steps of the methods of any of the previous or subsequent embodiments.
I2. The system of I1, comprising at least one of: (a) a station for obtaining a biological sample from a subject; (b) a station for performing a genotyping assay on the biological sample to identify a variant of unknown significance (VUS) at a target sequence; (c) a station for generating a population of cells containing the nucleotide modification corresponding to at least one VUS at the target sequence; and (d) a station for determining if the population of cells containing the nucleotide modification exhibit at least one different functional characteristic as compared to a population of cells not containing the nucleotide modification, wherein each of the stations may be a single station or a collection of stations.

That which is claimed is:

1. An in vitro method for assessing the functional effect of a somatic variation in a target sequence comprising:
(a) obtaining a biological sample from a subject;
(b) performing a genotyping assay on the biological sample to identify a variant of unknown significance at a target sequence;
(c) generating a population of cells containing the nucleotide modification at the target sequence; and
(d) determining if the population of cells containing the nucleotide modification exhibits at least one different functional characteristic as compared to a population of cells not containing the nucleotide modification.

2. The method of claim 1, wherein the target sequence is within a gene associated with chemosensitivity.

3. The method of claim 1, wherein the functional characteristic is chemosensitivity.

4. The method of claim 1, wherein the biological sample is cell-free nucleic acid, a solid tissue biopsy, a liquid biopsy, blood, bone marrow, urine, lymph, another bodily fluid, or a tissue sample.

5. The method of claim 1, wherein the biological sample includes genetic material from a cancerous cell.

6. The method of claim 1, wherein generating a population of cells containing the nucleotide modification at the target sequence comprises:
(a) providing a repair oligonucleotide, wherein the repair oligonucleotide comprises the sequence of the variant of unknown significance;
(b) providing a Cas9 guide RNA (gRNA) that individually recognize a portion of the gene recognized by the repair oligonucleotide;
(c) co-transfecting a population of cells with (i) an expression system capable of expressing Cas9 and the guide RNA, and (ii) the repair oligonucleotide and guide RNA, wherein the expression system is capable of introducing the oligonucleotide having the nucleotide modification into the target sequence in the population of cells; and
(d) confirming the presence of cells containing the nucleotide modification at the target sequence.

7. The method of claim 1, wherein generating a population of cells containing the nucleotide modification at the target sequence comprises expanding a cell line derived from the biological sample taken from the subject.

8. The method of claim 1, further comprising treating the subject based on the at least one different functional characteristic exhibited by the population of cells containing the nucleotide modification.

9. An in vitro method for assessing the functional effect of a genetic variant in a target sequence comprising:
introducing a plurality of nucleotide modifications, each comprising an individual variant of unknown significance, at a plurality of sites in a target sequence; and
determining for each of the plurality of variants of unknown significance, whether the nucleotide change is associated with a change in a functional characteristic for the target sequence.

10. The method of claim 9, further comprising generating a database of the plurality of variants of unknown significance.

11. The method of claim 9, wherein the plurality of variants of unknown significance are generated using saturation genome editing.

12. The method of claim 9, further comprising
(a) providing a plurality of a repair oligonucleotides, each comprising a portion of the target sequence and each individually containing a nucleotide modification at a different position of the target sequence;
(b) providing a library of Cas9 guide RNAs (gRNAs) that individually recognize a portion of the target sequence recognized by at least some the plurality of repair oligonucleotides;
(c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence;
(d) confirming the presence of cells containing at least one of the nucleotide modifications from the plurality of repair oligonucleotides in the population of cells; and
(e) determining if the cells containing the at least one nucleotide modification exhibit at least one different functional characteristic as compared to cells not containing the nucleotide modification.

13. The method of claim 9, further comprising:
obtaining a biological sample from a first subject; and
predicting the effect of the variant of unknown significance in the subject.

14. The method of claim 9, wherein the functional characteristic is chemosensitivity.

15. The method of claim 13, wherein the variant of unknown significance was a previously identified mutation in a biological sample from a second subject who is different than the first subject.

16. The method of claim 13, wherein the biological sample is cell-free nucleic acid, a liquid biopsy, blood, urine, lymph, another bodily fluid, or a tissue sample.

17. The method of claim 16, wherein the biological sample includes genetic material from a cancerous cell.

18. A composition comprising a library of cells for assessing the functional effect of a somatic variation in a target sequence, the library of cells comprising:
one or more populations of cells each containing a nucleotide modification at a target sequence, wherein the nucleotide modification exhibits at least one different functional characteristic as compared to a population of cells not containing the nucleotide modification.

19. The library of claim 18, wherein the library is generated by:
(a) providing a plurality of a repair oligonucleotides, each comprising a portion of the target sequence and each individually containing a nucleotide modification at a different position of the target sequence;
(b) providing a library of Cas9 guide RNAs (gRNAs) that individually recognize a portion of the target sequence recognized by a defined group of the repair oligonucleotides;
(c) co-transfecting a population of cells with (i) an expression system capable of expressing Cas9 and the plurality of guide RNAs and (ii) the plurality of the repair oligonucleotides, wherein the expression system is capable of introducing the repair oligonucleotides having the nucleotide modification into the target sequence;
(d) confirming the presence of cells containing at least one of the nucleotide modifications from the plurality of repair oligonucleotides in the population of cells; and
(e) determining if the cells containing at least one of the nucleotide modifications exhibit different a different functional characteristic than cells not containing the nucleotide modification.

20. The library of claim 18, wherein the functional characteristic is chemosensitivity.
